(19) United States
(12) Patent Application Publication
(10) Pub. No.: US 2005/0075326 A1
(43) Pub. Date: Apr. 7, 2005

(54) Spiro compounds and methods for the modulation of chemokine receptor activity

(76) Inventors: Laval Chan Chun Kong, Kirkland (CA); Ming Qiang Zhang, Cambridge (GB); Christophe Moinet, Montreal (CA); Melanie Proulx, Strasbourg (FR); Thumkunta Jagadeeswar Reddy, St. Laurent (CA); Marc Courchesne, Laval (CA)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/893,583

(22) Filed: Jul. 19, 2004

Related U.S. Application Data
(60) Provisional application No. 60/487,973, filed on Jul. 18, 2003.

(51) Int. Cl. A61K 31/55; A61K 31/4747; C07D 487/10

(52) U.S. Cl. 514/212.02; 514/278; 540/543; 546/16

(57) ABSTRACT

Novel compounds represented by formula I:

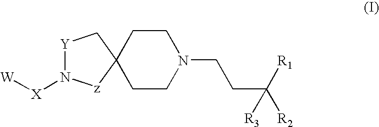

wherein Y, Z, X, W, R₁, R₂ and R₃ are as defined herein, or pharmaceutically acceptable salts, hydrates or solvates thereof, are useful for the modulation of CCR5 chemokine receptor activity and the treatment or prevention of diseases associated therewith.

This application claims the benefit of U.S. provisional application No. 60/487,973 filed Jul. 18, 2003, the entire disclosure of which is herein incorporated by reference.

### FIELD OF THE INVENTION

The present invention relates to novel spiro compounds and a method of modulating chemokine receptor activity using these compounds. The present invention is also directed to novel spiro compounds which are useful in the prevention or treatment of diseases associated with the modulation of CCR5 chemokine receptor activity. The present invention is further directed to a method of blocking cellular entry of HIV in a subject and to compositions using these compounds.

### BACKGROUND OF THE INVENTION

Chemokines are chemotactic cytokines that are released by a wide variety of cells to attract macrophages, T cells, eosinophils, basophils and neutrophils to sites of inflammation and they also play a role in the maturation of cells of the immune system. Chemokines play an important role in immune and inflammatory responses in various diseases and disorders, including asthma, rhinitis and allergic diseases, as well as autoimmune pathologies such as rheumatoid arthritis and atherosclerosis. Chemokines are small 70 to 80 amino acid proteins with well-characterized three-dimensional structures, usually stabilized by two disulfide bridges. They are divided into four families on the basis of pattern of conserved cysteine residues. Chemokine receptors have been designated such as, CCR1, CCR2, CCR2A, CCR2B, CCR3, CCR4, CCR5, CCR6, CCR7, CCR8, CCR9, CCR10, CXCR1, CXCR2, CXCR3, and CXCR4 and therefore agents which modulate these receptors may be useful in the prevention and treatment of diseases as mentioned above.

One of them, the C—C chemokines family, includes potent chemoattractants of monocytes and lymphocytes such as RANTES (Regulated on Activation, Normal T Expressed and Secreted), eotaxin, MIP-1α and MIP-1β (Macrophage Inflammatory Proteins) and human monocyte chemotactic proteins 1-3 (MCP-1, MCP-2 and MCP-3). More specifically, C—C chemokine receptor 5 (CCR5), a β-chemokine receptor with a seven-transmembrane-protein structure, was found to serve as a coreceptor for non-syncytium-inducing or macrophage-tropic HIV-1 (R5 viruses). It was also established that CCR5 is the principal chemokine receptor required for the entry of HIV into the cell during primary infection. Therefore, interfering with the interaction between the viral receptor CCR5 and HIV can block HIV entry into the cell. It would therefore be useful to provide novel compounds which are modulators of chemokine receptor activity.

### SUMMARY OF THE INVENTION

In one aspect, the present invention provides novel compounds represented by formula (I): 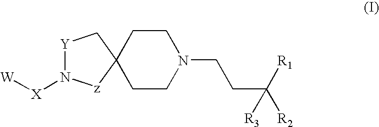
or pharmaceutically acceptable salts, hydrates or solvates thereof,

wherein Y, Z and X are each independently chosen from CH₂, C═O or CR₄R₅;

W is H, optionally substituted C₁₋₁₀ alkyl (e.g. C₁₋₆ alkyl) optionally substituted C₂₋₁₀ alkenyl (e.g. C₂₋₆ alkenyl), optionally substituted C₂₋₁₀ alkynyl (e.g. C₂₋₆ alkynyl), optionally substituted C₆₋₁₂ aryl, optionally substituted 3 to 10 membered heterocycle, optionally substituted C₆₋₁₂ aralkyl or optionally substituted C₃₋₁₀ heteroaralkyl;

R₁ is H, OH, optionally substituted C₁₋₁₀ alkyl (e.g. C₁₋₆ alkyl), optionally substituted C₂₋₁₀ alkenyl (e.g. C₂₋₆ alkenyl), optionally substituted C₂₋₁₀ alkynyl (e.g. C₂₋₆ alkynyl), optionally substituted C₆₋₁₂ aryl, NR₈R₉, optionally substituted O—C₁₋₆ alkyl, optionally substituted O—C₆₋₁₂ aryl, optionally substituted O—C₆₋₁₂ aralkyl,

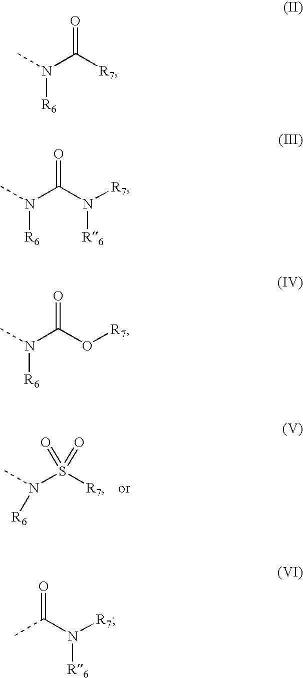

R₂ is optionally substituted C₁₋₁₀ alkyl, optionally substituted C₂₋₁₀ alkenyl, optionally substituted C₂₋₁₀ alkynyl, optionally substituted C₆₋₁₂ aryl or optionally substituted 3 to 10 membered heterocycle;

R₃ is H, optionally substituted C₁₋₁₀ alkyl (e.g. C₁₋₆ alkyl), optionally substituted C₂₋₁₀ alkenyl (e.g. C₂₋₆ alkenyl), optionally substituted C₂₋₁₀ alkynyl (e.g. C₂₋₆ alkynyl), or optionally substituted C₆₋₁₂ aryl;

R₄ and R₅ are each independently H, optionally substituted C₁₋₁₀ alkyl (e.g. C₁₋₆ alkyl), optionally substituted C₂₋₁₀ alkenyl (e.g. C₂₋₆ alkenyl), optionally substituted C₂₋₁₀ alkynyl (e.g. C₂₋₆ alkynyl), or optionally substituted C₆₋₁₂ aryl;

R₆ and R″₆ are each independently H, optionally substituted C₁₋₁₀ alkyl (e.g. C₁₋₄ alkyl), optionally substituted C₂₋₁₀ alkenyl (e.g. C₂₋₄ alkenyl), or optionally substituted C₂₋₁₀ alkynyl (e.g. C₂₋₄ alkynyl) and R₇ is H, optionally substituted C₁₋₁₀ alkyl, optionally substituted C₂₋₁₀ alkenyl, optionally substituted C₂₋₁₀ alkynyl, optionally substituted C₆₋₁₂ aryl, optionally substituted 3 to 10 membered heterocycle, optionally substituted C₆₋₁₂ aralkyl or optionally substituted 3 to 10 membered heteroaralkyl, or R″₆ and R₇ can be taken together to form an optionally substituted 3 to 10 membered heterocycle; and

R₈ and R₉ are each independently H, optionally substituted C₁₋₁₀ alkyl (e.g. C₁₋₆ alkyl), optionally substituted C₂₋₁₀ alkenyl (e.g. C₂₋₆ alkenyl), or optionally substituted C₂₋₁₀ alkynyl (e.g. C₂₋₆ alkynyl).

In another aspect, there is provided a method of modulating chemokine receptor activity in a subject comprising administering to the subject an effective amount of a compound of formula (I) or composition of the invention.

In still another aspect, there is provided a method for prevention or treatment of certain inflammatory diseases, immunoregulatory diseases, organ transplantation reactions and in the prevention and treatment of infectious diseases such as HIV infections in a subject in need of such treatment comprising administering to the subject a therapeutically effective amount of a compound of formula (I) or composition of the invention.

In still another aspect, there is provided a method for the prevention or treatment of diseases associated with the modulation of CCR5 chemokine receptor activity in a subject in need of such treatment comprising administering to the subject a therapeutically effective amount of a compound of formula (I) or composition of the invention.

In still another aspect, there is provided a method for blocking cellular entry of HIV in a subject comprising administering to the subject in need thereof an effective amount of a compound of formula (I) or composition of the invention to block HIV from cellular entry in said subject.

In still another aspect, there is provided a method for the prevention or treatment of diseases associated with the modulation of chemokine receptor activity in a subject in need of such treatment comprising administering to the subject a pharmaceutical combination comprising at least one compound of formula (I) and at least one further therapeutic agent.

In another aspect, there is provided a pharmaceutical formulation comprising the compound of the invention in combination with a pharmaceutically acceptable carrier or excipient.

In another aspect of the invention is the use of a compound according to formula (I), for the manufacture of a medicament for the prevention or treatment of diseases associated with the modulation of chemokine receptor activity.

### DETAILED DESCRIPTION OF THE INVENTION

In one embodiment, compounds of the present invention comprise those wherein the following embodiments are present, either independently or in combination.

In one embodiment, the present invention provides novel compounds represented by formula I: 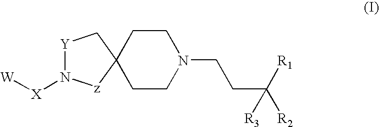
or pharmaceutically acceptable salts, hydrates or solvates thereof wherein Y, Z, X, W, R₁, R₂ and R₃ are defined above.

In one embodiment, the present invention provides novel compounds represented by formula (Ia): 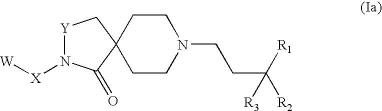
or pharmaceutically acceptable salts, hydrates or solvates thereof wherein Y, X, W, R₁, R₂ and R₃ are defined above.

In one embodiment, the present invention provides novel compounds represented by formula (Ib): 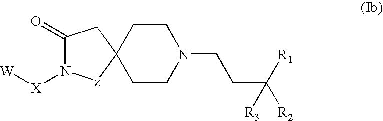
or pharmaceutically acceptable salts, hydrates or solvates thereof wherein Z, X, W, R₁, R₂ and R₃ are defined above.

In one embodiment, the present invention provides novel compounds represented by formula (Ic): 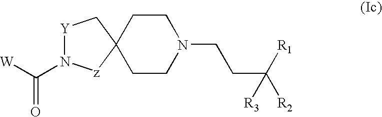
or pharmaceutically acceptable salts, hydrates or solvates thereof wherein Y, Z, W, R₁, R₂ and R₃ are defined above.

In one embodiment, the present invention provides novel compounds represented by formula (Id): 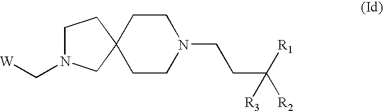
or pharmaceutically acceptable salts, hydrates or solvates thereof wherein W, R₁, R₂ and R₃ are defined above.

In one embodiment, the compounds of the present invention are in the (S)-enantiomer as represented by formula (Ie): 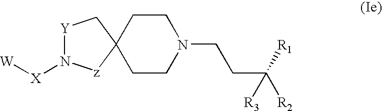
or pharmaceutically acceptable salts, hydrates or solvates thereof wherein Y, Z, X, W, R₁, R₂ and R₃ are defined above.

In one embodiment, W is chosen from optionally substituted C₆₋₁₂ aryl or optionally substituted 3 to 10 membered heterocycle.

In a further embodiment, W is optionally substituted C₆₋₁₂ aryl.

In a further embodiment, W is optionally substituted 3 to 10 membered heterocycle.

In further embodiments:

W is phenyl;

W is phenyl substituted with a halogen;

W is phenyl substituted with Br;

W is phenyl substituted with F;

W is phenyl substituted with Cl;

W is phenyl substituted with at least one halogen;

W is phenyl substituted with a C₁₋₃ alkoxy;

W is phenyl substituted with methoxy;

W is phenyl substituted with ethoxy;

W is phenyl substituted with SO₂C₁₋₃alkyl;

W is phenyl substituted with methanesulfonyl;

W is phenyl substituted with difluoromethoxy;

W is phenyl substituted with trifluoromethoxy;

W is phenyl substituted with trifluoromethyl;

W is phenyl substituted with CN;

W is phenyl substituted with pyrrazoyl;

W is phenyl optionally substituted in the para (p) position.

W is optionally substituted pyridine.

In a further embodiment, R₁ is chosen from:

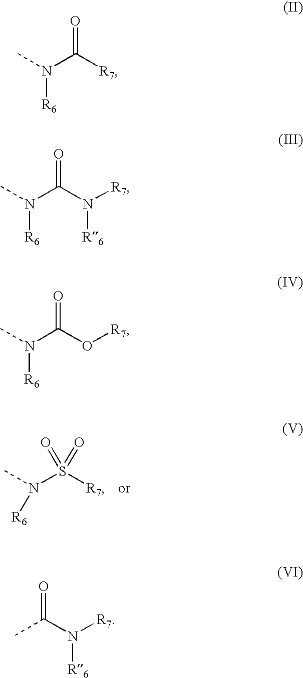

In a further embodiment, R₁ is: 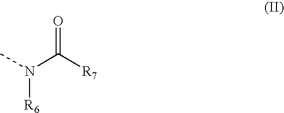
wherein R₆ is as defined above and R₇ is chosen from optionally substituted C₁₋₁₀ alkyl, optionally substituted C₆₋₁₂ aryl or optionally substituted 3 to 10 membered heterocycle.

In further embodiments, R₁ is: 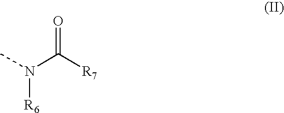
wherein:

R₇ is methyl;

R₇ is ethyl;

R₇ is isopropyl;

R₇ is cyclopropyl;

R₇ is cyclobutyl;

R₇ is cyclopentyl;

R₇ is cyclohexyl;

R₇ is cycloheptyl;

R₇ is 4,4-difluorocyclohexyl;

R₇ is CH₂-cyclopropyl;

R₇ is CH₂-cyclobutyl;

R₇ is CH₂-cyclopentyl;

R₇ is CH₂-cyclohexyl.

R₇ is phenyl;

R₇ is phenyl substituted with methyl;

R₇ is phenyl substituted with at least one methyl;

R₇ is phenyl substituted with a halogen;

R₇ is phenyl substituted with at least one halogen;

R₇ is phenyl substituted with Cl;

R₇ is phenyl substituted with Br;

R₇ is phenyl substituted with F;

R₇ is phenyl substituted with at least one Cl;

R₇ is phenyl substituted with methoxy.

R₇ is benzyl;

R₇ is benzyl substituted with methyl;

R₇ is benzyl substituted with at least one methyl;

R₇ is benzyl substituted with a halogen;

R₇ is benzyl substituted with at least one halogen;

R₇ is benzyl substituted with Cl;

R₇ is benzyl substituted with Br;

R₇ is benzyl substituted with F;

R₇ is benzyl substituted with at least one Cl;

R₇ is benzyl substituted with methoxy.

R₇ is optionally substituted pyridine.

In a further embodiment, R₁ is: 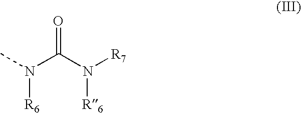
wherein R₆ and R₆ are as defined above and R₇ is optionally substituted C₆₋₁₂ aryl, or R₆ and R₇ can be taken together to form an optionally substituted 3 to 10 membered heterocycle.

In a further embodiment, R₁ is: 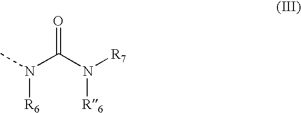
wherein:

R₇ is phenyl;

R₇ is phenyl substituted with methyl;

R₇ is phenyl substituted with at least one methyl;

R₇ is phenyl substituted with a halogen;

R₇ is phenyl substituted with at least one halogen;

R₇ is phenyl substituted with Cl;

R₇ is phenyl substituted with Br;

R₇ is phenyl substituted with F;

R₇ is phenyl substituted with at least one Cl;

R₇ is phenyl substituted with methoxy;

R₇ is naphthyl.

R″₆ and R₇ can be taken together to form an optionally substituted piperidine.

R₆ and R₇ can be taken together to form an optionally substituted morpholine.

R₆ and R₇ can be taken together to form a morpholine.

R₆ and R₇ can be taken together to form an optionally substituted pyrrolidine.

R₆ and R₇ can be taken together to form a 3,3-difluoropyrrolidine.

In a further embodiment, R₁ is: 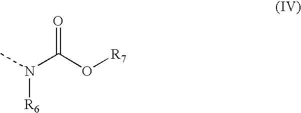
wherein R₆ is as defined above and R₇ is optionally substituted C₁₋₁₀ alkyl.

In further embodiments, R₁ is: 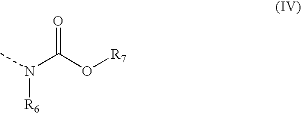
wherein:

R₇ is methyl;

R₇ is ethyl;

R₇ is tert-butyl;

R₇ is cyclobutyl;

R₇ is cyclopentyl;

R₇ is cyclohexyl.

In a further embodiment, R₁ is: 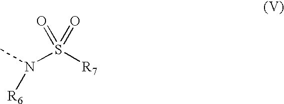
wherein R₆ is as defined above and R₇ is chosen from optionally substituted C₁₋₁₀ alkyl, optionally substituted C₆₋₁₂ aryl or optionally substituted 3 to 10 membered heterocycle.

In a further embodiment, R₁ is: 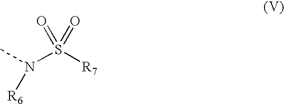
wherein:

R₇ is optionally substituted phenyl;

R₇ is optionally substituted C₁₋₁₀ alkyl;

R₇ is isopropyl.

In a further embodiment, R₁ is: 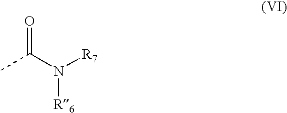
wherein R₆ is as defined above and R₇ is chosen from optionally substituted C₁₋₁₀ alkyl or optionally substituted C₆₋₁₂ aryl.

In a further embodiment, R₁ is: 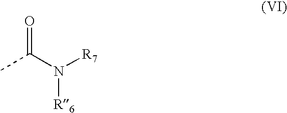
wherein:

R₇ is optionally substituted cyclohexyl.

R₇ is optionally substituted phenyl.

In a further embodiment, R₂ is chosen from optionally substituted C₆₋₁₂ aryl or optionally substituted 3 to 10 membered heterocycle.

In further embodiments:

R₂ is optionally substituted C₆₋₁₂ aryl.

R₂ is phenyl;

R₂ is phenyl substituted with halogen;

R₂ is phenyl substituted with Cl;

R₂ is phenyl substituted with at least one halogen;

R₂ is phenyl substituted with methoxy;

R₂ is phenyl substituted with at least one methoxy.

In a further embodiments:

R₂ is optionally substituted 3 to 10 membered heterocycle.

R₂ is optionally substituted thienyl.

R₂ is optionally substituted pyridyl.

In a further embodiment, R₃ is chosen from H or optionally substituted C₁₋₄ alkyl.

In one embodiment, R₃ is H.

In one embodiment, R₃ is methyl.

The compounds of the present invention may have an asymmetric center. As two optical isomers can independently be obtained from each asymmetric center, compounds of the invention having one asymmetric center can be in the form of the enantiomers, i.e., the (+) enantiomer or (−) enantiomer, in pure or partially purified form, as well as mixtures of enantiomers.

In one embodiment, the compounds of the present invention are the (+) enantiomer having an enantiomeric excess of 99%.

In one embodiment, the compounds of the present invention are the (+) enantiomer having an enantiomeric excess of 95%.

In one embodiment, the compounds of the present invention are the (+) enantiomer having an enantiomeric excess of 90%.

In one embodiment, the compounds of the present invention are the (−) enantiomer having an enantiomeric excess of 99%.

In one embodiment, the compounds of the present invention are the (−) enantiomer having an enantiomeric excess of 95%.

In one embodiment, the compounds of the present invention are the (−) enantiomer having an enantiomeric excess of 90%.

Compounds of the present invention have also two asymmetric centers. As two optical isomers can independently be obtained from each asymmetric center, compounds of the invention having two asymmetric centers can be in the form of the diastereomers. It is intended that all the possible diastereomers in mixtures and as pure or partially purified compounds are included in this invention.

In one embodiment, the compounds of the present invention are in the form of the (R,R)-diastereomer;

In one embodiment, the compounds of the present invention are in the form of the (S,R)-diastereomer;

In one embodiment, the compounds of the present invention are in the form of the (R,S)-diastereomer;

In one embodiment, the compounds of the present invention are in the form of the (S,S)-diastereomer.

In one embodiment, the compounds of the present invention are a (R,R)-diastereomer having an optical purity in excess of 99%.

In one embodiment, the compounds of the present invention are a (R,R)-diastereomer having an optical purity in excess of 95%.

In one embodiment, the compounds of the present invention are a (R,R)-diastereomer having an optical purity in excess of 90%.

In one embodiment, the compounds of the present invention are a (S,R)-diastereomer having an optical purity in excess of 99%.

In one embodiment, the compounds of the present invention are a (S,R)-diastereomer having an optical purity in excess of 95%.

In one embodiment, the compounds of the present invention are a (S,R)-diastereomer having an optical purity in excess of 90%.

In one embodiment, the compounds of the present invention are a (R,S)-diastereomer having an optical purity in excess of 99%.

In one embodiment, the compounds of the present invention are a (R,S)-diastereomer having an optical purity in excess of 95%.

In one embodiment, the compounds of the present invention are a (R,S)-diastereomer having an optical purity in excess of 90%.

In one embodiment, the compounds of the present invention are a (S,S)-diastereomer having an optical purity in excess of 99%.

In one embodiment, the compounds of the present invention are a (S,S)-diastereomer having an optical purity in excess of 95%.

In one embodiment, the compounds of the present invention are a (S,S)-diastereomer having an optical purity in excess of 90%.

In one embodiment, there is provided a method of modulating chemokine receptor activity in a subject comprising administering to the subject a therapeutically effective amount of a compound of formula (I) or composition of the invention.

In another embodiment, there is provided a method for the prevention or treatment of diseases associated with the modulation of chemokine receptor activity in a subject in need of such treatment comprising administering to the subject a therapeutically effective amount of a compound of formula (I) or composition of the invention.

In a further embodiment, there is provided a method for prevention or treatment of certain inflammatory diseases, immunoregulatory diseases, organ transplantation reactions and in the prevention and treatment of infectious diseases such as HIV infections in a subject in need of such treatment comprising administering to the subject a therapeutically effective amount of a compound of formula (I) or composition of the invention.

In another embodiment, there is provided a method for the prevention or treatment of diseases associated with the modulation of CCR5 chemokine receptor activity in a subject in need of such treatment comprising administering to the subject a therapeutically effective amount of a compound of formula (I) or composition of the invention.

In still another aspect, there is provided a method for blocking cellular entry of HIV in a subject in need thereof comprising administering to the subject a therapeutically effective amount of a compound of formula (I) to block HIV from cellular entry in said subject.

In still another aspect, there is provided a method for prevention or treatment of HIV infections in a subject in need of such treatment comprising administering to the subject a therapeutically effective amount of a compound of formula (I) or composition of the invention.

In still another aspect, there is provided a method for delaying the onset of AIDS or treating AIDS in a subject in need of such treatment comprising administering to the subject a therapeutically effective amount of a compound of formula (I) or composition of the invention.

In a further embodiment, there is provided a method for the prevention or treatment of diseases associated with the modulation of chemokine receptor activity in a subject in need of such treatment comprising administering to the subject a pharmaceutical combination comprising at least one compound of formula (I) and at least one further therapeutic agent.

In a further embodiment, there is provided a method for the prevention or treatment of diseases associated with the modulation of CCR5 chemokine receptor activity in a subject in need of such treatment comprising administering to the subject a pharmaceutical combination comprising at least one compound of formula (I) and at least one further therapeutic agent.

In still another aspect, there is provided a method for blocking cellular entry of HIV in a subject or for the prevention or treatment of HIV infections in a subject in need of such treatment comprising administering to the subject a pharmaceutical combination comprising at least one compound of formula (I) and at least one further therapeutic agent.

In still another aspect, there is provided a method for delaying the onset of AIDS or treating AIDS in a subject in need of such treatment comprising administering to the subject a pharmaceutical combination comprising at least one compound of formula (I) and at least one further therapeutic agent.

In another embodiment, there is provided a combination useful for the prevention or treatment of diseases associated with the modulation of chemokine receptor activity which is a therapeutically effective amount of a compound of formula (I) and therapeutically effective amount of at least one further therapeutic agent.

In one embodiment, combinations of the present invention comprise those wherein the following embodiments are present, either independently or in combination.

In a further embodiment, the pharmaceutical combinations of this invention may contain at least one further therapeutic agent chosen from an agent used in inflammatory diseases, immunoregulatory diseases and in organ transplantation reactions.

In another embodiment, the pharmaceutical combination of this invention may contain at least one further therapeutic agent which is an antiviral agent.

In one embodiment, the pharmaceutical combination of this invention may contain at least one further antiviral agent which is chosen from nucleoside and nucleotide analog reverse transcriptase inhibitors, non-nucleoside reverse transcriptase inhibitors, protease inhibitors, attachment and fusion inhibitors, integrase inhibitors or maturation inhibitors.

In one embodiment, the pharmaceutical combinations of this invention may contain at least one other antiviral agent which is a nucleoside and nucleotide analog reverse transcriptase inhibitors chosen from 3TC (lamivudine, Epivir®), AZT (zidovudine, Retrovir®), Emtricitabine (Coviracil®, formerly FTC), d4T (2′,3′-dideoxy-2′,3′-didehydro-thymidine, stavudine and Zerit®), tenofovir (Viread®), 2′,3′-dideoxyinosine (ddl, didanosine, Videx®), 2′,3′-dideoxycytidine (ddC, zalcitabine, Hivid®), Combivir® (AZT/3TC or zidovudine/lamivudine combination), Trivizir® (AZT/3TC/abacavir or zidovudine/lamivudine/abacavir combination), abacavir (1592U89, Ziagen®), SPD-754, ACH-126,443 (Beta-L-Fd4C), Alovudine (MIV-310), DAPD (amdoxovir), Racivir, 9-[(2-hydroxymethyl)-1,3-dioxolan-4-yl]guanine or 2-amino-9-[(2-hydroxymethyl)-1,3-dioxolan-4-yl]adenine.

In another embodiment, the pharmaceutical combination of this invention may contain at least one other antiviral agent which is a non-nucleoside reverse transcriptase inhibitor chosen from Nevirapine (Viramune®, NVP, BI-RG-587), delavirdine (Rescriptor®, DLV), efavirenz (DMP 266, Sustiva®), (+)-Calanolide A, Capravirine (AG1549, formerly S-1153), DPCO83, MIV-150, TMC120, TMC125 or BHAP (delavirdine), calanolides or L-697,661 (2-Pyridinone 3benzoxazolMeNH derivative).

In another embodiment, the pharmaceutical combination of this invention may contain at least one other antiviral agent which ia a protease inhibitor chosen from nelfinavir (Viracept®, NFV), amprenavir (141W94, Agenerase®), indinavir (MK-639, IDV, Crixivan®), saquinavir (Invirase®, Fortovase®, SQV), ritonavir (Norvir®, RTV), lopinavir (ABT-378, Kaletra®), Atazanavir (BMS232632), mozenavir (DMβ-450), fosamprenavir (GW433908), R0033-4649, Tipranavir (PNU-140690), TMC114 or VX-385.

In another embodiment, the pharmaceutical combination of this invention may contain at least one other antiviral agent which is an attachment and fusion inhibitor chosen from T-20 (enfuvirtide, Fuzeon®), T-1249, Schering C (SCH-C), Schering D (SCH-D), FP21399, PRO-140, PRO 542, PRO 452, TNX-355, GW873140 (AK602), TAK-220, UK-427,857 or soluble CD4, CD4 fragments, CD4-hybrid molecules, BMS-806, BMS-488043,AMD3100, AMD070 or KRH-2731.

In another embodiment, the pharmaceutical combination of this invention may contain at least one other antiviral agent which is an integrase inhibitor chosen from S-1360, L-870,810, L-870,812 or C-2507.

In another embodiment, the pharmaceutical combination of this invention may contain at least one other antiviral agent which is a maturation inhibitor and is PA-457.

In another embodiment, the pharmaceutical combination of this invention may contain at least one other antiviral agent which is a zinc finger inhibitor and is azodicarbonamide (ADA).

In another embodiment, the pharmaceutical combination of this invention may contain at least one other antiviral agent which is an antisense drug and is HGTV43.

In another embodiment, the pharmaceutical combination of this invention may contain at least one other antiviral agent which is an immunomodulator, immune stimulator or cytokine chosen from interleukin-2 (IL-2, Aldesleukin, Proleukin), granulocyte macrophage colony stimulating factor (GM-CSF), erythropoietin, Multikine, Ampligen, thymomodulin, thymopentin, foscarnet, HE2000, Reticulose, Murabutide, Resveratrol, HRG214, HIV-1 Immunogen (Remune) or EP HIV-1090.

In another embodiment, the pharmaceutical combination of this invention may contain at least one other antiviral agent chosen from 2′,3′-dideoxyadenosine, 3′-deoxythymidine, 2′,3′-dideoxy-2′,3′-didehydrocytidine and ribavirin; acyclic nucleosides such as acyclovir, ganciclovir; interferons such as alpha-, beta-and gamma-interferon; glucuronation inhibitors such as probenecid; or TIBO drugs, HEPT, TSAO derivatives.

The combinations referred to above may conveniently be presented for use in the form of a pharmaceutical formulation and thus pharmaceutical formulations comprising a combination as defined above together with a pharmaceutically acceptable carrier thereof comprises a further aspect of the invention.

The individual components of such combinations may be administered either sequentially or simultaneously in separate or combined pharmaceutical formulations.

In a further embodiment, the said compound of formula (I) and said therapeutic agent are administered sequentially.

In a further embodiment, the said compound of formula (I) and said therapeutic agent are administered simultaneously.

The subject to which the compounds are administered can be, for example, a mammal or a human. Preferably, the subject is a human.

In one embodiment, the present invention further provides a pharmaceutical composition comprising at least one compound having the formula (I) or pharmaceutically acceptable salts or pharmaceutically acceptable hydrates or pharmaceutically acceptable solvates thereof and at least one pharmaceutically acceptable carrier or excipient.

In another embodiment, the invention provides the use of a compound having the formula (I) for the manufacture of a medicament for prevention and treatment of diseases associated with the modulation of CCR5 chemokine receptor activity in a host comprising administering a therapeutically effective amount of a compound of formula (I).

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

The term “alkyl” represents a linear, branched or cyclic hydrocarbon moiety having, for example, 1 to 10 carbon atoms, which may have one or more double bonds or triple bonds in the chain, and is optionally substituted. For example, suitable substituents include halogen, amino, amidino, amido, azido, cyano, guanido, hydroxyl, nitro, nitroso, urea, OS(O)₂R₂₁ (wherein R₂₁ is selected from C₁₋₆ alkyl, C₆₋₁₂ aryl or 3 to 10 membered heterocycle), OS(O)₂OR₂₂ (wherein R₂₂ is selected from H, C₁₋₆ alkyl, C₆₋₁₂ aryl or 3 to 10 membered heterocycle), S(O)₂OR₂₃ (wherein R₂₃ is selected from H, C₁₋₆ alkyl, C₆₋₁₂ aryl or 3 to 10 membered heterocycle), S(O)₀₋₂R₂₄ (wherein R₂₄ is selected from H, C₁₋₆ alkyl, C₆₋₁₂ aryl or 3 to 10 membered heterocycle), OP(O)OR₂₅OR₂₆, P(O)OR₂₅OR₂₆ (wherein R₂₅ and R₂₆ are each independently selected from H or C₁₋₆ alkyl), C(O)R₂₇ (wherein R₂₇ is selected from H, C₁₋₆ alkyl, C₆₋₁₂ aryl or 3 to 10 membered heterocycle), C(O)OR₂₈ (wherein R₂₈ is selected from H, C₁₋₆ alkyl, C₆₋₁₂ aryl, C₆₋₁₂ aralkyl or 3 to 10 membered heterocycle), NR₂₉C(O)R₃₀, C(O)NR₂₉R₃₀ (wherein R₂₉ is H or C₁₋₆ alkyl and R₃₀ is selected from H, C₁₋₆ alkyl, C₆₋₁₂ aryl, C₆₋₁₂ aralkyl or 3 to 10 membered heterocycle, or R₂₉ and R₃₀ are taken together with the atoms to which they are attached to form a 3 to 10 membered heterocycle), SO₂NR₃₁R₃₂, NR₃₁SO₂R₃₂ (wherein R₃₁ and R₃₂ are each independently selected from the group consisting of H, C₁₋₆ alkyl, C₆₋₁₂ aryl, 3 to 10 membered heterocycle and C₆₋₁₂ aralkyl), C (R₃₃) NR₃₄ or C (R₃₃) NOR₃₄ (wherein R₃₃ and R₃₄ are each independently selected from the group consisting of H, C₁₋₆ alkyl, or C₆₋₁₂aryl).

Preferred substituents for the alkyl groups include halogen (Br, Cl, I or F), cyano, nitro, oxo, amino, COOH, COO—C₁₄ alkyl, CO—C₁₄ alkyl, and phenyl.

Examples of alkyl groups include but are not limited to methyl, ethyl, propyl, isopropyl, butyl, isobutyl, sec-butyl, tert-butyl, pentyl, isopentyl, neopentyl, tert-pentyl, hexyl, isohexyl, neohexyl, allyl, vinyl, acetylenyl, ethylenyl, propenyl, isopropenyl butenyl, isobutenyl, hexenyl, butadienyl, pentenyl, pentadienyl, hexenyl, hexadienyl, hexatrienyl, heptenyl, heptadienyl, heptatrienyl, octenyl, octadienyl, octatrienyl, octatetraenyl, propynyl, butynyl, pentynyl, hexynyl, cyclopropyl, cyclobutyl, cycloheptyl, cyclohexenyl, cyclohex-dienyl and cyclohexyl.

The term alkyl is also meant to include alkyls in which one or more hydrogen atom is replaced by a halogen, i.e. an alkylhalide. Examples include but are not limited to trifluoromethyl, difluoromethyl, fluoromethyl, trichloromethyl, dichloromethyl, chloromethyl, trifluoroethyl, difluoroethyl, fluoroethyl, trichloroethyl, dichloroethyl, chloroethyl, chlorofluoromethyl, chlorodifluoromethyl, dichlorofluoroethyl.

The term “alkenyl” refers to alkyl groups may have one or more double bonds in the chain. The term “alkynyl” refers to alkyl groups may have one or more triple bonds in their chain. The alkenyl and alkynyl groups can be optionally substituted as described above for the alkyl groups.

The term “alkoxy” represents an alkyl which is covalently bonded to the adjacent atom through an oxygen atom. Examples include but are not limited to methoxy, ethoxy, propoxy, isopropoxy, butoxy, isobutoxy, sec-butoxy, tert-butoxy, pentyloxy, isopentyloxy, neopentyloxy, tert-pentyloxy, hexyloxy, isohexyloxy and neohexyloxy.

The term “alkylamino” represents an alkyl which is covalently bonded to the adjacent atom through a nitrogen atom and may be monoalkylamino or dialkylamino, wherein the alkyl groups may be the same or different. Examples include but are not limited to methylamino, dimethylamino, ethylamino, diethylamino, methylethylamino, propylamino, isopropylamino, butylamino, isobutylamino, sec-butylamino, tert-butylamino, pentylamino, isopentylamino, neopentylamino, tert-pentylamino, hexylamino, isohexylamino and neohexylamino.

The term “alkyloxycarbonyl” represents an alkyloxy which is covalently bonded to the adjacent atom through carbonyl (C═O). Examples include but are not limited to methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, isopentyloxycarbonyl, neopentyloxycarbonyl, tert-pentyloxycarbonyl, hexyloxycarbonyl, isohexyloxycarbonyl and neohexyloxycarbonyl.

The term “amidino” represents —C(=NR₁₀)NR₁₁R₁₂, wherein R₁₀, R₁₁ and R₁₂ are each independently selected from H, C₁₋₆ alkyl, C₆₋₁₂ aryl or C₆₋₁₂ aralkyl, or R₁₁ and R₁₂ are taken together with the nitrogen to which they are attached to form a 3 to 10 membered heterocycle.

The term “amido” represents —CONH₂, —CONHR₁₃ and —CONR₁₃R₁₄ wherein R₁₃ and R₁₄ are each independently selected from C₁₋₆ alkyl, C₆₋₁₂ aryl, 3 to 10 membered heterocycle or C₆₋₁₂ aralkyl, or R₁₃ and R₁₄ are taken together with the nitrogen to which they are attached to form a 3 to 10 membered heterocycle.

The term “amino” represents a derivative of ammonia obtained by substituting one or more hydrogen atom and include —NH₂, —NHR₁₅ and —NR₁₅R₁₆, wherein R₁₅ and R₁₆ are each independently selected from C₁₋₆ alkyl, C₆₋₁₂ aryl or C₆₋₁₂ aralkyl, or R₁₅ and R₁₆ are taken together with the nitrogen to which they are attached to form a 3 to 10 membered heterocycle.

The term “aryl” represents a carbocyclic moiety containing at least one benzenoid-type ring (i.e. the aryl group may be monocyclic or polycyclic), and which is optionally substituted with one or more substituents. For example, suitable substituents include halogen, halogenated C₁₋₆ alkyl, halogenated C₁₋₆ alkoxy, amino, amidino, amido, azido, cyano, guanido, hydroxyl, nitro, nitroso, urea, OS(O)₂R₂₁ (wherein R₂₁ is selected from C₁₋₆ alkyl, C₆₋₁₂ aryl or 3 to 10 membered heterocycle), OS(O)₂OR₂₂ (wherein R₂₂ is selected from H, C₁₋₆ alkyl, C₆₋₁₂ aryl or 3 to 10 membered heterocycle), S(O)₂OR₂₃ (wherein R₂₃ is selected from H, C₁₋₆ alkyl, C₆₋₁₂ aryl or 3 to 10 membered heterocycle), S(O)₀₋₂R₂₄ (wherein R₂₄ is selected from H, C₁₋₆ alkyl, C₆₋₁₂ aryl or 3 to 10 membered heterocycle), OP(O)OR₂₅OR₂₆, P(O)OR₂₅OR₂₆ (wherein R₂₅ and R₂₆ are each independently selected from H or C₁₋₆ alkyl), C₁₋₆alkyl, C₆₋₁₂aralkyl, C₁₋₆alkoxy, C₆₋₁₂aralkyloxy, C₆₋₁₂aryloxy, 3 to 10 membered heterocycle, C(O)R₂₇ (wherein R₂₇ is selected from H, C₁₋₆ alkyl, C₆₋₁₂ aryl or 3 to 10 membered heterocycle), C(O)OR₂₈ (wherein R28 is selected from H, C₁₋₆ alkyl, C₆₋₁₂ aryl, C₆₋₁₂ aralkyl or 3 to 10 membered heterocycle), NR₂₉C(O)R₃₀, C(O)NR₂₉R₃₀ (wherein R₂₉ is H or C₁₋₆ alkyl and R₃₀ is selected from H, C₁₋₆ alkyl, C₆₋₁₂ aryl, C₆₋₁₂ aralkyl or 3 to 10 membered heterocycle, or R₂₉ and R₃₀ are taken together with the atoms to which they are attached to form a 3 to 10 membered heterocycle), SO₂NR₃₁R₃₂, NR₃₁SO₂R₃₂ (wherein R₃₁ and R₃₂ are each independently selected from the group consisting of H, C₁₋₆ alkyl, C₆₋₁₂ aryl, 3 to 10 membered heterocycle and C₆₋₁₂ aralkyl), C(R₃₃)NR₃₄ or C(R₃₃)NOR₃₄ (wherein R₃₃ and R₃₄ are each independently selected from the group consisting of H, C₁₋₆ alkyl, or C₆₋₁₂ aryl).

Preferred substituents for the aryl groups include halogen (Br, Cl, I or F), cyano, nitro, oxo, amino, C₁₋₄ alkyl (e.g., CH₃, C₂H₅, isopropyl), C₁₋₄ alkoxy (e.g., OCH₃, OC₂H₅), halogenated C₁₋₄ alkyl (e.g., CF₃, CHF₂), halogenated C₁₋₄ alkoxy (e.g., OCF₃, OC₂F₅), COOH, COO—C₁l₄ alkyl, CO—C₁₋₄ alkyl, C₁₋₄ alkyl-S—(e.g., CH₃S, C₂H₅S) , halogenated C₁₋₄ alkyl-S—(e.g., CF₃S, C₂F₅S), benzyloxy, and pyrazolyl.

Examples of aryl include but are not limited to phenyl, tolyl, dimethylphenyl, aminophenyl, anilinyl, naphthyl, anthryl, phenanthryl or biphenyl.

The term “aralkyl” represents an aryl group attached to the adjacent atom by a C₁₋₆alkyl. Examples include but are not limited to benzyl, benzhydryl, trityl, phenethyl, 3-phenylpropyl, 2-phenylpropyl, 4-phenylbutyl and naphthylmethyl. The aryl and alkyl portions can be optionally substituted as described above.

The term “aralkyloxy” represents an aralkyl which is covalently bonded to the adjacent atom through an oxygen atom. Examples include but are not limited to benzyloxy, benzhydryloxy, trityloxy, phenethyloxy, 3-phenylpropyloxy, 2-phenylpropyloxy, 4-phenylbutyloxy and naphthylmethoxy. The aryl and alkyl portions can be optionally substituted as described above.

The term “aryloxy” represents an aryl which is covalently bonded to the adjacent atom through an oxygen atom. Examples include but are not limited to phenoxy and naphthyloxy. The aryl portion can be optionally substituted as described above.

The term “guanidino” represents —NR₁₇C(═NR₁₈)NR₁₉R₂₀ wherein R₁₇, R₁₈, R₁₉ and R₂₀ are each independently selected from H, C₁₋₆ alkyl, C₆₋₁₂ aryl or C₆₋₁₂ aralkyl, or R₁₉ and R₂₀ are taken together with the nitrogen to which they are attached to form a 3 to 10 membered heterocycle.

The term “halogen” is specifically a fluoride atom, chloride atom, bromide atom or iodide atom.

The term “heterocycle” represents an optionally substituted saturated, unsaturated or aromatic cyclic moiety wherein said cyclic moiety is interrupted by at least one heteroatom selected from oxygen (O), sulfur (S) or nitrogen (N). Heterocycles may be monocyclic or polycyclic rings. For example, suitable substituents include halogen, halogenated C₁₋₆ alkyl, halogenated C₁₋₆ alkoxy, amino, amidino, amido, azido, cyano, guanido, hydroxyl, nitro, nitroso, urea, OS(O)₂R₂₁ (wherein R₂₁ is selected from C₁₋₆ alkyl, C₆₋₁₂ aryl or 3 to 10 membered heterocycle), OS(O)₂OR₂₂ (wherein R₂₂ is selected from H, C₁₋₆ alkyl, C₆₋₁₂ aryl or 3 to 10 membered heterocycle), S(O)₂OR₂₃ (wherein R₂₃ is selected from H, C₁₋₆ alkyl, C₆₋₁₂ aryl or 3 to 10 membered heterocycle), S(O)₀₋₂R₂₄ (wherein R₂₄ is selected from H, C₁₋₆ alkyl, C₆₋₁₂ aryl or 3 to 10 membered heterocycle), OP(O)OR₂₅OR₂₆, P(O)OR₂₅OR₂₆ (wherein R₂₅ and R₂₆ are each independently selected from H or C₁₋₆ alkyl), C₁₋₆alkyl, C₆₋₁₂aralkyl, C₁₋₆alkoxy, C₆₋₁₂ aryl, C₆₋₁₂aralkyloxy, C₆₋₁₂aryloxy, C(Q)R₂₇ (wherein R₂₇ is selected from H, C₁₋₆ alkyl, C₆₋₁₂ aryl or 3 to 10 membered heterocycle), C(O)OR₂₈ (wherein R₂₈ is selected from H, C₁₋₆ alkyl, C₆₋₁₂ aryl, C₆₋₁₂ aralkyl or 3 to 10 membered heterocycle), NR₂₉C(O)R₃₀, C(O)NR₂₉R₃₀ (wherein R₂₉ is H or C₁₋₆ alkyl and R₃₀ is selected from H, C₁₋₆ alkyl, C₆₋₁₂ aryl, C₆₋₁₂ aralkyl or 3 to 10 membered heterocycle, or R₂₉ and R₃₀ are taken together with the atoms to which they are attached to form a 3 to 10 membered heterocycle), SO₂NR₃₁R₃₂, NR₃₁SO₂R₃₂ (wherein R₃₁ and R₃₂ are each independently selected from the group consisting of H, C₁₋₆ alkyl, C₆₋₁₂ aryl, 3 to 10 membered heterocycle and C₆₋₁₂ aralkyl), C(R₃₃)NR₃₄ or C(R₃₃)NOR₃₄ (wherein R₃₃ and R₃₄ are each independently selected from the group consisting of H, C₁₋₆ alkyl, or C₆₋₁₂ aryl)

Preferred substituents for the heterocycle groups include halogen (Br, Cl, I or F), cyano, nitro, oxo, amino, C₁₋₄ alkyl (e.g., CH₃, C₂H₅, isopropyl), C₁₋₄ alkoxy (e.g., OCH₃, OC₂H₅), halogenated C₁₋₄ alkyl (e.g., CF₃, CHF₂), halogenated C₁₋₄ alkoxy (e.g., OCF₃, OC₂F₅), COOH, COO—C₁₋₄ alkyl, CO—C₁₋₄ alkyl, C₁₋₄ alkyl-S—(e.g., CH₃S, C₂H₅S), halogenated C₁₋₄ alkyl-S—(e.g., CF₃S, C₂F₅S), benzyloxy, and pyrazolyl.

Examples of heterocycles include but are not limited to azepinyl, aziridinyl, azetyl, azetidinyl, diazepinyl, dithiadiazinyl, dioxazepinyl, dioxolanyl, dithiazolyl, furanyl, isooxazolyl, isothiazolyl, imidazolyl, morpholinyl, morpholino, oxetanyl, oxadiazolyl, oxiranyl, oxazinyl, oxazolyl, piperazinyl, pyrazinyl, pyridazinyl, pyrimidinyl, piperidyl, piperidino, pyridyl, pyranyl ,pyrazolyl, pyrrolyl, pyrrolidinyl, thiatriazolyl, tetrazolyl, thiadiazolyl, triazolyl, thiazolyl, thienyl, tetrazinyl, thiadiazinyl, triazinyl, thiazinyl, thiopyranyl furoisoxazolyl, imidazothiazolyl, thienoisothiazolyl, thienothiazolyl, imidazopyrazolyl, cyclopentapyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienopyridyl, benzothienyl, cyclopentaoxazinyl, cyclopentafuranyl, benzoxazinyl, benzothiazinyl, quinazolinyl, naphthyridinyl, quinolinyl, isoquinolinyl, benzopyranyl, pyridopyridazinyl and pyridopyrimidinyl.

The term “heteroaralkyl” represents a heterocycle group attached to the adjacent atom by a C₁₋₆ alkyl. The heterocycle and alkyl portions can be optionally substituted as described above.

The term “urea” represents —N(R₃₅)CONR₃₆R₃₇ wherein R₃₅ is H or C₁₋₆ alkyl and wherein R₃₆ and R₃₇ are each independently selected from the group consisting of H, C₁₋₆ alkyl, C₆₋₁₂ aryl, 3 to 10 membered heterocycle and C₆₋₁₂ aralkyl, or R₃₆ and R₃₇ are taken together with the nitrogen to which they are attached to form a 3 to 10 membered heterocycle.

The term “independently” means that a substituent can be the same or a different definition for each item.

The term “optionally substituted” represents one or more halogen, halogenated C₁₋₆ alkyl, halogenated C₁₋₆ alkoxy, amino, amidino, amido, azido, cyano, guanido, hydroxyl, nitro, nitroso, urea, OS(O)₂R₂₁ (wherein R₂₁ is selected from C₁₋₆ alkyl, C₆₋₁₂ aryl or 3 to 10 membered heterocycle), OS(O)₂OR₂₂ (wherein R₂₂ is selected from H, C₁₋₆ alkyl, C₆₋₁₂ aryl or 3 to 10 membered heterocycle), S(O)₂OR₂₃ (wherein R₂₃ is selected from H, C₁₋₆ alkyl, C₆₋₁₂ aryl or 3 to 10 membered heterocycle), S(O)₀₋₂R₂₄ (wherein R₂₄ is selected from H, C₁₋₆ alkyl, C₆₋₁₂ aryl or 3 to 10 membered heterocycle), OP(O)OR₂₅OR₂₆, P(O)OR₂₅OR₂₆ (wherein R₂₅ and R₂₆ are each independently selected from H or C₁₋₆ alkyl), C₁₋₆alkyl, C₆₋₁₂aralkyl, C₆₋₁₂ aryl, C₁₋₆alkoxy, C₆₋₁₂aralkyloxy, C₆₋₁₂aryloxy, 3 to 10 membered heterocycle, C(O)R₂₇ (wherein R₂₇ is selected from H, C₁₋₆ alkyl, C₆₋₁₂ aryl or 3 to 10 membered heterocycle), C(O)OR₂₈ (wherein R₂₈ is selected from H, C₁₋₆ alkyl, C₆₋₁₂ aryl, C₆₋₁₂ aralkyl or 3 to 10 membered heterocycle), NR₂₉C(O)R₃₀, C(O)NR₂₉R₃₀ (wherein R₂₉ is H or C₁₋₆ alkyl and R₃₀ is selected from H, C₁₋₆ alkyl, C₆₋₁₂ aryl, C₆₋₁₂ aralkyl or 3 to 10 membered heterocycle, or R₂₉ and R₃₀ are taken together with the atoms to which they are attached to form a 3 to 10 membered heterocycle), SO₂NR₃₁R₃₂, NR₃₁SO₂R₃₂ (wherein R₃, and R₃₂ are each independently selected from the group consisting of H, C₁₋₆ alkyl, C₆₋₁₂ aryl, 3 to 10 membered heterocycle and C₆₋₁₂ aralkyl), C(R₃₃)NR₃₄ or C(R₃₃)NOR₃₄ (wherein R₃₃ and R₃₄ are each independently selected from the group consisting of H, C₁₋₆ alkyl, or C₆₋₁₂aryl).

There is also provided “enantiomers” and “diastereoisomers” of the present invention. It will be appreciated that the compounds in accordance with the present invention can contain one or more chiral centers. The compounds in accordance with the present invention may thus exist in the form of two different optical isomers, that is (+) or (−) enantiomers or in the form of different diastereomers. All such enantiomers, diastereomers and mixtures thereof, including racemic or other ratio mixtures of individual enantiomers and diastereomers, are included within the scope of the invention. The single diastereomer can be obtained by methods well known to those of ordinary skill in the art, such as HPLC, crystallization and chromatography. The single enantiomer can be obtained by methods well known to those of ordinary skill in the art, such as chiral HPLC, enzymatic resolution and chiral auxiliary derivatization.

The optical purity is numerically equivalent to the “enantiomeric excess”. The term “enantiomeric excess” is defined in percentage (%) value as follows: [mole fraction (major enantiomer)—mole fraction (minor enantiomer)] ×100. An example of enantiomeric excess of 99% represents a ratio of 99.5% of one enantiomer and 0.5% of the opposite enantiomer.

“Oxidation levels”: When there is a sulfur atom present, the sulfur atom can be at different oxidation levels, ie. S, SO, or SO₂. All such oxidation levels are within the scope of the present invention. When there is a nitrogen atom present, the nitrogen atom can be at different oxidation levels, ie. N or NO. All such oxidation levels are within the scope of the present invention.

There is also provided “pharmaceutically acceptable hydrates” of the compounds of the present invention. “Hydrates” exist when the compound of the invention incorporates water. The hydrate may contain one or more molecule of water per molecule of compound of the invention. Illustrative non-limiting examples include monohydrate, dihydrate, trihydrate and tetrahydrate. The hydrate may contain one or more molecule of compound of the invention per molecule of water. An illustrative non-limiting example include semi-hydrate. In one embodiment, the water may be held in the crystal in various ways and thus, the water molecules may occupy lattice positions in the crystal, or they may form bonds with salts of the compounds as described herein. The hydrate must be “acceptable” in the sense of not being deleterious to the recipient thereof. The hydration may be assessed by methods known in the art such as Loss on Drying techniques (LOD) and Karl Fisher titration.

There is also provided “pharmaceutically acceptable salts” of the compounds of the present invention. By the term “pharmaceutically acceptable salts” of compounds are meant those derived from pharmaceutically acceptable inorganic and organic acids and bases. Examples of suitable acids include but are not limited to hydrochloric, hydrobromic, sulphuric, nitric, perchloric, fumaric, maleic, phosphoric, glycollic, lactic, salicylic, succinic, toleune-p-sulphonic, tartaric, acetic, trifluoroacetic, citric, methanesulphonic, formic, benzoic, malonic, naphthalene-2-sulphonic and benzenesulphonic acids. Other acids such as oxalic, while not in themselves pharmaceutically acceptable, may be useful as intermediates in obtaining the compounds of the invention and their pharmaceutically acceptable acid addition salts.

Salts derived from appropriate bases include alkali metal, alkaline earth metal or ammonium salts. The salt(s) must be “acceptable” in the sense of not being deleterious to the recipient thereof. Non-limiting examples of such salts known by those of ordinary skill in the art include without limitation calcium, potassium, sodium, choline, ethylenediamine, tromethamine, arginine, glycinelycine, lycine, magnesium and meglumine.

There is also provided a “pharmaceutically acceptable solvates” of the compounds of the present invention. The term “solvate” means that the compound of the invention incorporates one or more pharmaceutically acceptable solvent. The solvate may contain one or more molecule of solvent per molecule of compound of the invention or may contain one or more molecule of compound of the invention per molecule of solvent. In one embodiment, the solvent may be held in the crystal in various ways and thus, the solvent molecule may occupy lattice positions in the crystal, or they may form bonds with salts of the compounds as described herein. The solvate(s) must be “acceptable” in the sense of not being deleterious to the recipient thereof. The solvation may be assessed by methods known in the art such as Loss on Drying techniques (LOD).

Reference hereinafter to a compound according to the invention includes compounds of the general formula (I) and their pharmaceutically acceptable salts, hydrates and solvates.

“Polymorphs”: It will be appreciated by those skilled in the art that the compounds in accordance with the present invention can exist in several different crystalline forms due to a different arrangement of molecules in the crystal lattice. This may include solvate or hydrate (also known as pseudopolymorphs) and amorphous forms. All such crystalline forms and polymorphs are included within the scope of the invention. The polymorphs may be characterized by methods well known in the art. Examples of analytical procedures that may be used to determine whether polymorphism occurs include: melting point (including hot-stage microscopy), infrared (not in solution), X-ray powder diffraction, thermal analysis methods (e.g. differential scanning calorimetry (DSC), differential thermal analysis (DTA), thermogravimetric analysis (TGA)), Raman spectroscopy, comparative intrinsic dissolution rate, scanning electron microscopy (SEM).

In one aspect, the present invention provides novel compounds including:

Compound 1
2-(4-bromobenzyl)-8-(3-phenyl-propyl)-2,8-diaza-spiro[4.5]decan-1-one hydrochloride

Compound 2
8-(3-phenylpropyl)-2-(4-trifluoromethyl-benzyl)-2,8-diaza-spiro[4.5]decan-1-one hydrochloride

Compound 3
2-(4-chlorobenzyl)-8-(3-phenyl-propyl)-2,8-diaza-spiro[4.5]decan-1-one hydrochloride

Compound 4
2-(4-fluorobenzyl)-8-(3-phenyl-propyl)-2,8-diaza-spiro[4.5]decan-1-one hydrochloride

Compound 5
8-(3-phenyl-propyl)-2-(4-trifluoromethoxy-benzyl)-2,8-diaza-spiro[4.5]decan-1-one hydrochloride

Compound 6
2-(4-methylbenzyl)-8-(3-phenyl-propyl)-2,8-diaza-spiro[4.5]decan-1-one hydrochloride

Compound 7
4-[l-oxo-8-(3-phenyl-propyl)-2,8-diaza-spiro[4.5]dec-2-ylmethyl]-benzonitrile hydrochloride

Compound 8
2-biphenyl-4-ylmethyl-8-(3-phenyl-propyl)-2,8-diaza-spiro[4.5]decan-1-one hydrochloride

Compound 9
2-naphthalen-2-ylmethyl-8-(3-phenyl-propyl)-2,8-diaza-spiro[4.5]decan-1-one hydrochloride

Compound 10
2-(4-bromobenzyl)-8-(3-phenyl-butyl)-2,8-diaza-spiro[4.5]decan-1-one hydrochloride

Compound 11
2-(4-bromobenzyl)-8-(3,3-diphenyl-propyl)-2,8-diaza-spiro[4.5]decan-1-one hydrochloride

Compound 12
8-(3,3-diphenyl-propyl)-2-(4-trifluoromethoxy-benzyl)-2,8-diaza-spiro[4.5]decan-1-one

Compound 13
2-(4-bromobenzyl)-8-(3,3-diphenyl-propyl)-2,8-diaza-spiro[4.5]decan-3-one hydrochloride

Compound 14
8-(3,3-diphenyl-propyl)-2-(3-phenyl-propyl)-2,8-diaza-spiro[4.5]decan-1-one hydrochloride

Compound 15
8-(3,3-diphenyl-propyl)-2-pyridin-4-ylmethyl-2,8-diaza-spiro[4.5]decan-1-one dihydrochloride

Compound 16
8-(3,3-diphenyl-propyl)-2-(4-methoxy-benzyl)-2,8-diaza-spiro[4.5]decan-1-one

Compound 17
8-(3,3-diphenyl-propyl)-2-(4-pyrazol-1-yl-benzyl)-2,8-diaza-spiro[4.5]decan-1-one hydrochloride

Compound 18
2-benzothiazol-2-ylmethyl-8-(3,3-diphenyl-propyl)-2,8-diaza-spiro[4.5]decan-1-one hydrochloride

Compound 19
8-(3,3-diphenyl-propyl)-2-(4-methanesulfonyl-benzyl)-2,8-diaza-spiro[4.5]decan-1-one hydrochloride

Compound 20
8-(3,3-diphenyl-propyl)-2-(3-phenyl-allyl)-2,8-diaza-spiro[4.5]decan-1-one hydrochloride

Compound 21
8-(3,3-diphenyl-propyl)-2-phenethyl-2,8-diaza-spiro[4.5]decan-1-one hydrochloride

Compound 22
2-(4-benzyloxy-benzyl)-8-(3,3-diphenyl-propyl)-2,8-diaza-spiro[4.5]decan-1-one

Compound 23
2-benzofuran-2-ylmethyl-8-(3,3-diphenyl-propyl)-2,8-diaza-spiro[4.5]decan-1-one

Compound 24
8-(3,3-diphenyl-propyl)-2-(4-isopropyl-benzyl)-2,8-diaza-spiro[4.5]decan-1-one

Compound 25
2-(5-chloro-benzo[b]thiophen-3-ylmethyl)-8-(3,3-diphenyl-propyl)-2,8-diaza-spiro[4.5]decan-1-one

Compound 26
8-(3,3-diphenyl-propyl)-2-(4-nitro-benzyl)-2,8-diaza-spiro[4.5]decan-1-one

Compound 27
2-(4-bromo-benzyl)-8-(3-pyridin-2-yl-propyl)-2,8-diaza-spiro[4.5]decan-1-one

Compound 28
2-[1-(4-bromophenyl)-ethyl]-8-(3,3-diphenyl-propyl)-2,8-diaza-spiro[4.5]decan-1-one hydrochloride

Compound 29
8-(3,3-diphenyl-propyl)-2-pyridin-3-ylmethyl-2,8-diaza-spiro[4.5]decan-1-one dihydrochloride

Compound 30
N-{4-[8-(3,3-diphenyl-propyl)-1-oxo-2,8-diaza-spiro[4.5]dec-2-ylmethyl]-phenyl}-acetamide hydrochloride

Compound 31
8-(3,3-diphenyl-propyl)-2-(6-trifluoromethyl-pyridin-3-ylmethyl)-2,8-diaza-spiro[4.5]decan-1-one dihydrochloride

Compound 32
4-[8-(3,3-diphenyl-propyl)-1-oxo-2,8-diaza-spiro[4.5]dec-2-ylmethyl]-benzoic acid hydrochloride

Compound 33
8-(3,3-diphenyl-propyl)-2-pyridin-2-ylmethyl-2,8-diaza-spiro[4.5]decan-1-one dihydrochloride

Compound 34
8-(3,3-diphenyl-propyl)-2-(4-trifluoromethylsulfanyl-benzyl)-2,8-diaza-spiro[4.5]decan-1-one hydrochloride

Compound 35
8-(3,3-diphenyl-propyl)-2-(4-methyl-cyclohexylmethyl)-2,8-diaza-spiro[4.5]decan-1-one hydrochloride

Compound 36
4-[8-(3,3-diphenyl-propyl)-1-oxo-2,8-diaza-spiro[4.5]dec-2-ylmethyl]-benzoic acid methyl ester hydrochloride

Compound 37
8-(3,3-diphenyl-propyl)-2-(5-trifluoromethyl-furan-2-ylmethyl)-2,8-diaza-spiro[4.5]decan-1-one hydrochloride

Compound 38
8-(3,3-diphenyl-propyl)-2-(4-iodo-benzyl)-2,8-diaza-spiro[4.5]decan-1-one

Compound 39
2-(4-methanesulfonylbenzyl)-8-(3-phenyl-butyl)-2,8-diaza-spiro[4.5]decan-1-one hydrochloride

Compound 40
2-(4-bromobenzyl)-8-[3-hydroxy-3-(2-methoxyphenyl)-3-phenyl-propyl]-2,8-diaza-spiro[4.5]decan-1-one

Compound 41
2-(4-bromobenzyl)-8-[3-hydroxy-3-(3-methoxyphenyl)-3-phenyl-propyl]-2,8-diaza-spiro[4.5]decan-1-one

Compound 42
2-(4-bromobenzyl)-8-(3-hydroxy-3-phenyl-3-thiophen-2-yl-propyl)-2,8-diaza-spiro[4.5]decan-1-one

Compound 43
2-(4-bromobenzyl)-8-(3-hydroxy-3-phenyl-butyl)-2,8-diaza-spiro[4.5]decan-1-one

Compound 44
2-(4-bromobenzyl)-8-[3-(2-methoxyphenyl)-3-phenyl-propyl]-2,8-diaza-spiro[4.5]decan-1-one

Compound 45
2-(4-bromobenzyl)-8-[3-(3-chlorophenyl)-3-hydroxy-3-phenyl-propyl]-2,8-diaza-spiro[4.5]decan-1-one

Compound 46
2-(4-bromobenzyl)-8-[3-(4-chlorophenyl)-3-hydroxy-3-phenyl-propyl]-2,8-diaza-spiro[4.5]decan-1-one

Compound 47
2-(4-bromobenzyl)-8-[3-(3-chlorophenyl)-3-phenyl-propyl)-2,8-diaza-spiro[4.5]decan-1-one

Compound 48
2-(4-bromobenzyl)-8-(3-phenyl-3-thiophen-2-yl-propyl)-2,8-diaza-spiro[4.5]decan-1-one

Compound 49
2-(4-bromobenzyl)-8-[3-(4-chlorophenyl)-3-phenyl-propyl]-2,8-diaza-spiro[4.5]decan-1-one

Compound 50
2-(4-Bromobenzyl)-8-(3-hydroxy-3-phenylpropyl)-2,8-diaza-spiro[4.5]decan-1-one

Compound 51
8-(3-Benzyloxy-3-phenylpropyl)-2-(4-bromobenzyl)-2,8-diaza-spiro[4.5]decan-1-one hydrochloride

Compound 52
2-(4-Bromobenzyl)-8-(3-phenoxy-3-phenylpropyl)-2,8-diaza-spiro[4.5]decan-1-one

Compound 53
{3-[2-(4-bromobenzyl)-1-oxo-2,8-diaza-spiro[4.5]dec-8-yl]-1-phenyl-propyl}-carbamic acid tert-butyl ester

Compound 54
N-{3-[2-(4-bromobenzyl)-1-oxo-2,8-diaza-spiro[4.5]dec-8-yl]-1-phenyl-propyl}-benzamide hydrochloride

Compound 55
N-{3-[2-(4-bromobenzyl)-1-oxo-2,8-diaza-spiro[4.5]dec-8-yl]-1-phenyl-propyl}-2,6-dimethyl-benzamide hydrochloride

Compound 56
Cyclohexanecarboxylic acid {3-[2-(4-bromobenzyl)-1-oxo-2,8-diaza-spiro[4.5]dec-8-yl]-1-phenyl-propyl}-amide hydrochloride

Compound 57
N-{3-[2-(4-bromobenzyl)-1-oxo-2,8-diaza-spiro[4.5]dec-8-yl]-1-phenyl-propyl}-2-phenyl-acetamide hydrochloride

Compound 58
N-{3-[2-(4-bromobenzyl)-1-oxo-2,8-diaza-spiro[4.5]dec-8-yl]-1-phenyl-propyl}-2-(2,4,6-trimethyl-phenyl)-acetamide hydrochloride

Compound 59
N-{3-[2-(4-bromobenzyl)-1-oxo-2,8-diaza-spiro[4.5]dec-8-yl]-1-phenyl-propyl}-3-phenyl-propionamide hydrochloride

Compound 60
{3-[2-(4-bromobenzyl)-1-oxo-2,8-diaza-spiro[4.5]dec-8-yl]-1-phenyl-propyl}-methyl-carbamic acid tert-butyl ester

Compound 61
N-{3-[2-(4-bromobenzyl)-1-oxo-2,8-diaza-spiro[4.5]dec-8-yl]-1-phenyl-propyl}-N-methyl-benzamide hydrochloride

Compound 62
Cyclohexanecarboxylic acid {3-[2-(4-bromo-benzyl)-1-oxo-2,8-diaza-spiro[4.5]dec-8-yl]-1-phenyl-propyl}-methyl-amide hydrochloride

Compound 63
N-{3-[2-(4-bromobenzyl)-1-oxo-2,8-diaza-spiro[4.5]dec-8-yl]-1-phenyl-propyl}-N-methyl-2-phenyl-acetamide hydrochloride

Compound 64
N-{3-[2-(4-bromo-benzyl)-1-oxo-2,8-diaza-spiro[4.5]dec-8-yl]-1-phenyl-propyl}-N-methyl-2-(2,4,6-trimethyl-phenyl)-acetamide hydrochloride

Compound 65
[3-(1 -oxo-2-pyridin-3-ylmethyl-2,8-diaza-spiro[4.5]dec-8-yl)-1-phenyl-propyl]-carbamic acid tert-butyl ester

Compound 66
{3-[2-(4-methanesulfonyl-benzyl)-1-oxo-2,8-diaza-spiro[4.5]dec-8-yl]-1-phenyl-propyl}-carbamic acid tert-butyl ester

Compound 67
[3-[2-(4-bromobenzyl)-1-oxo-2,8-diaza-spiro[4.5]dec-8-yl]-1-(3-chloro-phenyl)-propyl]-carbamic acid tert-butyl ester

Compound 68
N-{3-[2-(4-bromobenzyl)-1-oxo-2,8-diaza-spiro[4.5]dec-8-yl]-1-phenyl-propyl}-acetamide hydrochlroride

Compound 69
Cyclopropanecarboxylic acid {3-[2-(4-bromo-benzyl)-1-oxo-2,8-diaza-spiro[4.5]dec-8-yl]-1-phenyl-propyl}-amide hydrochloride

Compound 70
N-{3-[2-(4-bromo-benzyl)-1-oxo-2,8-diaza-spiro[4.5]dec-8-yl]-1-phenyl-propyl}-isobutyramide hydrochloride

Compound 71
N-{3-[2-(4-bromobenzyl)-1-oxo-2,8-diaza-spiro[4.5]dec-8-yl]-1-phenyl-propyl}-3-methyl-butyramide hydrochloride

Compound 72
N-{3-[2-(4-bromo-benzyl)-1-oxo-2,8-diaza-spiro[4.5]dec-8-yl]-1-phenyl-propyl}-2-chloro-benzamide hydrochloride

Compound 73
N-{3-[2-(4-bromo-benzyl)-1-oxo-2,8-diaza-spiro[4.5]dec-8-yl]-1-phenyl-propyl}-2-methoxy-benzamide hydrochloride

Compound 74
Pyridine-2-carboxylic acid {3-[2-(4-bromobenzyl)-1-oxo-2,8-diaza-spiro[4.5]dec-8-yl]-1-phenyl-propyl}-amide dihydrochloride

Compound 75
N-{3-[2-(4-bromobenzyl)-1-oxo-2,8-diaza-spiro[4.5]dec-8-yl]-1-phenyl-propyl}-3-chloro-benzamide hydrochloride

Compound 76
N-{3-[2-(4-bromo-benzyl)-1-oxo-2,8-diaza-spiro[4.5]dec-8-yl]-1-phenyl-propyl}-3-methoxy-benzamide hydrochloride

Compound 77
N-{3-[2-(4-bromobenzyl)-1-oxo-2,8-diaza-spiro[4.5]dec-8-yl]-1-phenyl-propyl}-nicotinamide dihydrochloride

Compound 78
N-{3-[2-(4-bromobenzyl)-1-oxo-2,8-diaza-spiro[4.5]dec-8-yl]-1-phenyl-propyl)-4-chloro-benzamide hydrochloride

Compound 79
N-{3-[2-(4-bromo-benzyl)-1-oxo-2,8-diaza-spiro[4.5]dec-8-yl]-1-phenyl-propyl}-4-methoxy-benzamide hydrochloride

Compound 80
N-{3-[2-(4-bromobenzyl)-1-oxo-2,8-diaza-spiro[4.5]dec-8-yl]-1-phenyl-propyl}-isonicotinamide dihydrochloride

Compound 81
N-{3-[2-(4-bromobenzyl)-1-oxo-2,8-diaza-spiro[4.5]dec-8-yl]-1-phenyl-propyl}-3,4-dichloro-benzamide hydrochloride

Compound 82
N-{3-[2-(4-bromo-benzyl)-1-oxo-2,8-diaza-spiro[4.5]dec-8-yl]-1-phenyl-propyl}-3,4-dimethoxy-benzamide hydrochloride

Compound 83
N-{3-[2-(4-bromo-benzyl)-1-oxo-2,8-diaza-spiro[4.5]dec-8-yl]-1-phenyl-propyl}-2-(2-chloro-phenyl)-acetamide hydrochloride

Compound 84
N-{3-[2-(4-bromobenzyl)-1-oxo-2,8-diaza-spiro[4.5]dec-8-yl]-1-phenyl-propyl}-2-(2-methoxy-phenyl)-acetamide hydrochloride

Compound 85
N-{3-[2-(4-bromobenzyl)-1-oxo-2,8-diaza-spiro[4.5]dec-8-yl]-1-phenyl-propyl}-2-(3-chloro-phenyl)-acetamide hydrochloride

Compound 86
N-{3-[2-(4-bromobenzyl)-1-oxo-2,8-diaza-spiro[4.5]dec-8-yl]-1-phenyl-propyl}-2-(3-methoxy-phenyl)-acetamide hydrochloride

Compound 87
N-{3-[2-(4-bromo-benzyl)-1-oxo-2,8-diaza-spiro[4.5]dec-8-yl]-1-phenyl-propyl}-2-pyridin-3-yl-acetamide dihydrochloride

Compound 88
N-{3-[2-(4-bromobenzyl)-1-oxo-2,8-diaza-spiro[4.5]dec-8-yl]-1-phenyl-propyl}-2-(4-methoxy-phenyl)-acetamide hydrochloride

Compound 89
N-{3-[2-(4-bromobenzyl)-1-oxo-2,8-diaza-spiro[4.5]dec-8-yl]-1-phenyl-propyl}-2-(3,4-dichloro-phenyl)-acetamide hydrochloride

Compound 90
Tetrahydro-pyran-4-carboxylic acid{3-[2-(4-bromobenzyl)-1-oxo-2,8-diaza-spiro[4.5]dec-8-yl]-1-phenyl-propyl}-amide hydrochloride

Compound 91
Cyclopentanecarboxylic acid {3-[2-(4-bromobenzyl)-1-oxo-2,8-diaza-spiro[4.5]dec-8-yl]-1-phenyl-propyl}-amide hydrochloride

Compound 92
Cyclobutanecarboxylic acid {3-[2-(4-bromobenzyl)-1-oxo-2,8-diaza-spiro[4.5]dec-8-yl]-1-phenyl-propyl}-amide hydrochloride

Compound 93
Cycloheptanecarboxylic acid {3-[2-(4-bromobenzyl)-1-oxo-2,8-diaza-spiro[4.5]dec-8-yl]-1-phenyl-propyl}-amide hydrochloride

Compound 94
N-{3-[2-(4-bromo-benzyl)-1-oxo-2,8-diaza-spiro[4.5]dec-8-yl]-1-phenyl-propyl}-2-cyclohexyl-acetamide hydrochloride

Compound 95
N-{3-[2-(4-methoxybenzyl)-1-oxo-2,8-diaza-spiro[4.5]dec-8-yl]-1-phenyl-propyl}-acetamide hydrochloride

Compound 96
Cyclopropanecarboxylic acid {3-[2-(4-methoxybenzyl)-1-oxo-2,8-diaza-spiro[4.5]dec-8-yl]-1-phenyl-propyl}-amide hydrochloride

Compound 97
N-{3-[2-(4-methoxy-benzyl)-1-oxo-2,8-diaza-spiro[4.5]dec-8-yl]-1-phenyl-propyl}-isobutyramide hydrochloride

Compound 98
N-{3-[2-(4-methoxybenzyl)-1-oxo-2,8-diaza-spiro[4.5]dec-8-yl]-1-phenyl-propyl}-3-methyl-butyramide hydrochloride

Compound 99
2-chloro-N-{3-[2-(4-methoxybenzyl)-1-oxo-2,8-diaza-spiro[4.5]dec-8-yl]-1-phenyl-propyl}-benzamide hydrochloride

Compound 100
2-methoxy-N-(3-[2-(4-methoxybenzyl)-1-oxo-2,8-diaza-spiro[4.5)dec-8-yl]-1-phenyl-propyl}-benzamide hydrochloride

Compound 101
Pyridine-2-carboxylic acid {3-[2-(4-methoxybenzyl)-1-oxo-2,8-diaza-spiro[4.5]dec-8-yl]-1-phenyl-propyl}-amide dihydrochloride

Compound 102
3-chloro-N-{3-[2-(4-methoxy-benzyl)-1-oxo-2,8-diaza-spiro[4.5]dec-8-yl]-1-phenyl-propyl}-benzamide hydrochloride

Compound 103
3-methoxy-N-{3-[2-(4-methoxy-benzyl)-1-oxo-2,8-diaza-spiro[4.5]dec-8-yl]-1-phenyl-propyl}-benzamide hydrochloride

Compound 104
N-{3-[2-(4-methoxybenzyl)-1-oxo-2,8-diaza-spiro[4.5]dec-8-yl]-1-phenyl-propyl}-nicotinamide dihydrochloride

Compound 105
4-chloro-N-{3-[2-(4-methoxybenzyl)-1-oxo-2,8-diaza-spiro[4.5]dec-8-yl]-1-phenyl-propyl}-benzamide hydrochloride

Compound 106
4-methoxy-N-{3-[2-(4-methoxy-benzyl)-1-oxo-2,8-diaza-spiro[4.5]dec-8-yl]-1-phenyl-propyl}-benzamide hydrochloride

Compound 107
N-{3-[2-(4-methoxybenzyl)-1-oxo-2,8-diaza-spiro[4.5]dec-8-yl]-1-phenyl-propyl}-isonicotinamide dihydrochloride

Compound 108
(R)-cyclohexanecarboxylic acid {3-[2-(4-bromobenzyl)-1-oxo-2,8-diaza-spiro [4.5]dec-8-yl]-1-phenyl-propyl}-amide

Compound 109
3,4-dichloro-N-{3-[2-(4-methoxybenzyl)-1-oxo-2,8-diaza-spiro[4.5]dec-8-yl]-1-phenyl-propyl}-benzamide hydrochloride

Compound 110
3,4-dimethoxy-N-{3-[2-(4-methoxybenzyl)-1-oxo-2,8-diaza-spiro[4.5]dec-8-yl]-1-phenyl-propyl}-benzamide hydrochloride

Compound 111
2-(2-chloro-phenyl)-N-{3-[2-(4-methoxybenzyl)-1-oxo-2,8-diaza-spiro[4.5]dec-8-yl]-1-phenyl-propyl}-acetamide hydrochloride

Compound 112
N-{3-[2-(4-methoxybenzyl)-1-oxo-2,8-diaza-spiro[4.5]dec-8-yl]-1-phenyl-propyl}-2-(2-methox-phenyl)-acetamide hydrochloride

Compound 113
2-(3-chlorophenyl)-N-{3-[2-(4-methoxybenzyl)-1-oxo-2,8-diaza-spiro[4.5]dec-8-yl]-1-phenyl-propyl}-acetamide hydrochloride

Compound 114
N-{3-[2-(4-methoxybenzyl)-1-oxo-2,8-diaza-spiro[4.5]dec-8-yl]-1-phenyl-propyl}-2-(3-methoxyphenyl)-acetamide hydrochloride

Compound 115
N-{3-[2-(4-methoxybenzyl)-1-oxo-2,8-diaza-spiro[4.5]dec-8-yl]-1-phenyl-propyl}-2-pyridin-3-yl-acetamide dihydrochloride

Compound 116
N-{3-[2-(4-methoxybenzyl)-1-oxo-2,8-diaza-spiro[4.5)dec-8-yl]-1-phenyl-propyl}-2-(4-methoxyphenyl)-acetamide hydrochloride

Compound 117
2-(3,4-dichlorophenyl)-N-{3-[2-(4-methoxybenzyl)-1-oxo-2,8-diaza-spiro[4.5]dec-8-yl]-1-phenyl-propyl}-acetamide hydrochloride

Compound 118
Tetrahydro-pyran-4-carboxylic acid{3-[2-(4-methoxybenzyl)-1-oxo-2,8-diaza-spiro[4.5]dec-8-yl]-1-phenyl-propyl}-amide hydrochloride

Compound 119
Cyclopentanecarboxylic acid {3-[2-(4-methoxybenzyl)-1-oxo-2,8-diaza-spiro[4.5]dec-8-yl]-1-phenyl-propyl}-amide hydrochloride

Compound 120
Cyclobutanecarboxylic acid {3-[2-(4-methoxybenzyl)-1-oxo-2,8-diaza-spiro[4.5]dec-8-yl]-1-phenyl-propyl}-amide hydrochloride

Compound 121
Cycloheptanecarboxylic acid {3-[2-(4-methoxybenzyl)-1-oxo-2,8-diaza-spiro[4.5]dec-8-yl]-1-phenyl-propyl}-amide hydrochloride

Compound 122
2-cyclohexyl-N-{3-[2-(4-methoxybenzyl)-1-oxo-2,8-diaza-spiro[4.5]dec-8-yl]-1-phenyl-propyl}-acetamide hydrochloride

Compound 123
(S)-cyclohexanecarboxylic acid 13-[2-(4-bromobenzyl)-1-oxo-2,8-diaza-spiro[4.5]dec-8-yl]-1-phenyl-propyl}-amide

Compound 124
N-{3-[2-(4-bromobenzyl)-1-oxo-2,8-diaza-spiro[4.5]dec-8-yl]-1-phenyl-propyl}-2-cyclopentyl-acetamide hydrochloride

Compound 125
Furan-2-carboxylic acid {3-[2-(4-bromobenzyl)-1-oxo-2,8-diaza-spiro[4.5]dec-8-yl]-1-phenyl-propyl}-amide hydrochloride

Compound 126
N-{3-[2-(4-bromobenzyl)-1-oxo-2,8-diaza-spiro[4.5]dec-8-yl]-1-phenyl-propyl}-2-ethyl-butyramide hydrochloride

Compound 127
Thiophene-2-carboxylic acid {3-[2-(4-brom-benzyl)-1-oxo-2,8-diaza-spiro[4.5]dec-8-yl]-1-phenyl-propyl}-amide hydrochloride

Compound 128
N-{3-[2-(4-bromobenzyl)-1-oxo-2,8-diaza-spiro[4.5]dec-8-yl]-1-phenyl-propyl}-2-(3,4-dimethoxyphenyl)-acetamide hydrochloride

Compound 129
2-cyclopentyl-N-{3-[2-(4-methoxybenzyl)-1-oxo-2,8-diaza-spiro[4.5]dec-8-yl]-1-phenyl-propyl}-acetamide hydrochloride

Compound 130
Furan-2-carboxylic acid {3-[2-(4-methox-benzyl)-1-oxo-2,8-diaza-spiro[4.5]dec-8-yl]-1-phenyl-propyl}-amide hydrochloride

Compound 131
2-ethyl-N-{3-[2-(4-methoxybenzyl)-1-oxo-2,8-diaza-spiro[4.5]dec-8-yl]-1-phenyl-propyl}-butyramide hydrochloride

Compound 132
Thiophene-2-carboxylic acid {3-[2-(4-methoxybenzyl)-1-oxo-2,8-diaza-spiro[4.5]dec-8-yl]-1-phenyl-propyl}-amide hydrochloride

Compound 133
2-(3,4-dimethoxy-phenyl)-N-{3-[2-(4-methoxybenzyl)-1-oxo-2,8-diaza-spiro[4.5]dec-8-yl]-1-phenyl-propyl}-acetamide hydrochloride

Compound 134
Cyclohexanecarboxylic acid {3-[2-(4-methoxybenzyl)-1-oxo-2,8-diaza-spiro[4.5]dec-8-yl]-1-phenyl-propyl}-amide hydrochloride

Compound 135
N-{3-[2-(4-methoxybenzyl)-1-oxo-2,8-diaza-spiro[4.5]dec-8-yl]-1-phenyl-propyl}-benzamide hydrochloride

Compound 136
N-{3-[2-(4-methoxybenzyl)-1-oxo-2,8-diaza-spiro[4.5]dec-8-yl]-1-phenyl-propyl}-2-phenyl-acetamide hydrochloride

Compound 137
N-{3-[2-(4-methanesulfonylbenzyl)-1-oxo-2,8-diaza-spiro[4.5]dec-8-yl]-1-phenyl-propyl}-acetamide hydrochloride

Compound 138
Cyclopropanecarboxylic acid {3-[2-(4-methanesulfonylbenzyl)-1-oxo-2,8-diaza-spiro[4.5]dec-8-yl]-1-phenyl-propyl}amide hydrochloride

Compound 139
N-{3-[2-(4-methanesulfonyl-benzyl)-1-oxo-2,8-diaza-spiro[4.5]dec-8-yl]-1-phenyl-propyl}-isobutyramide hydrochloride

Compound 140
N-{3-[2-(4-methanesulfonylbenzyl)-1-oxo-2,8-diaza-spiro[4.5]dec-8-yl]-1-phenyl-propyl}-3-methyl-butyramide hydrochloride

Compound 141
2-chloro-N-{3-[2-(4-methanesulfonylbenzyl)-1-oxo-2,8-diaza-spiro[4.5]dec-8-yl]-1-phenyl-propyl}-benzamide hydrochloride

Compound 142
N-{3-[2-(4-methanesulfonylbenzyl)-1-oxo-2,8-diaza-spiro[4.5]dec-8-yl]-1-phenyl-propyl}-2-methoxy-benzamide hydrochloride

Compound 143
Pyridine-2-carboxylic acid {3-[2-(4-methanesulfonylbenzyl)-1-oxo-2,8-diaza-spiro[4.5]dec-8-yl]-1-phenyl-propyl}-amide dihydrochloride

Compound 144
3-chloro-N-{3-[2-(4-methanesulfonylbenzyl)-1-oxo-2,8-diaza-spiro[4.5]dec-8-yl]-1-phenyl-propyl}-benzamide hydrochloride

Compound 145
N-{3-[2-(4-methanesulfonylbenzyl)-1-oxo-2,8-diaza-spiro[4.5]dec-8-yl]-1-phenyl-propyl}-3-methoxy-benzamide hydrochloride

Compound 146
N-{3-[2-(4-methanesulfonylbenzyl)-1-oxo-2,8-diaza-spiro[4.5]dec-8-yl]-1-phenyl-propyl}-nicotinamide dihydrochloride

Compound 147
4-chloro-N-{3-[2-(4-methanesulfonylbenzyl)-1-oxo-2,8-diaza-spiro[4.5]dec-8-yl]-1-phenyl-propyl}-benzamide hydrochloride

Compound 148
N-{3-[2-(4-methanesulfonylbenzyl)-1-oxo-2,8-diaza-spiro[4.5]dec-8-yl]-1-phenyl-propyl}-4-methoxy-benzamide hydrochloride

Compound 149
N-{3-[2-(4-methanesulfonylbenzyl)-1-oxo-2,8-diaza-spiro[4.5]dec-8-yl]-1-phenyl-propyl}-isonicotinamide dihydrochloride

Compound 150
3,4-dichloro-N-{3-[2-(4-methanesulfonylbenzyl)-1-oxo-2,8-diaza-spiro[4.5]dec-8-yl]-1-phenyl-propyl}-benzamide hydrochloride

Compound 151
N-{3-[2-(4-methanesulfonylbenzyl)-1-oxo-2,8-diaza-spiro[4.5]dec-8-yl]-1-phenyl-propyl}-3,4-dimethoxy-benzamide hydrochloride

Compound 152
2-(2-chlorophenyl)-N-{3-[2-(4-methanesulfonylbenzyl)-1-oxo-2,8-diaza-spiro[4.5]dec-8-yl]-1-phenyl-propyl}-acetamide hydrochloride

Compound 153
N-{3-[2-(4-methanesulfonylbenzyl)-1-oxo-2,8-diaza-spiro[4.5]dec-8-yl]-1-phenyl-propyl}-2-(2-methoxyphenyl)-acetamide hydrochloride

Compound 154
2-(3-chlorophenyl)-N-{3-[2-(4-methanesulfonylbenzyl)-1-oxo-2,8-diaza-spiro[4.5]dec-8-yl]-1-phenyl-propyl}-acetamide hydrochloride

Compound 155
N-{3-[2-(4-methanesulfonylbenzyl)-1-oxo-2,8-diaza-spiro[4.5]dec-8-yl]-1-phenyl-propyl}-2-(3-methoxyphenyl)-acetamide hydrochloride

Compound 156
N-{3-[2-(4-methanesulfonylbenzyl)-1-oxo-2,8-diaza-spiro[4.5]dec-8-yl]-1-phenyl-propyl}-benzamide hydrochloride

Compound 157
(S)-cyclohexanecarboxylic acid [3-(2-benzyl-1-oxo-2,8-diaza-spiro[4.5]dec-8-yl)-1-phenyl-propyl]-amide

Compound 158
N-{3-[2-(4-methanesulfonylbenzyl)-1-oxo-2,8-diaza-spiro[4.5]dec-8-yl]-1-phenyl-propyl}-2-(4-methoxyphenyl)-acetamide hydrochloride

Compound 159
N-{3-[2-(4-methanesulfonylbenzyl)-1-oxo-2,8-diaza-spiro[4.5]dec-8-yl]-1-phenyl-propyl}-2-phenyl-acetamide hydrochloride

Compound 160
2-(3,4-dichlorophenyl)-N-{3-[2-(4-methanesulfonylbenzyl)-1-oxo-2,8-diaza-spiro[4.5]dec-8-yl]-1-phenyl-propyl}-acetamide hydrochloride

Compound 161
Cyclopentanecarboxylic acid {3-[2-(4-methanesulfonylbenzyl)-1-oxo-2,8-diaza-spiro[4.5]dec-8-yl]-1-phenyl-propyl}-amide hydrochloride

Compound 162
Cyclobutanecarboxylic acid {3-[2-(4-methanesulfonylbenzyl)-1-oxo-2,8-diaza-spiro[4.5]dec-8-yl]-1-phenyl-propyl}-amide hydrochloride

Compound 163
Cycloheptanecarboxylic acid {3-[2-(4-methanesulfonylbenzyl)-1-oxo-2,8-diaza-spiro[4.5]dec-8-yl]-1-phenyl-propyl}-amide hydrochloride

Compound 164
2-cyclohexyl-N-{3-[2-(4-methanesulfonylbenzyl)-1-oxo-2,8-diaza-spiro[4.5]dec-8-yl]-1-phenyl-propyl}-acetamide hydrochloride

Compound 165
2-cyclopentyl-N-{3-[2-(4-methanesulfonylbenzyl)-1-oxo-2,8-diaza-spiro[4.5]dec-8-yl]-1-phenyl-propyl}-acetamide hydrochloride

Compound 166
Furan-2-carboxylic acid {3-[2-(4-methanesulfonylbenzyl)-1-oxo-2,8-diaza-spiro[4.5]dec-8-yl]-1-phenyl-propyl}-amide hydrochloride

Compound 167
2-ethyl-N-{3-[2-(4-methanesulfonylbenzyl)-1-oxo-2,8-diaza-spiro[4.5]dec-8-yl]-1-phenyl-propyl}-butyramide hydrochloride

Compound 168
Thiophene-2-carboxylic acid {3-[2-(4-methanesulfonylbenzyl)-1-oxo-2,8-diaza-spiro[4.5]dec-8-yl]-1-phenyl-propyl}-amide hydrochloride

Compound 169
2-(3,4-dimethoxyphenyl)-N-{3-[2-(4-methanesulfonyl-benzyl)-1-oxo-2,8-diaza-spiro[4.5]dec-8-yl]-1-phenyl-propyl}-acetamide hydrochloride

Compound 170
Cyclohexanecarboxylic acid {3-[2-(4-methanesulfonylbenzyl)-1-oxo-2,8-diaza-spiro[4.5]dec-8-yl]-1-phenyl-propyl}-amide hydrochloride

Compound 171
4-methyl-cyclohexanecarboxylic acid {3-[2-(4-methanesulfonylbenzyl)-1-oxo-2,8-diaza-spiro[4.5]dec-8-yl]-1-phenyl-propyl}-amide hydrochloride

Compound 172
2-methoxy-N-[3-(1-oxo-2-pyridin-3-ylmethyl-2,8-diaza-spiro[4.5]dec-8-yl)-1-phenyl-propyl]-benzamide dihydrochloride

Compound 173
3-chloro-N-[3-(1-oxo-2-pyridin-3-ylmethyl-2,8-diaza-spiro[4.5]dec-8-yl)-1-phenyl-propyl]-benzamide dihydrochloride

Compound 174
4-chloro-N-[3-(1-oxo-2-pyridin-3-ylmethyl-2,8-diaza-spiro[4.5]dec-8-yl)-1-phenyl-propyl]-benzamide dihydrochloride

Compound 175
4-methoxy-N-[3-(1-oxo-2-pyridin-3-ylmethyl-2,8-diaza-spiro[4.5]dec-8-yl)-1-phenyl-propyl]-benzamide dihydrochloride

Compound 176
Cyclohexanecarboxylic acid [3-[2-(4-bromobenzyl)-1-oxo-2,8-diaza-spiro[4.5]dec-8-yl]-1-(3-chloro-phenyl)-propyl]-amide hydrochloride

Compound 177
N-[3-[2-(4-bromobenzyl)-1-oxo-2,8-diaza-spiro[4.5]dec-8-yl]-1-(3-chlorophenyl)-propyl]-benzamide hydrochloride

Compound 178
N-[3-[2-(4-bromobenzyl)-1-oxo-2,8-diaza-spiro[4.5]dec-8-yl]-1-(3-chlorophenyl)-propyl]-2-phenyl-acetamide hydrochloride

Compound 179
{1-(3-chlorophenyl)-3-[2-(4-methoxybenzyl)-1-oxo-2,8-diaza-spiro[4.5]dec-8-yl]-propyl}-carbamic acid tert-butyl ester

Compound 180
{1-(3,4-dichlorophenyl)-3-[2-(4-methoxybenzyl)-1-oxo-2,8-diaza-spiro[4.5]dec-8-yl]-propyl}-carbamic acid tert-butyl ester

Compound 181
N-[3-(1-oxo-2-pyridin-3-ylmethyl-2,8-diaza-spiro[4.5]dec-8-yl)-1-phenyl-propyl]-acetamide dihydrochloride

Compound 182
Cyclopropanecarboxylic acid [3-(1-oxo-2-pyridin-3-ylmethyl-2,8-diaza-spiro[4.5]dec-8-yl)-1-phenyl-propyl]-amide dihydrochloride

Compound 183
N-[3-(1-oxo-2-pyridin-3-ylmethyl-2,8-diaza-spiro[4.5]dec-8-yl)-1-phenyl-propyl]-isobutyramide dihydrochloride

Compound 184
3-methyl-N-[3-(1-oxo-2-pyridin-3-ylmethyl-2,8-diaza-spiro[4.5]dec-8-yl)-1-phenyl-propyl]-butyramide dihydrochloride

Compound 185
2-chloro-N-[3-(1-oxo-2-pyridin-3-ylmethyl-2,8-diaza-spiro[4.5]dec-8-yl)-1-phenyl-propyl]-benzamide dihydrochloride

Compound 186
Pyridine-2-carboxylic acid [3-(1-oxo-2-pyridin-3-ylmethyl-2,8-diaza-spiro[4.5]dec-8-yl)-1-phenyl-propyl]-amide trihydrochloride

Compound 187
3-methoxy-N-[3-(1-oxo-2-pyridin-3-ylmethyl-2,8-diaza-spiro[4.5]dec-8-yl)-1-phenyl-propyl]-benzamide dihydrochloride

Compound 188
N-[3-(1-oxo-2-pyridin-3-ylmethyl-2,8-diaza-spiro[4.5]dec-8-yl)-1-phenyl-propyl]-nicotinamide trihydrochloride

Compound 189
N-[3-(1-oxo-2-pyridin-3-ylmethyl-2,8-diaza-spiro[4.5]dec-8-yl)-1-phenyl-propyl]-isonicotinamide trihydrochloride

Compound 190
3,4-dichloro-N-[3-(1-oxo-2-pyridin-3-ylmethyl-2,8-diaza-spiro[4.5]dec-8-yl)-1-phenyl-propyl]-benzamide dihydrochloride

Compound 191
3,4-dimethoxy-N-[3-(1-oxo-2-pyridin-3-ylmethyl-2,8-diaza-spiro[4.5]dec-8-yl)-1-phenyl-propyl]-benzamide dihydrochloride

Compound 192
2-(2-chlorophenyl)-N-[3-(1-oxo-2-pyridin-3-ylmethyl-2,8-diaza-spiro[4.5]dec-8-yl)-1-phenyl-propyl]-acetamide dihydrochloride

Compound 193
2-(2-methoxyphenyl)-N-[3-(1-oxo-2-pyridin-3-ylmethyl-2,8-diaza-spiro[4.5]dec-8-yl)-1-phenyl-propyl]-acetamide dihydrochloride

Compound 194
N-[3-(1-oxo-2-pyridin-3-ylmethyl-2,8-diaza-spiro[4.5]dec-8-yl)-1-phenyl-propyl]-benzamide dihydrochloride

Compound 195
2-(3-chloro-phenyl)-N-[3-(1-oxo-2-pyridin-3-ylmethyl-2,8-diaza-spiro[4.5]dec-8-yl)-1-phenyl-propyl]-acetamide dihydrochloride

Compound 196
2-(3-methoxyphenyl)-N-[3-(1-oxo-2-pyridin-3-ylmethyl-2,8-diaza-spiro[4.5]dec-8-yl)-1-phenyl-propyl]-acetamide dihydrochloride

Compound 197
2-(4-methoxyphenyl)-N-[3-(1-oxo-2-pyridin-3-ylmethyl-2,8-diaza-spiro[4.5]dec-8-yl)-1-phenyl-propyl]-acetamide dihydrochloride

Compound 198
N-[3-(1-oxo-2-pyridin-3-ylmethyl-2,8-diaza-spiro[4.5]dec-8-yl)-1-phenyl-propyl]-2-phenyl-acetamide dihydrochloride

Compound 199
2-(3,4-dichloro-phenyl)-N-[3-(1-oxo-2-pyridin-3-ylmethyl-2,8-diaza-spiro[4.5]dec-8-yl)-1-phenyl-propyl]-acetamide dihydrochloride

Compound 200
Cyclopentanecarboxylic acid [3-(1-oxo-2-pyridin-3-ylmethyl-2,8-diaza-spiro[4.5]dec-8-yl)-1-phenyl-propyl]-amide dihydrochloride

Compound 201
{1-(3-chlorophenyl)-3-[2-(4-methanesulfonylbenzyl)-1-oxo-2,8-diaza-spiro[4.5]dec-8-yl]-propyl}-carbamic acid tert-butyl ester

Compound 202
Cyclobutanecarboxylic acid [3-(1-oxo-2-pyridin-3-ylmethyl-2,8-diaza-spiro[4.5)dec-8-yl)-1-phenyl-propyl]-amide dihydrochloride

Compound 203
Cycloheptanecarboxylic acid [3-(1-oxo-2-pyridin-3-ylmethyl-2,8-diaza-spiro[4.5]dec-8-yl)-1-phenyl-propyl]-amide dihydrochloride

Compound 204
2-cyclohexyl-N-[3-(1-oxo-2-pyridin-3-ylmethyl-2,8-diaza-spiro[4.5]dec-8-yl)-1-phenyl-propyl]-acetamide dihydrochloride

Compound 205
2-cyclopentyl-N-[3-(1-oxo-2-pyridin-3-ylmethyl-2,8-diaza-spiro[4.5]dec-8-yl)-1-phenyl-propyl]-acetamide dihydrochloride

Compound 206
Furan-2-carboxylic acid [3-(1-oxo-2-pyridin-3-ylmethyl-2,8-diaza-spiro[4.5]dec-8-yl)-1-phenyl-propyl]-amide dihydrochloride

Compound 207
2-ethyl-N-[3-(1-oxo-2pyridin-3-ylmethyl-2,8-diaza-spiro[4.5]dec-8-yl)-1-phenyl-propyl]-butyramide dihydrochloride

Compound 208
Thiophene-2-carboxylic acid [3-(1-oxo-2-pyridin-3-ylmethyl-2,8-diaza-spiro[4.5]dec-8-yl)-1-phenyl-propyl]-amide dihydrochloride

Compound 209
2-(3,4-dimethoxyphenyl)-N-[3-(l-oxo-2-pyridin-3-ylmethyl-2,8-diaza-spiro[4.5]dec-8-yl)-1-phenyl-propyl]-acetamide dihydrochloride

Compound 210
Cyclohexanecarboxylic acid [3-(1-oxo-2-pyridin-3-ylmethyl-2,8-diaza-spiro[4.5]dec-8-yl)-1-phenyl-propyl)-amide dihydrochloride

Compound 211
4-methyl-cyclohexanecarboxylic acid [3-(1-oxo-2-pyridin-3-ylmethyl-2,8-diaza-spiro[4.5]dec-8-yl)-1-phenyl-propyl]-amide dihydrochloride

Compound 212
[3-[2-(4-bromobenzyl)-1-oxo-2,8-diaza-spiro[4.5]dec-8-yl]-1-(3-methoxyphenyl)-propyl]-carbamic acid tert-butyl ester

Compound 213
[3-[2-(4-methoxybenzyl)-1-oxo-2,8-diaza-spiro[4.5]dec-8-yl]-1-(3-methoxyphenyl)-propyl]-carbamic acid tert-butyl ester

Compound 214
(S)-N-{3-[2-(4-bromobenzyl)-1-oxo-2,8-diaza-spiro[4.5]dec-8-yl]-1-phenyl-propyl}-acetamide hydrochloride

Compound 215
(S)-cyclopropanecarboxylic acid {3-[2-(4-bromobenzyl)-1-oxo-2,8-diaza-spiro[4.5]dec-8-yl]-1-phenyl-propyl}-amide hydrochloride

Compound 216
(S)-N-{3-[2-(4-bromobenzyl)-1-oxo-2,8-diaza-spiro[4.5]dec-8-yl]-1-phenyl-propyl}-isobutyramide hydrochloride

Compound 217
(S)-N-{3-[2-(4-bromobenzyl)-1-oxo-2,8-diaza-spiro[4.5]dec-8-yl]-1-phenyl-propyl)-3-methyl-butyramide hydrochloride

Compound 218
(S)-cyclopentanecarboxylic acid {3-[2-(4-bromobenzyl)-1-oxo-2,8-diaza-spiro[4.5]dec-8-yl]-1-phenyl-propyl}-amide hydrochloride

Compound 219
(S)-cyclobutanecarboxylic acid {3-[2-(4-bromobenzyl)-1-oxo-2,8-diaza-spiro[4.5]dec-8-yl]-1-phenyl-propyl}-amide hydrochloride

Compound 220
[3-[2-(4-methoxybenzyl)-1-oxo-2,8-diaza-spiro[4.5]dec-8-yl]-1-(4-methoxyphenyl)-propyl]-carbamic acid tert-butyl ester

Compound 221
{3-[2-(4-bromobenzyl)-1-oxo-2,8-diaza-spiro[4.5]dec-8-yl]-1-pyridin-2-yl-propyl}-carbamic acid tert-butyl ester

Compound 222
{3-[2-(4-methoxybenzyl)-1-oxo-2,8-diaza-spiro[4.5]dec-8-yl]-1-pyridin-2-yl-propyl}-carbamic acid tert-butyl ester

Compound 223
{1-(3,4-dichlorophenyl)-3-[2-(4-methanesulfonylbenzyl)-1-oxo-2,8-diaza-spiro[4.5]dec-8-yl]-propyl}-carbamic acid tert-butyl ester

Compound 224
2-cyclopropyl-N-{3-[2-(4-methoxybenzyl)-1-oxo-2,8-diaza-spiro[4.5]dec-8-yl]-1-phenyl-propyl}-acetamide hydrochloride

Compound 225
2-cyclopropyl-N-{3-[2-(4-methanesulfonylbenzyl)-1-oxo-2,8-diaza-spiro[4.5]dec-8-yl]-1-phenyl-propyl}-acetamide hydrochloride

Compound 226
[3-[2-(4-bromobenzyl)-1-oxo-2,8-diaza-spiro[4.5]dec-8-yl]-1-(4-methoxyphenyl)-propyl]-carbamic acid tert-butyl ester

Compound 227
N-{3-[2-(4-bromobenzyl)-1-oxo-2,8-diaza-spiro[4.5]dec-8-yl-1-phenyl-propyl}-2-cyclopropyl-acetamide hydrochloride

Compound 228
[3-[2-(4-bromobenzyl)-1-oxo-2,8-diaza-spiro[4.5]dec-8-yl]-1(3,4-dimethoxyphenyl)-propyl]-carbamic acid tert-butyl ester

Compound 229
{1-(3,4-dimethoxyphenyl)-3-[2-(4-methoxybenzyl)-1-oxo-2,8-diaza-spiro[4.5]dec-8-yl]-propyl}-carbamic acid tert-butyl ester

Compound 230
Tetrahydro-pyran-4-carboxylic acid (3-[2-(4-methanesulfonylbenzyl)-1-oxo-2,8-diaza-spiro[4.5]dec-8-yl]-1-phenyl-propyl}-amide hydrochloride

Compound 231
[3-[2-(4-methanesulfonylbenzyl)-1-oxo-2,8-diaza-spiro[4.5]dec-8-yl]-1-(3-methoxy-phenyl)-propyl]-carbamic acid tertbutyl ester

Compound 232
(S)-{3-[2-(4-methanesulfonylbenzyl)-1-oxo-2,8-diaza-spiro[4.5]dec-8-yl]-1-phenyl-propyl}-carbamic acid tert-butyl ester

Compound 233
{3-[2-(4-methanesulfonylbenzyl)-1-oxo-2,8-diaza-spiro[4.5]dec-8-yl]-1-pyridin-2-yl-propyl}-carbamic acid tert-butyl ester

Compound 234
{1-(3,4-dimethoxyphenyl)-3-[2-(4-methanesulfonylbenzyl)-1-oxo-2,8-diaza-spiro[4.5]dec-8-yl]-propyl}-carbamic acid tert-butyl ester

Compound 235
{1-(4-chlorophenyl)-3-[2-(4-methoxybenzyl)-1-oxo-2,8-diaza-spiro[4.5]dec-8-yl]-propyl}-carbamic acid tert-butyl ester

Compound 236
{1-(2-chlorophenyl)-3-[2-(4-methoxybenzyl)-1-oxo-2,8-diaza-spiro[4.5]dec-8-yl]-propyl}-carbamic acid tert-butyl ester

Compound 237
{3-[2-(4-methoxybenzyl)-1-oxo-2,8-diaza-spiro[4.5]dec-8-yl]-1-thiophen-2-yl-propyl}-carbamic acid tert-butyl ester

Compound 238
[3-[2-(4-methanesulfonylbenzyl)-1-oxo-2,8-diaza-spiro[4.5]dec-8-yl]-1-(4-methoxyphenyl)-propyl]-carbamic acid tert-butyl ester

Compound 239
[3-[2-(4-bromobenzyl)-1-oxo-2,8-diaza-spiro[4.5]dec-8-yl]-1-(4-chlorophenyl)-propyl]-carbamic acid tert-butyl ester

Compound 240
[3-[2-(4-bromobenzyl)-1-oxo-2,8-diaza-spiro[4.5]dec-8-yl]-1-(2-chlorophenyl)-propyl]-carbamic acid tert-butyl ester

Compound 241
{3-[2-(4-bromobenzyl)-1-oxo-2,8-diaza-spiro[4.5]dec-8-yl]-1-thiophen-2-yl-propyl}-carbamic acid tert-butyl ester

Compound 242
{1-(4-chlorophenyl)-3-[2-(4-methanesulfonylbenzyl)-1-oxo-2,8-diaza-spiro[4.5]dec-8-yl]-propyl}-carbamic acid tert-butyl ester

Compound 243
{1-(2-chlorophenyl)-3-[2-(4-methanesulfonylbenzyl)-1-oxo-2,8-diaza-spiro[4.5]dec-8-yl]-propyl}-carbamic acid tert-butyl ester

Compound 244
{3-[2-(4-methanesulfonylbenzyl)-1-oxo-2,8-diaza-spiro[4.5]dec-8-yl]-1-thiophen-2-yl-propyl}-carbamic acid tert-butyl ester

Compound 245
(S)-N-{3-[2-(4-methanesulfonylbenzyl)-1-oxo-2,8-diaza-spiro[4.5]dec-8-yl]-1-phenyl-propyl}-isobutyramide

Compound 246
[3-[2-(4-bromobenzyl)-1-oxo-2,8-diaza-spiro[4.5]dec-8-yl]-1-(2-methoxyphenyl)-propyl]-carbamic acid tert-butyl ester

Compound 247
[3-[2-(4-methoxy-benzyl)-1-oxo-2,8-diaza-spiro[4.5]dec-8-yl]-1-(2-methoxy-phenyl)-propyl]-carbamic acid tert-butyl ester

Compound 248
[1-(2-chlorophenyl)-3-(1-oxo-2-pyridin-3-ylmethyl-2,8-diaza-spiro[4.5]dec-8-yl)-propyl]-carbamic acid tert-butyl ester

Compound 249
[1-(3-chlorophenyl)-3-(1-oxo-2-pyridin-3-ylmethyl-2,8-diaza-spiro[4.5]dec-8-yl)-propyl]-carbamic acid tert-butyl ester

Compound 250
[1-(3,4-dichlorophenyl)-3-(1-oxo-2-pyridin-3-ylmethyl-2,8-diaza-spiro[4.5]dec-8-yl)-propyl]-carbamic acid tert-butyl ester

Compound 251
[3-(1-oxo-2-pyridin-3-ylmethyl-2,8-diaza-spiro[4.5)dec-8-yl)-1-thiophen-2-yl-propyl]-carbamic acid tert-butyl ester

Compound 252
(S)-8-[3-(cyclopropanecarbonyl-amino)-3-phenyl-propyl]-2-(4-methanesulfonylbenzyl)-1-oxo-2,8-diaza-spiro[4.5]decane hydrochloride

Compound 253
(S)-8-[3-(cyclopentanecarbonyl-amino)-3-phenyl-propyl]-2-(4-methanesulfonylbenzyl)-1-oxo-2,8-diaza-spiro[4.5]decane hydrochloride

Compound 254
(S)-8-[3-(cyclohexanecarbonyl-amino)-3-phenyl-propyl]-2-(4-methanesulfonylbenzyl)-1-oxo-2,8-diaza-spiro[4.5]decane hydrochloride

Compound 255
(S)-8-[3-(cyclopropanecarbonyl-amino)-3-phenyl-propyl]-2-(4-methoxybenzyl)-1-oxo-2,8-diaza-spiro[4.5)decane hydrochloride

Compound 256
(S)-8-(3-isobutyrylamino-3-phenyl-propyl)-2-(4-methoxybenzyl)-1-oxo-2,8-diaza-spiro[4.5]decane hydrochloride

Compound 257
(S)-8-[3-(cyclopentanecarbonyl-amino)-3-phenyl-propyl]-2-(4-methoxybenzyl)-1-oxo-2,8-diaza-spiro[4.5]decane hydrochloride

Compound 258
(S)-8-[3-(cyclohexanecarbonyl-amino)-3-phenyl-propyl]-2-(4-methoxybenzyl)-1-oxo-2,8-diaza-spiro[4.5)decane hydrochloride

Compound 259
N-{3-[2-(4-bromobenzyl)-1-oxo-2,8-diaza-spiro[4.5]dec-8-yl]-1-thiophen-2-yl-propyl}-isobutyramide hydrochloride

Compound 260
Cyclobutanecarboxylic acid {3-[2-(4-bromobenzyl)-1-oxo-2,8-diaza-spiro[4.5]dec-8-yl]-1-thiophen-2-yl-propyl}-amide hydrochloride

Compound 261
Cyclopentanecarboxylic acid {3-[2-(4-bromobenzyl)-1-oxo-2,8-diaza-spiro[4.5]dec-8-yl]-1-thiophen-2-yl-propyl}-amide hydrochloride

Compound 262
N-{3-[2-(4-bromo-enzyl)-1-oxo-2,8-diaza-spiro[4.5]dec-8-yl]-1-thiophen-2-yl-propyl}-propionamide hydrochloride

Compound 263
N-{3-[2-(4-bromobenzyl)-1-oxo-2,8-diaza-spiro[4.5]dec-8-yl]-1-thiophen-2-yl-propyl)-2-methoxy-acetamide hydrochloride

Compound 264
Cyclohexanecarboxylic acid {3-[2-(4-bromobenzyl)-1-oxo-2,8-diaza-spiro[4.5]dec-8-yl]-1-thiophen-2-yl-propyl}-amide hydrochloride

Compound 265
Cyclopropanecarboxylic acid {3-[2-(4-methoxybenzyl)-1-oxo-2,8-diaza-spiro[4.5]dec-8-yl]-1-thiophen-2-yl-propyl}-amide hydrochloride

Compound 266
N-{3-[2-(4-methoxybenzyl)-1-oxo-2,8-diaza-spiro[4.5]dec-8-yl]-1-thiophen-2-yl-propyl}-isobutyramide hydrochloride

Compound 267
[3-[2-(4-methanesulfonylbenzyl)-1-oxo-2,8-diaza-spiro[4.5]dec-8-yl]-1-(2-methoxyphenyl)-propyl]-carbamic acid tert-butyl ester

Compound 268
Cyclobutanecarboxylic acid {3-[2-(4-methoxybenzyl)-1-oxo-2,8-diaza-spiro[4.5]dec-8-yl]-1-thiophen-2-yl-propyl}-amide hydrochloride

Compound 269
Cyclopentanecarboxylic acid {3-[2-(4-methoxybenzyl)-1-oxo-2,8-diaza-spiro[4.5]dec-8-yl]-1-thiophen-2-yl-propyl}-amide hydrochloride

Compound 270
N-{3-[2-(4-methoxybenzyl)-1-oxo-2,8-diaza-spiro[4.5]dec-8-yl]-1-thiophen-2-yl-propyl}-propionamide hydrochloride

Compound 271
2-methoxy-N-{3-[2-(4-methoxybenzyl)-1-oxo-2,8-diaza-spiro[4.5]dec-8-yl]-1-thiophen-2-yl-propyl}-acetamide hydrochloride

Compound 272
Cyclohexanecarboxylic acid {3-[2-(4-methoxybenzyl)-1-oxo-2,8-diaza-spiro[4.5]dec-8-yl]-1-thiophen-2-yl-propyl}-amide hydrochloride

Compound 273
Cyclopropane carboxylic acid{3-[2-(4-methanesulfonylbenzyl)-1-oxo-2,8-diaza-spiro[4.5]dec-8-yl]-1-thiophen-2-yl-propyl}-amide hydrochloride

Compound 274
N-{3-[2-(4-methanesulfonylbenzyl)-1-oxo-2,8-diaza-spiro[4.5]dec-8-yl]-1-thiophen-2-yl-propyl}-isobutyramide hydrochloride

Compound 275
Cyclobutanecarboxylic acid {3-[2-(4-methanesulfonylbenzyl)-1-oxo-2,8-diaza-spiro[4.5]dec-8-yl]-1-thiophen-2-yl-propyl}-amide hydrochloride

Compound 276
Cyclopentanecarboxylic acid {3-[2-(4-methanesulfonylbenzyl)-1-oxo-2,8-diaza-spiro[4.5]dec-8-yl]-1-thiophen-2-yl-propyl}-amide hydrochloride

Compound 277
N-{3-[2-(4-methanesulfonylbenzyl)-1-oxo-2,8-diaza-spiro[4.5]dec-8-yl]-1-thiophen-2-yl-propyl}-propionamide hydrochloride

Compound 278
N-{3-[2-(4-methanesulfonylbenzyl)-1-oxo-2,8-diaza-spiro[4.5]dec-8-yl]-1-thiophen-2-yl-propyl}-2-methoxy-acetamide hydrochloride

Compound 279
Cyclohexanecarboxylic acid {3-[2-(4-methanesulfonylbenzyl)-1-oxo-2,8-diaza-spiro[4.5]dec-8-yl]-1-thiophen-2-yl-propyl}-amide hydrochloride

Compound 280
Cyclohexanecarboxylic acid [3-(1-oxo-2-pyridin-3-ylmethyl-2,8-diaza-spiro[4.5]dec-8-yl)-1-thiophen-2-yl-propyl]-amide dihydrochloride

Compound 281
N-[3-(1-oxo-2-pyridin-3-ylmethyl-2,8-diaza-spiro[4.5]dec-8-yl)-1-thiophen-2-yl-propyl]-isobutyramide dihydrochloride

Compound 282
Cyclobutanecarboxylic acid [3-(1-oxo-2-pyridin-3-ylmethyl-2,8-diaza-spiro[4.5]dec-8-yl)-1-thiophen-2-yl-propyl)-amide dihydrochloride

Compound 283
Cyclopentanecarboxylic acid [3-(1-oxo-2-pyridin-3-ylmethyl-2,8-diaza-spiro[4.5]dec-8-yl)-1-thiophen-2-yl-propyl]-amide dihydrochloride

Compound 284
N-[3-(l-oxo-2-pyridin-3-ylmethyl-2,8-diaza-spiro[4.5]dec-8-yl)-1-thiophen-2-yl-propyl]-propionamide dihydrochloride

Compound 285
Cyclohexanecarboxylic acid [3-(1-oxo-2-pyridin-3-ylmethyl-2,8-diaza-spiro[4.5]dec-8-yl)-1-thiophen-2-yl-propyl]-amide dihydrochloride

Compound 286
Cyclopropanecarboxylic acid [3-[2-(4-bromobenzyl)-1-oxo-2,8-diaza-spiro[4.5]dec-8-yl]-1-(3-chlorophenyl)-propyl]-amide hydrochloride

Compound 287
N-[3-[2-(4-bromobenzyl)-1-oxo-2,8-diaza-spiro[4.5]dec-8-yl]-1-(3-chlorophenyl)-propyl]-isobutyramide hydrochloride

Compound 288
Cyclobutanecarboxylic acid [3-[2-(4-bromobenzyl)-1-oxo-2,8-diaza-spiro[4.5]dec-8-yl]-1-(3-chlorophenyl)-propyl]-amide hydrochloride

Compound 289
Cyclopentanecarboxylic acid [3-[2-(4-bromobenzyl)-1-oxo-2,8-diaza-spiro[4.5]dec-8-yl]-1-(3-chlorophenyl)-propyl]-amide hydrochloride

Compound 290
N-[3-[2-(4-bromobenzyl)-1-oxo-2,8-diaza-spiro[4.5]dec-8-yl]-1-(3-chlorophenyl)-propyl]-propionamide hydrochloride

Compound 291
N-[3-[2-(4-bromobenzyl)-1-oxo-2,8-diaza-spiro[4.5]dec-8-yl]-1-(3-chlorophenyl)-propyl]-2-methoxy-acetamide hydrochloride

Compound 292
2-Cyclopropyl-N-{(S)-3-[2-(4-methanesulfonyl-benzyl)-1-oxo-2,8-diaza-spiro[4.5]dec-8-yl]-1-phenyl-propyl}-acetamide hydrochloride

Compound 293
2-Cyclopropyl-N-{(S)-3-[2-(4-methoxybenzyl)-1-oxo-2,8-diaza-spiro[4.5]dec-8-yl]-1-phenyl-propyl}-acetamide hydrochloride

Compound 294
Cyclopentanecarboxylic acid [(S)-3-(2-benzyl-1-oxo-2,8-diaza-spiro[4.5]dec-8-yl)-1-phenyl-propyl]-amide hydrochloride

Compound 295
Cyclopropanecarboxylic acid [(S)-3-(2-benzyl-1-oxo-2,8-diaza-spiro[4.5]dec-8-yl)-1-phenyl-propyl]-amide hydrochloride

Compound 296
Cyclohexanecarboxylic acid [3-[2-(4-bromobenzyl)-1-oxo-2,8-diaza-spiro[4.5]dec-8-yl]-1-(3-chlorophenyl)-propyl]-amide hydrochloride

Compound 297
Cyclopropanecarboxylic acid {l-(3-chloro-phenyl)-3-[2-(4-methoxy-benzyl)-1-oxo-2,8-diaza-spiro[4.5]dec-8-yl]-propyl}-amide hydrochloride

Compound 298
N-{1-(3-Chloro-phenyl)-3-[2-(4-methoxy-benzyl)-1-oxo-2,8-diaza-spiro[4.5]dec-8-yl]-propyl}-isobutyramide hydrochloride

Compound 299
Cyclobutanecarboxylic acid {l-(3-chlorophenyl)-3-[2-(4-methoxybenzyl)-1-oxo-2,8-diaza-spiro[4.5]dec-8-yl]-propyl}-amide hydrochloride

Compound 300
Cyclopentanecarboxylic acid {1-(3-chlorophenyl)-3-[2-(4-methoxybenzyl)-1-oxo-2,8-diaza-spiro[4.5]dec-8-yl]-propyl}-amide hydrochloride

Compound 301
N-{l-(3-Chlorophenyl)-3-[2-(4-methoxybenzyl)-1-oxo-2,8-diaza-spiro[4.5]dec-8-yl]-propyl}-propionamide hydrochloride

Compound 302
N-{1-(3-Chlorophenyl)-3-[2-(4-methoxybenzyl)-1-oxo-2,8-diaza-spiro[4.5]dec-8-yl]-propyl}-2-methoxy-acetamide hydrochloride

Compound 303
Cyclohexanecarboxylic acid {1-(3-chlorophenyl)-3-[2-(4-methoxybenzyl)-1-oxo-2,8-diaza-spiro[4.5]dec-8-yl]-propyl}-amide hydrochloride

Compound 304
Cyclopropanecarboxylic acid {l-(3-chlorophenyl)-3-[2-(4-methanesulfonyl-benzyl)-1-oxo-2,8-diaza-spiro[4.5]dec-8-yl]-propyl}-amide hydrochloride

Compound 305
N-{l-(3-Chlorophenyl)-3-[2-(4-methanesulfonylbenzyl)-1-oxo-2,8-diaza-spiro[4.5]dec-8-yl]-propyl}-isobutyramide hydrochloride

Compound 306
Cyclobutanecarboxylic acid {1-(3-chlorophenyl)-3-[2-(4-methanesulfonylbenzyl)-1-oxo-2,8-diaza-spiro[4.5]dec-8-yl]-propyl}-amide hydrochloride

Compound 307
Cyclopentanecarboxylic acid {1-(3-chlorophenyl)-3-[2-(4-methanesulfonylbenzyl)-1-oxo-2,8-diaza-spiro[4.5]dec-8-yl]-propyl}-amide hydrochloride

Compound 308
N-{l-(3-Chlorophenyl)-3-[2-(4-methanesulfonylbenzyl)-1-oxo-2,8-diaza-spiro[4.5]dec-8-yl]-propyl}-propionamide hydrochloride

Compound 309
N-{l-(3-Chlorophenyl)-3-[2-(4-methanesulfonylbenzyl)-1-oxo-2,8-diaza-spiro[4.5]dec-8-yl]-propyl}-2-methoxy-acetamide hydrochloride

Compound 310
Cyclopropanecarboxylic acid [1-(3-chlorophenyl)-3-(1-oxo-2-pyridin-3-ylmethyl-2,8-diaza-spiro[4.5]dec-8-yl)-propyl]-amide dihydrochloride

Compound 311
N-[1-(3-Chlorophenyl)-3-(1-oxo-2-pyridin-3-ylmethyl-2,8-diaza-spiro[4.5]dec-8-yl)-propyl]-isobutyramide dihydrochloride

Compound 312
Cyclobutanecarboxylic acid [1-(3-chlorophenyl)-3-(1-oxo-2-pyridin-3-ylmethyl-2,8-diaza-spiro[4.5]dec-8-yl)-propyl]-amide dihydrochloride

Compound 313
Cyclopentanecarboxylic acid [1-(3-chloro-phenyl)-3-(1-oxo-2-pyridin-3-ylmethyl-2,8-diaza-spiro[4.5]dec-8-yl)-propyl]-amide dihydrochloride

Compound 314
N-[1-(3-Chlorophenyl)-3-(1-oxo-2-pyridin-3-ylmethyl-2,8-diaza-spiro[4.5]dec-8-yl)-propyl]-propionamide dihydrochloride

Compound 315
N-[1-(3-Chlorophenyl)-3-(1-oxo-2-pyridin-3-ylmethyl-2,8-diaza-spiro[4.5]dec-8-yl)-propyl]-2-methoxy-acetamide dihydrochloride

Compound 316
Cyclohexanecarboxylic acid [1-(3-chlorophenyl)-3-(1-oxo-2-pyridin-3-ylmethyl-2,8-diaza-spiro[4.5]dec-8-yl)-propyl]-amide dihydrochloride

Compound 317
Cyclopropanecarboxylic acid [(S)-3-(1-oxo-2-pyridin-2-ylmethyl-2,8-diaza-spiro[4.5]dec-8-yl)-1-phenyl-propyl]-amide dihydrochloride

Compound 318
Cyclopentanecarboxylic acid [(S)-3-(1-oxo-2-pyridin-2-ylmethyl-2,8-diaza-spiro[4.5]dec-8-yl)-1-phenyl-propyl]-amide dihydrochloride

Compound 319
Cyclopropanecarboxylic acid [(S)-3-(1-oxo-2-pyridin-3-ylmethyl-2,8-diaza-spiro[4.5]dec-8-yl)-1-phenyl-propyl]-amide dihydrochloride

Compound 320
Cyclopentanecarboxylic acid [(S)-3-(1-oxo-2-pyridin-3-ylmethyl-2,8-diaza-spiro[4.5]dec-8-yl)-1-phenyl-propyl]-amide dihydrochloride

Compound 321
Cyclopropanecarboxylic acid [(S)-3-(1-oxo-2-pyridin-4-ylmethyl-2,8-diaza-spiro[4.5]dec-8-yl)-1-phenyl-propyl]-amide dihydrochloride

Compound 322
Cyclopentanecarboxylic acid [(S)-3-(1-oxo-2-pyridin-4-ylmethyl-2,8-diaza-spiro[4.5]dec-8-yl)-1-phenyl-propyl]-amide dihydrochloride

Compound 323
Cyclopropanecarboxylic acid {(S)-3-[1-oxo-2-(1-oxy-pyridin-2-ylmethyl)-2,8-diaza-spiro[4.5)dec-8-yl]-1-phenyl-propyl}-amide hydrochloride

Compound 324
Cyclopentanecarboxylic acid {(S)-3-[1-oxo-2-(1-oxy-pyridin-2-ylmethyl)-2,8-diaza-spiro[4.5]dec-8-yl]-1-phenyl-propyl}-amide hydrochloride

Compound 325
Cyclopropanecarboxylic acid {(S)-3-[1-oxo-2-(1-oxy-pyridin-3-ylmethyl)-2,8-diaza-spiro(4.5]dec-8-yl]-1-phenyl-propyl}-amide hydrochloride

Compound 326
Cyclopentanecarboxylic acid {(S)-3-[1-oxo-2-(1-oxy-pyridin-3-ylmethyl)-2,8-diaza-spiro[4.5]dec-8-yl]-1-phenyl-propyl}-amide hydrochloride

Compound 327
Cyclopropanecarboxylic acid {(S)-3-[1-oxo-2-(1-oxy-pyridin-4-ylmethyl)-2,8-diaza-spiro[4.5)dec-8-yl]-1-phenyl-propyl}-amide hydrochloride

Compound 328
Cyclopentanecarboxylic acid { (S)-3-[1-oxo-2-(1-oxy-pyridin-4-ylmethyl)-2,8-diaza-spiro[4.5]dec-8-yl]-1-phenyl-propyl}-amide hydrochloride

Compound 329
Cyclopentanecarboxylic acid {3-[2-(4-bromo-benzyl)-1-oxo-2,8-diaza-spiro[4.5]dec-8-yl]-1-pyridin-2-yl-propyl}-amide dihydrochloride

Compound 330
N-{3-[2-(4-Bromo-benzyl)-1-oxo-2,8-diaza-spiro[4.5]dec-8-yl]-1-pyridin-2-yl-propyl}-propionamide dihydrochloride

Compound 331
N-{3-[2-(4-Bromo-benzyl)-1-oxo-2,8-diaza-spiro[4.5]dec-8-yl]-1-pyridin-2-yl-propyl}-2-methoxy-acetamide dihydrochloride

Compound 332
Cyclopropanecarboxylic acid {3-[2-(4-methoxy-benzyl)-1-oxo-2,8-diaza-spiro[4.5)dec-8-yl]-1-pyridin-2-yl-propyl}-amide dihydrochloride

Compound 333
N-{3-[2-(4-Methoxy-benzyl)-1-oxo-2,8-diaza-spiro[4.5]dec-8-yl]-1-pyridin-2-yl-propyl}-isobutyramide dihydrochloride

Compound 334
Cyclobutanecarboxylic acid {3-[2-(4-methoxy-benzyl)-1-oxo-2,8-diaza-spiro[4.5]dec-8-yl]-1-pyridin-2-yl-propyl}-amide dihydrochloride

Compound 335
Cyclopentanecarboxylic acid {3-[2-(4-methoxy-benzyl)-1-oxo-2,8-diaza-spiro[4.5]dec-8-yl]-1-pyridin-2-yl-propyl}-amide dihydrochloride

Compound 336
N-{3-[2-(4-Methoxy-benzyl)-1-oxo-2,8-diaza-spiro[4.5]dec-8-yl]-1-pyridin-2-yl-propyl}-propionamide dihydrochloride

Compound 337
2-Methoxy-N-{3-[2-(4-methoxy-benzyl)-1-oxo-2,8-diaza-spiro[4.5]dec-8-yl]-1-pyridin-2-yl-propyl}-acetamide dihydrochloride

Compound 338
Cyclopropanecarboxylic acid {3-[2-(4-methanesulfonyl-benzyl)-1-oxo-2,8-diaza-spiro[4.5]dec-8-yl]-1-pyridin-2-yl-propyl}-amide dihydrochloride

Compound 339
N-{3-[2-(4-Methanesulfonyl-benzyl)-1-oxo-2,8-diaza-spiro[4.5]dec-8-yl]-1-pyridin-2-yl-propyl)-isobutyramide dihydrochloride

Compound 340
Cyclobutanecarboxylic acid {3-[2-(4-methanesulfonyl-benzyl)-1-oxo-2,8-diaza-spiro[4.5]dec-8-yl]-1-pyridin-2-yl-propyl}-amide dihydrochloride

Compound 341
Cyclopropanecarboxylic acid {3-[2-(4-bromo-benzyl)-1-oxo-2,8-diaza-spiro[4.5]dec-8-yl]-1-pyridin-2-yl-propyl}-amide dihydrochloride

Compound 342
N-{3-[2-(4-Bromo-benzyl)-1-oxo-2,8-diaza-spiro[4.5]dec-8-yl]-1-pyridin-2-yl-propyl}-isobutyramide dihydrochloride

Compound 343
Cyclobutanecarboxylic acid {3-[2-(4-bromo-benzyl)-1-oxo-2,8-diaza-spiro[4.5]dec-8-yl]-1-pyridin-2-yl-propyl}-amide dihydrochloride

Compound 344
Cyclopentanecarboxylic acid {3-[2-(4-methanesulfonyl-benzyl)-1-oxo-2,8-diaza-spiro[4.5]dec-8-yl]-1-pyridin-2-yl-propyl}-amide dihydrochloride

Compound 345
N-{3-[2-(4-Methanesulfonyl-benzyl)-1-oxo-2,8-diaza-spiro[4.5]dec-8-yl]-1-pyridin-2-yl-propyl)-propionamide dihydrochloride

Compound 346
N-{(S)-3-[2-(4-Methanesulfonyl-benzyl)-1-oxo-2,8-diaza-spiro[4.5]dec-8-yl]-1-phenyl-propyl}-nicotinamide

Compound 347
(R)-Tetrahydro-furan-2-carboxylic acid {(S)-3-[2-(4-methanesulfonyl-benzyl)-1-oxo-2,8-diaza-spiro[4.5]dec-8-yl]-1-phenyl-propyl}-amide

Compound 348
(S)-Tetrahydro-furan-2-carboxylic acid {(S)-3-[2-(4-methanesulfonyl-benzyl)-1-oxo-2,8-diaza-spiro[4.5]dec-8-yl]-1-phenyl-propyl}-amide

Compound 349
Tetrahydro-furan-3-carboxylic acid {(S)-3-[2-(4-methanesulfonyl-benzyl)-1-oxo-2,8-diaza-spiro[4.5]dec-8-yl]-1-phenyl-propyl}-amide

Compound 350
(R)-N-{(S)-3-[2-(4-Methanesulfonyl-benzyl)-1-oxo-2,8-diaza-spiro[4.5]dec-8-yl]-1-phenyl-propyl}-2-phenyl-propionamide

Compound 351
3-Oxo-cyclopentanecarboxylic acid {(S)-3-[2-(4-methanesulfonyl-benzyl)-1-oxo-2,8-diaza-spiro[4.5]dec-8-yl]-1-phenyl-propyl}-amide

Compound 352
(S)-N-{(S)-3-[2-(4-Methanesulfonyl-benzyl)-1-oxo-2,8-diaza-spiro[4.5]dec-8-yl]-1-phenyl-propyl}-2-phenyl-propionamide

Compound 353
(R)-N-[(S)-3-(1-Oxo-2-pyridin-3-ylmethyl-2,8-diaza-spiro[4.5]dec-8-yl)-1-phenyl-propyl]-2-phenyl-propionamide

Compound 354
(S)-N-[(S)-3-(1-Oxo-2-pyridin-3-ylmethyl-2,8-diaza-spiro[4.5]dec-8-yl)-1-phenyl-propyl]-2-phenyl-propionamide

Compound 355
(R)-Tetrahydro-furan-2-carboxylic acid [(S)-3-(1-oxo-2-pyridin-3-ylmethyl-2,8-diaza-spiro[4.5]dec-8-yl)-1-phenyl-propyl]-amide

Compound 356
(S)-Tetrahydro-furan-2-carboxylic acid [(S)-3-(1-oxo-2-pyridin-3-ylmethyl-2,8-diaza-spiro[4.5]dec-8-yl)-1-phenyl-propyl]-amide

Compound 357
Tetrahydro-furan-3-carboxylic acid [(S)-3-(1-oxo-2-pyridin-3-ylmethyl-2,8-diaza-spiro[4.5]dec-8-yl)-1-phenyl-propyl]-amide

Compound 358
3-Oxo-cyclopentanecarboxylic acid [(S)-3-(1-oxo-2-pyridin-3-ylmethyl-2,8-diaza-spiro[4.5]dec-8-yl)-1-phenyl-propyl]-amide

Compound 359
N-[(S)-3-(1-Oxo-2-pyridin-2-ylmethyl-2,8-diaza-spiro[4.5]dec-8-yl)-1-phenyl-propyl]-isobutyramide

Compound 360
N-[(S)-3-(1-Oxo-2-pyridin-3-ylmethyl-2,8-diaza-spiro[4.5]dec-8-yl)-1-phenyl-propyl]-isobutyramide

Compound 361
N-[(S)-3-(1-Oxo-2-pyridin-4-ylmethyl-2,8-diaza-spiro[4.5)dec-8-yl)-1-phenyl-propyl]-isobutyramide

Compound 362
N-{(S)-3-[1-Oxo-2-(1-oxy-pyridin-4-ylmethyl)-2,8-diaza-spiro[4.5]dec-8-yl]-1-phenyl-propyl}-isobutyramide

Compound 363
N-{(S)-3-[1-Oxo-2-(1-oxy-pyridin-2-ylmethyl)-2,8-diaza-spiro[4.5]dec-8-yl]-1-phenyl-propyl}-isobutyramide

Compound 364
N-{(S)-3-[1-Oxo-2-(1-oxy-pyridin-3-ylmethyl)-2,8-diaza-spiro[4.5]dec-8-yl]-1-phenyl-propyl}-isobutyramide

Compound 365
1-Methyl-cyclopentanecarboxylic acid {(S)-3-[2-(4-methanesulfonyl-benzyl)-1-oxo-2,8-diaza-spiro[4.5]dec-8-yl]-1-phenyl-propyl}-amide

Compound 366
1-Methyl-cyclohexanecarboxylic acid{(S)-3-[2-(4-methanesulfonyl-benzyl)-1-oxo-2,8-diaza-spiro[4.5]dec-8-yl]-1-phenyl-propyl}-amide

Compound 367
2-Cyclopentyl-N-{(S)-3-[2-(4-methanesulfonyl-benzyl)-1-oxo-2,8-diaza-spiro[4.5]dec-8-yl]-1-phenyl-propyl}-propionamide

Compound 368
2-Cyclopentyl-N-{(S)-3-[2-(4-methanesulfonyl-benzyl)-1-oxo-2,8-diaza-spiro[4.5]dec-8-yl]-1-phenyl-propyl}-propionamide

Compound 369
1-Methyl-cyclopentanecarboxylic acid [(S)-3-(1-oxo-2-pyridin-3-ylmethy-2,8-diaza-spiro[4.5]dec-8-yl)-1-phenyl-propyl]-amide

Compound 370
1-Methyl-cyclohexanecarboxylic acid[(S)-3-(1-oxo-2-pyridin-3-ylmethyl-2,8-diaza-spiro[4.5]dec-8-yl)-1-phenyl-propyl]-amide

Compound 371
2-Cyclopentyl-N-[(S)-3-(1-oxo-2-pyridin-3-ylmethyl-2,8-diaza-spiro[4.5)dec-8-yl)-1-phenyl-propyl]-propionamide

Compound 372
2-Cyclopentyl-N-[(S)-3-(1-oxo-2-pyridin-3-ylmethyl-2,8-diaza-spiro[4.5]dec-8-yl)-1-phenyl-propyl]-propionamide

Compound 373
Cyclopropanecarboxylic acid (3-[2-(4-bromo-benzyl)-1-oxo-2,8-diaza-spiro[4.5]dec-8-yl]-1-(2-methoxy-phenyl)-propyl]-amide hydrochloride

Compound 374
N-[3-[2-(4-Bromo-benzyl)-1-oxo-2,8-diaza-spiro[4.5]dec-8-yl]-1-(2-methoxy-phenyl)-propyl]-isobutyramide hydrochloride

Compound 375
Cyclobutanecarboxylic acid [3-[2-(4-bromo-benzyl)-1-oxo-2,8-diaza-spiro[4.5]dec-8-yl]-1-(2-methoxy-phenyl)-propyl]-amide hydrochloride

Compound 376
Tetrahydro-pyran-2-carboxylic acid {(S)-3-[2-(4-methanesulfonyl-benzyl)-1-oxo-2,8-diaza-spiro[4.5]dec-8-yl]-1-phenyl-propyl}-amide

Compound 377
N-{3-[2-(4-Methoxy-benzyl)-1-oxo-2,8-diaza-spiro[4.5]dec-8-yl]-1-thiophen-2-yl-propyl}-2,2-dimethyl-propionamide hydrochloride

Compound 378
N-{3-[2-(4-Methanesulfonyl-benzyl)-1-oxo-2,8-diaza-spiro[4.5]dec-8-yl]-1-thiophen-2-yl-propyl}-2,2-dimethyl-propionamide hydrochloride

Compound 379
N-{l-(3-Chloro-phenyl)-3-[2-(4-methoxy-benzyl)-1-oxo-2,8-diaza-spiro[4.5]dec-8-yl]-propyl}-2,2-dimethyl-propionamide hydrochloride

Compound 380
N-{1-(3-Chloro-phenyl)-3-[2-(4-methanesulfonyl-benzyl)-1-oxo-2,8-diaza-spiro[4.5]dec-8-yl]-propyl}-2,2-dimethyl-propionamide hydrochloride

Compound 381
N-[1-(3-Chloro-phenyl)-3-(1-oxo-2-pyridin-3-ylmethyl-2,8-diaza-spiro[4.5]dec-8-yl)-propyl]-2,2-dimethyl-propionamide dihydrochloride

Compound 382
N-{1-(2-Chloro-phenyl)-3-[2-(4-methanesulfonyl-benzyl)-1-oxo-2,8-diaza-spiro[4.5)dec-8-yl]-propyl}-2,2-dimethyl-propionamide hydrochloride

Compound 383
N-{1-(4-Chloro-phenyl)-3-[2-(4-methanesulfonyl-benzyl)-1-oxo-2,8-diaza-spiro[4.5]dec-8-yl]-propyl}-2,2-dimethyl-propionamide hydrochloride

Compound 384
1-Methyl-cyclopropanecarboxylic acid {3-[2-(4-methoxy-benzyl)-1-oxo-2,8-diaza-spiro[4.5]dec-8-yl]-1-thiophen-2-yl-propyl}-amide hydrochloride

Compound 385
1-Methyl-cyclopropanecarboxylic acid {3-[2-(4-methanesulfonyl-benzyl)-1-oxo-2,8-diaza-spiro[4.5]dec-8-yl]-1-thiophen-2-yl-propyl}-amide hydrochloride

Compound 386
1-Methyl-cyclopropanecarboxylic acid [3-(1-oxo-2-pyridin-3-ylmethyl-2,8-diaza-spiro[4.5]dec-8-yl)-1-thiophen-2-yl-propyl]-amide dihydrochloride

Compound 387
1-Methyl-cyclopropanecarboxylic acid {1-(3-chloro-phenyl)-3-[2-(4-methoxy-benzyl)-1-oxo-2,8-diaza-spiro[4.5]dec-8-yl]-propyl}-amide hydrochloride

Compound 388
1-Methyl-cyclopropanecarboxylic acid {1-(3-chloro-phenyl)-3-[2-(4-methanesulfonyl-benzyl)-1-oxo-2,8-diaza-spiro[4.5]dec-8-yl]-propyl}-amide hydrochloride

Compound 389
1-Methyl-cyclopropanecarboxylic acid [1-(3-chloro-phenyl)-3-(l-oxo-2-pyridin-3-ylmethyl-2,8-diaza-spiro[4.5]dec-8-yl)-propyl]-amide dihydrochloride

Compound 390
1-Methyl-cyclopropanecarboxylic acid {1-(2-chloro-phenyl)-3-[2-(4-methanesulfonyl-benzyl)-1-oxo-2,8-diaza-spiro[4.5)dec-8-yl]-propyl}-amide hydrochloride

Compound 391
1-Methyl-cyclopropanecarboxylic acid {1- (4-chloro-phenyl)-3-[2-(4-methanesulfonyl-benzyl)-1-oxo-2,8-diaza-spiro[4.5]dec-8-yl]-propyl}-amide hydrochloride

Compound 392
Tetrahydro-pyran-2-carboxylic acid [(S)-3-(1-oxo-2-pyridin-3-ylmethyl-2,8-diaza-spiro[4.5]dec-8-yl)-1-phenyl-propyl]-amide

Compound 393
Cyclopentanecarboxylic acid [3-[2-(4-bromo-benzyl)-1-oxo-2,8-diaza-spiro[4.5]dec-8-yl]-1-(2-methoxy-phenyl)-propyl]-amide hydrochloride

Compound 394
N-[3-[2-(4-Bromo-benzyl)-1-oxo-2,8-diaza-spiro[4.5]dec-8-yl]-1-(2-methoxy-phenyl)-propyl]-propionamide hydrochloride

Compound 395
N-[3-[2-(4-Bromo-benzyl)-1-oxo-2,8-diaza-spiro[4.5]dec-8-yl]-1-(2-methoxy-phenyl)-propyl]-2-methoxy-acetamide hydrochloride

Compound 396
N-[3-[2-(4-Bromo-benzyl)-1-oxo-2,8-diaza-spiro[4.5]dec-8-yl]-1-(2-methoxy-phenyl)-propyl]-2-cyclopropyl-acetamide hydrochloride

Compound 397
Cyclohexanecarboxylic acid [3-[2-(4-bromo-benzyl)-1-oxo-2,8-diaza-spiro[4.5]dec-8-yl]-1-(2-methoxy-phenyl)-propyl]-amide hydrochloride

Compound 398
Cyclopropanecarboxylic acid [3-[2-(4-methoxy-benzyl)-1-oxo-2,8-diaza-spiro[4.5]dec-8-yl]-1-(2-methoxy-phenyl)-propyl]-amide hydrochloride

Compound 399
N-[3-[2-(4-Methoxy-benzyl)-1-oxo-2,8-diaza-spiro[4.5]dec-8-yl]-1-(2-methoxy-phenyl)-propyl]-isobutyramide hydrochloride

Compound 400
Cyclobutanecarboxylic acid [3-[2-(4-methoxy-benzyl)-1-oxo-2,8-diaza-spiro[4.5]dec-8-yl]-1-(2-methoxy-phenyl)-propyl]-amide hydrochloride

Compound 401
Cyclopentanecarboxylic acid [3-[2-(4-methoxy-benzyl)-1-oxo-2,8-diaza-spiro[4.5]dec-8-yl]-1-(2-methoxy-phenyl)-propyl]-amide hydrochloride

Compound 402
N-[3-[2-(4-Methoxy-benzyl)-1-oxo-2,8-diaza-spiro[4.5]dec-8-yl]-1-(2-methoxy-phenyl)-propyl]-propionamide hydrochloride

Compound 403
2-Methoxy-N-[3-[2-(4-methoxy-benzyl)-1-oxo-2,8-diaza-spiro[4.5]dec-8-yl]-1-(2-methoxy-phenyl)-propyl]-acetamide hydrochloride

Compound 404
2-Cyclopropyl-N-[3-[2-(4-methoxy-benzyl)-1-oxo-2,8-diaza-spiro[4.5]dec-8-yl]-1(2-methoxy-phenyl)-propyl]-acetamide hydrochloride

Compound 405
Cyclohexanecarboxylic acid [3-[2-(4-methoxy-benzyl)-1-oxo-2,8-diaza-spiro[4.5]dec-8-yl)-1-(2-methoxy-phenyl)-propyl]-amide hydrochloride

Compound 406
Cyclopropanecarboxylic acid [3-[2-(4-methanesulfonyl-benzyl)-1-oxo-2,8-diaza-spiro[4.5]dec-8-yl]-1-(2-methoxy-phenyl)-propyl]-amide hydrochloride

Compound 407
N-[3-[2-(4-Methanesulfonyl-benzyl)-1-oxo-2,8-diaza-spiro[4.5]dec-8-yl]-1-(2-methoxy-phenyl)-propyl]-isobutyramide hydrochloride

Compound 408
Cyclobutanecarboxylic acid [3-[2-(4-methanesulfonyl-benzyl)-1-oxo-2,8-diaza-spiro[4.5]dec-8-yl]-1-(2-methoxy-phenyl)-propyl]-amide hydrochloride

Compound 409
Cyclopentanecarboxylic acid (3-[2-(4-methanesulfonyl-benzyl)-1-oxo-2,8-diaza-spiro[4.5]dec-8-yl]-1-(2-methoxy-phenyl)-propyl]-amide hydrochloride

Compound 410
N-[3-[2-(4-Methanesulfonyl-benzyl)-1-oxo-2,8-diaza-spiro[4.5]dec-8-yl]-1-(2-methoxy-phenyl)-propyl]-propionamide hydrochloride

Compound 411
N-[3-[2-(4-Methanesulfonyl-benzyl)-1-oxo-2,8-diaza-spiro[4.5]dec-8-yl]-1-(2-methoxy-phenyl)-propyl]-2-methoxy-acetamide hydrochloride

Compound 412
2-Cyclopropyl-N-[3-[2-(4-methanesulfonyl-benzyl)-1-oxo-2,8-diaza-spiro[4.5]dec-8-yl]-1-(2-methoxy-phenyl)-propyl]-acetamide hydrochloride

Compound 413
Cyclopropanecarboxylic acid [3-[2-(4-bromo-benzyl)-1-oxo-2,8-diaza-spiro[4.5]dec-8-yl]-1-(4-methoxy-phenyl)-propyl]-amide hydrochloride

Compound 414
N-[3-[2-(4-Bromo-benzyl)-1-oxo-2,8-diaza-spiro[4.5]dec-8-yl]-1-(4-methoxy-phenyl)-propyl]-isobutyramide hydrochloride

Compound 415
Cyclobutanecarboxylic acid [3-[2-(4-bromo-benzyl)-1-oxo-2,8-diaza-spiro[4.5]dec-8-yl]-1-(4-methoxy-phenyl)-propyl]-amide hydrochloride

Compound 416
Cyclopentanecarboxylic acid [3-[2-(4-bromo-benzyl)-1-oxo-2,8-diaza-spiro[4.5]dec-8-yl]-1-(4-methoxy-phenyl)-propyl]-amide hydrochloride

Compound 417
N-[3-[2-(4-Bromo-benzyl)-1-oxo-2,8-diaza-spiro[4.5]dec-8-yl]-1-(4-methoxy-phenyl)-propyl)-propionamide hydrochloride

Compound 418
N-[3-[2-(4-Bromo-benzyl)-1-oxo-2,8-diaza-spiro[4.5]dec-8-yl]-1-(4-methoxy-phenyl)-propyl]-2-methoxy-acetamide hydrochloride

Compound 419
Cyclopropanecarboxylic acid [3-[2-(4-methoxy-benzyl)-1-oxo-2,8-diaza-spiro[4.5]dec-8-yl]-1-(4-methoxy-phenyl)-propyl]-amide hydrochloride

Compound 420
N-[3-[2-(4-Methoxy-benzyl)-1-oxo-2,8-diaza-spiro[4.5]dec-8-yl]-1-(4-methoxy-phenyl)-propyl]-isobutyramide hydrochloride

Compound 421
Cyclobutanecarboxylic acid [3-[2-(4-methoxy-benzyl)-1-oxo-2,8-diaza-spiro[4.5]dec-8-yl]-1-(4-methoxy-phenyl)-propyl]-amide hydrochloride

Compound 422
Cyclopentanecarboxylic acid [3-[2-(4-methoxy-benzyl)-1-oxo-2,8-diaza-spiro[4.5]dec-8-yl]-1-(4-methoxy-phenyl)-propyl]-amide hydrochloride

Compound 423
N-[3-[2-(4-Methoxy-benzyl)-1-oxo-2,8-diaza-spiro[4.5]dec-8-yl]-1-(4-methoxy-phenyl)-propyl]-propionamide hydrochloride

Compound 424
Cyclohexanecarboxylic acid [3-[2-(4-methanesulfonyl-benzyl)-1-oxo-2,8-diaza-spiro[4.5]dec-8-yl]-1-(2-methoxy-phenyl)-propyl]-amide hydrochloride

Compound 425
Cyclopentanecarboxylic acid [1-(3-methoxy-phenyl)-3-(1-oxo-2-pyridin-3-ylmethyl-2,8-diaza-spiro[4.5]dec-8-yl)-propyl]-amide dihydrochloride

Compound 426
N-[1-(3-Methoxy-phenyl)-3-(1-oxo-2-pyridin-3-ylmethyl-2,8-diaza-spiro[4.5]dec-8-yl)-propyl]-propionamide dihydrochloride

Compound 427
2-Methoxy-N-[1-(3-methoxy-phenyl)-3-(1-oxo-2-pyridin-3-ylmethyl-2,8-diaza-spiro[4.5]dec-8-yl)-propyl]-acetamide dihydrochloride

Compound 428
3-Hydroxy-cyclopentanecarboxylic acid {(S)-3-[2-(4-methanesulfonyl-benzyl)-1-oxo-2,8-diaza-spiro[4.5]dec-8-yl]-1-phenyl-propyl}-amide

Compound 429
3-Hydroxy-cyclopentanecarboxylic acid [(S)-3-(1-oxo-2-pyridin-3-ylmethyl-2,8-diaza-spiro[4.5]dec-8-yl)-1-phenyl-propyl]-amide

Compound 430
Cyclopropanecarboxylic acid {1-(2-chloro-phenyl)-3-[2-(4-methoxy-benzyl)-1-oxo-2,8-diaza-spiro[4.5]dec-8-yl]-propyl}-amide hydrochloride

Compound 431
N-{1-(2-Chloro-phenyl)-3-[2-(4-methoxy-benzyl)-1-oxo-2,8-diaza-spiro[4.5]dec-8-yl]-propyl}-isobutyramide hydrochloride

Compound 432
Cyclobutanecarboxylic acid {1-(2-chloro-phenyl)-3-[2-(4-methoxy-benzyl)-1-oxo-2,8-diaza-spiro[4.5]dec-8-yl]-propyl}-amide hydrochloride

Compound 433
Cyclopentanecarboxylic acid {1-(2-chloro-phenyl)-3-[2-(4-methoxy-benzyl)-1-oxo-2,8-diaza-spiro[4.5]dec-8-yl]-propyl}-amide hydrochloride

Compound 434
N-{1-(2-Chloro-phenyl)-3-[2-(4-methoxy-benzyl)-1-oxo-2,8-diaza-spiro[4.5]dec-8-yl]-propyl}-propionamide hydrochloride

Compound 435
N-{1-(2-Chloro-phenyl)-3-[2-(4-methoxy-benzyl)-1-oxo-2,8-diaza-spiro[4.5]dec-8-yl]-propyl}-2-methoxy-acetamide hydrochloride

Compound 436
Cyclohexanecarboxylic acid {1-(2-chloro-phenyl)-3-[2-(4-methoxy-benzyl)-1-oxo-2,8-diaza-spiro[4.5]dec-8-yl]-propyl}-amide hydrochloride

Compound 437
Cyclopropanecarboxylic acid {1-(2-chloro-phenyl)-3-[2-(4-methanesulfonyl-benzyl)-1-oxo-2,8-diaza-spiro[4.5]dec-8-yl]-propyl}-amide hydrochloride

Compound 438
N-{1-(2-Chloro-phenyl)-3-[2-(4-methanesulfonyl-benzyl)-1-oxo-2,8-diaza-spiro[4.5]dec-8-yl]-propyl}-isobutyramide hydrochloride

Compound 439
Cyclobutanecarboxylic acid {1-(2-chloro-phenyl)-3-[2-(4-methanesulfonyl-benzyl)-1-oxo-2,8-diaza-spiro[4.5]dec-8-yl]-propyl}-amide hydrochloride

Compound 440
Cyclopentanecarboxylic acid {1-(2-chloro-phenyl)-3-[2-(4-methanesulfonyl-benzyl)-1-oxo-2,8-diaza-spiro[4.5]dec-8-yl]-propyl}-amide hydrochloride

Compound 441
N-{1-(2-Chloro-phenyl)-3-[2-(4-methanesulfonyl-benzyl)-1-oxo-2,8-diaza-spiro[4.5]dec-8-yl]-propyl}-propionamide hydrochloride

Compound 442
N-{1-(2-Chloro-phenyl)-3-[2-(4-methanesulfonyl-benzyl)-1-oxo-2,8-diaza-spiro[4.5]dec-8-yl]-propyl}-2-methoxy-acetamide hydrochloride

Compound 443
Cyclohexanecarboxylic acid {1-(2-chloro-phenyl)-3-[2-(4-methanesulfonyl-benzyl)-1-oxo-2,8-diaza-spiro[4.5]dec-8-yl]-propyl}-amide hydrochloride

Compound 444
Cyclopropanecarboxylic acid [1-(2-chloro-phenyl)-3-(1-oxo-2-pyridin-3-ylmethyl-2,8-diaza-spiro[4.5]dec-8-yl)-propyl]-amide dihydrochloride

Compound 445
N-[1-(2-Chloro-phenyl)-3-(1-oxo-2-pyridin-3-ylmethyl-2,8-diaza-spiro[4.5]dec-8-yl)-propyl]-isobutyramide dihydrochloride

Compound 446
Cyclobutanecarboxylic acid [1-(2-chloro-phenyl)-3-(1-oxo-2-pyridin-3-ylmethyl-2,8-diaza-spiro[4.5]dec-8-yl)-propyl]-amide dihydrochloride

Compound 447
Cyclopentanecarboxylic acid [1-(2-chloro-phenyl)-3-(1-oxo-2-pyridin-3-ylmethyl-2,8-diaza-spiro[4.5]dec-8-yl)-propyl]-amide dihydrochloride

Compound 448
N-[1-(2-Chloro-phenyl)-3-(1-oxo-2-pyridin-3-ylmethyl-2,8-diaza-spiro[4.5]dec-8-yl)-propyl]-propionamide dihydrochloride

Compound 449
N-[1-(2-Chloro-phenyl)-3-(1-oxo-2-pyridin-3-ylmethyl-2,8-diaza-spiro[4.5]dec-8-yl)-propyl]-2-methoxy-acetamide dihydrochloride

Compound 450
Cyclohexanecarboxylic acid [1-(2-chloro-phenyl)-3-(1-oxo-2-pyridin-3-ylmethyl-2,8-diaza-spiro[4.5]dec-8-yl)-propyl]-amide dihydrochloride

Compound 451
Cyclopropanecarboxylic acid {1-(4-chloro-phenyl)-3-[2-(4-methoxy-benzyl)-1-oxo-2,8-diaza-spiro[4.5]dec-8-yl]-propyl}-amide hydrochloride

Compound 452
N-{1-(4-Chloro-phenyl)-3-[2-(4-methoxy-benzyl)-1-oxo-2,8-diaza-spiro[4.5]dec-8-yl]-propyl}-isobutyramide hydrochloride

Compound 453
Cyclobutanecarboxylic acid {1-(4-chloro-phenyl)-3-[2-(4-methoxy-benzyl)-1-oxo-2,8-diaza-spiro[4.5]dec-8-yl]-propyl}-amide hydrochloride

Compound 454
Cyclopentanecarboxylic acid {1-(4-chloro-phenyl)-3-[2-(4-methoxy-benzyl)-1-oxo-2,8-diaza-spiro[4.5]dec-8-yl]-propyl}-amide hydrochloride

Compound 455
N-{1-(4-Chloro-phenyl)-3-[2-(4-methoxy-benzyl)-1-oxo-2,8-diaza-spiro[4.5]dec-8-yl]-propyl}-propionamide hydrochloride

Compound 456
N-{1-(4-Chloro-phenyl)-3-[2-(4-methoxy-benzyl)-1-oxo-2,8-diaza-spiro[4.5]dec-8-yl]-propyll-2-methoxy-acetamide hydrochloride

Compound 457
Cyclohexanecarboxylic acid {1-(4-chloro-phenyl)-3-[2-(4-methoxy-benzyl)-1-oxo-2,8-diaza-spiro[4.5]dec-8-yl]-propyl}-amide hydrochloride

Compound 458
N-{1-(4-Chloro-phenyl)-3-[2-(4-methanesulfonyl-benzyl)-1-oxo-2,8-diaza-spiro[4.5]dec-8-yl]-propyl}-propionamide hydrochloride

Compound 459
N-{1-(4-Chloro-phenyl)-3-[2-(4-methanesulfonyl-benzyl)-1-oxo-2,8-diaza-spiro[4.5]dec-8-yl]-propyl}-2-methoxy-acetamide hydrochloride

Compound 460
Cyclohexanecarboxylic acid {1-(4-chloro-phenyl)-3-[2-(4-methanesulfonyl-benzyl)-1-oxo-2,8-diaza-spiro[4.5]dec-8-yl]-propyl}-amide hydrochloride

Compound 461
Cyclopropanecarboxylic acid [1-(4-chloro-phenyl)-3-(1-oxo-2-pyridin-3-ylmethyl-2,8-diaza-spiro[4.5]dec-8-yl)-propyl]-amide dihydrochloride

Compound 462
N-[1-(4-Chloro-phenyl)-3-(1-oxo-2-pyridin-3-ylmethyl-2,8-diaza-spiro[4.5]dec-8-yl)-propyl]-isobutyramide dihydrochloride

Compound 463
Cyclobutanecarboxylic acid [1-(4-chloro-phenyl)-3-(1-oxo-2-pyridin-3-ylmethyl-2,8-diaza-spiro[4.5]dec-8-yl)-propyl)-amide dihydrochloride

Compound 464
Cyclopentanecarboxylic acid [1-(4-chloro-phenyl)-3-(1-oxo-2-pyridin-3-ylmethyl-2,8-diaza-spiro[4.5]dec-8-yl)-propyl]-amide dihydrochloride

Compound 465
N-[1-(4-Chloro-phenyl)-3-(1-oxo-2-pyridin-3-ylmethyl-2,8-diaza-spiro[4.5]dec-8-yl)-propyl]-propionamide dihydrochloride

Compound 466
N-[1-(4-Chloro-phenyl)-3-(1-oxo-2-pyridin-3-ylmethyl-2,8-diaza-spiro[4.5]dec-8-yl)-propyl]-2-methoxy-acetamide dihydrochloride

Compound 467
Cyclohexanecarboxylic acid [1-(4-chloro-phenyl)-3-(1-oxo-2-pyridin-3-ylmethyl-2,8-diaza-spiro[4.5]dec-8-yl)-propyl]-amide dihydrochloride

Compound 468
Cyclopropanecarboxylic acid [3-[2-(4-bromo-benzyl)-1-oxo-2,8-diaza-spiro[4.5]dec-8-yl]-1-(2-chloro-phenyl)-propyl]-amide hydrochloride

Compound 469
Cyclopropanecarboxylic acid [3-[2-(4-bromo-benzyl)-1-oxo-2,8-diaza-spiro[4.5]dec-8-yl]-1-(4-chloro-phenyl)-propyl]-amide hydrochloride

Compound 470
Cyclopropanecarboxylic acid {l-(3,4-dichloro-phenyl)-3-[2-(4-methoxy-benzyl)-1-oxo-2,8-diaza-spiro[4.5]dec-8-yl]-propyl}-amide hydrochloride

Compound 471
Cyclopropanecarboxylic acid {l-(3,4-dichloro-phenyl)-3-[2-(4-methanesulfonyl-benzyl)-1-oxo-2,8-diaza-spiro[4.5]dec-8-yl]-propyl}-amide hydrochloride

Compound 472
Cyclopropanecarboxylic acid [1-(3,4-dichloro-phenyl)-3-(1-oxo-2-pyridin-3-ylmethyl-2,8-diaza-spiro[4.5]dec-8-yl)-propyl]-amide dihydrochloride

Compound 473
Cyclopentanecarboxylic acid [3-[2-(4-bromo-benzyl)-1-oxo-2,8-diaza-spiro[4.5]dec-8-yl]-1-(2-chloro-phenyl)-propyl]-amide hydrochloride

Compound 474
Cyclopentanecarboxylic acid [3-[2-(4-bromo-benzyl)-1-oxo-2,8-diaza-spiro[4.5]dec-8-yl]-1-(4-chloro-phenyl)-propyl]-amide hydrochloride

Compound 475
Cyclopentanecarboxylic acid {(S)-3-[2-(4-fluoro-benzyl)-1-oxo-2,8-diaza-spiro[4.5]dec-8-yl]-1-phenyl-propyl}-amide hydrochloride

Compound 476
Cyclopentanecarboxylic acid {(S)-3-[2-(4-chloro-benzyl)-1-oxo-2,8-diaza-spiro[4.5]dec-8-yl]-1-phenyl-propyl}-amide hydrochloride

Compound 477
Cyclopentanecarboxylic acid {(S)-3-[2-(4-cyano-benzyl)-1-oxo-2,8-diaza-spiro[4.5]dec-8-yl]-1-phenyl-propyl}-amide hydrochloride

Compound 478
Cyclopentanecarboxylic acid {(S)-3-[2-(4-difluoromethoxy-benzyl)-1-oxo-2,8-diaza-spiro[4.5]dec-8-yl]-1-phenyl-propyl}-amide hydrochloride

Compound 479
Cyclopentanecarboxylic acid {(S)-3-[l-oxo-2-(4-trifluoromethoxy-benzyl)-2,8-diaza-spiro[4.5]dec-8-yl]-1-phenyl-propyl}-amide hydrochloride

Compound 480
Cyclopentanecarboxylic acid {(S)-3-[2-(4-methylsulfanyl-benzyl)-1-oxo-2,8-diaza-spiro[4.5]dec-8-yl]-1-phenyl-propyl}-amide hydrochloride

Compound 481
Cyclopentanecarboxylic acid {(S)-3-[1-oxo-2-(4-pyrazol-1-yl-benzyl)-2,8-diaza-spiro[4.5]dec-8-yl]-1-phenyl-propyl}-amide hydrochloride

Compound 482
Cyclopentanecarboxylic acid [(S)-3-(2-isobutyl-1-oxo-2,8-diaza-spiro[4.5]dec-8-yl)-1-phenyl-propyl]-amide hydrochloride

Compound 483
Cyclopentanecarboxylic acid {(S)-3-[2-(4-methanesulfonyl-benzyl)-3-oxo-2,8-diaza-spiro[4.5]dec-8-yl]-1-phenyl-propyl}-amide hydrochloride

Compound 484
Cyclopentanecarboxylic acid { (S)-3-[2-(4-methoxy-benzyl)-3-oxo-2,8-diaza-spiro[4.5]dec-8-yl]-1-phenyl-propyl}-amide hydrochloride

Compound 485
Cyclopropanecarboxylic acid {(S)-3-[2-(4-fluoro-benzyl)-1-oxo-2,8-diaza-spiro[4.5)dec-8-yl]-1-phenyl-propyl}-amide hydrochloride

Compound 486
Cyclopropanecarboxylic acid {(S)-3-[2-(4-chloro-benzyl)-1-oxo-2,8-diaza-spiro[4.5]dec-8-yl]-1-phenyl-propyl}-amide hydrochloride

Compound 487
Cyclopropanecarboxylic acid {(S)-3-[2-(4-cyano-benzyl)-1-oxo-2,8-diaza-spiro[4.5)dec-8-yl]-1-phenyl-propyl}-amide hydrochloride

Compound 488
Cyclopropanecarboxylic acid {(S)-3-[2- (4-difluoromethoxy-benzyl)-1-oxo-2,8-diaza-spiro[4.5]dec-8-yl]-1-phenyl-propyl}-amide hydrochloride

Compound 489
Cyclopropanecarboxylic acid {(S)-3-[1-oxo-2-(4-pyrazol-1-yl-benzyl)-2,.8-diaza-spiro[4.5]dec-8-yl]-1-phenyl-propyl}-amide hydrochloride

Compound 490
Cyclopropanecarboxylic acid [(S)-3-(2-cyclohexylmethyl-1-oxo-2,8-diaza-spiro[4.5]dec-8-yl)-1-phenyl-propyl]-amide hydrochloride

Compound 491
Cyclopropanecarboxylic acid [(S)-3-(2-isobutyl-1-oxo-2,8-diaza-spiro[4.5]dec-8-yl)-1-phenyl-propyl]-amide hydrochloride

Compound 492
Cyclopropanecarboxylic acid {(S)-3-[2-(4-methanesulfonyl-benzyl)-3-oxo-2,8-diaza-spiro[4.5]dec-8-yl]-1-phenyl-propyl}-amide hydrochloride

Compound 493
Cyclopropanecarboxylic acid {(S)-3-[2-(4-methoxy-benzyl)-3-oxo-2,8-diaza-spiro[4.5]dec-8-yl]-1-phenyl-propyl}-amide hydrochloride

Compound 494
N-[(S)-3-(2-Benzyl-1-oxo-2,8-diaza-spiro[4.5]dec-8-yl)-1-phenyl-propyl]-isobutyramide hydrochloride

Compound 495
N-{(S)-3-[2-(4-Fluoro-benzyl)-1-oxo-2,8-diaza-spiro[4.5]dec-8-yl]-1-phenyl-propyl}-isobutyramide hydrochloride

Compound 496
Cyclopropanecarboxylic acid {(S)-3-[1-oxo-2-(4-trifluoromethoxy-benzyl)-2,8-diaza-spiro[4.5]dec-8-yl)-1-phenyl-propyl}-amide hydrochloride

Compound 497
Cyclopropanecarboxylic acid {(S)-3-[2-(4-methylsulfanyl-benzyl)-1-oxo-2,8-diaza-spiro[4.5]dec-8-yl]-1-phenyl-propyl}-amide hydrochloride

Compound 498
N-{(S)-3-[2-(4-Chloro-benzyl)-1-oxo-2,8-diaza-spiro[4.5]dec-8-yl]-1-phenyl-propyl}-isobutyramide hydrochloride

Compound 499
N-{(S)-3-[2-(4-Cyano-benzyl)-1-oxo-2,8-diaza-spiro[4.5]dec-8-yl]-1-phenyl-propyl}-isobutyramide hydrochloride

Compound 500
N-{(S)-3-[2-(4-Difluoromethoxy-benzyl)-1-oxo-2,8-diaza-spiro[4.5]dec-8-yl)-1-phenyl-propyl}-isobutyramide hydrochloride

Compound 501
N-{(S)-3-[1-Oxo-2-(4-trifluoromethoxy-benzyl)-2,8-diaza-spiro[4.5]dec-8-yl]-1-phenyl-propyl}-isobutyramide hydrochloride

Compound 502
N-{(S)-3-[2-(4-Methylsulfanyl-benzyl)-1-oxo-2,8-diaza-spiro[4.5)dec-8-yl]-1-phenyl-propyl}-isobutyramide hydrochloride

Compound 503
N-{(S)-3-[2-(4-Methoxy-benzyl)-3-oxo-2,8-diaza-spiro[4.5]dec-8-yl]-1-phenyl-propyl}-isobutyramide hydrochloride

Compound 504
2-Methoxy-cyclopent-1-enecarboxylic acid {(S)-3-[2-(4-methanesulfonyl-benzyl)-1-oxo-2,8-diaza-spiro[4.5]dec-8-yl]-1-phenyl-propyl}-amide

Compound 505
2-Methoxy-cyclopent-1-enecarboxylic acid [(S)-3-(1-oxo-2-pyridin-3-ylmethyl-2,8-diaza-spiro[4.5]dec-8-yl)-1-phenyl-propyl]-amide

Compound 506
Cyclopentanecarboxylic acid {1-(3,4-dichloro-phenyl)-3-[2-(4-methoxy-benzyl)-1-oxo-2,8-diaza-spiro[4.5]dec-8-yl]-propyl}-amide hydrochloride

Compound 507
Cyclopentanecarboxylic acid {1-(3,4-dichloro-phenyl)-3-[2-(4-methanesulfonyl-benzyl)-1-oxo-2,8-diaza-spiro[4.5]dec-8-yl]-propyl}-amide hydrochloride

Compound 508
Cyclopentanecarboxylic acid [1-(3,4-dichloro-phenyl)-3-(1-oxo-2-pyridin-3-ylmethyl-2,8-diaza-spiro[4.5]dec-8-yl)-propyl]-amide dihydrochloride

Compound 509
N-{(S)-3-[2-(4-Methoxy-benzyl)-1-oxo-2,8-diaza-spiro[4.5]dec-8-yl]-1-phenyl-propyl}-propionamide hydrochloride

Compound 510
N-{(S)-3-[2-(4-Methoxy-benzyl)-1-oxo-2,8-diaza-spiro[4.5]dec-8-yl]-1-phenyl-propyl}-2,2-dimethyl-propionamide hydrochloride

Compound 511
Thiophene-2-carboxylic acid {(S)-3-[2-(4-methoxy-benzyl)-1-oxo-2,8-diaza-spiro[4.5]dec-8-yl]-1-phenyl-propyl}-amide hydrochloride

Compound 512
Thiophene-3-carboxylic acid {(S)-3-[2-(4-methoxy-benzyl)-1-oxo-2,8-diaza-spiro[4.5]dec-8-yl]-1-phenyl-propyl}-amide hydrochloride

Compound 513
(R)-Tetrahydro-furan-2-carboxylic acid {(S)-3-[2-(4-methoxy-benzyl)-1-oxo-2,8-diaza-spiro[4.5]dec-8-yl]-1-phenyl-propyl}-amide hydrochloride

Compound 514
(S)-Tetrahydro-furan-2-carboxylic acid {(S)-3-[2-(4-methoxy-benzyl)-1-oxo-2,8-diaza-spiro[4.5]dec-8-yl]-1-phenyl-propyl}-amide hydrochloride

Compound 515
Tetrahydro-furan-3-carboxylic acid {(S)-3-[2-(4-methoxy-benzyl)-1-oxo-2,8-diaza-spiro[4.5]dec-8-yl]-1-phenyl-propyl}-amide hydrochloride

Compound 516
3-Oxo-cyclopentanecarboxylic acid {(S)-3-[2-(4-methoxy-benzyl)-1-oxo-2,8-diaza-spiro[4.5]dec-8-yl]-1-phenyl-propyl}-amide hydrochloride

Compound 517
4,4-Difluoro-cyclohexanecarboxylic acid {(S)-3-[2-(4-methoxy-benzyl)-1-oxo-2,8-diaza-spiro[4.5]dec-8-yl]-1-phenyl-propyl}-amide hydrochloride

Compound 518
Tetrahydro-pyran-4-carboxylic acid {(S)-3-[2-(4-methoxy-benzyl)-1-oxo-2,8-diaza-spiro[4.5]dec-8-yl]-1-phenyl-propyl}-amide hydrochloride

Compound 519
4,6-Dimethyl-pyrimidine-5-carboxylic acid {(S)-3-[2-(4-methoxy-benzyl)-1-oxo-2,8-diaza-spiro[4.5]dec-8-yl]-1-phenyl-propyl}-amide trihydrochloride

Compound 520
Adamantane-1-carboxylic acid {(S)-3-[2-(4-methoxy-benzyl)-1-oxo-2,8-diaza-spiro[4.5]dec-8-yl]-1-phenyl-propyl}-amide hydrochloride

Compound 521
N-{(S)-3-[2-(4-Methanesulfonyl-benzyl)-1-oxo-2,8-diaza-spiro[4.5]dec-8-yl]-1-phenyl-propyl}-propionamide hydrochloride

Compound 522
N-{(S)-3-[2-(4-Methanesulfonyl-benzyl)-1-oxo-2,8-diaza-spiro[4.5]dec-8-yl]-1-phenyl-propyl}-2,2-dimethyl-propionamide hydrochloride

Compound 523
Thiophene-2-carboxylic acid {(S)-3-[2-(4-methanesulfonyl-benzyl)-1-oxo-2,8-diaza-spiro[4.5]dec-8-yl]-1-phenyl-propyl}-amide hydrochloride

Compound 524
Thiophene-3-carboxylic acid {(S)-3-[2-(4-methanesulfonyl-benzyl)-1-oxo-2,8-diaza-spiro[4.5]dec-8-yl]-1-phenyl-propyl}-amide hydrochloride

Compound 525
4,4-Difluoro-cyclohexanecarboxylic acid {(S)-3-[2-(4-methanesulfonyl-benzyl)-1-oxo-2,8-diaza-spiro[4.5]dec-8-yl]-1-phenyl-propyl}-amide hydrochloride

Compound 526
Tetrahydro-pyran-4-carboxylic acid {(S)-3-[2-(4-methanesulfonyl-benzyl)-1-oxo-2,8-diaza-spiro[4.5]dec-8-yl]-1-phenyl-propyl}-amide hydrochloride

Compound 527
N-{(S)-3-[2-(4-Methanesulfonyl-benzyl)-1-oxo-2,8-diaza-spiro[4.5]dec-8-yl]-1-phenyl-propyl}-nicotinamide dihydrochloride

Compound 528
Pyrimidine-5-carboxylic acid {(S)-3-[2-(4-methanesulfonyl-benzyl)-1-oxo-2,8-diaza-spiro[4.5]dec-8-yl]-1-phenyl-propyl}-amide trihydrochloride

Compound 529
4,6-Dimethyl-pyrimidine-5-carboxylic acid {(S)-3-[2-(4-methanesulfonyl-benzyl)-1-oxo-2,8-diaza-spiro[4.5]dec-8-yl]-1-phenyl-propyl}-amide trihydrochloride

Compound 530
Adamantane-1-carboxylic acid {(S)-3-[2- (4-methanesulfonyl-benzyl)-1-oxo-2,8-diaza-spiro[4.5]dec-8-yl]-1-phenyl-propyl}-amide hydrochloride

Compound 531
N-{(S)-3-[2-(4-Ethoxy-benzyl)-1-oxo-2,8-diaza-spiro[4.5]dec-8-yl]-1-phenyl-propyl}-isobutyramide hydrochloride

Compound 532
Cyclopropanecarboxylic acid {(S)-3-[2-(4-ethoxy-benzyl)-1-oxo-2,8-diaza-spiro[4.5]dec-8-yl]-1-phenyl-propyl}-amide hydrochloride

Compound 533
4,4-Difluoro-cyclohexanecarboxylic acid {(S)-3-[2-(4-methoxy-benzyl)-3-oxo-2,8-diaza-spiro[4.5]dec-8-yl]-1-phenyl-propyl}-amide hydrochloride

Compound 534
2-Cyclopropyl-N-{(S)-3-[2-(4-methanesulfonyl-benzyl)-3-oxo-2,8-diaza-spiro[4.5]dec-8-yl]-1-phenyl-propyl}-acetamide hydrochloride

Compound 535
4,4-Difluoro-cyclohexanecarboxylic acid {(S)-3-[2-(4-methanesulfonyl-benzyl)-3-oxo-2,8-diaza-spiro[4.5]dec-8-yl]-1-phenyl-propyl}-amide hydrochloride

Compound 536
N-{3-[2-(4-Bromobenzyl)-1-oxo-2,8-diaza-spiro[4.5]dec-8-yl]-1-phenylpropyl}-benzenesulfonamide hydrochloride

Compound 537
Propane-2-sulfonic acid {(S)-3-[2-(4-methoxy-benzyl)-1-oxo-2,8-diaza-spiro[4.5]dec-8-yl]-1-phenyl-propyl}-amide hydrochloride

Compound 538
Propane-2-sulfonic acid {(S)-3-[2-(4-methanesulfonyl-benzyl)-1-oxo-2,8-diaza-spiro[4.5]dec-8-yl]-1-phenyl-propyl}-amide hydrochloride

Compound 539
Piperidine-1-carboxylic acid {3-[2-(4-bromobenzyl)-1-oxo-2,8-diaza-spiro[4.5]dec-8-yl]-1-phenyl-propyl}-amide hydrochloride

Compound 540
Piperidine-1-carboxylic acid {3-[2-(4-methoxybenzyl)-1-oxo-2,8-diaza-spiro[4.5]dec-8-yl]-1-phenyl-propyl}-amide hydrochloride

Compound 541
Piperidine-1-carboxylic acid {3-[2-(4-methanesulfonylbenzyl)-1-oxo-2,8-diaza-spiro[4.5]dec-8-yl]-1-phenyl-propyl}amide hydrochloride

Compound 542
1-{3-[2-(4-bromobenzyl)-1-oxo-2,8-diaza-spiro[4.5]dec-8-yl]-1-phenyl-propyl}-3-phenyl-urea hydrochloride

Compound 543
1-{3-[2-(4-bromobenzyl)-1-oxo-2,8-diaza-spiro[4.5]dec-8-yl]-1-phenyl-propyl}-3-(2-chloro-phenyl)-urea hydrochloride

Compound 544
1-{3-[2-(4-bromobenzyl)-1-oxo-2,8-diaza-spiro[4.5]dec-8-yl]-1-phenyl-propyl}-3-(2-methoxy-phenyl)-urea hydrochloride

Compound 545
1-{3-[2-(4-bromobenzyl)-1-oxo-2,8-diaza-spiro[4.5]dec-8-yl]-1-phenyl-propyl}-3-(3-chloro-phenyl)-urea hydrochloride

Compound 546
1-{3-[2-(4-bromobenzyl)-1-oxo-2,8-diaza-spiro[4.5]dec-8-yl]-1-phenyl-propyl}-3-(3-methoxy-phenyl)-urea hydrochloride

Compound 547
1-{3-[2-(4-bromobenzyl)-1-oxo-2,8-diaza-spiro[4.5]dec-8-yl]-1-phenyl-propyl}-3-(4-chloro-phenyl)-urea hydrochloride

Compound 548
1-{3-[2-(4-bromobenzyl)-1-oxo-2,8-diaza-spiro[4.5]dec-8-yl]-1-phenyl-propyl}-3-(4-methoxy-phenyl)-urea hydrochloride

Compound 549
1-{3-[2-(4-bromobenzyl)-1-oxo-2,8-diaza-spiro[4.5]dec-8-yl]-1-phenyl-propyl}-3-(2,6-dimethyl-phenyl)-urea hydrochloride

Compound 550
1-{3-[2-(4-bromobenzyl)-1-oxo-2,8-diaza-spiro[4.5]dec-8-yl]-1-phenyl-propyl}-3-naphthalen-1-yl-urea hydrochloride

Compound 551
1-{3-[2-(4-methoxybenzyl)-1-oxo-2,8-diaza-spiro[4.5]dec-8-yl]-1-phenyl-propyl}-3-phenyl-urea hydrochloride

Compound 552
1-(2-chlorophenyl)-3-{3-[2-(4-methoxybenzyl)-1-oxo-2,8-diaza-spiro[4.5]dec-8-yl]-1-phenyl-propyl}-urea hydrochloride

Compound 553
1-{3-[2-(4-methoxybenzyl)-1-oxo-2,8-diaza-spiro[4.5]dec-8-yl]-1-phenyl-propyl}-3-(2-methoxyphenyl)-urea hydrochloride

Compound 554
1-(3-chlorophenyl)-3-{3-[2-(4-methoxybenzyl)-1-oxo-2,8-diaza-spiro[4.5]dec-8-yl]-1-phenyl-propyl}-urea hydrochloride

Compound 555
1-{3-[2-(4-methoxybenzyl)-1-oxo-2,8-diaza-spiro[4.5]dec-8-yl]-1-phenyl-propyl}-3-(3-methoxyphenyl)-urea hydrochloride

Compound 556
-{3-[2-(4-methoxybenzyl)-1-oxo-2,8-diaza-spiro[4.5]dec-8-yl]-1-phenyl-propyl}-3-(4-methoxyphenyl)-urea hydrochloride

Compound 557
1-(2,6-dimethylphenyl)-3-{3-[2-(4-methoxybenzyl)-1-oxo-2,8-diaza-spiro[4.5]dec-8-yl]-1-phenyl-propyl}-urea hydrochloride

Compound 558
1-{3-[2-(4-methoxybenzyl)-1-oxo-2,8-diaza-spiro[4.5]dec-8-yl]-1-phenyl-propyl}-3-naphthalen-1-yl-urea hydrochloride

Compound 559
1-{3-[2-(4-methanesulfonylbenzyl)-1-oxo-2,8-diaza-spiro[4.5]dec-8-yl]-1-phenyl-propyl}-3-phenyl-urea hydrochloride

Compound 560
1-(2-chlorophenyl)-3-{3-[2-(4-methanesulfonylbenzyl)-1-oxo-2,8-diaza-spiro[4.5]dec-8-yl]-1-phenyl-propyl}-urea hydrochloride

Compound 561
1-{3-[2-(4-methanesulfonylbenzyl)--oxo-2,8-diaza-spiro[4.5]dec-8-yl]-1-phenyl-propyl}-3-(2-methoxyphenyl)-urea hydrochloride

Compound 562
1-(3-chlorophenyl)-3-{3-[2-(4-methanesulfonylbenzyl)-1-oxo-2,8-diaza-spiro[4.5]dec-8-yl]-1-phenyl-propyl}-urea hydrochloride

Compound 563
1-{3-[2-(4-methanesulfonylbenzyl)-1-oxo-2,8-diaza-spiro[4.5]dec-8-yl]-1-phenyl-propyl}-3-(3-methoxyphenyl)-urea hydrochloride

Compound 564
1-(4-chlorophenyl)-3-{3-[2-(4-methanesulfonylbenzyl)-1-oxo-2,8-diaza-spiro[4.5]dec-8-yl]-1-phenyl-propyl}-urea hydrochloride

Compound 565
1-{3-[2-(4-methanesulfonylbenzyl)-1-oxo-2,8-diaza-spiro[4.5)dec-8-yl]-1-phenyl-propyl}-3-(4-methoxyphenyl)-urea hydrochloride

Compound 566
1-(2,6-dimethylphenyl)-3-{3-[2-(4-methanesulfonylbenzyl)-1-oxo-2,8-diaza-spiro[4.5]dec-8-yl]-1-phenyl-propyl}-urea hydrochloride

Compound 567
1-{3-[2-(4-methanesulfonylbenzyl)-1-oxo-2,8-diaza-spiro[4.5]dec-8-yl]-1-phenyl-propyl}-3-naphthalen-1-yl-urea hydrochloride

Compound 568
Morpholine-4-carboxylic acid {(S)-3-[2-(4-methoxy-benzyl)-1-oxo-2,8-diaza-spiro[4.5]dec-8-yl]-1-phenyl-propyl}-amide hydrochloride

Compound 569
Morpholine-4-carboxylic acid {(S)-3-[2-(4-methanesulfonyl-benzyl)-1-oxo-2,8-diaza-spiro[4.5]dec-8-yl]-1-phenyl-propyl}-amide hydrochloride

Compound 570
3,3-Difluoro-pyrrolidine-1-carboxylic acid {(S)-3-[2-(4-methoxy-benzyl)-1-oxo-2,8-diaza-spiro[4.5]dec-8-yl]-1-phenyl-propyl}-amide hydrochloride

Compound 571
3,3-Difluoro-pyrrolidine-1-carboxylic acid {(S)-3-[2-(4-methanesulfonyl-benzyl)-1-oxo-2,8-diaza-spiro[4.5]dec-8-yl]-1-phenyl-propyl}-amide hydrochloride

Compound 572
{3-[2-(4-methanesulfonylbenzyl)-1-oxo-2,8-diaza-spiro[4.5]dec-8-yl]-1-phenyl-propyl}-carbamic acid methyl ester hydrochloride

Compound 573
{3-[2-(4-methanesulfonylbenzyl)-1-oxo-2,8-diaza-spiro[4.5]dec-8-yl]-1-phenyl-propyl}-carbamic acid ethyl ester hydrochloride

Compound 574
{3-[2-(4-Methanesulfonyl-benzyl)-1-oxo-2,8-diaza-spiro[4.5]dec-8-yl]-1-pyridin-2-yl-propyl}-carbamic acid cyclohexyl ester

Compound 575
{(S)-3-[2-(4-Methanesulfonyl-benzyl)-1-oxo-2,8-diaza-spiro[4.5]dec-8-yl]-1-phenyl-propyl}-carbamic acid cyclohexyl ester

Compound 576
{(S)-3-[2-(4-Methanesulfonyl-benzyl)-1-oxo-2,8-diaza-spiro[4.5]dec-8-yl]-1-phenyl-propyl}-carbamic acid cyclobutyl ester

Compound 577
{(S)-3-[2-(4-Methanesulfonyl-benzyl)-1-oxo-2,8-diaza-spiro[4.5]dec-8-yl]-1-phenyl-propyl}-carbamic acid cyclopentyl ester

Compound 578
[(S)-3-(1-Oxo-2-pyridin-3-ylmethyl-2,8-diaza-spiro[4.5]dec-8-yl)-1-phenyl-propyl]-carbamic acid cyclobutyl ester

Compound 579
[(S)-3-(1-Oxo-2-pyridin-3-ylmethyl-2,8-diaza-spiro[4.5]dec-8-yl)-1-phenyl-propyl]-carbamic acid cyclopentyl ester

Compound 580
[(S)-3-(1-Oxo-2-pyridin-3-ylmethyl-2,8-diaza-spiro[4.5]dec-8-yl)-1-phenyl-propyl]-carbamic acid 1-methyl-cyclopentyl ester

Compound 581
[(S)-3-(1-Oxo-2-pyridin-3-ylmethyl-2,8-diaza-spiro[4.5]dec-8-yl)-1-phenyl-propyl]-carbamic acid cyclohexyl ester

Compound 582
{3-[2-(4-Bromo-benzyl)-1-oxo-2,8-diaza-spiro[4.5]dec-8-yl]-1-thiophen-2-yl-propyl}-carbamic acid ethyl ester hydrochloride

Compound 583
{3-[2-(4-Methoxy-benzyl)-1-oxo-2,8-diaza-spiro[4.5]dec-8-yl]-1-thiophen-2-yl-propyl}-carbamic acid ethyl ester hydrochloride

Compound 584
[3-(1-Oxo-2-pyridin-3-ylmethyl-2,8-diaza-spiro[4.5]dec-8-yl)-1-thiophen-2-yl-propyl]-carbamic acid ethyl ester dihydrochloride

Compound 585
{1-(2-Chloro-phenyl)-3-[2-(4-methoxy-benzyl)-1-oxo-2,8-diaza-spiro[4.5]dec-8-yl]-propyl}-carbamic acid ethyl ester hydrochloride

Compound 586
{1-(2-Chloro-phenyl)-3-[2-(4-methanesulfonyl-benzyl)-1-oxo-2,8-diaza-spiro[4.5]dec-8-yl]-propyl}-carbamic acid ethyl ester hydrochloride

Compound 587
[1-(2-Chloro-phenyl)-3-(1-oxo-2-pyridin-3-ylmethyl-2,8-diaza-spiro[4.5]dec-8-yl)-propyl]-carbamic acid ethyl ester dihydrochloride

Compound 588
[3-[2-(4-Bromo-benzyl)-1-oxo-2,8-diaza-spiro[4.5]dec-8-yl]-1-(3-chloro-phenyl)-propyl]-carbamic acid ethyl ester hydrochloride

Compound 589
{1-(3-Chloro-phenyl)-3-[2-(4-methoxy-benzyl)-1-oxo-2,8-diaza-spiro[4.5]dec-8-yl]-propyl}-carbamic acid ethyl ester hydrochloride

Compound 590
{1-(3-Chloro-phenyl)-3-[2-(4-methanesulfonyl-benzyl)-1-oxo-2,8-diaza-spiro[4.5]dec-8-yl]-propyl}-carbamic acid ethyl ester hydrochloride

Compound 591
[1-(3-Chloro-phenyl)-3-(1-oxo-2-pyridin-3-ylmethyl-2,8-diaza-spiro[4.5]dec-8-yl)-propyl]-carbamic acid ethyl ester dihydrochloride

Compound 592
{1-(4-Chloro-phenyl)-3-[2-(4-methoxy-benzyl)-1-oxo-2,8-diaza-spiro[4.5]dec-8-yl]-propyl}-carbamic acid ethyl ester hydrochloride

Compound 593
[1-(4-Chloro-phenyl)-3-(1-oxo-2-pyridin-3-ylmethyl-2,8-diaza-spiro[4.5]dec-8-yl)-propyl]-carbamic acid ethyl ester dihydrochloride

Compound 594
{3-[2-(4-Bromo-benzyl)-1-oxo-2,8-diaza-spiro[4.5]dec-8-yl]-1-thiophen-2-yl-propyl}-carbamic acid methyl ester hydrochloride

Compound 595
{3-[2-(4-Methoxy-benzyl)-1-oxo-2,8-diaza-spiro[4.5]dec-8-yl]-1-thiophen-2-yl-propyl}-carbamic acid methyl ester hydrochloride

Compound 596
{3-[2-(4-Methanesulfonyl-benzyl)-1-oxo-2,8-diaza-spiro[4.5]dec-8-yl]-1-thiophen-2-yl-propyl}-carbamic acid methyl ester hydrochloride

Compound 597
3-(1-Oxo-2-pyridin-3-ylmethyl-2,8-diaza-spiro[4.5]dec-8-yl)-1-thiophen-2-yl-propyl]-carbamic acid methyl ester dihydrochloride

Compound 598
{1-(2-Chloro-phenyl)-3-[2-(4-methoxy-benzyl)-1-oxo-2,8-diaza-spiro[4.5]dec-8-yl]-propyl}-carbamic acid methyl ester hydrochloride

Compound 599
{1-(2-Chloro-phenyl)-3-[2-(4-methanesulfonyl-benzyl)-1-oxo-2,8-diaza-spiro[4.5]dec-8-yl]-propyl}-carbamic acid methyl ester hydrochloride

Compound 600
[1-(2-Chloro-phenyl)-3-(1-oxo-2-pyridin-3-ylmethyl-2,8-diaza-spiro[4.5]dec-8-yl)-propyl]-carbamic acid methyl ester dihydrochloride

Compound 601
[3-[2-(4-Bromo-benzyl)-1-oxo-2,8-diaza-spiro[4.5]dec-8-yl]-1-(3-chloro-phenyl)-propyl]-carbamic acid methyl ester hydrochloride

Compound 602
{1-(3-Chloro-phenyl)-3-[2-(4-methoxy-benzyl)-1-oxo-2,8-diaza-spiro[4.5]dec-8-yl]-propyl}-carbamic acid methyl ester hydrochloride

Compound 603
{3-[2-(4-Bromo-benzyl)-1-oxo-2,8-diaza-spiro[4.5]dec-8-yl]-1-pyridin-2-yl-propyl}-carbamic acid ethyl ester hydrochloride

Compound 604
{3-[2-(4-Methoxy-benzyl)-1-oxo-2,8-diaza-spiro[4.5]dec-8-yl]-1-pyridin-2-yl-propyl}-carbamic acid ethyl ester dihydrochloride

Compound 605
3-[2-(4-Bromo-benzyl)-1-oxo-2,8-diaza-spiro[4.5]dec-8-yl]-1-(4-methoxy-phenyl)-propyl]-carbamic acid ethyl ester hydrochloride

Compound 606
[3-[2-(4-Methoxy-benzyl)-1-oxo-2,8-diaza-spiro[4.5]dec-8-yl]-1-(4-methoxy-phenyl)-propyl]-carbamic acid ethyl ester hydrochloride

Compound 607
[3-[2-(4-Methanesulfonyl-benzyl)-1-oxo-2,8-diaza-spiro[4.5]dec-8-yl]-1-(4-methoxy-phenyl)-propyl]-carbamic acid ethyl ester hydrochloride

Compound 608
[1-(4-Methoxy-phenyl)-3-(1-oxo-2-pyridin-3-ylmethyl-2,8-diaza-spiro[4.5]dec-8-yl)-propyl]-carbamic acid ethyl ester dihydrochloride

Compound 609
[3-[2-(4-Bromo-benzyl)-1-oxo-2,8-diaza-spiro[4.5]dec-8-yl]-1-(3,4-dimethoxy-phenyl)-propyl]-carbamic acid ethyl ester hydrochloride

Compound 610
{1-(3,4-Dimethoxy-phenyl)-3-[2-(4-methoxy-benzyl)-1-oxo-2,8-diaza-spiro[4.5]dec-8-yl]-propyl}-carbamic acid ethyl ester hydrochloride

Compound 611
{1-(3,4-Dimethoxy-phenyl)-3-[2-(4-methanesulfonyl-benzyl)-1-oxo-2,8-diaza-spiro[4.5]dec-8-yl]-propyl}-carbamic acid ethyl ester hydrochloride

Compound 612
[1-(3,4-Dimethoxy-phenyl)-3-(1-oxo-2-pyridin-3-ylmethyl-2,8-diaza-spiro[4.5]dec-8-yl)-propyl]-carbamic acid ethyl ester dihydrochloride

Compound 613
[3-[2-(4-Bromo-benzyl)-1-oxo-2,8-diaza-spiro[4.5]dec-8-yl]-1-(3-methoxy-phenyl)-propyl]-carbamic acid ethyl ester hydrochloride

Compound 614
[3-[2-(4-Methoxy-benzyl)-1-oxo-2,8-diaza-spiro[4.5]dec-8-yl]-1-(3-methoxy-phenyl)-propyl]-carbamic acid ethyl ester hydrochloride

Compound 615
[3-[2-(4-Methanesulfonyl-benzyl)-1-oxo-2,8-diaza-spiro[4.5]dec-8-yl]-1-(3-methoxy-phenyl)-propyl]-carbamic acid ethyl ester hydrochloride

Compound 616
[1-(3-Methoxy-phenyl)-3-(1-oxo-2-pyridin-3-ylmethyl-2,8-diaza-spiro[4.5]dec-8-yl)-propyl]-carbamic acid ethyl ester dihydrochloride

Compound 617
[3-[2-(4-Bromo-benzyl)-1-oxo-2,8-diaza-spiro[4.5]dec-8-yl]-1-(4-methoxy-phenyl)-propyl]-carbamic acid methyl ester hydrochloride

Compound 618
[3-[2-(4-Methoxy-benzyl)-1-oxo-2,8-diaza-spiro[4.5]dec-8-yl]-1-(4-methoxy-phenyl)-propyl]-carbamic acid methyl ester hydrochloride

Compound 619
[3-[2-(4-Methanesulfonyl-benzyl)-1-oxo-2,8-diaza-spiro[4.5]dec-8-yl]-1-(4-methoxy-phenyl)-propyl]-carbamic acid methyl ester hydrochloride

Compound 620
[3-[2-(4-Bromo-benzyl)-1-oxo-2,8-diaza-spiro[4.5]dec-8-yl]-1-(3,4-dimethoxy-phenyl)-propyl]-carbamic acid methyl ester hydrochloride

Compound 621
(1-(3,4-Dimethoxy-phenyl)-3-[2-(4-methoxy-benzyl)-1-oxo-2,8-diaza-spiro[4.5]dec-8-yl]-propyl}-carbamic acid methyl ester hydrochloride

Compound 622
{1-(3,4-Dimethoxy-phenyl)-3-[2-(4-methanesulfonyl-benzyl)-1-oxo-2,8-diaza-spiro[4.5]dec-8-yl]-propyl}-carbamic acid methyl ester hydrochloride

Compound 623
[1-(3,4-Dimethoxy-phenyl)-3-(1-oxo-2-pyridin-3-ylmethyl-2,8-diaza-spiro[4.5]dec-8-yl)-propyl]-carbamic acid methyl ester dihydrochloride

Compound 624
[3-[2-(4-Bromo-benzyl)-1-oxo-2,8-diaza-spiro[4.5]dec-8-yl]-1-(3-methoxy-phenyl)-propyl]-carbamic acid methyl ester hydrochloride

Compound 625
[3-[2-(4-Methoxy-benzyl)-1-oxo-2,8-diaza-spiro[4.5]dec-8-yl]-1-(3-methoxy-phenyl)-propyl]-carbamic acid methyl ester hydrochloride

Compound 626
[3-[2-(4-Methanesulfonyl-benzyl)-1-oxo-2,8-diaza-spiro[4.5]dec-8-yl]-1-(3-methoxy-phenyl)-propyl]-carbamic acid methyl ester hydrochloride

Compound 627
[1-(3-Methoxy-phenyl)-3-(1-oxo-2-pyridin-3-ylmethyl-2,8-diaza-spiro[4.5]dec-8-yl)-propyl]-carbamic acid methyl ester dihydrochloride

Compound 628
{1-(3-Chloro-phenyl)-3-[2-(4-methanesulfonyl-benzyl)-1-oxo-2,8-diaza-spiro[4.5]dec-8-yl]-propyl)-carbamic acid methyl ester hydrochloride

Compound 629
1-(3-Chloro-phenyl)-3-(1-oxo-2-pyridin-3-ylmethyl-2,8-diaza-spiro[4.5]dec-8-yl)-propyl]-carbamic acid methyl ester dihydrochloride

Compound 630
{1-(4-Chloro-phenyl)-3-[2-(4-methoxy-benzyl)-1-oxo-2,8-diaza-spiro[4.5]dec-8-yl]-propyl}-carbamic acid methyl ester hydrochloride

Compound 631
{1-(4-Chloro-phenyl)-3-[2-(4-methanesulfonyl-benzyl)-1-oxo-2,8-diaza-spiro[4.5]dec-8-yl]-propyl}-carbamic acid methyl ester hydrochloride

Compound 632
[1-(4-Chloro-phenyl)-3-(1-oxo-2-pyridin-3-ylmethyl-2,8-diaza-spiro[4.5]dec-8-yl)-propyl]-carbamic acid methyl ester dihydrochloride

Compound 633
[3-[2-(4-Bromo-benzyl)-1-oxo-2,8-diaza-spiro[4.5]dec-8-yl]-1-(2-methoxy-phenyl)-propyl}-carbamic acid methyl ester hydrochloride

Compound 634
[3-[2-(4-Bromo-benzyl)-1-oxo-2,8-diaza-spiro[4.5]dec-8-yl]-1-(2-methoxy-phenyl)-propyl]-carbamic acid ethyl ester hydrochloride

Compound 635
[3-[2-(4-Methoxy-benzyl)-1-oxo-2,8-diaza-spiro[4.5]dec-8-yl]-1-(2-methoxy-phenyl)-propyl]-carbamic acid methyl ester hydrochloride

Compound 636
[3-[2-(4-Methoxy-benzyl)-1-oxo-2,8-diaza-spiro[4.5]dec-8-yl]-1-(2-methoxy-phenyl)-propyl]-carbamic acid ethyl ester hydrochloride

Compound 637
[3-[2-(4-Methanesulfonyl-benzyl)-1-oxo-2,8-diaza-spiro[4.5]dec-8-yl]-1-(2-methoxy-phenyl)-propyl]-carbamic acid methyl ester hydrochloride

Compound 638
[3-[2-(4-Methanesulfonyl-benzyl)-1-oxo-2,8-diaza-spiro[4.5]dec-8-yl]-1-(2-methoxy-phenyl)-propyl]-carbamic acid ethyl ester hydrochloride

Compound 639
(4-Bromophenyl)-[8-(3,3-diphenylpropyl)-2,8-diaza-spiro[4.5]dec-2-yl]-methanone hydrochloride

Compound 640
4-[2-(4-bromobenzyl)-1-oxo-2,8-diaza-spiro[4.5]dec-8-yl]-2-phenyl-butyric acid methyl ester

Compound 641
4-[2-(4-methanesulfonylbenzyl)-1-oxo-2,8-diaza-spiro[4.5]dec-8-yl]-2-phenyl-butyric acid methyl ester

Compound 642
4-[2-(4-methoxybenzyl)-1-oxo-2,8-diaza-spiro[4.5]dec-8-yl]-2-phenyl-butyric acid methyl ester

Compound 643
4-[2-(4-bromobenzyl)-1-oxo-2,8-diaza-spiro[4.5]dec-8-yl]-2,N-diphenyl-butyramide hydrochloride

Compound 644
N-benzyl-4-[2-(4-bromobenzyl)-1-oxo-2,8-diaza-spiro[4.5]dec-8-yl]-2-phenyl-butyramide hydrochloride

Compound 645
2-(4-bromobenzyl)-8-(4-oxo-3-phenyl-4-piperidin-1-yl-butyl)-2,8-diaza-spiro[4.5]decan-1-one hydrochloride

Compound 646
4-[2-(4-bromobenzyl)-1-oxo-2,8-diaza-spiro[4.5]dec-8-yl]-N-cyclohexyl-2-phenyl-butyramide hydrochloride

Compound 647
4-[2-(4-bromobenzyl)-1-oxo-2,8-diaza-spiro[4.5]dec-8-yl]-N-cyclohexyl-n-methyl-2-phenyl-butyramide hydrochloride

Compound 648
4-[2-(4-bromobenzyl)-1-oxo-2,8-diaza-spiro[4.5]dec-8-yl]-N-cyclopropyl-2-phenyl-butyramide hydrochloride

Compound 649
4-[2-(4-bromobenzyl)-1-oxo-2,8-diaza-spiro[4.5]dec-8-yl]-N-cyclobutyl-2-phenyl-butyramide hydrochloride

Compound 650
N-cyclohexyl-4-[2-(4-methanesulfonylbenzyl)-1-oxo-2,8-diaza-spiro[4.5]dec-8-yl]-n-methyl-2-phenyl-butyramide hydrochloride

Compound 651
N-cyclopropyl-4-[2-(4-methanesulfonylbenzyl)-1-oxo-2,8-diaza-spiro[4.5]dec-8-yl]-2-phenyl-butyramide hydrochloride

Compound 652
N-cyclobutyl-4-[2-(4-methanesulfonylbenzyl)-1-oxo-2,8-diaza-spiro[4.5]dec-8-yl]-2-phenyl-butyramide hydrochloride

Compound 653
4-[2-(4-bromobenzyl)-1-oxo-2,8-diaza-spiro[4.5]dec-8-yl]-N-cyclopentyl-2-phenyl-butyramide hydrochloride

Compound 654
4-[2-(4-bromobenzyl)-1-oxo-2,8-diaza-spiro[4.5]dec-8-yl]-N-isopropyl-2-phenyl-butyramide hydrochloride

Compound 655
N-benzyl-4-[2-(4-methanesulfonylbenzyl)-1-oxo-2,8-diaza-spiro[4.5]dec-8-yl]-2-phenyl-butyramide hydrochloride

Compound 656
2-(4-methanesulfonylbenzyl)-8-(4-oxo-3-phenyl-4-piperidin-1-yl -butyl)-2,8-diaza-spiro[4.5]decan-1-one hydrochloride

Compound 657
N-cyclohexyl-4-[2-(4-methanesulfonylbenzyl)-1-oxo-2,8-diaza-spiro[4.5]dec-8-yl]-2-phenyl-butyramide hydrochloride

Compound 658
N-cyclopentyl-4-[2-(4-methanesulfonylbenzyl)-1-oxo-2,8-diaza-spiro[4.5]dec-8-yl]-2-phenyl-butyramide hydrochloride

Compound 659
N-isopropyl-4-[2-(4-methanesulfonylbenzyl)-1-oxo-2,8-diaza-spiro[4.5]dec-8-yl]-2-phenyl-butyramide hydrochloride

Compound 660
4-[2-(4-methoxybenzyl)-1-oxo-2,8-diaza-spiro[4.5]dec-8-yl]-2,N-diphenyl-butyramide hydrochloride

Compound 661
N-benzyl-4-[2-(4-methoxybenzyl)-1-oxo-2,8-diaza-spiro[4.5]dec-8-yl]-2-phenyl-butyramide hydrochloride

Compound 662
2-(4-methoxybenzyl)-8-(4-oxo-3-phenyl-4-piperidin-1-yl-butyl)-2,8-diaza-spiro[4.5]decan-1-one hydrochloride

Compound 663
N-cyclohexyl-4-[2-(4-methoxybenzyl)-1-oxo-2,8-diaza-spiro[4.5]dec-8-yl]-2-phenyl-butyramide hydrochloride

Compound 664
N-cyclopropyl-4-[2-(4-methoxybenzyl)-1-oxo-2,8-diaza-spiro[4.5]dec-8-yl]-2-phenyl-butyramide hydrochloride

Compound 665
N-cyclobutyl-4-[2-(4-methoxybenzyl)-1-oxo-2,8-diaza-spiro[4.5]dec-8-yl]-2-phenyl-butyramide hydrochloride

Compound 666
N-cyclopentyl-4-[2-(4-methoxybenzyl)-1-oxo-2,8-diaza-spiro[4.5]dec-8-yl]-2-phenyl-butyramide hydrochloride

Compound 667
N-isopropyl-4-[2-(4-methoxybenzyl)-1-oxo-2,8-diaza-spiro[4.5]dec-8-yl]-2-phenyl-butyramide hydrochloride

Compound 668
Cyclopropanecarboxylic acid {(S)-1-(3-chloro-phenyl)-3-[2-(4-methoxy-benzyl)-1-oxo-2,8-diaza-spiro[4.5]dec-8-yl]-propyl}-amide hydrochloride

Compound 669
Cyclopropanecarboxylic acid {(S)-1-(3-fluoro-phenyl)-3-[2-(4-methoxy-benzyl)-1-oxo-2,8-diaza-spiro[4.5]dec-8-yl]-propyl}-amide hydrochloride

Compound 670
Cyclopropanecarboxylic acid {(S)-3-[2- (4-methoxy-benzyl)-1-oxo-2,8-diaza-spiro[4.5]dec-8-yl]-1-thiophen-2-yl-propyl}-amide hydrochloride

Compound 671
Cyclopropanecarboxylic acid {(S)-3-(2-(4-methoxy-benzyl)-1-oxo-2,8-diaza-spiro[4.5]dec-8-yl]-1-thiophen-3-yl-propyl}-amide hydrochloride

Compound 672
N-{(S)-1-(3-Chloro-phenyl)-3-[2-(4-methoxy-benzyl)-1-oxo-2,8-diaza-spiro[4.5]dec-8-yl]-propyl}-isobutyramide hydrochloride

Compound 673
N-{(S)-1-(3-Fluoro-phenyl)-3-[2-(4-methoxy-benzyl)-1-oxo-2,8-diaza-spiro[4.5]dec-8-yl]-propyl}-isobutyramide hydrochloride

Compound 674
N-{(S)-3-[2-(4-Methoxy-benzyl)-1-oxo-2,8-diaza-spiro[4.5]dec-8-yl]-1-thiophen-2-yl-propyl}-isobutyramide hydrochloride

Compound 675
N-{(S)-3-[2-(4-Methoxy-benzyl)-1-oxo-2,8-diaza-spiro[4.5]dec-8-yl]-1-thiophen-3-yl-propyl}-isobutyramide hydrochloride

Compound 676
N-{(S)-3-[2-(4-Chloro-benzyl)-3-oxo-2,8-diaza-spiro[4.5]dec-8-yl]-1-phenyl-propyl}-isobutyramide hydrochloride

Compound 677
N-{(S)-3-[2-(4-Fluoro-benzyl)-3-oxo-2,8-diaza-spiro[4.5]dec-8-yl]-1-phenyl-propyl}-isobutyramide hydrochloride

Compound 678
N-{(S)-3-[2-(4-Cyano-benzyl)-3-oxo-2,8-diaza-spiro[4.5]dec-8-yl]-1-phenyl-propyl}-isobutyramide hydrochloride

Compound 679
N-{(S)-3-[2-(4-Ethoxy-benzyl)-3-oxo-2,8-diaza-spiro[4.5]dec-8-yl]-1-phenyl-propyl}-isobutyramide hydrochloride

Compound 680
N-{(S)-3-[2-(4-Difluoromethoxy-benzyl)-3-oxo-2,8-diaza-spiro[4.5]dec-8-yl)-1-phenyl-propyl}-isobutyramide hydrochloride

Compound 681
N-{(S)-3-[3-Oxo-2-(4-trifluoromethoxy-benzyl)-2,8-diaza-spiro[4.5]dec-8-yl]-1-phenyl-propyl}-isobutyramide hydrochloride

Compound 682
N-{(S)-3-[3-Oxo-2-(4-trifluoromethyl-benzyl)-2,8-diaza-spiro[4.5]dec-8-yl]-1-phenyl-propyl}-isobutyramide hydrochloride

Compound 683
N-{(S)-3-[3-Oxo-2-(4-pyrazol-1-yl-benzyl)-2,8-diaza-spiro[4.5]dec-8-yl]-1-phenyl-propyl}-isobutyramide hydrochloride

Compound 684
Cyclopropanecarboxylic acid {(S)-3-[2-(4-chloro-benzyl)-3-oxo-2,8-diaza-spiro[4.5]dec-8-yl]-1-phenyl-propyl}-amide hydrochloride

Compound 685
Cyclopropanecarboxylic acid {(S)-3-[2-(4-fluoro-benzyl)-3-oxo-2,8-diaza-spiro[4.5]dec-8-yl]-1-phenyl-propyl}-amide hydrochloride

Compound 686
Cyclopropanecarboxylic acid {(S)-3-[2-(4-cyano-benzyl)-3-oxo-2,8-diaza-spiro[4.5]dec-8-yl]-1-phenyl-propyl}-amide hydrochloride

Compound 687
Cyclopropanecarboxylic acid {(S)-3-[2-(4-ethoxy-benzyl)-3-oxo-2,8-diaza-spiro[4.5]dec-8-yl]-1-phenyl-propyl}-amide hydrochloride

Compound 688
Cyclopropanecarboxylic acid {(S)-3-[2-(4-difluoromethoxy-benzyl)-3-oxo-2,8-diaza-spiro[4.5]dec-8-yl]-1-phenyl-propyl}-amide hydrochloride

Compound 689
Cyclopropanecarboxylic acid {(S)-3-[2-(4-trifluoromethoxy-benzyl)-3-oxo-2,8-diaza-spiro[4.5]dec-8-yl]-1-phenyl-propyl}-amide hydrochloride

Compound 690
Cyclopropanecarboxylic acid {(S)-3-[2-(4-trifluoromethyl-benzyl)-3-oxo-2,8-diaza-spiro[4.5]dec-8-yl]-1-phenyl-propyl}-amide hydrochloride

Compound 691
Cyclopropanecarboxylic acid {(S)-3-[3-oxo-2-(4-pyrazol-1-yl-benzyl)-2,8-diaza-spiro[4.5]dec-8-yl]-1-phenyl-propyl}-amide hydrochloride

Compound 692
2-Cyclopropyl-N-{(S)-3-[2-(4-methoxy-benzyl)-3-oxo-2,8-diaza-spiro[4.5]dec-8-yl]-1-phenyl-propyl}-acetamide hydrochloride

Compound 693
N-{(S)-3-[2-(4-Chloro-benzyl)-3-oxo-2,8-diaza-spiro[4.5]dec-8-yl]-1-phenyl-propyl}-2-cyclopropyl-acetamide hydrochloride

Compound 694
N-{(S)-3-[2-(4-Fluoro-benzyl)-3-oxo-2,8-diaza-spiro[4.5]dec-8-yl]-1-phenyl-propyl}-2-cyclopropyl-acetamide hydrochloride

Compound 695
N-{(S)-3-[2-(4-Cyano-benzyl)-3-oxo-2,8-diaza-spiro[4.5]dec-8-yl]-1-phenyl-propyl}-2-cyclopropyl-acetamide hydrochloride

Compound 696
N-{(S)-3-[2-(4-Ethoxy-benzyl)-3-oxo-2,8-diaza-spiro[4.5]dec-8-yl]-1-phenyl-propyl}-2-cyclopropyl-acetamide hydrochloride

Compound 697
N-{(S)-3-[2-(4-Difluoromethoxy-benzyl)-3-oxo-2,8-diaza-spiro[4.5]dec-8-yl]-1-phenyl-propyl}-2-cyclopropyl-acetamide hydrochloride

Compound 698
N-{(S)-3-[2-(4-Trifluoromethoxy-benzyl)-3-oxo-2,8-diaza-spiro[4.5)dec-8-yl]-1-phenyl-propyl}-2-cyclopropyl-acetamide hydrochloride

Compound 699
N-{(S)-3-[2-(4-Trifluoromethyl-benzyl)-3-oxo-2,8-diaza-spiro[4.5)dec-8-yl]-1-phenyl-propyl}-2-cyclopropyl-acetamide hydrochloride

Compound 700
2-Cyclopropyl-N-{(S)-3-[3-oxo-2-(4-pyrazol-1-yl-benzyl)-2,8-diaza-spiro[4.5]dec-8-yl]-1-phenyl-propyl}-acetamide hydrochloride

Compound 701
4,4-Difluoro-cyclohexanecarboxylic acid {(S)-3-[2-(4-chloro-benzyl)-3-oxo-2,8-diaza-spiro[4.5]dec-8-yl]-1-phenyl-propyl}-amide hydrochloride

Compound 702
4,4-Difluoro-cyclohexanecarboxylic acid {(S)-3-[2-(4-fluoro-benzyl)-3-oxo-2,8-diaza-spiro[4.5]dec-8-yl]-1-phenyl-propyl}-amide hydrochloride

Compound 703
4,4-Difluoro-cyclohexanecarboxylic acid {(S)-3-[2-(4-cyano-benzyl)-3-oxo-2,8-diaza-spiro[4.5)dec-8-yl]-1-phenyl-propyl}-amide hydrochloride

Compound 704
4,4-Difluoro-cyclohexanecarboxylic acid {((S)-3-[2-(4-ethoxy-benzyl)-3-oxo-2,8-diaza-spiro[4.5]dec-8-yl]-1-phenyl-propyl}-amide hydrochloride

Compound 705
4,4-Difluoro-cyclohexanecarboxylic acid {(S)-3-[3-oxo-2-(4-pyrazol-1-yl-benzyl)-2,8-diaza-spiro[4.5]dec-8-yl]-1-phenyl-propyl}-amide hydrochloride

Compound 706
Cyclopropanecarboxylic acid {(S)-1-(3-chloro-phenyl)-3-[2-(4-methoxy-benzyl)-3-oxo-2,8-diaza-spiro[4.5]dec-8-yl]-propyl}-amide hydrochloride

Compound 707
Cyclopropanecarboxylic acid {(S)-1-(3-fluoro-phenyl)-3-[2-(4-methoxy-benzyl)-3-oxo-2,8-diaza-spiro[4.5]dec-8-yl]-propyl}-amide hydrochloride

Compound 708
Cyclopropanecarboxylic acid {(S)-3-[2-(4-methoxy-benzyl)-3-oxo-2,8-diaza-spiro[4.5]dec-8-yl]-1-thiophen-2-yl-propyl}-amide hydrochloride

Compound 709
Cyclopropanecarboxylic acid {(S)-3-[2-(4-methoxy-benzyl)-3-oxo-2,8-diaza-spiro[4.5]dec-8-yl]-1-thiophen-3-yl-propyl}-amide hydrochloride

Compound 710
N-{(S)-1-(3-Chloro-phenyl)-3-[2-(4-methoxy-benzyl)-3-oxo-2,8-diaza-spiro[4.5]dec-8-yl]-propyl}-isobutyramide hydrochloride

Compound 711
N-{(S)-1-(3-Fluoro-phenyl)-3-[2-(4-methoxy-benzyl)-3-oxo-2,8-diaza-spiro[4.5]dec-8-yl]-propyl}-isobutyramide hydrochloride

Compound 712
N-{(S)-3-[2-(4-Methoxy-benzyl)-3-oxo-2,8-diaza-spiro[4.5]dec-8-yl]-1-thiophen-2-yl-propyl}-isobutyramide hydrochloride

Compound 713
N-{(S)-3-[2-(4-Methoxy-benzyl)-3-oxo-2,8-diaza-spiro[4.5]dec-8-yl]-1-thiophen-3-yl-propyl}-isobutyramide hydrochloride

Compound 714
Propane-2-sulfonic acid {(S)-3-[2-(4-methoxy-benzyl)-3-oxo-2,8-diaza-spiro[4.5]dec-8-yl]-1-phenyl-propyl}-amide hydrochloride

Compound 715 3-{(S)-3-[2-(4-Methoxy-benzyl)-3-oxo-2,8-diaza-spiro[4.5]dec-8-yl]-1-phenyl-propyl}-1,1-dimethyl-urea hydrochloride

Compound 716
Morpholine-4-carboxylic acid {(S)-3-[2-(4-methoxy-benzyl)-3-oxo-2,8-diaza-spiro[4.5]dec-8-yl]-1-phenyl-propyl}-amide hydrochloride

Compound 717
3,3-Difluoro-pyrrolidine-1-carboxylic acid {(S)-3-[2-(4-methoxy-benzyl)-3-oxo-2,8-diaza-spiro[4.5]dec-8-yl]-1-phenyl-propyl}-amide hydrochloride

Compound 718
{(S)-3-[2-(4-Methoxy-benzyl)-3-oxo-2,8-diaza-spiro[4.5]dec-8-yl]-1-phenyl-propyl}-carbamic acid methyl ester hydrochloride

Compound 719
(S)-3-[2-(4-Methoxy-benzyl)-3-oxo-2,8-diaza-spiro[4.5]dec-8-yl]-1-phenyl-propyl}-carbamic acid ethyl ester hydrochloride
   and pharmaceutically acceptable salts, hydrates or solvates thereof. While the compounds are listed above as their hydrochloride salts, this aspect of the invention includes their non-salt forms, as well as pharmaceutically acceptable salts, hydrates and solvates thereof.

It will be appreciated that the amount of a compound of the invention required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, preferably in the range of 0.5 to 60 mg/kg/day, most preferably in the range of 1 to 20 mg/kg/day.

The desired dose may conveniently be presented in a single dose or as divided dose administered at appropriate intervals, for example as two, three, four or more doses per day.

The compound is conveniently administered in unit dosage form; for example containing 10 to 1500 mg, conveniently 20 to 1000 mg, most conveniently 50 to 700 mg of active ingredient per unit dosage form.

Ideally the active ingredient should be administered to achieve peak plasma concentrations of the active compound of from about 1 to about 75 μM, preferably about 2 to 50 μM, most preferably about 3 to about 30 μM. This may be achieved, for example, by the intravenous injection of a 0.1 to 5% solution of the active ingredient, optionally in saline, or orally administered as a bolus containing about 1 to about 500 mg of the active ingredient. Desirable blood levels may be maintained by a continuous infusion to provide about 0.01 to about 5.0 mg/kg/hour or by intermittent infusions containing about 0.4 to about 15 mg/kg of the active ingredient.

While it is possible that, for use in therapy, a compound of the invention may be administered as the raw chemical it is preferable to present the active ingredient as a pharmaceutical formulation. The invention thus further provides a pharmaceutical formulation comprising a compound of formula (I) or a pharmaceutically acceptable derivative thereof together with one or more pharmaceutically acceptable carriers therefor and, optionally, other therapeutic and/or prophylactic ingredients. The carrier(s) must be “acceptable” in the sense of being compatible with the other ingredients of the formulation and not deleterious to the recipient thereof.

Pharmaceutical formulations include those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical formulation suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

For topical administration to the epidermis, the compounds according to the invention may be formulated as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Formulations suitable for topical administration in the mouth include lozenges comprising active ingredient in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical formulations suitable for rectal administration wherein the carrier is a solid are most preferably presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

Formulations suitable for vaginal administration may be presented as pessaries, tampons, creams, gels, pastes, foams or sprays containing in addition to the active ingredient such carriers as are known in the art to be appropriate.

For intra-nasal administration the compounds of the invention may be used as a liquid spray or dispersible powder or in the form of drops. Drops may be formulated with an aqueous or non-aqueous base also comprising one more dispersing agents, solubilising agents or suspending agents. Liquid sprays are conveniently delivered from pressurized packs.

For administration by inhalation the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the compounds according to the invention may take the form of a dry powder composition, for example a powder mix of the compound and a suitable powder base such as lactose or starch. The powder composition may be presented in unit dosage form in, for example, capsules or cartridges or e.g. gelatin or blister packs from which the powder may be administered with the aid of an inhalator or insufflator.

When desired the above described formulations adapted to give sustained release of the active ingredient may be employed.

When the compound (I) or a pharmaceutically acceptable salt, hydrate or solvate thereof is used in combination with a second therapeutic active agent, the dose of each compound may be either the same as or different from that when the compound is used alone. Conventional doses and regimens are readily appreciated by those skilled in the art, including doses described in the Physicians Desk Reference, 56^{th} edition, 2002.

The present invention is directed to the use of the compounds as modulators of CCR5 chemokine receptor activity. In particular, the compounds of the invention have been found to have activity in binding to the CCR5 receptor in the biological assay, as described in Example 15, generally with an IC₅₀ value of less than 25 μM. The terms “modulator” or “modulation” are meant to include antagonism, agonism, mixed and partial antagonism and agonism.

Certain compounds of the present invention have also been tested in an assay for HIV activity, as described in Example 15, and generally having an IC₅₀ value of less than 1 μM.

The purity and mass of the following examples were characterized by mass spectra (LC/MS) and or NMR spectra.

The following general schemes and examples are provided to illustrate various embodiments of the present invention and shall not be considered as limiting in scope.

The following abbreviations may be used as follows:
br broad
DCC 1,3-dicyclohexylcarbodiimide
DCE 1,2-dichloroethane
DCM dichloromethane
DIPEA N,N-diisopropylethylamine
DMF N,N-dimethylformamide
Hal halogen
LAH lithium aluminium hydride
TFA trifluoroacetic acid
THF tetrahydrofuran

The semi-preparative HPLC purification procedures used are described below:

Column: Phenomenex Luna C₁₈(2), 5 microns, 10×250 mm

Buffer A: 3 mM HCl in H₂O (pH 2.4-2.6)

Buffer B: acetonitrile

Method A: 15-55% B in 30 min. (1.4%/min)

Method B: 10-60% B in 50 min. (1%/min)

Method C: 20-50% B in 21 min. (1.4%/min)

Method D: 10-60% B in 42 min. (1.2%/min)

Method E: 15-45% B in 21 min. (1.4%/min) or

Buffer A: H₂O

Buffer B: acetonitrile

Method F: 15-55% B in 40 min. (1%/min)

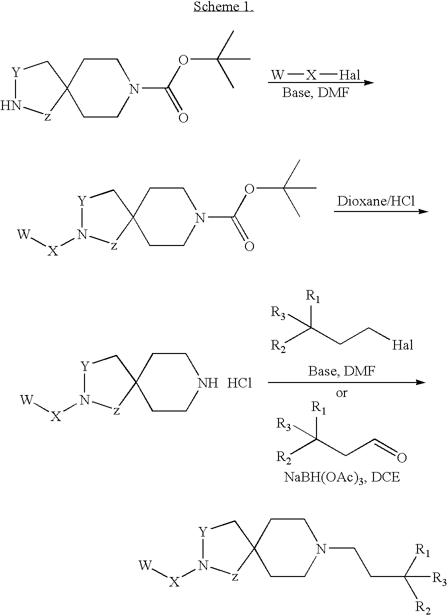

### Preparation 1

### 2-(4-Bromobenzyl)-1-oxo-2,8-diaza-spiro[4.5]decane-8-carboxylic acid tert-butyl ester

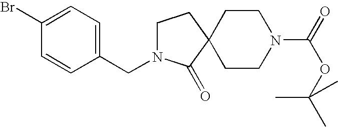 Sodium hydride 600 mg (14.7 mmol) (60% suspension in mineral oil) was added in a 500 mL round bottom flask under nitrogen followed by 20 mL of anhydrous DMF and 2.5 g (9.8 mmol) of 1-oxo-2,8-diaza-spiro[4.5]decane-8-carboxylic acid tert-butyl ester previously dissolved in 20 mL of anhydrous DMF. After agitating one hour at room temperature, 2.5 g (9.8 mmol) of 4-bromobenzylbromide diluted in 20 mL of anhydrous DMF were added and the reaction mixture was agitated an additional hour at room temperature. Then 100 mL of water were added and the solution was extracted with diethyl ether (2×150 mL). The combined organic layers were dried (Na₂SO₄), filtered and evaporated under reduced pressure to yield 4.63 g 2-(4-bromobenzyl)-1-oxo-2,8-diaza-spiro[4.5]decane-8-carboxylic acid tert-butyl ester as a yellow oil.

¹H NMR (400 MHz, DMSO-d₆): δ[ppm] 7.51 (d, 2H), 7.12 (d, 2H), 4.31 (s, 2H), 3.8 (br d, 2H), 3.14 (t, 2H), 2.86 (br s, 2H), 1.89 (t, 2H), 1.54 (t×d, 2H), 1.37 (s, 9H), 1.32 (br d, 2H).

### Preparation 2

### 2-(4-Bromobenzyl)-2,8-diaza-spiro[4.5]decan-1-one hydrochloride

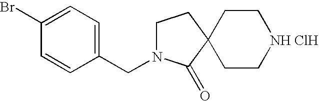 To 4.62 g of crude 2-(4-bromobenzyl)-1-oxo-2,8-diaza-spiro[4.5]decane-8-carboxylic acid tert-butyl ester from preparation 1 was added 50 mL of 4N solution of dioxane/HCl. The reaction mixture was agitated 15 minutes at room temperature and 3.05 g (77.8%) of 2-(4-bromobenzyl)-2,8-diaza-spiro[4.5]decan-1-one hydrochloride was collected, as a colorless solid by filtration followed by trituration with diethyl ether.

1H NMR (400 MHz, DMSO-d₆): δ[ppm] 9.15 (br s, 1H), 8.83 (br s, 1H), 7.51 (d, 2H), 7.14 (d, 2H), 4.31 (s, 2H), 3.24 (br d, 2H), 3.15 (t, 2H), 2.92 (q, 2H), 1.95-1.84 (m, 4H), 1.56 (br d, 2H).

### Preparation 3

### 2-(4-Methylsulfanylbenzyl)-1-oxo-2,8-diaza-spiro[4.5]decane-8-carboxylic acid tert-butyl ester

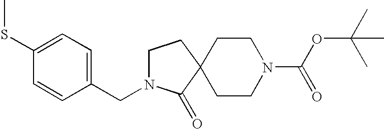 This spiro compound was prepared as described in preparation 1, starting from 7 g (27.5 mmol) of 1-oxo-2,8-diaza-spiro[4.5]decane-8-carboxylic acid tert-butyl ester, excepted it was purified by flash chromatography on silica gel (ethyl acetate/hexanes 0:100 to 20:80) yielding 8.05 g (74.9%) of 2-(4-methylsulfanylbenzyl)-1-oxo-2,8-diaza-spiro[4.5]decane-8-carboxylic acid tert-butyl ester as a pale yellow solid.

¹H NMR (400 MHz, DMSO-d₆): δ[ppm] 7.21 (d, 2H), 7.11 (d, 2H), 4.3 (s, 2H), 3.8 (br d, 2H), 3.13 (t, 2H), 2.88 (br s, 2H), 2.43 (s, 3H), 1.89 (t, 2H), 1.54 (t×d, 2H), 1.38 (s, 9H), 1.31 (br d, 2H).

### Preparation 4

### 2-(4-Methanesulfonylbenzyl)-1-oxo-2,8-diaza-spiro[4.5]decane-8-carboxylic acid tert-butyl ester

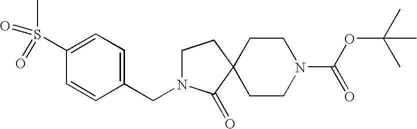 To a solution of 7.73 g (19.8 mmol) of 2-(4-ethylsulfanylbenzyl)-1-oxo-2,8-diaza-spiro[4.5]decane-8-arboxylic acid tert-butyl ester in 100 mL of THF, was added 18.2 g (29.7 mmol) of Oxone® in 100 mL of water. The reaction mixture was agitated overnight at room temperature. An aqueous solution of sodium hydroxide (1N, 100 mL) was added and the solution was extracted with DCM (2×200 mL). The combined organic layers were dried (Na₂SO₄), filtered and evaporated under reduced pressure to yield 6.62 g (79.1%) of 2-(4-methanesulfonylbenzyl)-1-oxo-2,8-diaza-spiro[4.5]decane-8-carboxylic acid tert-butyl ester as a white solid.

¹H NMR (400 MHz, DMSO-d₆): δ[ppm] 7.81 (d, 2H), 7.37 (d, 2H), 4.41 (s, 2H), 3.76 (br d, 2H), 3.15 (t, 2H), 3.14 (s, 3H), 2.86 (br s, 2H), 1.89 (t, 2H), 1.52 (t×d, 2H), 1.33 (s, 9H), 1.29 (br d, 2H).

LC/MS: m/z 423.2 (MH⁺).

### Preparation 5

### 2-(4-Methanesulfonylbenzyl)-2,8-diaza-spiro[4.5]decan-1-one hydrochloride

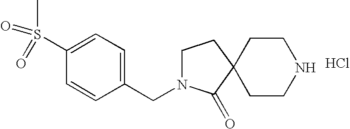 As described in preparation 2, 6.62 g (15.6 mmol) of 2-(4-methanesulfonylbenzyl)-1-oxo-2,8-diaza-spiro[4.5]decane-8-carboxylic acid tert-butyl ester was deprotected under acidic conditions giving access to 5.25 g (93.7%) of 2-(4- methanesulfonylbenzyl)-2,8-diaza-spiro[4.5]decan-1-one hydrochloride as a white solid.

¹H NMR (400 MHz, DMSO-d₆): δ[ppm] 9.04 (br s, 1H), 8.74 (br s, 1H), 7.83 (d, 2H), 7.39 (d, 2H), 4.42 (s, 2H), 3.21 (br d, 2H), 3.15 (t, 2H), 3.13 (s, 3H), 2.89 (q, 2H), 1.92 (t, 2H), 1.84 (t×d, 2H), 1.55 (br d, 2H).

### Preparation 6

### 2-(4-Bromobenzyl)-3-oxo-2,8-diaza-spiro[4.5]decane-8-carboxylic acid tert-butyl ester

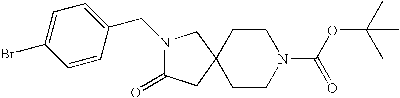 This spiro compound was prepared as described in preparation 1, starting from 300 mg (1.18 mmol) of 3-oxo-2,8-diaza-spiro[4.5]decane-8-carboxylic acid tert-butyl ester which was purified by flash chromatography on silica gel (ethyl acetate/hexanes 0:100 to 60:40) yielding 290 mg (58%) of 2-(4-bromobenzyl)-3-oxo-2,8-diaza-spiro[4.5]decane-8-carboxylic acid tert-butyl ester as a colorless oil.

¹H NMR (400 MHz, DMSO-d₆): δ[ppm] 7.52 (d, 2H), 7.16 (d, 2H), 4.31 (s, 2H), 3.32 (m, 2H), 3.16 (br s, 2H), 3.02 (s, 2H), 2.25 (s, 2H), 1.4 (m, 4H), 1.35 (s, 9H).

### Preparation 7

### 2-(4-Bromobenzyl)-2,8-diaza-spiro[4.5]decan-3-one hydrochloride

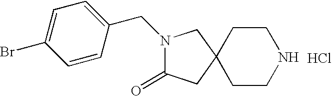 As described in preparation 2, 290 mg (0.68 mmol) of 2-(4-bromobenzyl)-3-oxo-2,8-diaza-spiro[4.5]decane-8-carboxylic acid tert-butyl ester was deprotected under acidic conditions giving to 212 mg (86.6%) of 2-(4-bromobenzyl)-2,8-diaza-spiro[4.5]decan-3-one hydrochloride as a white solid.

¹H NMR (400 MHz, DMSO-d₆): δ[ppm] 8.63 (br s, 2H), 7.52 (d, 2H), 7.17 (d, 2H), 4.32 (s, 2H), 3.07 (s, 2H), 3.00 (m, 4H), 2.33 (s, 2H), 1.65 (m, 4H).

### EXAMPLE 1

### 2-(4-Bromobenzyl)-8-(3,3-diphenyl-propyl)-2,8-diaza-spiro[4.5]decan-3-one hydrochloride (Compound 13)

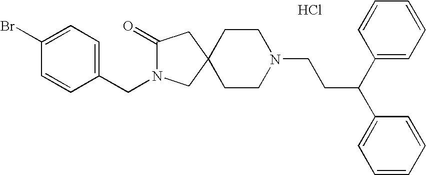 A mixture of 28.7 mg (80 μmol) of 2-(4-bromobenzyl)-2,8-diaza-spiro[4.5]decan-3-one-hydrochloride, 24.4 mg (88 μmol) of 3,3-diphenylpropyl bromide and 33.1 mg (240 μmol) of potassium carbonate in 1.5 mL of anhydrous DMF was heated overnight at 60° C. After cooling to room temperature, 0.5 mL of water was added and the solution was extracted with DCM (2×2 mL). The crude material was purified by semi-preparative HPLC (method A) yielding 22.6 mg (51%) of Compound 13 as a white solid.

¹H NMR (400 MHz, DMSO-d₆): δ[ppm] 9.96 (br s, 1H), 7.52 (d, 2H), 7.34-7.25 (m, 8H), 7.19-7.13 (m, 4H), 4.31 (s, 2H), 3.96 (q, 1H), 3.36 (m, 4H), 3.05 (d, 1H), 2.96-2.83 (m, 4H), 2.47 (s, 2H), 2.3 (d, 1H), 1.75 (m, 4H).

LC/MS: m/z 519.0 (MH⁺).

Table 1 of compounds illustrates some of the compounds of the present invention that were synthesized using the procedure described in scheme 1.

TABLE 1

| CPD | | | | |
|---|---|---|---|---|
| # | MOL STRUCTURE | COMPOUND NAME | MOL WT | PURITY |
| 1 | 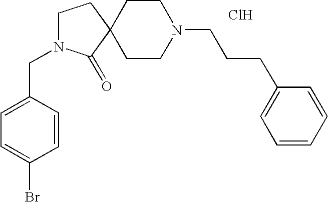 | 2-(4-bromobenzyl)-8-(3-phenyl- propyl)-2,8-diaza-spiro[4.5]decan-1-one hydrochloride | 477.871 | 95.1% (LC/MS) |
| 2 | 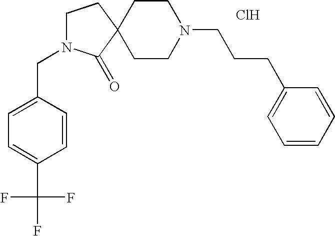 | 8-(2-phenylpropyl)-2-(4- trrifluoromethyl-benzyl)-2,8-diaza- spiro[4.5]decan-1-one hydrochloride | 466.972 | 99+(LC/MS) |
| 3 | 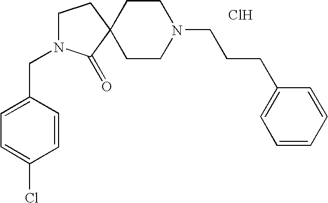 | 2-(4-chlorobenzyl)-8-(3-phenyl- propyl)-2,8-diaza-spiro[4.5]decan- 1-one hydrochloride | 433.42 | 94.8% (HPLC) |
| 4 | 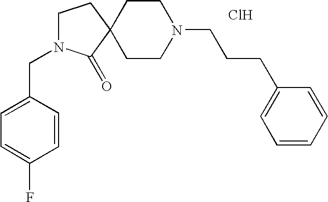 | 2-(4-fluorobenzyl)-8-(3-phenyl- propyl)-2,8-diaza-spiro[4.5]decan- 1-one hydrochloride | 416.965 | 90.5% (HPLC) |
| 5 | 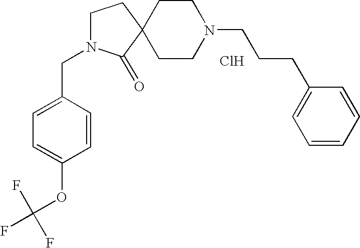 | 8-(3-phenyl-prropyl)-2-(4- trifluoromethoxy-benzyl)-2,8- diaza-spiro[4.5]decan-1-one hydrochloride | 482.971 | 92.2% (HPLC) |
| 6 | 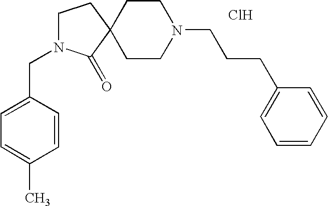 | 2-(4-methylbenzyl)-8-(3-phenyl- propyl)-2,8-diaza-spiro[4.5]decan- 1-one hydrochloride | 413.002 | 93.7% (HPLC) |
| 7 | 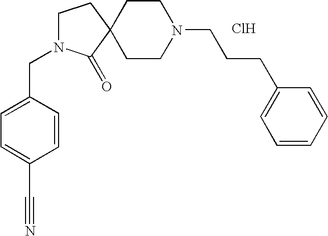 | 4-[1-oxo-8-(3-phenyl-propyl)-2,8- diaza-spiro[4.5]dec-2-ylmethyl]- benzonitrile hydrochloiride | 423.985 | 94.1% (HPLC) |
| 8 | 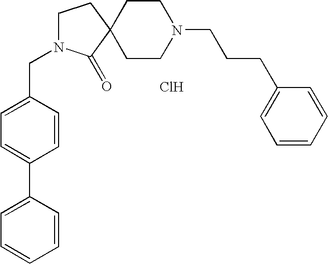 | 2-biphenyl-4-ylmethyl-8-(3-phenyl- propyl)-2,8-diaza-spiro[4.5]decan- 1-one hydrochloride | 475.073 | 99+(HPLC) |
| 9 | 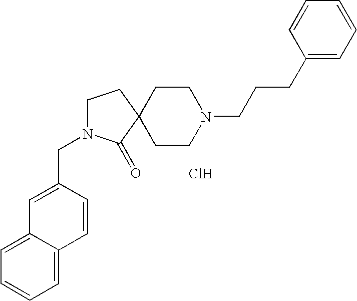 | 2-naphthalen-2-ylmethyl-8-(3- phenyl-propyl)-2,8-diaza- spiro[4.5]decan-1-one hydrochloride | 449.035 | 99+(HPLC) |
| 10 | 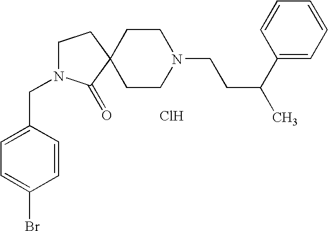 | 2-(4-bromobenzyl)-8-(3-phenyl- butyl)-2,8-diaza-spiro[4.5]decan-1- one hydrochloride | 491.898 | 90+(HPLC) |
| 11 | 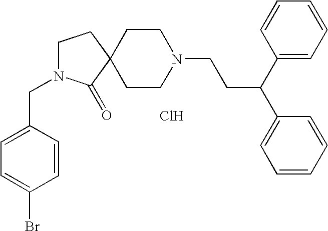 | 2-(4-bromobenzyl)-8-(3,3- diphenyl-propyl)-2,8-diaza- spiro[4.5]decan-1-one hydrochloride | 553.969 | 99+(HPLC) |
| 12 | 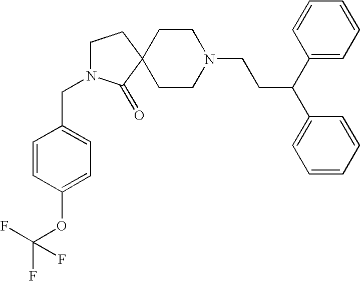 | 8-(3,3-diphenyl-propyl)-2-(4- trifluoromethoxy-benzyl)-2,8- diaza-spiro[4.5]decan-1-one | 522.608 | 100% (LC/MS) |
| 13 | 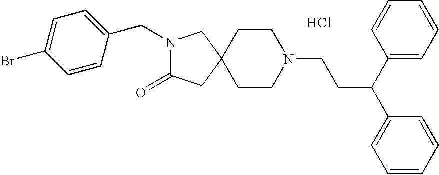 | 2-(4-bromobenzyl)-8-(3,3- diphenyl-propyl)-2,8-diaza- spiro[4.5]decan-3-one hydrochloride | 553.969 | >95% (LC/MS) |
| 14 | 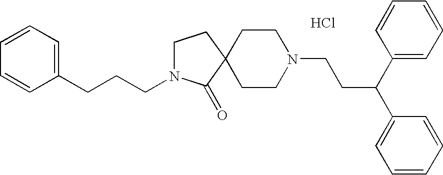 | 8-(3,3-diphenyl-propyl)-2-(3- phenyl-propyl)-2,8-diaza- spiro[4.5]decan-1-one hydrochloride | 503.126 | 100% (LC/MS) |
| 15 | 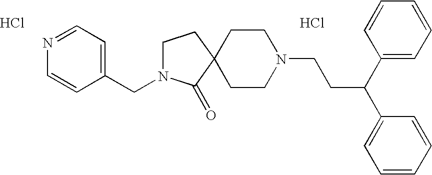 | 8-(3,3-diphenyl-propyl)-2-pyridin- 4-ylmethyl-2,8-diaza- spiro[4.5]decan-1-one dihydrochloride | 512.521 | 100% (LC/MS) |
| 16 | 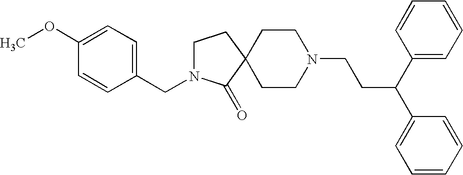 | 8-(3,3-dipheenyl-propyl)-2-(4- methoxy-benzyl)-2,8-diaza- spiro[4.5]decan-1-one | 468.637 | 93% (LC/MS) |
| 17 | 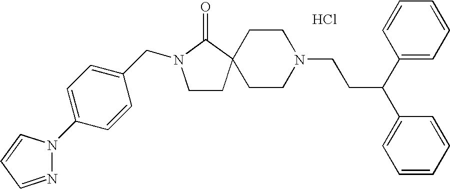 | 8-(3,3-diphenyl-propyl)-2-(4- pyrazol-1-yl-benzyl)-2,8-diaza- spiro[4.5]decan-1-one hydrochloride | 541.135 | 98% (LC/MS) |
| 18 | 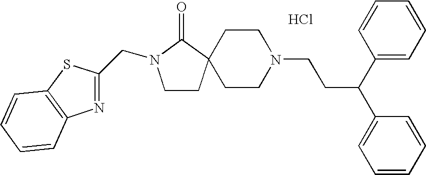 | 2-benzothiazol-2-ylmethyl-8-(3,3- diphenyl-propyl)-2,8-diaza- spiro[4.5]decan-1-one hydrochloride | 532.149 | 93% (LC/MS) |
| 19 | 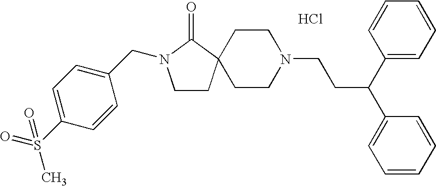 | 8-(3,3-diphenyl-propyl)-2-(4- methanesulfonyl-benzyl)-2,8- diaza-spiro[4.5]decan-1-one hydrochloride | 553.163 | 98+(LC/MS) |
| 20 | 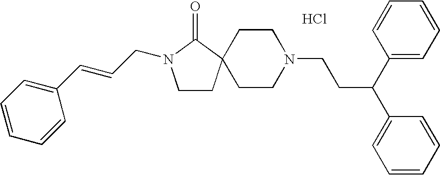 | 8-(3,3-diphenyl-propyl)-2-(3- phenyl-allyl)-2,8-diaza- spiro[4.5]decan-1-one hydrochloride | 501.11 | 98% (LC/MS) |
| 21 | 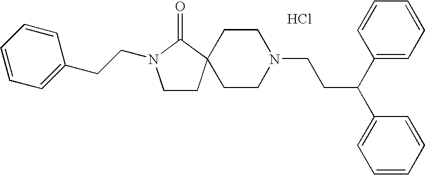 | 8-(3,3-diphenyl-propyl)-2- phenethyl-2,8-diaza- spiro[4.5]decan-1-one hydrochloride | 489.099 | 98% (LC/MS) |
| 22 | 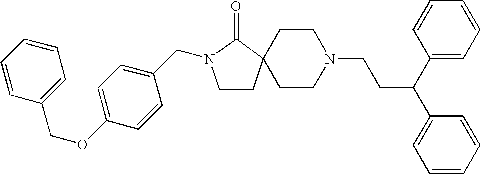 | 2-(4-benzyloxy-benzyl)-8-(3,3- diphenyl-propyl)-2,8-diaza- spiro[4.5]decan-1-one | 544.735 | 100% (LC/MS) |
| 23 | 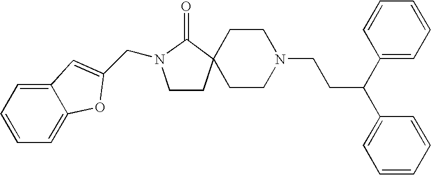 | 2-benzofuran-2-ylmethyl-8-(3,3- diphenyl-propyl)-2,8-diaza- spiro[4.5]decan-1-one | 478.633 | 90% (LC/MS) |
| 24 | 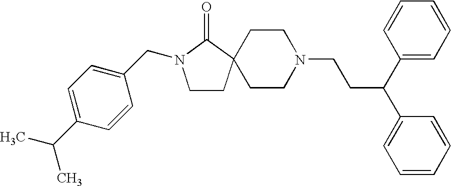 | 8-(3,3-diphenyl-propyl)-2-(4- isopropyl-benzyl)-2,8-diaza- spiro[4.,5]decan-1-one | 480.692 | 100% (LC/MS) |
| 25 | 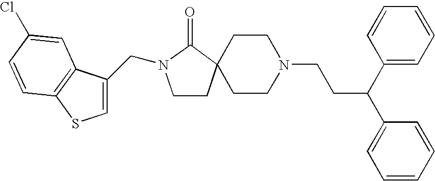 | 2-(5-chloro-benzo[b]thiophen-3- ylmethyl)-8-(3,3-diphenyl-propyl)- 2,8-diaza-spiro[4.5]decan-1-one | 529.145 | 100% (LC/MS) |
| 26 | 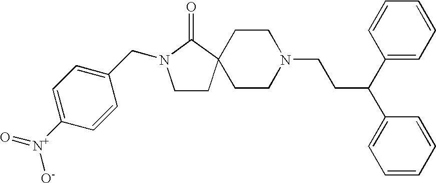 | 8-(3,3-diphenyl-propyl)-2-(4-nitro- benzyl)-2,8-diaza-spiro[4.5]decan- 1-one | 483.609 | 100% (LC/MS) |
| 27 | 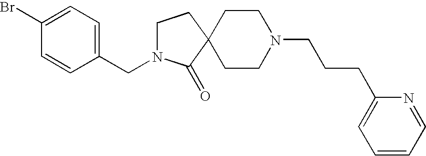 | 2-(4-bromo-benzyl)-8-(3-pyridin-2- yl-propyl)-2,8-diaza- spiro[4.5]decan-1-one | 442.398 | 97% (HPLC) |
| 28 | 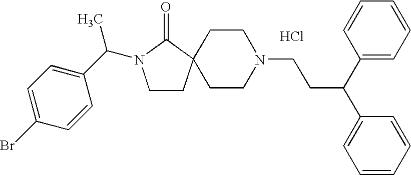 | 2-[1-(4-bromopheenyl)-ethyl]-8-(3,3- diphenyl-propyl)-2,8-diaza- spiro[4.5]decan-1-one hydrochloride | 567.995 | 98+(LC/MS) |
| 29 | 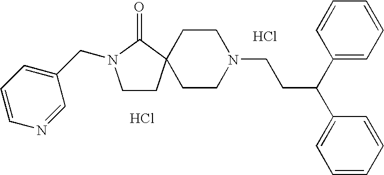 | 8-(3,3-diphenyl-propyl)-2-pyridin- 3-ylmethyl-2,8-diaza- spiro[4.5]decan-1-one dihydrochloride | 512.521 | 98+(LC/MS) |
| 30 | 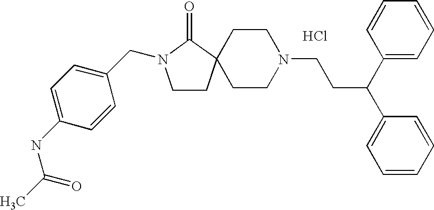 | N-{4-[8-(3,3-diphenyl-propyl)-1- oxo-2,8-diaza-spiro[4.5]dec-2- ylmethyl]-phenyl}-acetamide hydrochloride | 532.124 | 98+(LC/MS) |
| 31 | 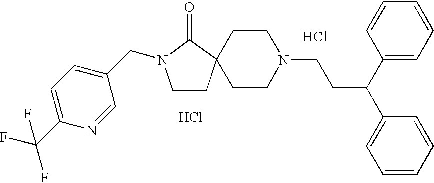 | 8-(3,3-diphenyl-propyl)-2-(6- trifluoromethyl-pyridin-3-ylmethyl)- 2,8-diaza-spiro[4.5]decan-1-one dihydrochloride | 580.519 | 90+(LC/MS) |
| 32 | 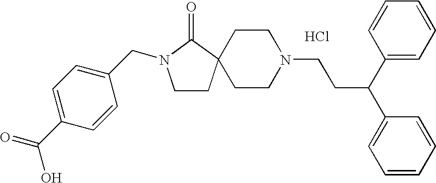 | 4-[8-(3,3-diphenyl-propyl)-1-oxo- 2,8-diaza-spiro[4.5]dec-2- ylmethyl]-benzoic acid hydrochloride | 519.081 | 98+(LC/MS) |
| 33 | 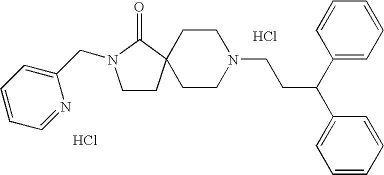 | 8-(3,3-diphenyl-propyl)-2-pyridin- 2-ylmethyl-2,8-diaza- spiro[4.5]decan-1-one dihydrochloride | 512.521 | 98+(LC/MS) |
| 34 | 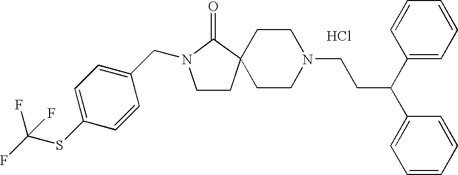 | 8-(3,3-diphenyl-propyl)-2-(4- trifluoromethylsulfanyl-benzyl)-2,8- diaza-spiro[4.5]decan-1-one hydrochloride | 575.136 | 90+(LC/MS) |
| 35 | 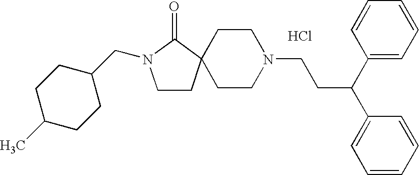 | 8-(3,3-diphenyl-propyl)-2-(4- methyl-cyclohexylmethyl)-2,8- diaza-spiro[4.5]decan-1-one hydrochloride | 495.147 | 98+(LC/MS) |
| 36 | 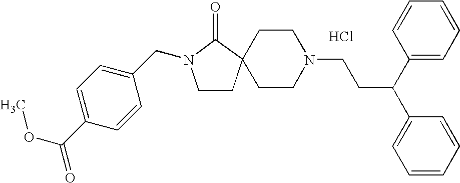 | 4-[8-(3,3-diphenyl-propyl)-1-oxo- 2,8-diaza-spiro[4.5]dec-2- ylmethyl)-benzoic acid methyl ester hydrochloride | 533.108 | 90.7% (LC/MS) |
| 37 | 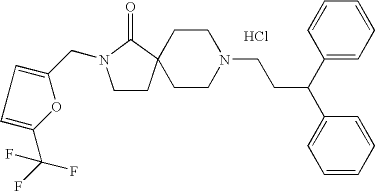 | 8-(3,3-diphenyl-propyl)-2-(5- trifluoromethyl-furan-2-ylmethyl)- 2,8-diaza-spiro[4.5]decan-1-one hydrochloride | 533.031 | 98+(LC/MS) |
| 38 | 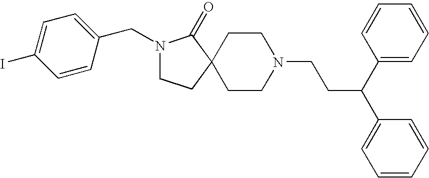 | 8-(3,3-diphenyl-propyl)-2-(4-iodo- benzyl)-2,8-diaza-spiro[4.5]decan- 1-one | 564.504 | >95% (1H NMR) |
| 39 | 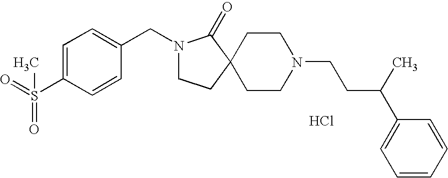 | 2-(4-methanesulfonylbenzyl)-8-(3- phenyl-butyl)-2,8-diaza- spiro[4.5]decan-1-one hydrochloride | 491.092 | 98+(LC/MS) |

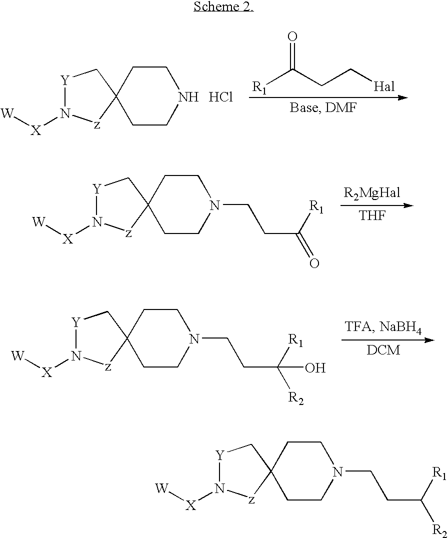

### Preparation 8

### 2-(4-Bromobenzyl)-8-(3-oxo-3-phenylpropyl)-2,8-diaza-spiro[4.5]decan-1-one

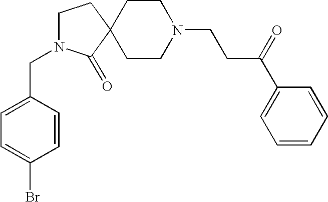 To a stirred solution of 2-(4-bromobenzyl)-2,8-diaza-spiro[4.5]decan-1-one hydrochloride (2.30 g, 6.39 mmol) in DMF (43 mL) was added DIPEA (4.2 mL, 3.80 mmol) followed by 3-chloro-1-phenyl-propan-1-one (1.08 g, 6.39 mmol). The reaction mixture was stirred at room temperature for 18 hours and then a saturated solution of NaHCO₃ was added and the mixture was extracted with ethyl acetate (3×30 mL). The combined organic extracts were washed with water (3×30 mL) and brine (30 mL), dried over Na₂SO₄, filtered and concentrated. The crude product was purified by flash chromatography on silica gel (0% to 5% methanol/DCM) to give 2.53 g (87%) of 2-(4-bromobenzyl)-8-(3-oxo-3-phenylpropyl)-2,8-diaza-spiro(4.5]decan-1-one.

¹H NMR (400 MHz, CDCl₃): δ[ppm] 7.98-7.95 (m, 2H), 7.59-7.54 (m, 1H), 7.49-7.42 (m, 4H), 7.10-7.07 (m, 2H), 4.39 (s, 2H), 3.21 (t, 2H), 3.14 (t, 2H), 2.93-2.90 (m, 2H), 2.84 (t, 2H), 2.19-2.14 (m, 2H), 2.05-1.98 (m, 2H), 1.90 (t, 2H), 1.44-1.41 (m, 2H).

### EXAMPLE 2

### 2-(4-Bromobenzyl)-8-[3-hydroxy-3-(2-methoxyphenyl)- 3-phenylpropyl]-2,8-diaza-spiro[4.5]decan-1-one (Compound 40)

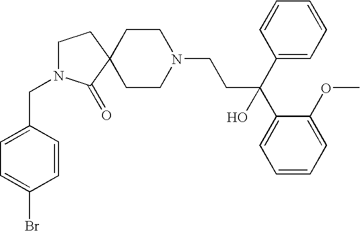 To a stirred solution of 2-(4-bromobenzyl)-8-(3-oxo-3-phenylpropyl)-2,8-diaza-spiro[4.5]decan-1-one (157 mg, 0.35 mmol) in THF (3.5 mL) at 0° C. was added a 1.0 M solution of 2-methoxyphenylmagnesium bromide (1.4 mL, 1.4 mmol). The reaction mixture was warmed to room temperature and stirred for 18 hours. Water was added and the mixture was extracted with ethyl acetate (3×10 mL). The combined organic extracts were washed with brine (10 mL), dried over Na₂SO₄, filtered and concentrated. The crude product was purified by semi-preparative HPLC (method F) to give 133 mg (69%) of Compound 40 as a yellow oil.

¹H NMR (400 MHz, CDCl₃): δ[ppm] 7.90 (d, 1H), 7.52-7.39 (m, 4H), 7.24 (m, 3H), 7.15 (m, 1H), 7.08-7.01 (m, 3H), 6.79 (d, 1H), 4.42-4.33 (m, 2H), 3.48 (s, 3H), 3.11 (t, 2H), 3.05 (m, 1H), 2.78 (m, 1H), 2.64 (m, 1H), 2.50-2.45 (m, 2H), 2.34 (m, 1H), 2.23 (m, 1H), 2.00-1.88 (m, 3H), 1.85 (m, 2H), 1.45-1.40 (m, 2H).

### EXAMPLE 3

### 2-(4-Bromobenzyl)-8-[3-(2-methoxyphenyl)-3-phenylpropyl]-2,8-diaza-spiro[4.5]decan-1-one (Compound 44)

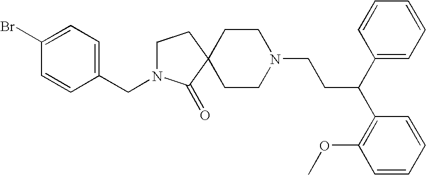 To trifluoroacetic acid (1.4 mL) at room temperature was added portion wise sodium borohydride (67 mg, 1.775 mmol). This mixture was stirred at room temperature for 30 minutes and then a solution of 2-(4-bromobenzyl)-8-[3-hydroxy-3-(2-methoxyphenyl)-3-phenylpropyl)-2,8-diaza-spiro[4.5]decan-1-one (40 mg, 0.071 mmol) in DCM (0.5 mL) was slowly added. The reaction mixture was stirred at room temperature for 20 hours and then poured into an ice cold solution of sodium hydroxide (5 mL). The mixture was extracted with ethyl acetate (3×5 mL) and the combined organic extracts were washed with brine (5 mL), dried over Na₂SO₄, filtered and concentrated. The crude product was purified by flash chromatography on silica gel (0% to 4% methanol/DCM) to give 24 mg (61%) of Compound 44 as a yellow oil.

¹H NMR (400 MHz, CDCl₃): δ[ppm] 7.43 (d, 2H), 7.38-7.32 (m, 1H), 7.26-7.2 (m, 5H), 7.12-6.94 (m, 5H), 6.35 (t, 1H), 4.36 (s, 2H), 3.7 (s, 3H), 3.2-2.9 (m, 5H), 2.1-1.9 (m, 2H), 1.85 (t, 2H).

Table 2 of compounds illustrates some of the compounds of the present invention that were synthesized using the procedure described in scheme 2.

TABLE 2

| CPD | | | MOL | |
|---|---|---|---|---|
| # | MOL STRUCTURE | COMPOUND NAME | WT | PURITY |
| 40 | 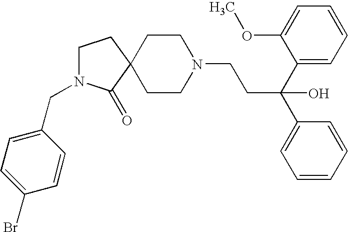 | 2-(4-bromobenzyl)-8-[3-hydroxy-3- (2-methoxyphenyl)-3-phenyl- propyl]-2,8-diaza-spiro[4.5]decan- 1-one | 563.532 | 93% BY HPLC |
| 41 | 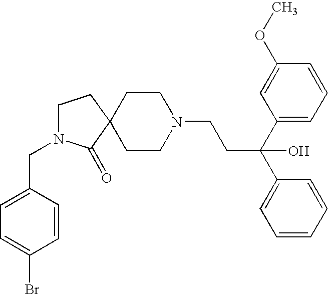 | 2-(4-bromobenzyl)-8-[3-hydroxy-3- (3-methoxyphenyl)-3-phenyl- propyl]-2,8-diaza-spiro[4.5]decan- 1-one | 563,532 | 99% BY HPLC |
| 42 | 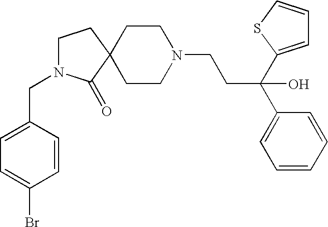 | 2-(4-bromobenzyl)-8-(3-hydroxy-3- phenyl-3-thiophen-2-yl-propyl)-2,8- diaza-spiro[4.5]decan-1-one | 539.535 | 97% BY HPLC |
| 43 | 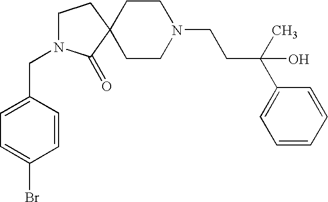 | 2-(4-bromobenzyl)-8-(3-hydroxy-3- phenyl-butyl)-2,8-diaza- spiro[4.5]decan-1-one | 471.436 | 99% BY HPLC |
| 44 | 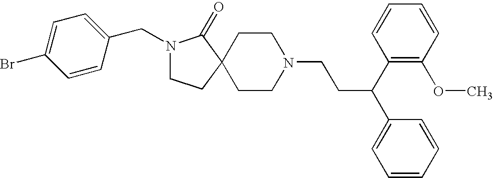 | 2-(4-bromobenzyl)-8-[3-(2- methoxyphenyl)-3-phenyl-propyl]- 2,8-diaza-spiro[4.5]decan-1-one | 547.534 | 99% BY HPLC |
| 45 | 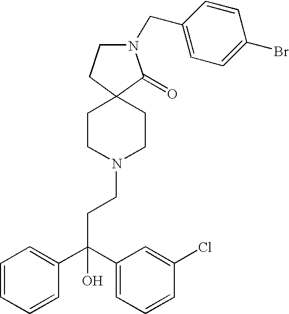 | 2-(4-bromobenzyl)-8-[3-(3- chlorophenyl)-3-hydroxy-3-phenyl- propyl]-2,8-diaza-spiro[4.5]decan- 1-one | 567.952 | 94% BY HPLC |
| 46 | 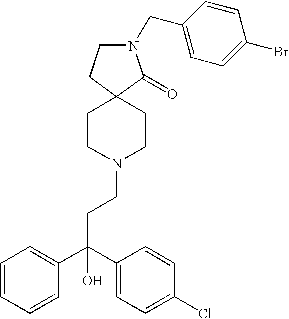 | 2-(4-bromobenzyl)-8-[3-(4- chlorophenyl)-3-hydroxy-3-phenyl- propyl]-2,8-diaza-spiro[4.5]decan- 1-one | 567.952 | 97% BY HPLC |
| 47 | 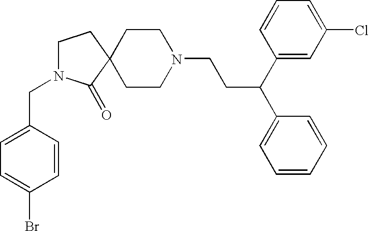 | 2-(4-bromobenzyl)-8-[3-(3- chlorophenyl)-3-phenyl-propyl]-2,8- diaza-spiro[4.5]decan-1-one | 551.953 | 96% BY HPLC |
| 48 | 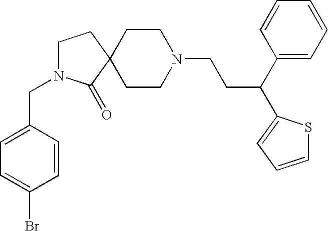 | 32-(4-bromobenzyl)-8-(3-phenyl-3- thiophen-2-yl-propyl)-2,8-diaza- spiro[4.5]decan-1-one | 523.536 | 89% BY HPLC |
| 49 | 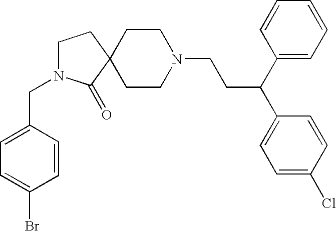 | 2-(4-bromobenzyl)-8-[3-(4- chlorophenyl)-3-phenyl-propyl]-2,8- diaza-spiro[4.5]decan-1-one | 551.953 | 96% BY HPLC |

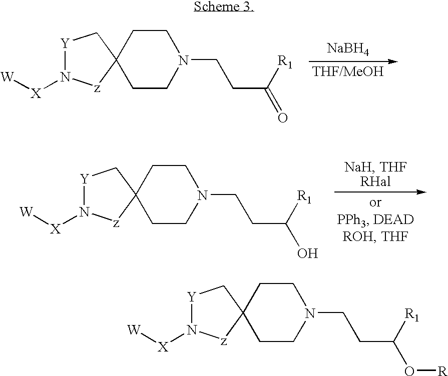

### EXAMPLE 4

### 2-(4-Bromobenzyl)-8-(3-hydroxy-3-phenylpropyl)-2,8-diaza-spiro[4.5]decan-1-one (Compound 50)

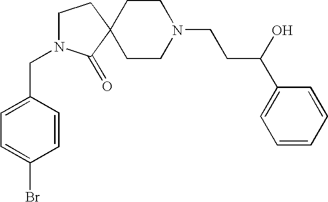 To a cold stirred solution of 2-(4-bromobenzyl)-8-(3-oxo-3-phenylpropyl)-2,8-diaza-spiro[4.5]decan-1-one (350 mg, 0.769 mmol) in THF-methanol (7:3, 4.0 mL) was added NaBH₄ (85 mg, 2.3 mmol). After stirring for one hour, the reaction mixture was then quenched with an aqueous solution of sodium hydroxide (1N). The reaction mixture was portioned in a separating funnel and the aqueous solution was then extracted with ethyl acetate (3×10 mL). The combined organic extracts were washed with brine and dried over sodium sulfate. Evaporation of the solvent gave

Compound 50 as an oil (300 mg, 85.4%).

¹H NMR (400 MHz, CDCl₃): δ[ppm] 7.44 (d×d, 2H), 7.40-7.3 (m, 4H), 7.26-7.23 (m, 1H), 7.09 (d×d, 2H), 4.94 (d×d, 1H), 4.39 (s, 2H), 3.14 (t, 2H), 3.1-2.9 (m, 2H), 2.7-2.5 (m, 2H), 2.3-1.8 (m, 9H), 1.6-1.4 (m, 2H).

### EXAMPLE 5

### 8-(3-Benzyloxy-3-phenylpropyl)-2-(4-bromobenzyl)-2,8-diaza-spiro[4.5]decan-1-one hydrochloride (Compound 51)

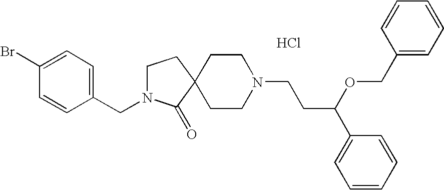 To an ice-cold stirred suspension of sodium hydride (23 mg, 60% in mineral oil, 0.6 mmol) in THF (0.5 mL) was added dropwise a solution of 2-(4-bromobenzyl)-8-(3-hydroxy-3-phenylpropyl)-2,8-diaza-spiro[4.5]decan-1-one (91.4 mg, 0.2 mmol) in THF (0.6 mL). After stirring the reaction mixture at 0° C. for 45 minutes benzyl bromide (0.071 mL, 0.6 mmol) was added and the mixture was then stirred overnight. The mixture was quenched with water and extracted with ethyl acetate (3×5 mL). The combined organic extracts were washed with brine, dried (Na₂SO₄), concentrated, and purified by semi-preparative HPLC (method B) to yield Compound 51 as a white solid (13.0 mg, 22%).

¹H NMR (400 MHz, DMSO-d₆): δ[ppm] 7.53-7.41 (m, 9H), 7.37 (t, 2H), 7.29 (m, 1H), 7.14 (t, 2H), 5.65 (d×d, 1H), 4.69 (m, 1H), 4.59 (d, 2H), 4.31 (d, 2H), 3.62 (m, 1H), 3.45 (m, 2H), 3.32 (m, 2H), 3.17 (t×d, 2H), 2.15 (m, 2H), 2.02-1.89 (m, 5H), 1.75 (m, 1H).

### EXAMPLE 6

### 2-(4-Bromobenzyl)-8-(3-phenoxy-3-phenylpropyl)-2,8-diaza-spiro[4.5]decan-1-one (Compound 52)

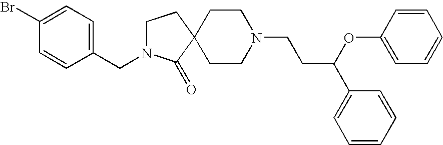 To a stirred solution of 2-(4-bromobenzyl)-8-(3-hydroxy-3-phenylpropyl)-2,8-diaza-spiro[4.5]decan-1-one (65 mg, 0.142 mol) and phenol (13.3 mg, 0.142 mmol) in THF was added triphenylphosphine (37 mg, 0.142 mmol) followed by diethylazodicarboxylate (DEAD) (0.023 mL, 0.142 mmol). After stirring for 24 hours, the reaction mixture was then concentrated and purified on silica gel preparative TLC using 20% ethyl acetate-hexanes as eluent. Compound 52 was isolated as oil (12 mg, 15.8%).

¹H NMR (CDCl₃, 400 MHz): δ[ppm] 7.40-7.0 (m, 11H), 6.82-6.76 (m, 3H), 5.19 (d×d, 1H), 4.32 (s, 2H), 3.06 (t, 2H), 2.72 (m, 2H) , 2.5-1.7 (m, 10H) , 1.4-1.25 (m, 2H)

LC/MS: m/z 534.5 (MH⁺).

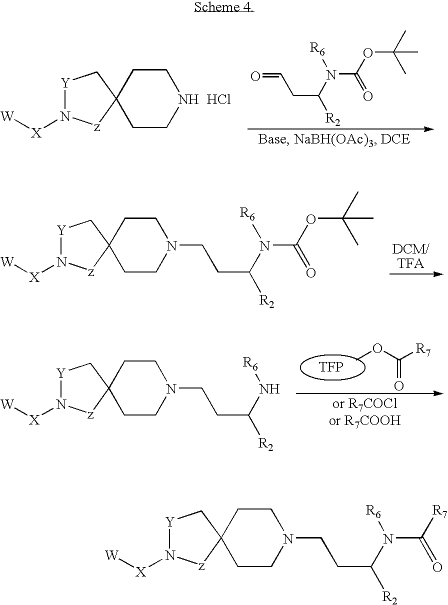

### EXAMPLE 7

### {3-[2-(4-Bromobenzyl)-1-oxo-2,8-diaza-spiro[4.5]dec-8-yl]-1-phenylpropyl}-carbamic acid tert-butyl ester (Compound 53)

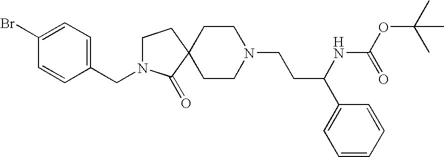 To a solution of 2.16 g (6 mmol) of 2-(4-bromobenzyl)-2,8-diaza-spiro[4.5]decan-1-one hydrochloride in 100 mL of anhydrous DCE were added successively 1.5 g (6 mmol) of (3-oxo-1-phenylpropyl)-carbamic acid tert-butyl ester and 836 μL (6 mmol) of triethylamine. The reaction mixture was agitated at room temperature for 10 minutes before adding 2 g (9 mmol) of sodium triacetoxyborohydride. After an overnight agitation, 60 mL of satured solution of sodium bicarbonate was added. The solution was then extracted with DCM, dried over sodium sulfate, filtered and concentrated in vacuo. The crude mixture was purified by flash chromatography on silica gel eluting with methanol/DCM (0% to 5%) giving

Compound 53 as a white solid (2.97 g, 88.9%).

¹H NMR (400 MHz, DMSO-d₆): δ[ppm] 7.51 (d, 2H), 7.45 (d, 1H), 7.27 (m, 4H), 7.19 (m, 1H), 7.12 (d, 2H), 4.52 (q, 1H), 4.32 (s, 2H), 3.11 (t, 2H), 2.69 (m, 2H), 2.17 (m, 2H), 1.91 (br t, 2H), 1.82 (t, 2H), 1.71 (m, 4H), 1.33 (s, 9H), 1.29 (m, 2H).

### Preparation 9

### 8-(3-Amino-3-phenylpropyl)-2-(4-bromobenzyl)-2,8-diaza-spiro[4.5]decan-1-one

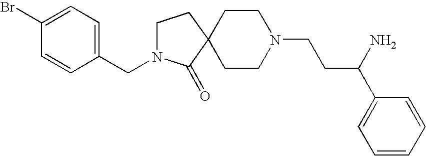 To 2.97 g (5.33 mmol) of {3-[2-(4-bromobenzyl)-1-oxo-2,8-diaza-spiro[4.5]dec-8-yl]-1-phenylpropyl}-carbamic acid tert-butyl ester was added 48 mL of a 20% TFA solution in DCM. The reaction mixture was agitated one hour at room temperature before neutralizing with 120 mL of an aqueous solution of sodium hydroxide (1N). The solution was then extracted with DCM, dried over sodium sulfate, filtered and evaporated in vacuo yielding 8-(3-amino-3-phenylpropyl)-2-(4-bromobenzyl)-2,8-diaza-spiro[4.5]decan-1-one as a pale yellow oil (2.43 g, 100%).

¹H NMR (400 MHz, DMSO-d₆): δ[ppm] 7.51 (d, 2H), 7.32-7.24 (m, 4H), 7.17 (m, 1H), 7.12 (d, 2H), 4.31 (s, 2H), 3.81 (t, 1H), 3.1 (t, 2H), 2.72 (d×d, 2H), 2.25-2.16 (m, 2H), 1.98 (br s, 2H), 1.86 (br q, 2H), 1.81 (t, 2H), 1.73-1.59 (m, 4H), 1.29 (br d, 2H).

### EXAMPLE 8

### N-{3-[2-(4-Bromobenzyl)-1-oxo-2,8-diaza-spiro[4.5]dec-8-yl]-1-phenylpropyl}-benzamide hydrochloride (Compound 54)

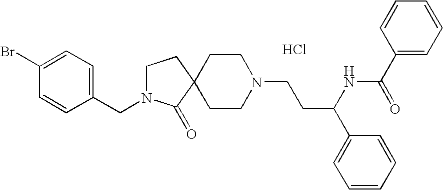 To 100 mg (100 μmol, loading of 1 mmol/g) of phenylcarboxyl activated ester on polymeric 4-hydroxy-2,3,5,6-tetrafluorobenzamido (TFP) resin (see preparation in J. M. Salvino et al. J. Comb. Chem. 2000, 2, 691-697), preswollen with 0.5 mL of anhydrous DMF, was added 27.3 mg (60 μmol) of 8-(3-amino-3-phenylpropyl)-2-(4-bromobenzyl)-2,8-diaza-spiro[4.5]decan-1-one dissolved in 1 mL of DMF. The reaction was agitated overnight at room temperature. The mixture was filtered and washed with DCM (2×2 mL) . The filtrates were collected and evaporated in vacuo. The crude mixture was purified by semi-preparative HPLC (method A) and 14.1 mg (39.4%) of Compound 54 was isolated as a colorless solid.

¹H NMR (400 MHz, DMSO-d₆): δ[ppm] 9.64 (br s, 1H), 8.91 (d, 1H), 7.88 (d, 2H), 7.55-7.42 (m, 7H), 7.35 (t, 2H), 7.25 (t, 1H), 7.14 (d, 2H), 5.12 (m, 1H), 4.33 (s, 2H), 3.46 (m, 2H), 3.16 (m, 4H), 2.97 (m, 2H), 2.35 (m, 1H), 2.2 (m, 1H), 1.98 (m, 3H), 1.83 (t, 1H), 1.6 (br d, 2H).

LC/MS: m/z 562.0 (MH⁺).

Table 3 of compounds illustrates some of the compounds of the present invention that were synthesized using the procedure described in scheme 4.

TABLE 3

| | | | | |
|---|---|---|---|---|
| CPD # | MOL STRUCTURE | COMPOUND NAME | MOL WT | PURITY |

| | | | | |
|---|---|---|---|---|
| 53 | 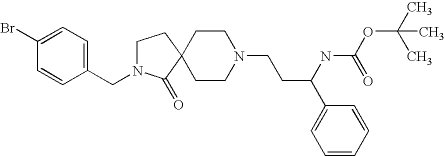 | {3-[2-(4-bromobvenzyl)-1-oxo-2,8-diaza- spiro[4.5]dec-8-yl]-1-phenyl-propyl}- carbamic acid tert-butyl ester | 556.541 | >95% (1H NMR) |
| 54 | 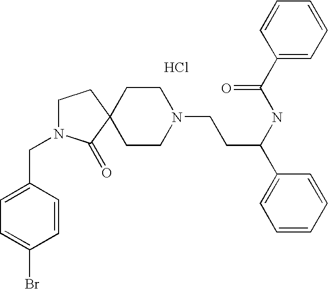 | N-{3-[2-(4-bromobenzyl)-1-oxo-2,8- diaza-spiro[4.5]dec-8-yl]-1-phenyl- propyl}-benzamide hydrochloride | 596.994 | 98+(LC/MS) |
| 55 | 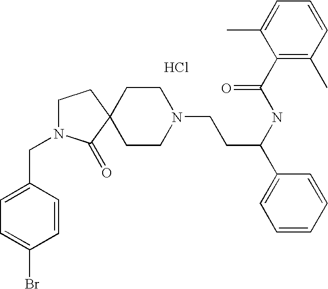 | N-{3-[2-(4-bromobenzyl)-1-oxo-2,8- diaza-spiro[4.5υdec-8-yl]-1-phenyl- propyl}-2,6-dimethyl-benzamide hydrochloride | 625.047 | 98+(LC/MS) |
| 56 | 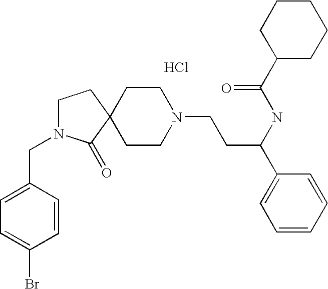 | Cyclohexanecarboxylic acid {3-[2-(4- bromobenzyl)-1-oxo-2,8-diaza- spiro[4.5]dec-8-yl]-1-phenyl-propyl}- amide hydrochloride | 603.041 | 98+(LC/MS) |
| 57 | 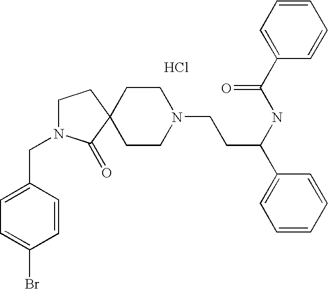 | N-{3-[2-(4-bromobenzyl)-1-oxo-2,8- diaza-spiro[4.5]dec-8-yl]-1-phenyl- propyl}-2-phenyl-acetamide hydrochloride | 611.02 | 98+(LC/MS) |
| 58 | 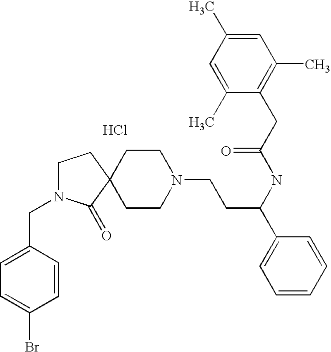 | N-{3-[2-(4-bromobenzyl)-1-oxo-2,8- diaza-spiro[4.5]dec-8-yl]-1-phenyl- propyl}-2-(2,4,6-trimethyl-phenyl)- acetamide hydrochloride | 653.101 | 98+(LC/MS) |
| 59 | 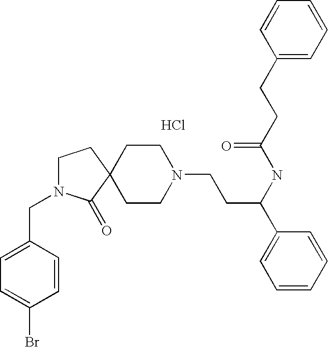 | N-{3-[2-(4-bromobenzyl)-1-oxo-2,8- diaza-spiro[4.5]dec-8-yl]-1-phenyl- propyl}-3-phenyl-propionamide hydrochloride | 625.047 | 98+(LC/MS) |
| 60 | 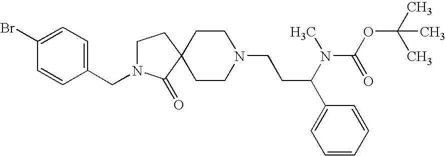 | {3-[2-(4-bromobenzyl)-1-oxo-2,8-diaza- spiro[4.5]dec-8-yl]-1-phenyl-propyl}- methyl-carbamic acid tert-butyl ester | 570.568 | >95% (1H NMR) |
| 61 | 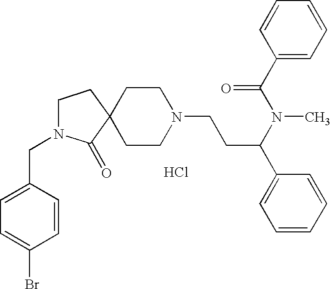 | N-{3-[2-(4-bromobenzyl)-1-oxo-2,8- diaza-spiro[4.5]dec-8-yl]-1-phenyl- propyl}-N-methyl-benzamide hydrochloride | 611.02 | 98+(LC/MS) |
| 62 | 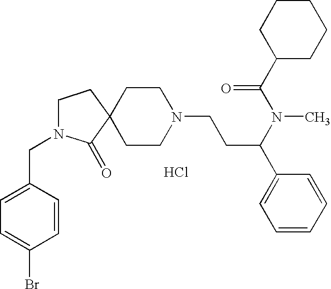 | Cyclohexanecarboxylic acid {3-[2-(4- bromo-benzyl)-1-oxo-2,8-diaza- spiro[4.5]dec-8-yl]-1-phenyl-propyl}- methyl-amide hydrochloride | 617.068 | 98+(LC/MS) |
| 63 | 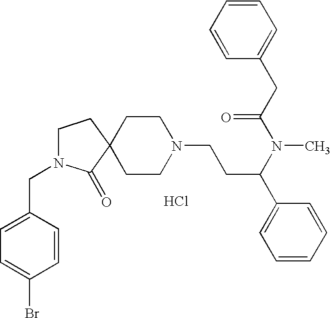 | N-{3-[2-(4-bromobenzyl)-1-oxo-2,8- diaza-spiro[4.5]dec-8-yl]-1-phenyl- propyl}-N-methyl-2-phenyl-acetamide hydrochloride | 625.047 | 98+(LC/MS) |
| 64 | 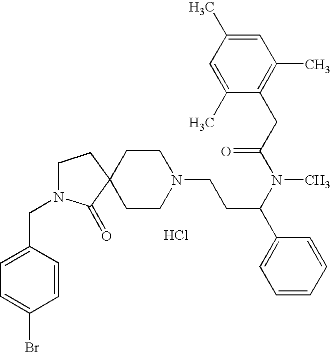 | N-{3-[2-(4-bromo-benzyl)-1-oxo-2,8- diaza-spirto[4.5]dec-8-yl]-1-phenyl- propyl}-N-methyl-2-(2,4,6-trimethyl- phenyl)-acetamide hydrochloride | 667.128 | 98+(LC/MS) |
| 65 | 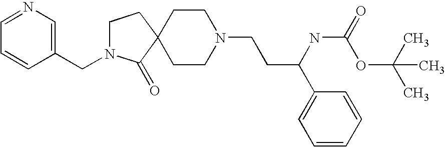 | [3-(1-oxo-2-pyridin-3-ylmethyl-2,8- diaza-spiro[4.5]dec-8-yl)-1-phenyl- propyl]-carbamic acid tert-butyl ester | 478.633 | 95% BY HPLC |
| 66 | 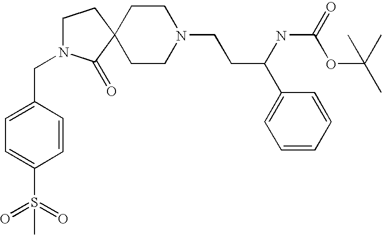 | {3-[2-(4-methanesulfonyl-benzyl)-1- -oxo-2,8-diaza-spiro[4.5]dec-8-yl]-1- phenyl-propyl}-carbamic acid tert-butyl ester | 555.736 | 94% (LC- MS) |
| 67 | 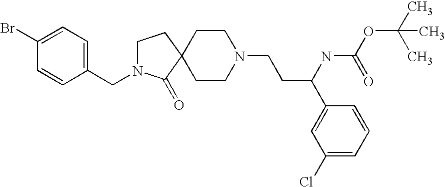 | [3-[2-(4-bromobenzyl)-1-oxo-2,8-diaza- spiro[4.5]dec-8-yl]-1-(3-chloro-phenyl)- propyl]-carbamic acid tert-butyl ester | 590.986 | >95% (1H NMR) |
| 68 | 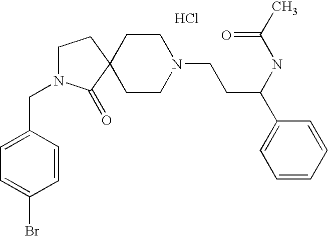 | N-{3-[2-(4-bromobenzyl)-1-oxo-2,8- diaza-spiro[4.5]dec-8-yl]-1-phenyl- propyl}-acetamide hydrochloride | 534.923 | 94.6% (LC/MS) |
| 69 | 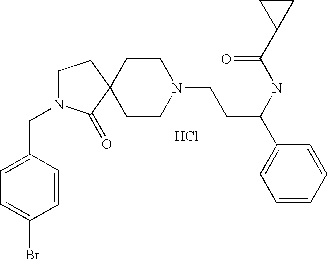 | Cyclopropanecarboxylic acid {3-[2-(4- bromo-benzyl)-1-oxo-2,8-diaza- spiro[4.5]dec-8-yl]-1-phenyl-propyl}- amide hydrochloride | 560.961 | 95.2% (LC/MS) |
| 70 | 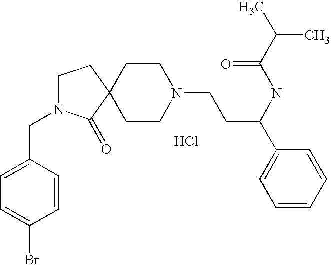 | N-{3-[2-(4-bromo-benzyl)-1-oxo-2,8- diaza-spiro[4.5]dec-8-yl]-1-phenyl- propyl}-issobutyramide hydrochloride | 562.976 | 95% (LC/MS) |
| 71 | 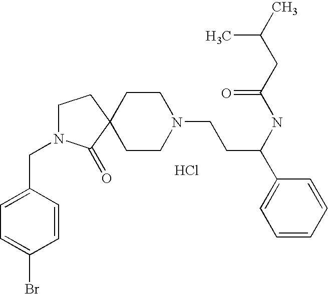 | N-{3-[2-(4-bromobenzyl)-1-oxo-2,8- diaza-spiro[4.5]dec-8-yl]-1-phenyl- propyl}-3-methyl-butyramide hydrochloride | 577.003 | 98+(LC/MS) |
| 72 | 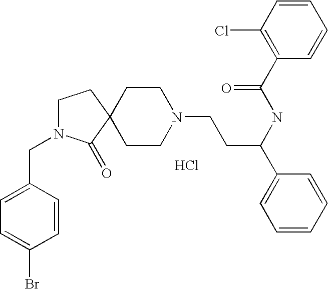 | N-{3-[2-(4-bromo-benzyl)-1-oxo-2,8- diaza-spiro[4.5]dec-8-yl]-1-phenyl- propyl}-2-chloro-benzmaide hydrochloride | 631.439 | 98+(LC/MS) |
| 73 | 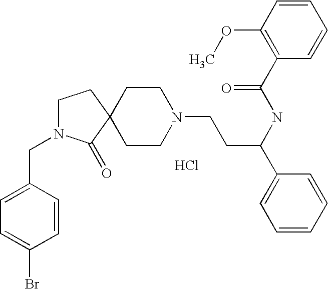 | N-{3-[2-(4-bromo-benzyl)-1-oxo-2,8- diaza-spiro[4.5]dec-8-yl]-1-phenyl- propyl}-2-methoxy-benzamide hydrochloride | 627.019 | 98+(LC/MS) |
| 74 | 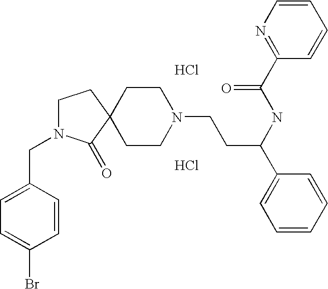 | Pyridine-2-carboxylic acid {3-[2-(4- bromobenzyl)-1-oxo-2,8-diaza- spiro[4.5υdec-8-yl]-1-phenyl-propyl}- amide hydrochloride | 634.443 | 93.7% (LC/MS) |
| 75 | 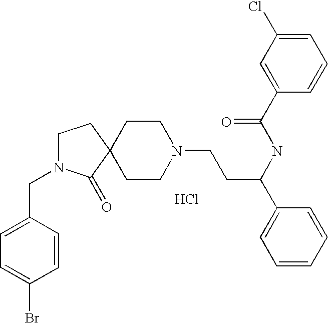 | N-{3-[2-(4-bromobenzyl)-1-oxo-2,8- diaza-spiro[4.5]dec-8-yl]-1-phenyl- propyl}-3-methoxy-benzamide hydrochloride | 631.439 | 98+(LC/MS) |
| 76 | 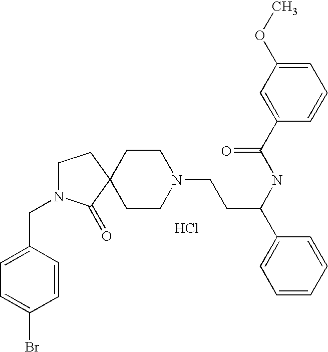 | N-{3-[2-(4-bromo-benzyl)-1-oxo-2,8- diaza-spiro[4.5]dec-8-yl]-1-phenyl- propyl}-3-chloro-benzamide hydrochloride | 627.019 | 98+(LC/MS) |
| 77 | 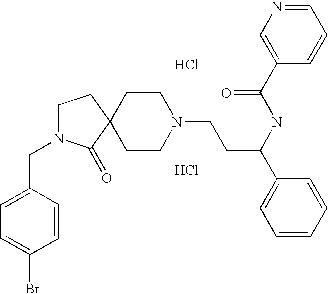 | N-{3-[2-(4-bromobenzyl)-1-oxo-2,8- diaza-spiro[4.5]dec-8-yl]-1-phenyl- propyl}-nicotinamide dihydrochloride | 634.443 | 98+(LC/MS) |
| 78 | 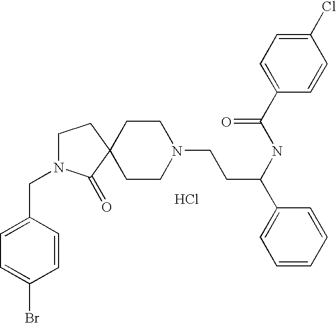 | N-{3-[2-(4-bromobenzyl)-1-oxo-2,8- diaza-spiro[4.5]dec-8-yl]-1-phenyl- propyl}-4-chloro-benzamidehydrochloride | 631.439 | 98+(LC/MS) |
| 79 | 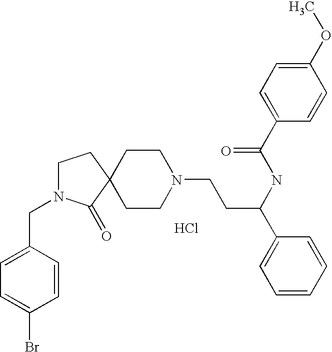 | N-{3-[2-(4-bromo-benzyl)-1-oxo-2,8- diaza-spiro[4.5]dec-8-yl]-1-phenyl- propyl}-4-methoxy-benzamide hydrochloride | 627.019 | 98+(LC/MS) |
| 80 | 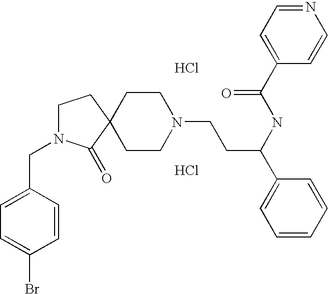 | N-{3-[2-(4-bromobenzyl)-1-oxo-2,8- diaza-spiro[4.5]dec-8-yl]-1-phenyl- propyl}-isonicotinamide dihydrochloride | 634.443 | 98+(LC/MS) |
| 81 | 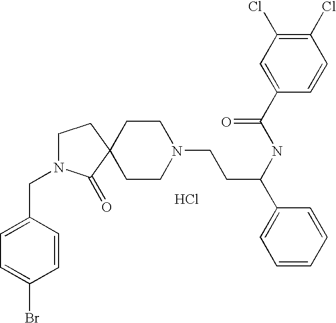 | N-{3-[2-(4-bromobenzyl)-1-oxo-2,8- diaza-spiro[4.5]dec-8-yl]-1-phenyl- propyl}-3,4-dichloro-benzamide hydrochloride | 665.884 | 98+(LC/MS) |
| 82 | 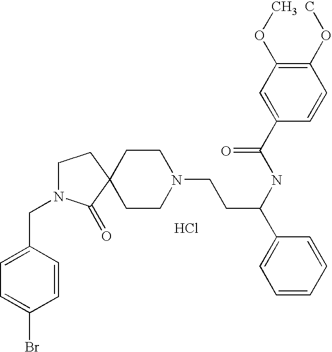 | N-{3-[2-(4-bromo-benzyl)-1-oxo-2,8- diaza-spiro[4.5]dec-8-yl]-1-phenyl- propyl}-3,4-dimethoxy-benzamide hydrochloride | 657.045 | 98+(LC/MS) |
| 83 | 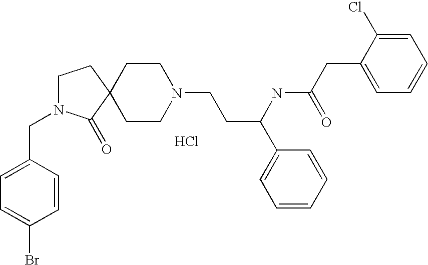 | N-{3-[2-(4-bromo-benzyl)-1-oxo-2,8- diaza-spiro[4.5]dec-8-yl]-1-phenyl- propyl}-2-(2-chloro-phenyl)-acetamide hydrochloride | 645.465 | 94.8% (LC/MS) |
| 84 | 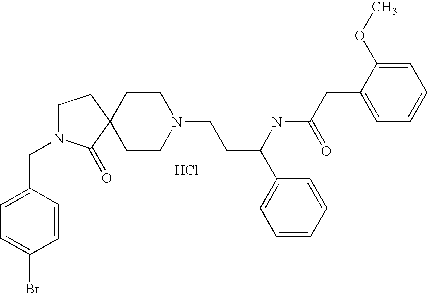 | N-{3-[2-(4-bromobenzyl)-1-oxo-2,8- diaza-spiro[4.5]dec-8-yl]-1-phenyl- propyl}-2-(2-methoxy-phenyl)- acetamide hydrochloride | 641.046 | 98+(LC/MS) |
| 85 | 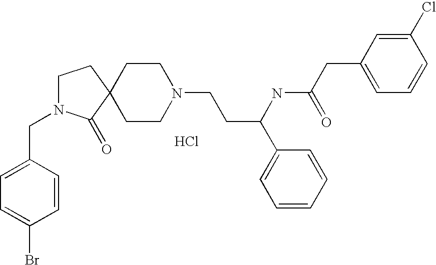 | N-{3-[2-(4-bromobenzyl)-1-oxo-2,8- diaza-spiro[4.5]dec-8-yl]-1-phenyl- propyl}-2-(3-chloro-phenyl)-acetamide hydrochloride | 645.465 | 92.9% (LC/MS) |
| 86 | 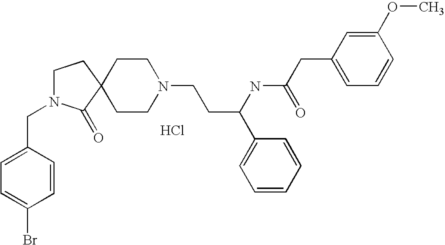 | N-{3-[2-(4-bromobenzyl)-1-oxo-2,8- diaza-spiro[4.5]dec-8-yl]-1-phenyl- propyl}-2-(3-methoxy-phenyl)- acetamide hydrochloride | 641.046 | 98+(LC/MS) |
| 87 | 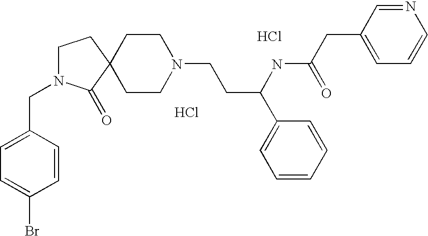 | N-{3-[2-(4-bromo-benzyl)-1-oxo-2,8- diaza-spiro[4.5]dec-8-yl]-1-phenyl- propyl}-2-pyridin-3-yl-acetamide dihydrochloride | 648.469 | 95+(LC/MS) |
| 88 | 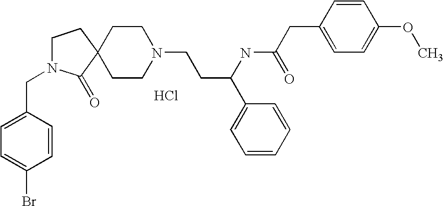 | N-{3-[2-(4-bromobenzyl)-1-oxo-2,8- diaza-spiro[4.5]dec-8-yl]-1-phenyl- propyl}-2-(4-methoxy-phenyl)- acetamide hydrochloride | 641.046 | 98+(LC/MS) |
| 89 | 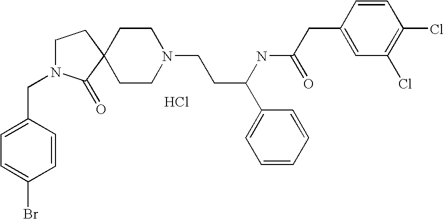 | N-{3-[2-(4-bromobenzyl)-1-oxo-2,8- diaza-spiro[4.5]dec-8-yl]-1-phenyl- propyl}-2-(3,4-dichloro-phenyl)- acetamide hydrochloride | 679.911 | 95+(LC/MS) |
| 90 | 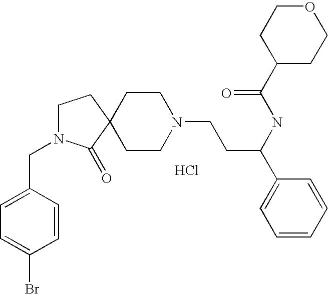 | Tetrahydro-pyran-4-carboxylic acid{3- [2-(4-bromobenzyl)-1-oxo-2,8-diaza- spiro[4.5]dcec-8-yl]-1-phenyl-propyl}- amide hydrochloride | 605.013 | 95+(LC/MS) |
| 91 | 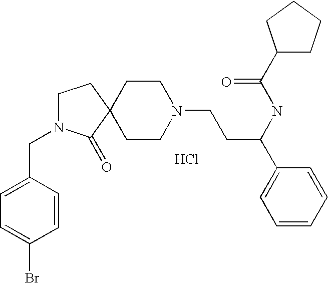 | Cyclopentanecarboxylic acid {3-[2-(4- bromobenzyl)-1-oxo-2,8-diaza- spiro[4.5]dec-8-yl]-1-phenyl-propyl}- amide hydrochloride | 589.014 | 98+(LC/MS) |
| 92 | 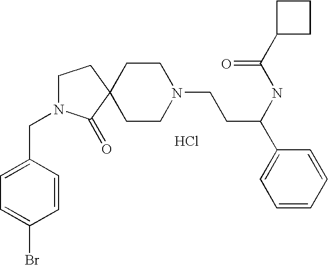 | Cyclobutanecarboxylic acid {3-[2-(4- bromobenzyl)-1-oxo-2,8-diaza- spiro[4.5]dec-8-yl]-1-phenyl-propyl}- amide hydrochloride | 574.987 | 98+(LC/MS) |
| 93 | 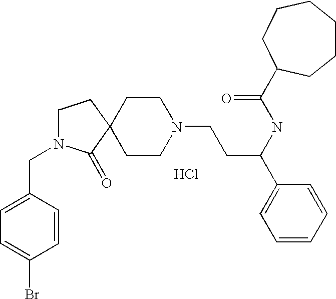 | Cycloheptanecarboxylic acid {3-[2-(4- bromobenzyl)-1-oxo-2,8-diaza- spiro[4.5]dec-8-yl]-1-phenyl-propyl}- amide hydrochloride | 617.068 | 98+(LC/MS) |
| 94 | 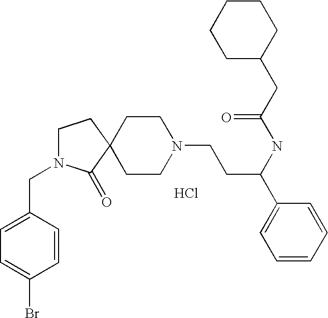 | N-{3-[2-(4-bromo-benzyl)-1-oxo-2,8- diaza-spiro[4.5]dec-8-yl]-1-phenyl- propyl}-2-cyclohexyl-acetamide hydrochloride | 617.068 | 98+(LC/MS) |
| 95 | 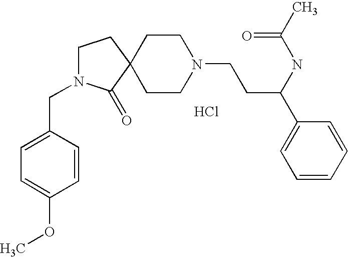 | N-{3-[2-(4-methoxybenzyl)-1-oxo-2,8- diaza-spiro[4.5]dec-8-yl]-1-phenyl- propyl}-acetamide hydrochloride | 486.052 | 98+(LC/MS) |
| 96 | 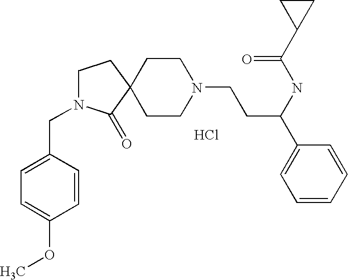 | Cyclopropanecarboxylic acid {3-[2-(4- methoxybenzyl)-1-oxo-2,8-diaza- spiro[4.5]dec-8-yl]-1-phenyl-propyl}- amide hydrochloride | 512.09 | 98+(LC/MS) |
| 97 | 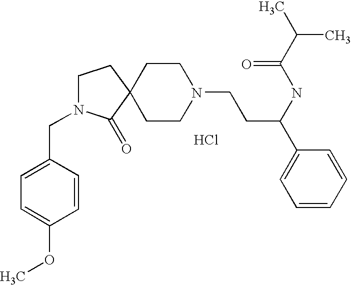 | N-{3-[2-(4-methoxy-benzyl)-1-oxo-2,8- diaza-spiro[4.5]dec-8-yl]-1-phenyl- propyl}-isobutyramide hydrochlooride | 514.106 | 98+(LC/MS) |
| 98 | 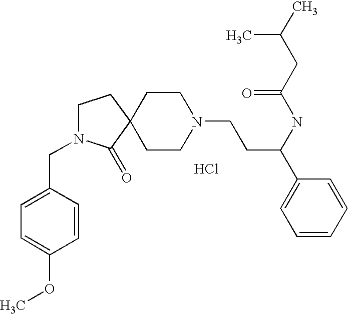 | N-{3-[2-(4-methoxybenzyl)-1-oxo-2,8- diaza-spiro[4.5]dec-8-yl]-1-phenyl- propyl}-3-methyl-butyramide hydrochloride | 528.133 | 98+(LC/MS) |
| 99 | 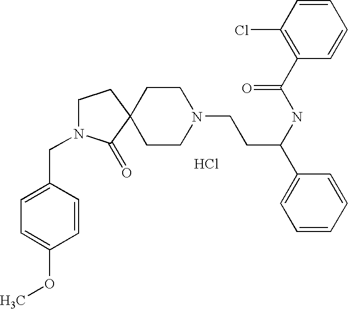 | 2-chloro-N-{3-[2-(4-methoxybenzyl)-1- oxo-2,8-diaza-spiro[4.5]dec-8-yl]-1- phenyl-propyl}-benzamide hydrochloride | 582.568 | 95+(LC/MS) |
| 100 | 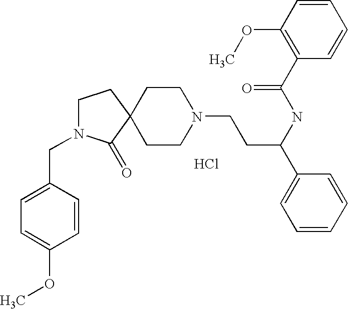 | 2-methoxy-N-{3-[2-(4-methoxybenzyl)- 1-oxo-2,8-diaza-spiro[4.5]dec-8-yl]-1- phenyl-propyl}-benzamide hydrochloride | 578.149 | 98+(LC/MS) |
| 101 | 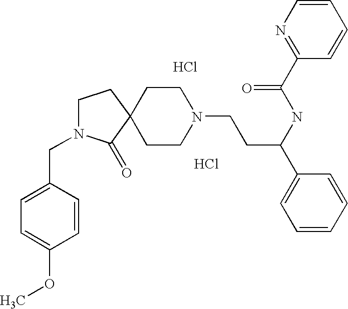 | Pyridine-2-carboxylic acid {3-[2-(4- methoxybenzyl)-1-oxo-2,8-diaza- spiro[4.5]dec-8-yl]-1-phenyl-propyl}- amide dihydrochloride | 585.572 | 98+(LC/MS) |
| 102 | 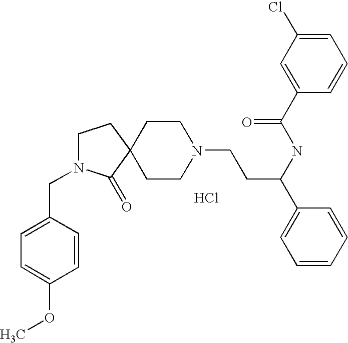 | 3-chloro-N-{3-[2-(4-methoxy-benzyl)-1- oxo-2,8-diaza-spiro[4.5]dec-8-yl]-1- phenyl-propyl}-benzamide hydrochloride | 582.568 | 98+(LC/MS) |
| 103 | 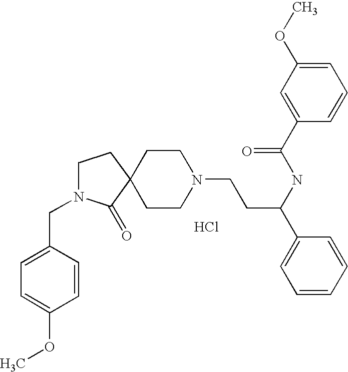 | 3-methoxy-N-{3-[2-(4-methoxy-benzyl)- 1-oxo-2,8-diaza-spiro[4.5]dec-8-yl]-1- phenyl-propyl}-benzamide hydrochloride | 578.149 | 98+(LC/MS) |
| 104 | 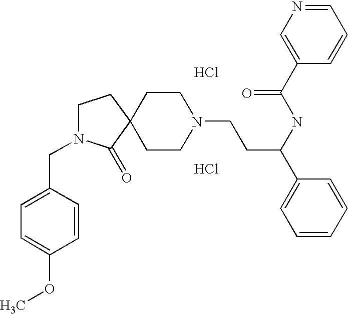 | N-{3-[2-(4-methoxybenzyl)-1-oxo-2,8- diaza-spiro[4.5]dec-8-yl]-1-phenyl- propyl}-nicotinamide dihydrochloride | 585.572 | 98+(LC/MS) |
| 105 | 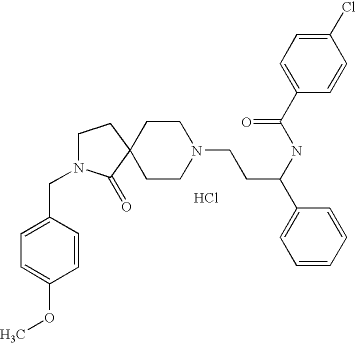 | 4-chloro-N-{3-[2-(4-methoxybenzyl)-1- oxo-2,8-diaza-spiro[4.5]dec-8-yl]-1- phenyl-propyl}-benzamide hydrochloride | 582.568 | 98+(LC/MS) |
| 106 | 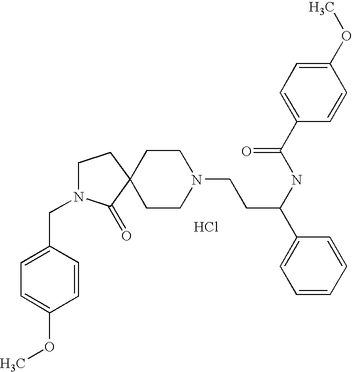 | 4-methoxy-N-{3-[2-(4-methoxy-benzyl)- 1-0oxo-2,8-diaza-spiro[4.5]dec-8-yl]-1- phenyl-propyl}-benzamide hydrochloride | 578.149 | 98+(LC/MS) |
| 107 | 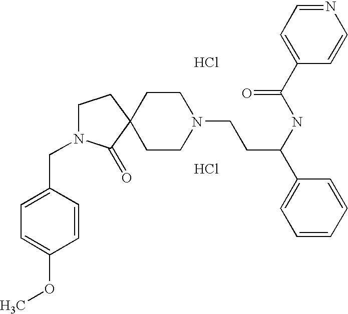 | N-{3-[2-(4-methoxybenzyl)-1-oxxo-2,8- diaza-spiro[4.5]dec-8-yl]-1-phenyl- propyl}-isonicotinamide dihydrochloride | 585.572 | 98+(LC/MS) |
| 108 | 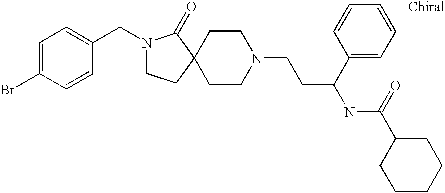 | (R)-cyclohexanecarboxylicc acid {3-[2- (4-bromobenzyl)-1-oxo-2,8-diaza-spiro [4.5]dec-8-yl]-1-phenyl-propyl}-asmide | 566.58 | 100% (LC/MS) |
| 109 | 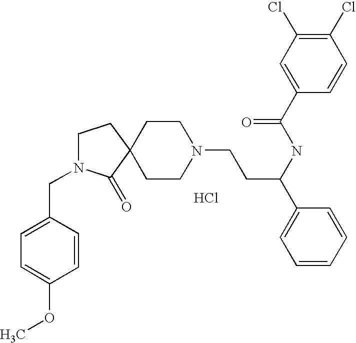 | 3,4-dichloro-N-{3-[1-(4- methoxybenzyl)-1-oxo-2,8-diaza- spiro[4.5]dec-8-yl]-1-phenyl-propyl}- benzamide hydrochloride | 617..013 | 98+(LC/MS) |
| 110 | 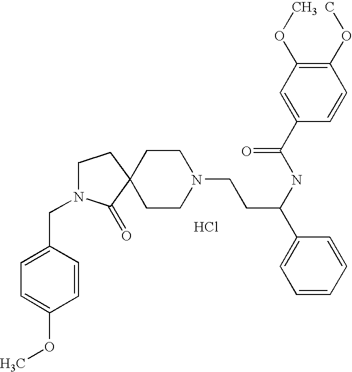 | 3,4-dimethoxy-N-{3-[2-(4- methoxybenzyl)-1-oxo-2,8-diaza- spiro[4.5]dec-8-yl]-1-phenyl-propyl}- benzamide hydrochloride | 608.175 | 98+(LC/MS) |
| 111 | 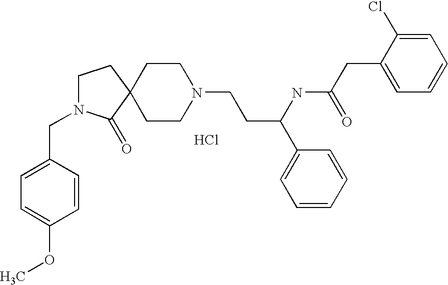 | 2-(2-chloro-phenyl)-N-{3-[2-(4- methoxybenzyl)-1-oxo-2,8-diaza- spiro[4.5]dec-8-yl]-1-phenyl-propyl}- acetamide hydrochloride | 596.595 | 98+(LC/MS) |
| 112 | 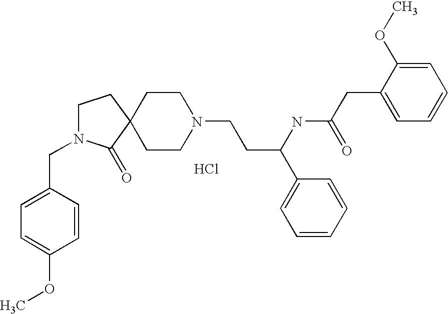 | N-{3-[2-(4-methoxybenzyl)-1-oxo-2,8- diaza-spiro[4.5]dec-8-yl]-1-phenyl- propyl}-2-(2-methox-phenyl)-acetamide hydrochloride | 592.176 | 98+(LC/MS) |
| 113 | 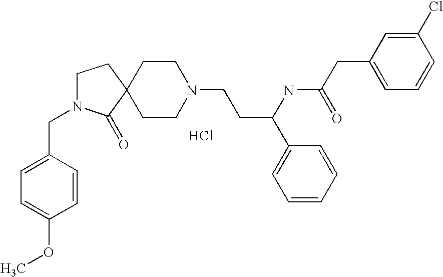 | 2-(3-chlorophenyl)-N-{3-[2-(4- methoxybenzyl)-1-oxo-2,8-diaza- dspiro[4.5]dec-8-yl]-1-phenyl-propyl}- acetamide hydrochloride | 5596.595 | 98+(LC/MS) |
| 114 | 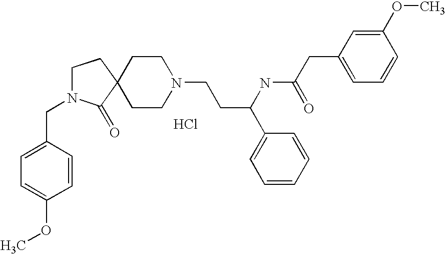 | N-{3-[2-(4-methoxybenzyl)-1-oxo-2,8- diaza-spiro[4.5]dec-8-yl]-1-phenyl- propyl}-2-(3-methoxyphenyl)-acetamide hydrochloride | 592.176 | 98+(LC/MS) |
| 115 | 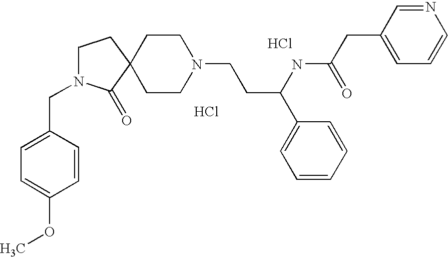 | N-{3-[2-(4-methoxybenzyl)-1-oxo-2,8- diaza-spiro[4.5]dec-8-yl]-1-phenyl- propyl}-2-pyridin-3-yl-acetamide dihydrochloride | 599.599 | 90+(LC/MS) |
| 116 | 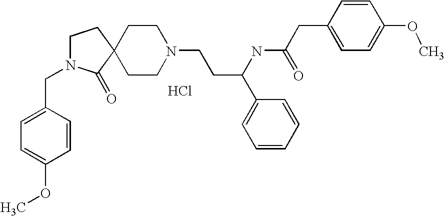 | N-{3-[2-(4-methoxybenzyl)-1-oxo-2,8- diaza-spiro[4.5]dec-8-yl]-1-phenyl- propyl}-2-(4-methoxyphenyl)-acetamide hydrochloride | 592.176 | 98+(LC/MS) |
| 117 | 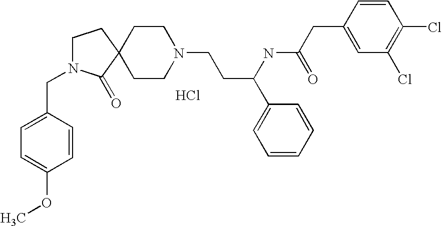 | 2-(3,4-dichlorophenyl)-N-{3-[2-(4- methoxybenzyl)-1-oxo-2,8-diaza- spiro[4.5]dec-8-yl]-1-phenyl-propyl}- acetamide hydrochloride | 631.04 | 95+(LC/MS) |
| 118 | 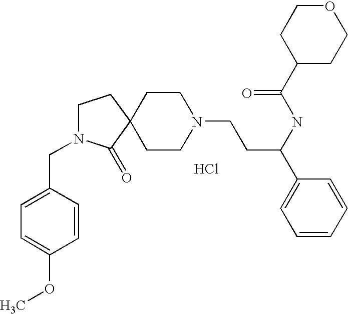 | Tetrahydro-pyran-4-carboxylic acid{3- [2-(4-methoxybeenzyl)-1-oxo-2,8-diaza- spiro[4.5]dec-8-yl]-1-phenyl-propyl}- amide hydrochloride | 556.143 | 95+(LC/MS) |
| 119 | 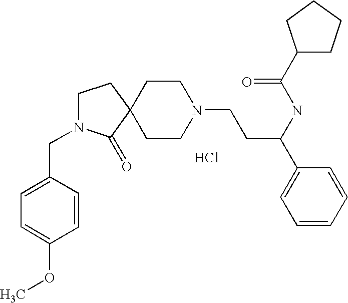 | Cyclopentanecarboxylic acid {3-[2-(4- methoxybenzyl)-1-oxo-2,8-diaza- spiro[4.5]dec-8-yl]-1-phenyl-propyl}- amide hydrochloride | 540.144 | 98+(LC/MS) |
| 120 | 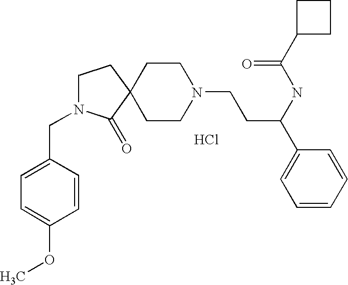 | Cyclobutanecarboxylic acid {3-[2-(4- methoxybenzyl)-1-oxo-2,8-diaza- spiro[4.5]dec-8-yl]-1-phenyl-propyl}- amide hydrochloride | 526.117 | 95+(LC/MS) |
| 121 | 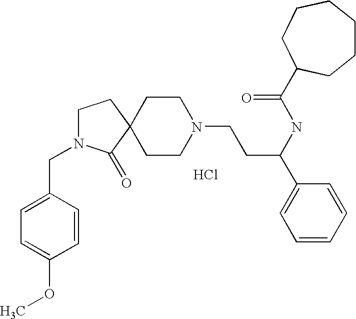 | Cycloheptanecarboxylic acid {3-[2-(4- methoxybenzyl)-1-oxo-2,8-diaza- spiro[4.5]dec-8-yl]-1-phenyl-prropyl}- amide hydrochloride | 568.197 | 98+(LC/MS) |
| 122 | 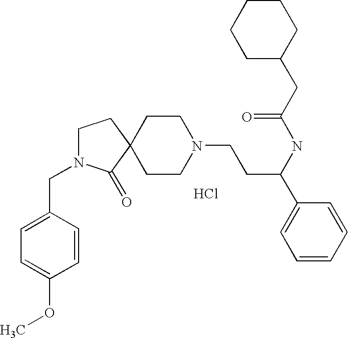 | 2-cyclohexyl-N-{3-[2-(4- methoxybenzyl)-1-oxo-2,8-diaza- spiro[4.5]dec-8-yl]-1-phenyl-propyl}- acetamide hydrochloride | 568.197 | 98+(LC/MS) |
| 123 | 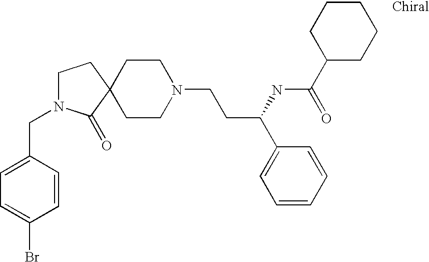 | (S)-cyclohexanecarboxylic acid {3-[2- (4-bromobenzyl)-1-oxo-2,8-diaza- spirro[44.5]dec-8-yl]-1-phenyl-propyl}- amide | 566.58 | 99% BY HPLC |
| 124 | 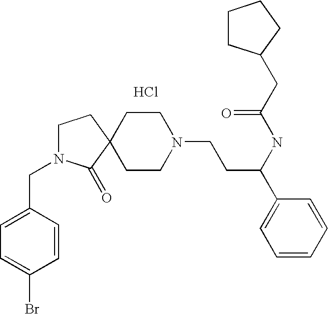 | N-{3-[2-(4-bromobenzyl)-1-oxo-2,8- diaza-spiro[4.5]dec-8-yl]-1-phenyl- propyl}-2-cyclopentyl-acetamide hydrochloride | 603.041 | 98+(LC/MS) |
| 125 | 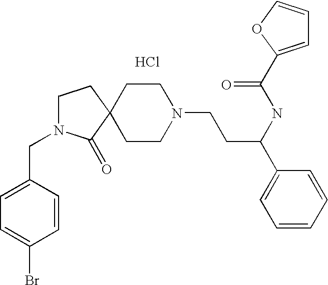 | Furan-2-carboxylic acid {3-[2-(4- bromobenzyl)-1-oxo-2,8-diaza- spiro[4.5]dec-8-yl]-1-phenyl-propyl}- amide hydrochloride | 586.955 | 98+(LC/MS) |
| 126 | 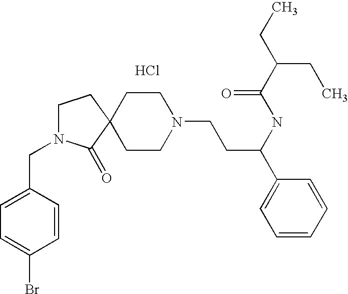 | N-{3-[2-(4-bromobenzyl)-1-oxo-2,8- diaza-spirro[4.5]dec-8-yl]-1-phenyl- propyl}-2-ethyl-butyramide hydrochloride | 591.03 | 98+(LC/MS) |
| 127 | 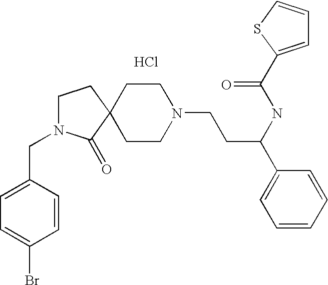 | Thiophene-2-carboxylic acid {3-[2-(4- brom-benzyl)-1-oxo-2,8-diaza- spiro[4.5]dec-8-yl]-1-phenyl-propyl}- amide hydrochloride | 603.022 | 98+(LC/MS) |
| 128 | 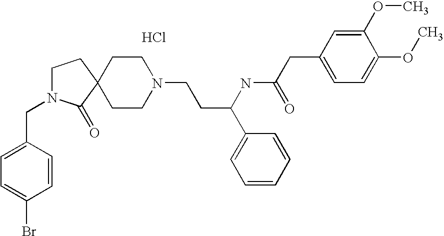 | N-{3-[2-(4-bromobenzyl)-1-oxo-2,8- diaza-spiro[4.5]dec-8-yl]-1-phenyl- propyl}-2-(3,4-dimethoxyphenyl)- acetamide hydrochloride | 671.072 | 98+(LC/MS) |
| 129 | 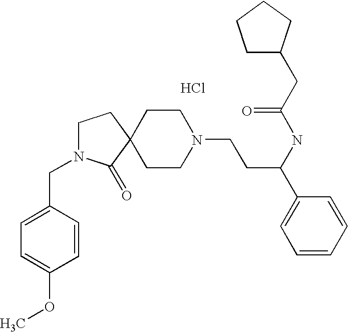 | 2-cyclopentyl-N-{3-[2-(4- methoxybenzyl)-1-oxo-2,8-diaza- spiro[4.5]dec-8-yl]-1-phenyl-propyl}- acetamide hydrochloride | 554.171 | 98+(LC/MS) |
| 130 | 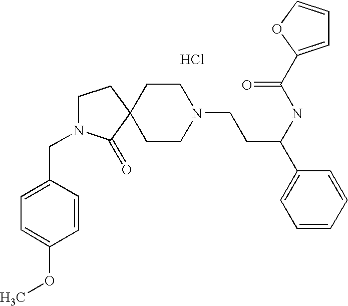 | Furan-2-carboxylic acid {3-[2-(4 methox-benzyl)-1-oxxo-2,8-diaza- spiro[4.5]dec-8-yl]-1-phenyl-propyl}- amide hydrochloride | 538.084 | 98+(LC/MS) |
| 131 | 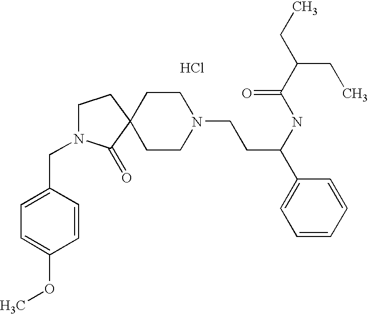 | 2-ethyl-N-{3-[2-(4-methoxybenzyl)-1- oxo-2,8-diaza-spiro[4.5]dec-8-yl]-1- phenyl-propyl}-butyramide hydrochloride | 542.16 | 98+(LC/MS) |
| 132 | 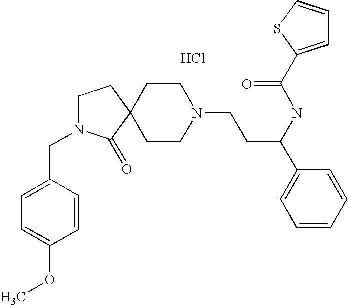 | Thiophene-2-carbopxylic acid {3-[2-(4- methoxybenzyl)-1-oxo-2,8-diaza- spiro[4.5]dec-8-yl]-1-phenyl-propyl}- amide hydrochloride | 554.151 | 98+(LC/MS) |
| 133 | 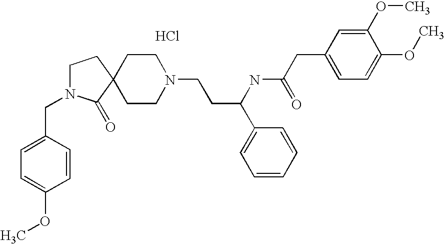 | 2-(3,4-dimethoxy-phenyl)-N-{3-[2-(4- methoxybenzyl)-1-oxo-2,8-diaza- spiro[4.5]dec-8-yl]-1-phenyl-propyl}- acetamide hydrochloride | 622.202 | 98+(LC/MS) |
| 134 | 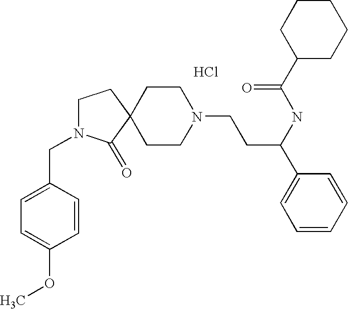 | Cyclohexanecarboxylic acid {3-[2-(4- methoxybenzyl)-1-oxo-2,8-diaza- spirro[4.5]dec-8-yl]-1-phenyl-propyl}- amide hydrochloride | 554.171 | 98+(LC/MS) |
| 135 | 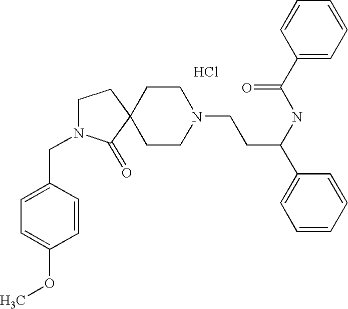 | N-{3-[2-(4-methoxybenzyl)-1-oxo-2,8- diaza-spiro[4.5]dec-8-yl]-1-phenyl- propyl}-beenzamide hydrochloride | 548.123 | 98+(LC/MS) |
| 136 | 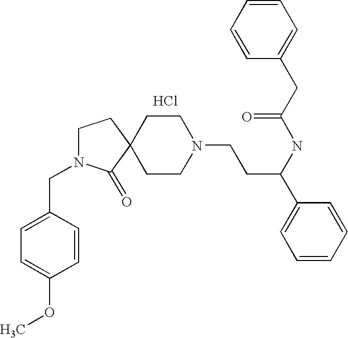 | N-{3-[2-(4-methoxybenzyl)-1-oxo-2,8- diaza-spiro[4.5]dec-8-yl]-1-phenyl- propyl}-2-phenyl-acetamide hydrochloride | 562.15 | 98+(LC/MS) |
| 137 | 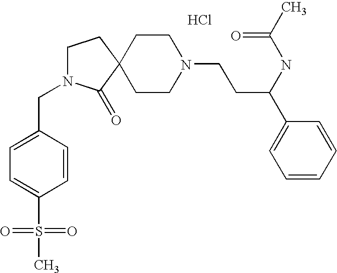 | N-{3-[2-(4-methanesulfonylbenzyl)-1- oxo-2,8-diaza-spiro[4.5]dec-8-yl]-1- phenyl-propyl}-acetamide hydrochloride | 534.117 | 98+(LC/MS) |
| 138 | 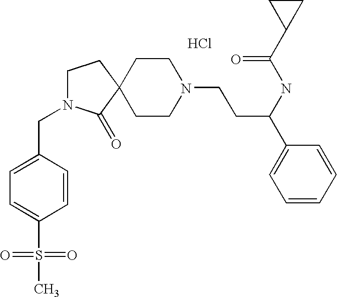 | Cyclopropanecarboxylic acid {3-[2-(4- methanesulfonylben zyl)-1-oxxo-2,8- diaza-spirro[4.5]dec-8-yl]-1-phenyl- propyl} amide hydrochloride | 560.155 | 98+(LC/MS) |
| 139 | 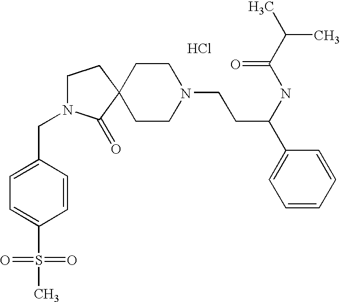 | N-{3-[2-(4-methanesulfonyl-benzyl)-1- oxo-2,8-diaza-spiro[4.5]dec-8-yl]-1- phenyl-propyl}-isobutyramide hydrochloride | 562.171 | 95% (LC/MS) |
| 140 | 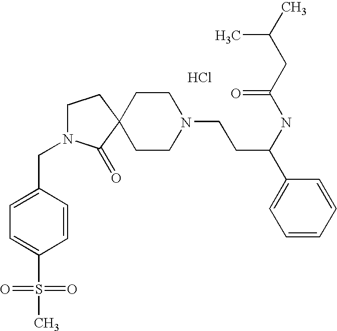 | N-{3-[2-(4-methanesulfonylbenzyl)-1- oxo-2,8-diaza-spiro[4.5]dec-8-yl]-1- phenyl-propyl}-3-methyl-butyramide hydrochloride | 576.198 | 98+(LC/MS) |
| 141 | 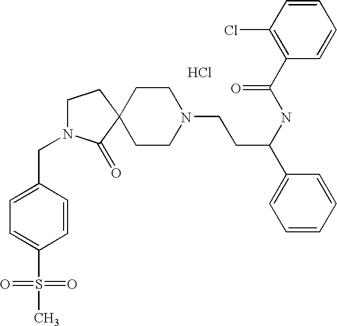 | 2-chloro-N-{3-[2-(4- methanesulfonylbenzyl)-1-oxo-2,8- diaza-spiro[4.5]dec-8-yl]-1-phenyl- propyl}-benzamide hydrochloride | 630.633 | 98+(LC/MS) |
| 142 | 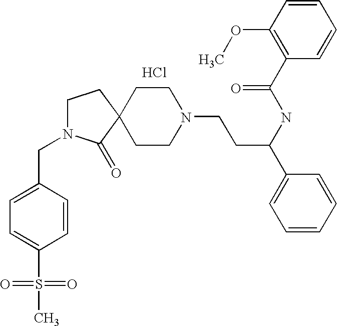 | N-{3-[2-(4-methanesulfonylbenzyl)-1- oxo-2,8-diaza-spiro[4.5]dec-8-yl]-1- phenyl-propyl}-2-methoxy-benzamide hydrochloride | 626.214 | 98+(LC/MS) |
| 143 | 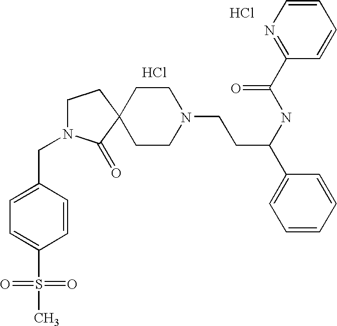 | Pyridine-2-carboxylic acid {3-[2-(4- methanesulfonylbenzyl)-1-oxo-2,8- diaza-spiro[4.5]dec-8-yl]-1-phenyl- propyl}-amide dihydrochloride | 633.637 | 94.4% (LC/MS) |
| 144 | 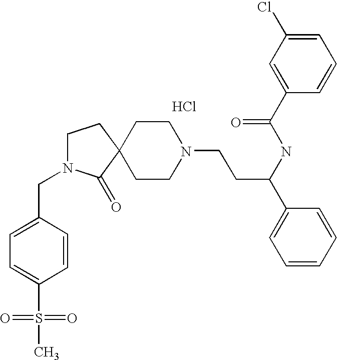 | 3-chloro-N-{3-[2-(4- methanesulfonylbenzyl)-1-oxo-2,8- diaza-spiro[4.5]dec-8-yl]-1-phenyl- propyl}-benzamide hydrochloride | 630.633 | 94.1% (LC/MS) |
| 145 | 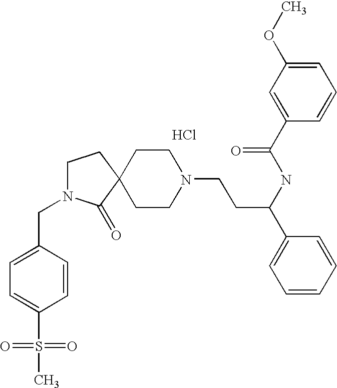 | N-{3-[2-(4-methanesulfonylbenzyl)-1- oxo-2,8-diaza-spiro[4.5]dec-8-yl]-1- phenyl-propyl}-3-methoxy-benzamide hydrochloride | 626.214 | 98+(LC/MS) |
| 146 | 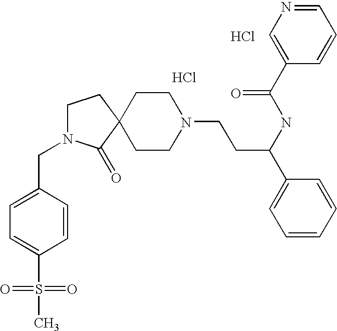 | N-{3-[2-(4-methanesulfonylbenzyl)-1- oxo-2,8-diaza-spiro[4.5]dec-8-yl]-1- phenyl-propyl}-nictotinamide dihydrochloride | 633.637 | 98+(LC/MS) |
| 147 | 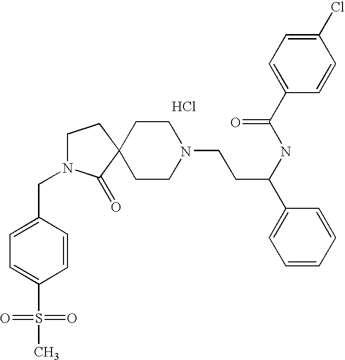 | 4-chloro-N-{3-[2-(4- methanesulfonylbenzyl)-1-oxo-2,8- diaza-spiro[4.5]dec-8-yl]-1-phenyl- propyl}-benzamide hydrochloride | 630.633 | 98+(LC/MS) |
| 148 | 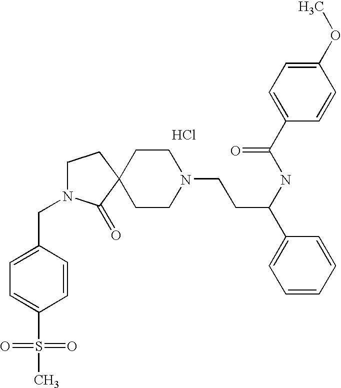 | N-{3-[2-(4-methanesulfonylbenzyl)-1- oxo-2,8-diaza-spiro[4.5]dec-8-yl]-1- phenyl-propyl}-4-methoxy-benzamide hydrochloride | 626.214 | 98+(LC/MS) |
| 149 | 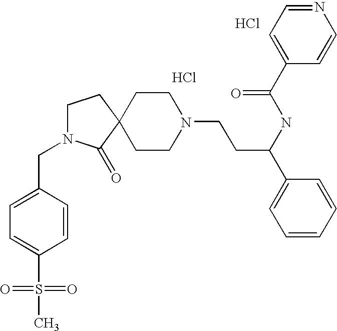 | N-{3-[2-(4-methanesulfonylbenzyl)-1- oxo-2,8-diaza-spiro[4.5]dec-8-yl]-1- phenyl-propyl}-isonicotinamide dihydrochloride | 633.637 | 98+(LC/MS) |
| 150 | 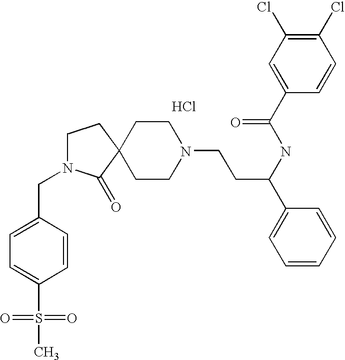 | 3,4-dichloro-N-{3-[2-(4- methanesulfonylbenzyl)-1-oxo-2,8- diaza-spiro[4.5]dec-8-yl]-1-phenyl- propyl}-benzamide hydrochloride | 665.078 | 98+(LC/MS) |
| 151 | 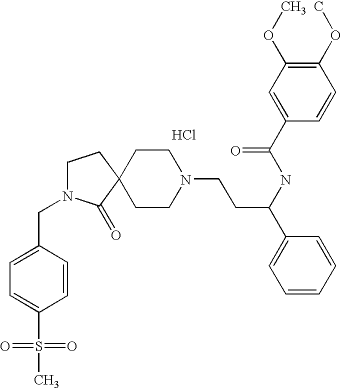 | N-{3-[2-(4-methanesulfonylbenzyl)-1- oxo-2,8-diaza-spiro[4.5]dec-8-yl]-1- pheenyl-propyl}-3,4-dimethoxy- benzamide hydrochloride | 656.24 | 98+(LC/MS) |
| 152 | 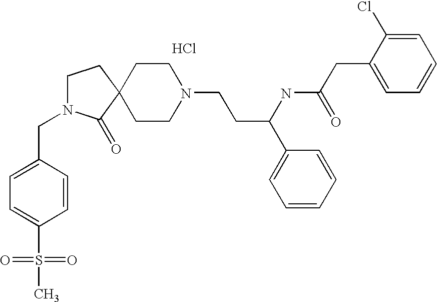 | 2-(2-chlorophenyl)-N-{3-[2-(4- methanesulfonylbenzyl)-1-oxo-2,8- diaza-spiro[4.5]dec-8-yl]-1-phenyl- propyl}-acetamide hydrochloride | 644.66 | 88.1% (LC/MS) |
| 153 | 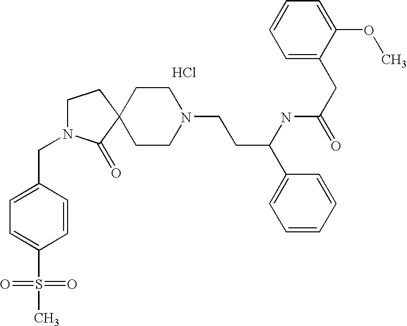 | N-{3-[2-(4-methanesulfonylbenzyl)-1- oxo-2,8-diaza-spiro[4.5]dec-8-yl]-1- phenyl-propyl}-2-(2-methoxyphenyl)- acetamide hydrochloride | 640.241 | 98+(LC/MS) |
| 154 | 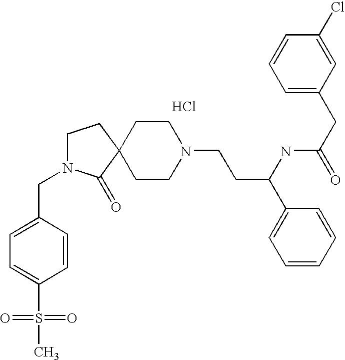 | 2-(3-chlorophenyl)-N-{3-[2-(4- methanesulfonylbenzyl)-1-oxo-2,8- diaza-spiro[4.5]dec-8-yl]-1-phenyl- propyl}-acetamide hydrochloride | 644.66 | 93% (LC/MS) |
| 155 | 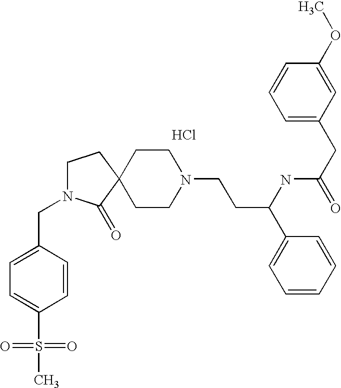 | N-{3-[2-(4-methanesulfonylbenzyl)-1- oxo-2,8-diaza-spiro[4.5]dec-8-yl]-1- phenyl-propyl}-2-(3-methoxyphenyl)- acetamide hydrochloride | 640.241 | 98+(LC/MS) |
| 156 | 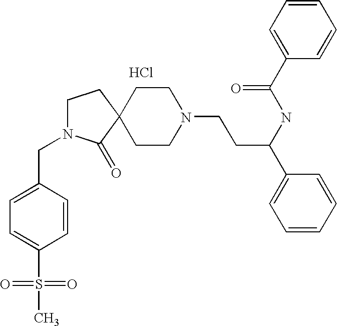 | N-{3-[2-(4-methanesulfonylbenzyl)-1- oxo-2,8-diaza-spiro[4.5]dec-8-yl]-1- phenyl-propyl}-benzamide hydrochloride | 596.188 | 98+(LC/MS) |
| 157 | 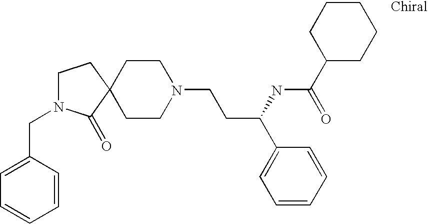 | (S)-cyclohexanecarboxylic acid [3-(2- benzyl-1-oxo-2,8-diaza-spiro[4.5]dec-8- yl)-1-phenyl-propyl]-amide | 487.684 | >95% (HPLC) |
| 158 | 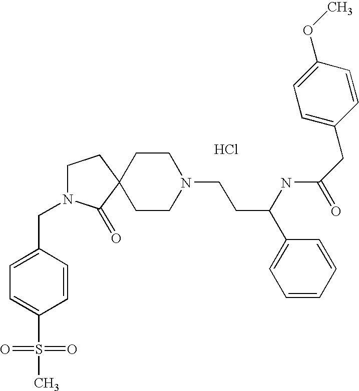 | N-{3-[2-(4-methanesulfonylbenzyl)-1- oxo-2,8-diaza-spiro[4.5]dec-8-yl]-1- phenyl-propyl}-2-(4-methoxyphenyl)- acetamide hydrochloride | 640.241 | 98+(LC/MS) |
| 159 | 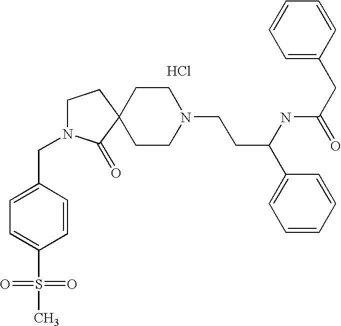 | N-{3-[2-(4-methanesulfonylbenzyl)-1- oxo-2,8-diaza-spiro[4.5]dec-8-yl]-1- phenyl-propyl}-2-phenyl-acetamide hydrochloride | 610.215 | 98+(LC/MS) |
| 160 | 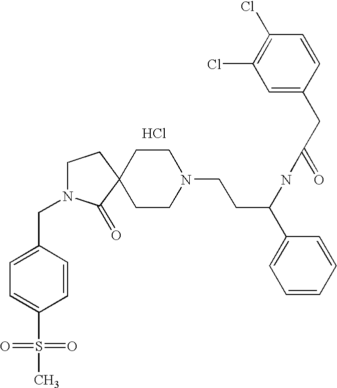 | 2-(3,4-dichlorophenyl)-N-{3-[2-(4- methanesulfonylbenzyl)-1-oxo-2,8- diaza-spiro[4.5]dec-8-yl]-1-phenyl- propyl}-acetamide hydrochloride | 679.105 | 94+(LC/MS) |
| 161 | 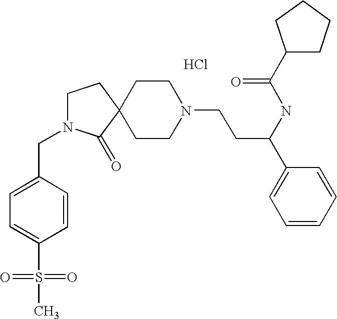 | Cyclopentanecarboxylic acid {3-[2-(4- methanesulfonylbenzyl)-1-oxo-2,8- diaza-spiro[4.5]dec-8-yl]-1-phenyl- propyl}-amide hydrochloride | 588.209 | 98+(LC/MS) |
| 162 | 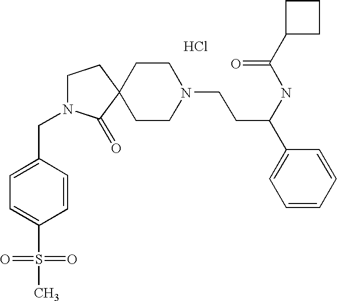 | Cyclobutanecarboxylic acid {3-[2-(4- methanesulfonylbenzyl)-1-oxo-2,8- diaza-spiro[4.5]dec-8-yl]-1-phenyl- propyl}-amide hydrochloride | 574.182 | 98+(LC/MS) |
| 163 | 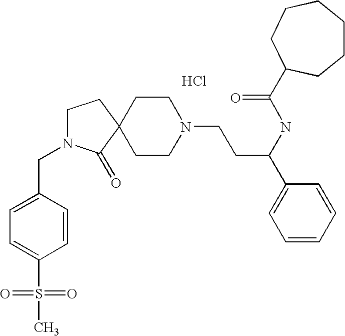 | Cycloheptanecarboxylic acid {3-[2-(4- methanesulfonylbenzyl)-1-oxo-2,8- diaza-spiro[4.5]dec-8-yl]-1-phenyl- propyl}-amide hydrochloride | 616.262 | 98+(LC/MS) |
| 164 | 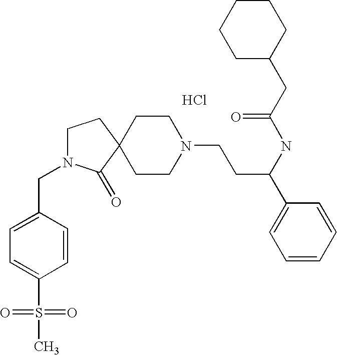 | 2-cyclohexyl-N-{3-[2-(4- methanesulfonylbenzyl)-1-oxo-2,8- diaza-spiro[4.5]dec-8-yl]-1-phenyl- propyl}-acetamide hydrochloride | 616.262 | 98+(LC/MS) |
| 165 | 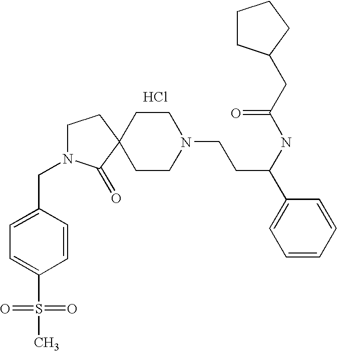 | 2-cyclopentyl-N-{3-[2-(4- methanesulfonylbenzyl)-1-oxo-2,8- diaza-spiro[4.5]dec-8-yl]-1-phenyl- propyl}-acetamide hydrochloride | 602.236 | 98+(LC/MS) |
| 166 | 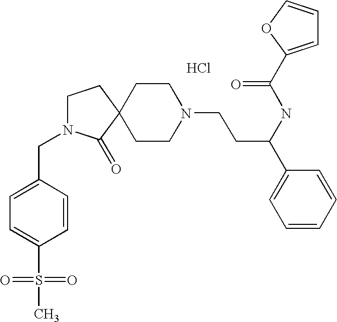 | Furan-2-carboxylic acid {3-[2-(4- meethanesulfonylbenzyl)-1-oxo-2,8- diaza-spiro[4.5]dec-8-yl]-1-phenyl- propyl}-amide hydrochloride | 586.149 | 98+(LC/MS) |
| 167 | 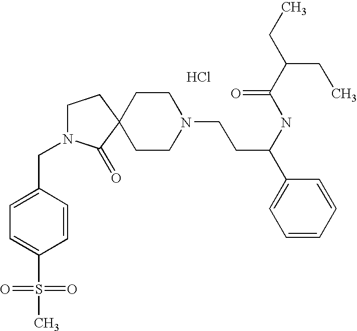 | 2-ethyl-N-{3-[2-(4- methanesulfonylbenzyl)-1-oxo-2,8- diaza-spiro[4.5]dec-8-yl]-1-phenyl- propyl}-butyramide hydrochloride | 590.225 | 98+(LC/MS) |
| 168 | 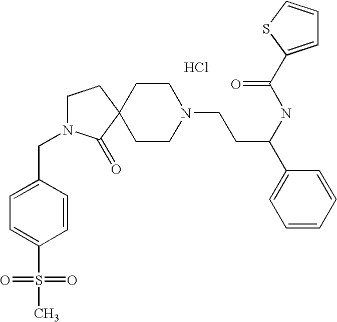 | Thiophene-2-carboxylic acid {3-[2-(4- methanesulfonylbenzyl)-1-oxo-2,8- diaza-spiro[4.5]dec-8-yl]-1-phenyl- propyl}-amide hydrochloride | 602.216 | 98+(LC/MS) |
| 169 | 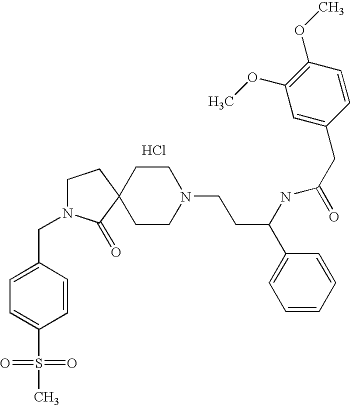 | 2-(3,4-dimethoxyphenyl)-N-{3-[2-(4- methanesulfonyl-benzyl)-1-oxo-2,8- diaza-spiro[4.5]dec-8-yl]-1-phenyl- propyl}-acetamide hydrochloride | 670.267 | 98+(LC/MS) |
| 170 | 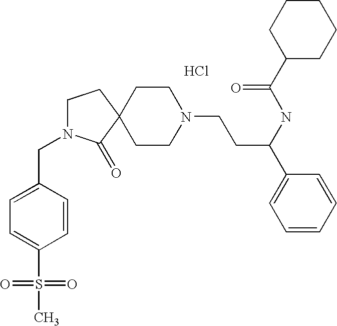 | Cyclohexanecarboxylic acid {3-[2-(4- methanesulfonylbenzyl)-1-oxo-2,8- diaza-spiro[4.5]dec-8-yl]-1-phenyl- propyl}-amide hydrochloride | 602.236 | 98+(LC/MS) |
| 171 | 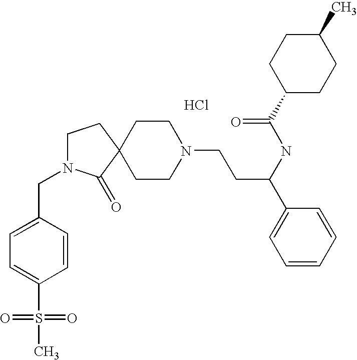 | 4-methyl-cyclohexanecarboxylic acid {3-[2-(4-methanesulfonylbenzyl)-1-oxo- 2,8-diaza-spiro[4.5]dec-8-yl]-1-phenyl- propyl}-amide hydrochloride | 616.262 | 98+(LC/MS) |
| 172 | 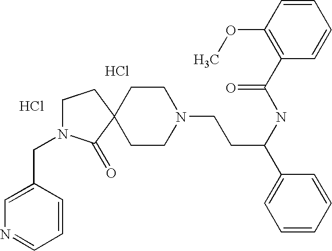 | 2-methoxy-N-[3-(1-oxo-2-pyridin-3- ylmethyl-2,8-diaza-spiro[4.5]dec-8-ylk)- 1-phenyl-propyl]-benzamide dihydrochloride | 585.572 | 98+(LC/MS) |
| 173 | 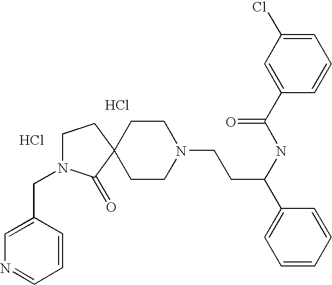 | 3-chloro-N-[3-(1-oxo-2-pyridin-3- ylmethyl-2,8-diaza-spiro[4.5]dec-8-yl)- 1-phenyl-propyl]-benzamide dihydrochloride | 589.992 | 98+(LC/MS) |
| 174 | 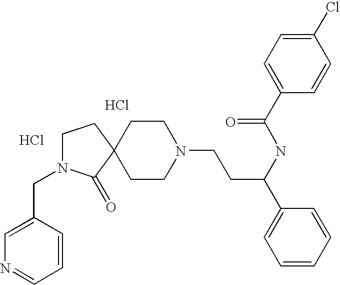 | 4-chloro-N-[3-(1-oxo-2-pyridin-3- ylmethyl-2,8-diaza-spiro[4.5]dec-8-yl)- 1-phenyl-propyl]-benzamide dihydrochloride | 589.992 | 98+(LC/MS) |
| 175 | 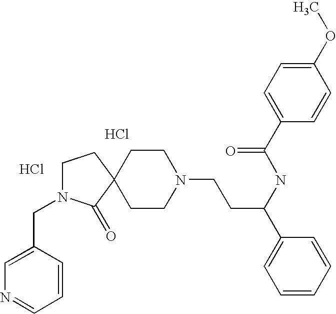 | 4-methoxy-N-[3-(1-oxo-2-pyridin-3- ylmeethyl-2,8-diaza-spiro[4.5]dec-8-yl)- 1-phenyl-propyl]-benzamide dihydrochloride | 585.572 | 98+(LC/MS) |
| 176 | 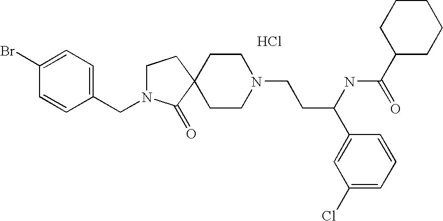 | Cyclohexanecarboxylic acid [3-[2-(4- bromobernzyl)-1-oxo-2,8-diaza- spiro[4.5]dec-8-yl]-1-(3-chloro-phenyl)- propyl]-amide hydrochloride | 637.486 | 94.3% (LC/MS) |
| 177 | 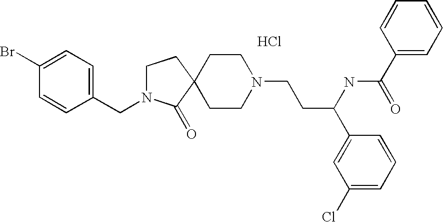 | N-[3-[2-(4-bromobenzyl)-1-oxo-2,8- diaza-spiro[4.5]dec-8-yl]-1-(3- chlorophenyl)-propyl]-benzamide hydrochloride | 631.439 | 98+(LC/MS) |
| 178 | 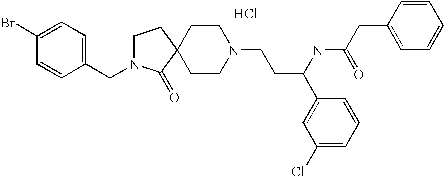 | N-[3-[2-(4-bromobenzyl)-1-oxo-2,8- diaza-spiro[4.5]dec-8-yl]-1-(3- chlorophenyl)-propyl]-2-phenyl- acetamide hydrochloride | 645.465 | 98+(LC/MS) |
| 179 | 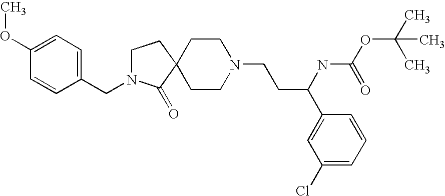 | {1-(3-chlorophenyl)-3-[2-(4- methoxybenzyl)-1-oxo-2,8-diaza- spiro[4.5]dec-8-yl]-propyl}carbamic acid tert-butyl ester | 542.116 | >95% (1H NMR) |
| 180 | 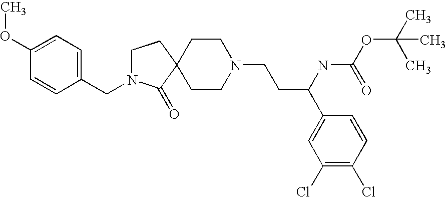 | {1-(3,4-dichlorophenyl)-3-[2-(4- methoxybenzyl)-1-oxo-2,8-diaza- spiro[4.5]dec-8-yl]-propyl}-carbamic acid tert-butyl ester | 576.561 | >95% (1H NMR) |
| 181 | 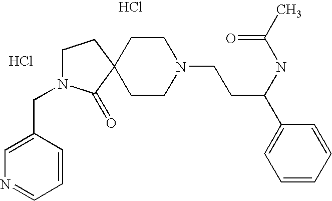 | N-[3-(1-oxo-2-pyridin-3-ylmethyl-2,8- diaza-spiro[4.5]dec-8-yl)-1-phenyl- propyl]-acetamide dihydrochloride | 493.476 | 90+(LC/MS) |
| 182 | 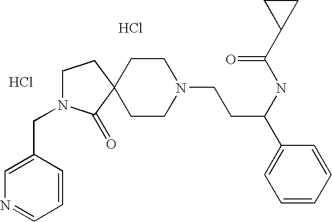 | Cyclopropanecarboxylic acid [3-(1-oxo- 2-pyrridin-3-ylmethyl-2,8-diaza- spiro[4.5]dec-8-yl)-1-phenyl-propyl]- amide dihydrochloride | 519.513 | 98+(LC/MS) |
| 183 | 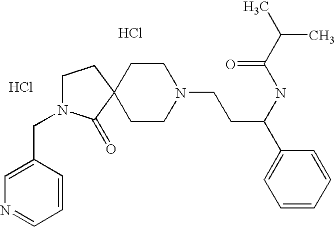 | N-[3-(1-oxo-2-pyridin-3-ylmethyl-2,8-0 diaza-spiro[4.5]dec-8-yl)-1-phenyl- propyl]-issobutyramide dihydrochloride | 521.529 | 98+(LC/MS) |
| 184 | 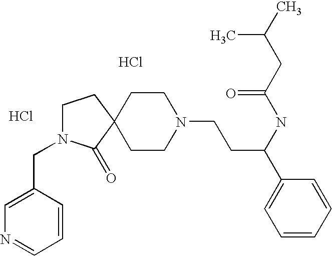 | 3-methyl-N-[3-(1-oxo-2-pyridin-3- ylmethyl-2,8-diaza-spiro[4.5]dec-8-yl)- 1-phenyl-propyl]-butyramide dihydrochloride | 535.556 | 98+(LC/MS) |
| 185 | 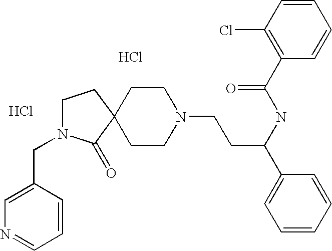 | 2-chloro-NM-[3-(1-oxo-2-pyridin-3- ylmethyl-2,8-diaza-spiro[4.5]dec-8-yl)- 1-phenyl-propyl]-benzamide dihydrochloride | 589.992 | 98+(LC/M S) |
| 186 | 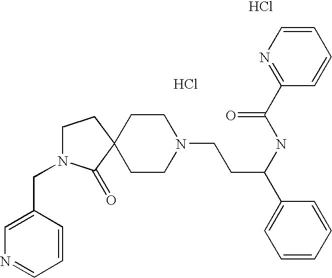 | Pyridin-2-carboxylic acid [3-(1-oxo-2- pyridin-3-ylmethyl-2,8-diaza- spiro[4.5]dec-8-yl)-1-phenyl-propyl]- amide trihydrochloride | 592.995 | 98+(LC/MS) |
| 187 | 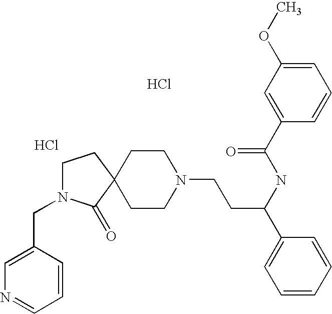 | 3-methoxy-N-[3-(1-oxo-2-pyridin-3- ylmethyl-2,8-diaza-spiro[4.5]dec-8-yl)- 1-phenyl-propyl]-benzamide dihydrochloride | 585.572 | 98+(LC/MS) |
| 188 | 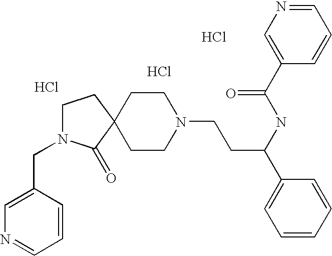 | N-[3-(1-oxo-2-pyridin-3-ylmethyl-2,8- diaza-spiro[4.5]dec-8-yl)-1-phenyl- propyl]-nicotinamide trihydrochloride | 592.995 | 98+(LC/MS) |
| 189 | 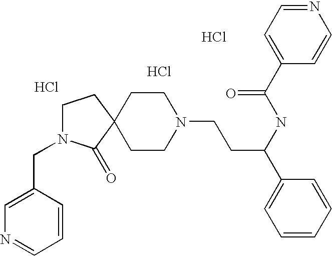 | N-[3-(1-oxo-2-pyridin-3-ylmethyl-2,8- diaza-spiro[4.5]dec-8-yl)-1-phenyl- propyl]-isonicotinamide trihydrochloride | 592.995 | 98+(LC/MS) |
| 190 | 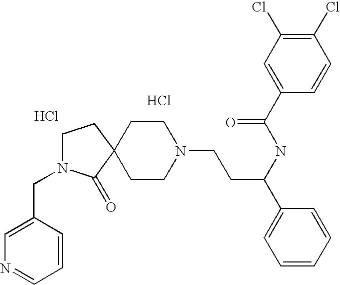 | 3,4-dichloro-N-[3-(1-oxo-2-pyridin-3- ylmethyl-2,8-diaza-spiro[4.5]dec-8-yl)- 1-phenyl-propyl]-benzamide dihydrochloride | 624.437 | 98+(LC/MS) |
| 191 | 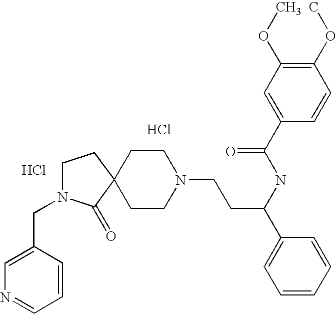 | 3,4-dimethoxy-N-[3-(1-oxo-2-pyridin-3- ylmethyl-2,8-diaza-spiro[4.5]dec-8-yl)- 1-phenyl-propyl]-benzamide dihydrochloride | 615.598 | 98+(LC/MS) |
| 192 | 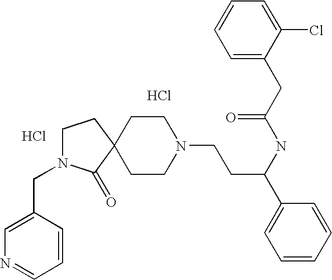 | 2-(2-chlorophenyl)-N-[3-(1-oxo-2- pyridin-3-ylmethyl-2,8-diaza- spiro[4.5]dec-8-yl)-1-phenyl-propyl]- acetamide dihydrochloride | 604.018 | 98+(LC/MS) |
| 193 | 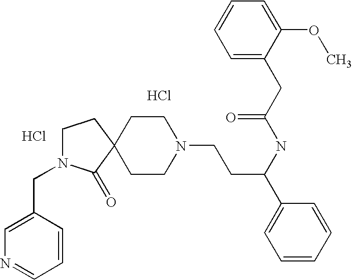 | 2-(2-methoxyphenyl)-N-[3-(1-oxo-2- pyridin-3-ylmethyl-2,8-diaza- spiro[4.5]dec-8-yl)-1-phenyl-propyl]- acetamide dihydrochloride | 599.599 | 98+(LC/MS) |
| 194 | 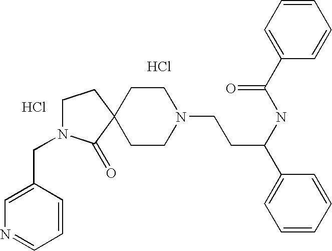 | N-[3-(1-oxo-2-pyridin-3-ylmethyl)-2,8- diaza-spiro[4.5]dec-8-yl)-1-phenyl- propyl]-benzamide dihydrochloride | 555.546 | 98+(LC/MS) |
| 195 | 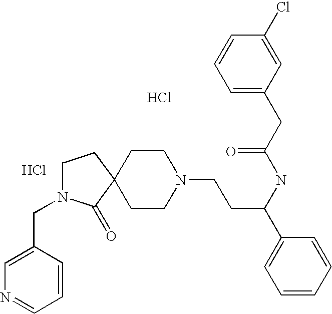 | 2-(3-chloro-phenyl)-N-[3-(1-oxo-2- pyridin-3-ylmethyl-2,8-diaza- spiro[4.5]dec-8-yl)-1-phenyl-propyl]- acetamide dihydrochloride | 604.018 | 98+(LC/MS) |
| 196 | 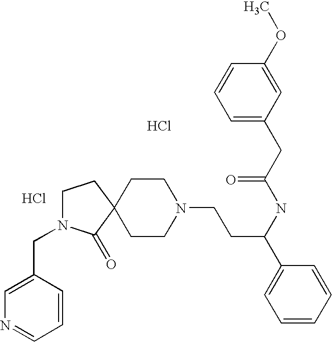 | 2-(3-methoxyphenyl)-N-[3-(1-oxo-2- pyridin-3-ylmethyl-2,8-diaza- spiro[4.5]dec-8-yl)-1-phenyl-propyl]- acetamide dihydrochloride | 599.599 | 93.3% (LC/MS) |
| 197 | 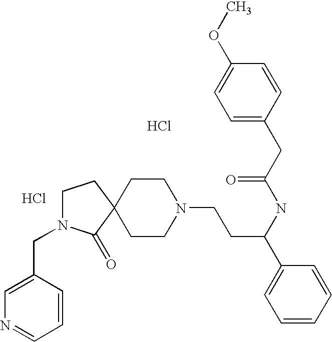 | 2-(4-methoxyphenyl)-N-[3-(1-oxo-2- pyridin-3-ylmethyl-2,8-diaza- spiro[4.5]dec-8-yl)-1-phenyl-propyl]- acetamide dihydrochloride | 599.599 | 98+(LC/MS) |
| 198 | 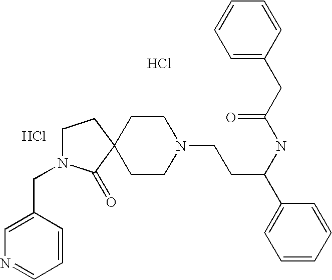 | N-[3-(1-oxo-2-pyridin-3-ylmethyl-2,8- diaza-spiro[45]dec-8-yl)-1-phenyl- propyl]-2-phenyl-acetamide dihydrochloride | 569.573 | 93.3% (LC/MS) |
| 199 | 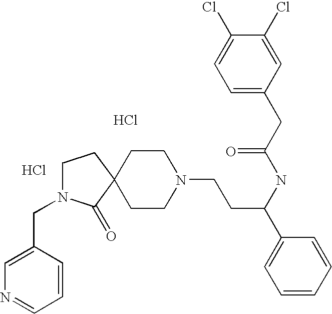 | 2-(3,4-dichloro-phenyl)-N-[3-(1-oxo-2- pyridin-3-ylmethyl-2,8-diaza- spiro[4.5]dec-8-yl)-1-phenyl-propyl]- acetamide dihydrochloride | 638.463 | 91.5% (LC/MS) |
| 200 | 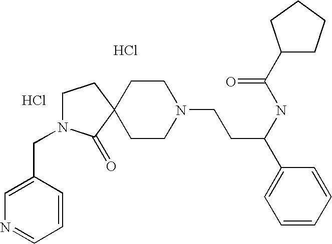 | Cyclopentanecarboxylic acid [3-(1-oxo- 2-pyrridin-3-ylmethyl-2,8-diaza- spiro[4.5]dec-8-yl)-1-phenyl-propyl]- amide dihydrochloride | 547.567 | 98+(LC/MS) |
| 201 | 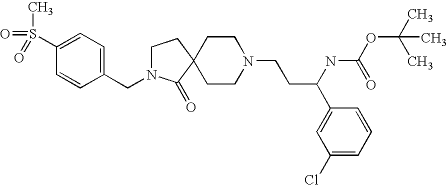 | {1-(3-chlorophenyl)-3-[2-(4- methanesulfonylbenzyl)-1-oxo-2,8- diaza-spiro[4.5]dec-8-yl]-propyl}- carbamic acid tert-butyl ester | 590.181 | >95% (1H NMR) |
| 202 | 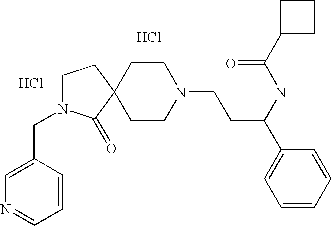 | Cyclobutanecarboxylic acid [3-(1-oxo- 2-pyridin-3-ylmethyl-2,8-diaza- spiro[4.5]dec-8-yl)-1-phenyl-propyl]- amide dihydrochloride | 533.54 | 95+(LC/MS) |
| 203 | 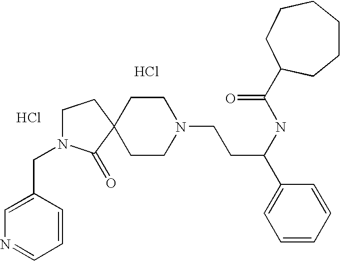 | Cycloheptanecarboxylic acid [3-(1-oxo- 2-pyridin-3-ylmethyl-2,8-diaza- spiro[4.5]dec-8-yl)-1-phenyl-propyl}- amide dihydrochloride | 575.621 | 94.5% (LC/MS) |
| 204 | 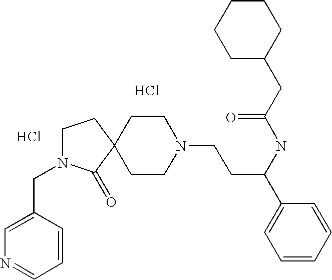 | 2-cyclohexyl-N-[3-(1-oxo-2-pyridin-3- ylmethyl-2,8-diaza-spiro[4.5]dec-8-yl)- 1-phenyl-propyl]-acetamide dihydrochloride | 575.621 | 93.4% (LC/MS) |
| 205 | 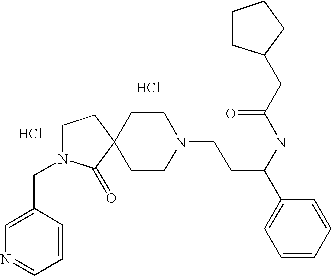 | 2-cyclopentyl-N-[3-(1-oxo-2-pyridin-3- ylmethyl-2,8-diaza-spiro[4.5]dec-8-yl)- 1-phenyl-propyl]-acetamide dihydrochloride | 561.594 | 94.6% (LC/MS) |
| 206 | 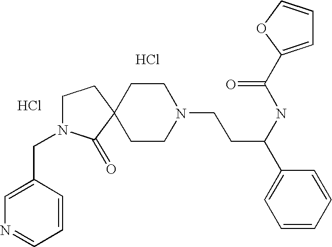 | Furan-2-carboxylic acid [3-(1-oxo-2- pyridin-3-ylmethyl-2,8-diaza- spiro[4.5]dec-8-yl)-1-phenyl-propyl]- amide dihydrochloride | 545.508 | 98+(LC/MS) |
| 207 | 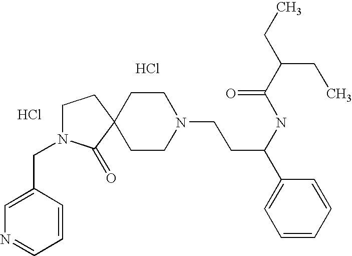 | 2-ethyl-N-[3-(1-oxo-2-pyridin-3- ylmethyl-2,8-diaza-spiro[4.5]dec-8-yl)- 1-phenyl-propyl]-butyramide dihydrochloride | 549.583 | 93.9% (LC/MS) |
| 208 | 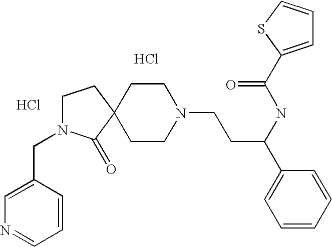 | Thiophene-2-carboxylic acid [3-(1-oxo- 2-pyridin-3-ylmethyl-2,8-diaza- spiro[4.5]dec-8-yl)-1-phenyl-phenyl- propyl]-amide dihydrochloride | 561.575 | 98+(LC/MS) |
| 209 | 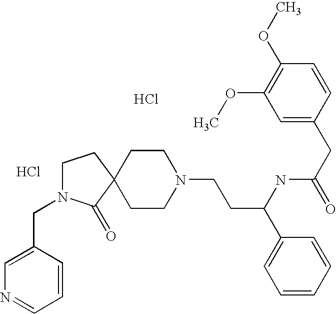 | 2-(3,4-dimethoxyphenyl)-N-[3-(1-oxo-2- pyridin-3-ylmethyl-2,8-diaza- spiro[4.5]dec-8-yl)-1-phenyl-propyl]- acetamide dihydrochloride | 629.625 | 98+(LC/MS) |
| 210 | 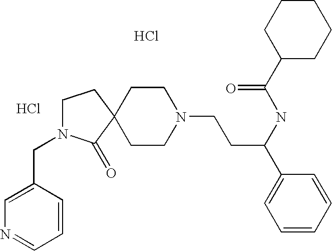 | Cyclohexanecarboxylic acid [3-(1-oxo- 2-pyridin-3-ylmethyl-2,8-diaza- spiro[44.5]dec-8-yl)-1-phenyl-propyl]- amide dihydrochloride | 561.594 | 98+(LC/MS) |
| 211 | 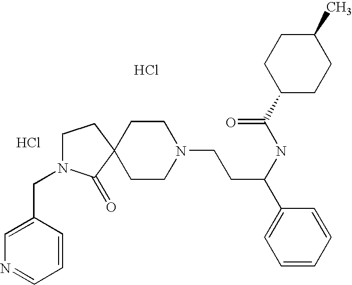 | 4-methyl-cyclohexanecarboxylic acid [3-(1-oxo-2-pyridin-3-ylmethyl-2,8- diaza-spiro[4.5]dec-8-yl)-1-phenyl- propyl]-amide dihydrochloride | 575.621 | 92.1% (LC/MS) |
| 212 | 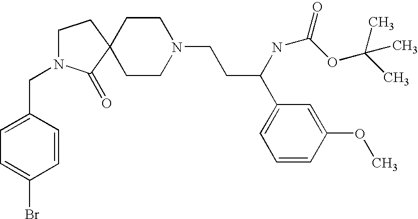 | [3-[2-(4-bromobenzyl)-1-oxo-2,8-diaza- spiro[4.5]dec-8-yl]-1-(3- methoxyphenyl)-propyl]-carbamic acid tert-butyl ester | 586.567 | >99% (LC/MS) |
| 213 | 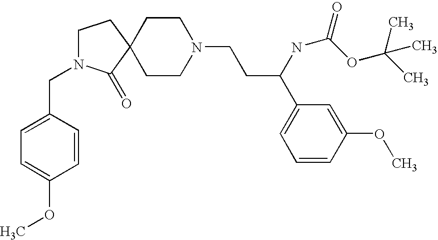 | [3-[2-(4-methoxybenzyl)-1-oxo-2,8- diaza-spiro[4.5]dec-8-yl]-1-(3- methoxyphenyl)-propyl]-carbamic acid tert-butyl ester | 537.697 | >99% (LC/MS) |
| 214 | 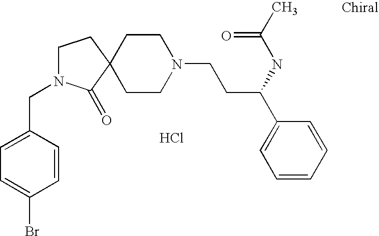 | (S)-N-{3-[2-(4-methoxybenzyl)-1-oxo-2,8- diaza-spiro[4.5]dec-8-yl]-1-phenyl- propyl}-acetamide hydrochloride | 534.923 | 98+(LC/MS) |
| 215 | 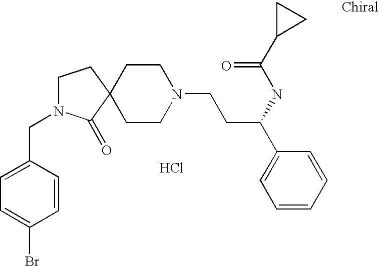 | (S)-cyclopropanecarboxylic acid {3-[2- (4-bromobenzyl)-1-oxo-2,8-diaza- spiro[4.5]dec-8-yl]-1-phenyl-propyl}- amide hydrochloride | 560.961 | 98+(LC/MS) |
| 216 | 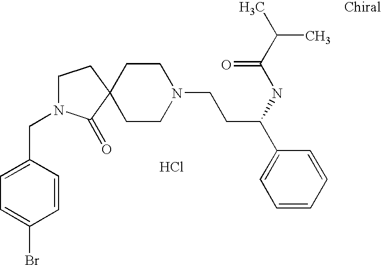 | (S)-N-{3-[2-(4-bromobenzyl)-1-oxo-2,8- diaza-spiro[4.5]dec-8-yl]-1-phenyl- propyl}-isobutyramide hydrochloride | 562.876 | 98+(LC/MS) |
| 217 | 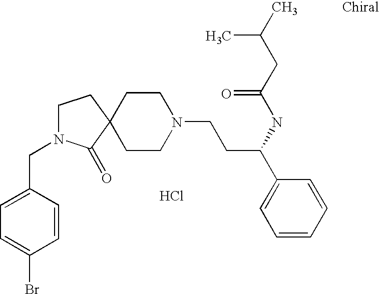 | (S)-N-{3-[2-(4-bromobenzyl)-1-oxo-2,8- diaza-spiro[4.5]dec-8-yl]-1-phenyl- propyl}-3-methyl-butyramide hydrochloride | 577.003 | 98+(LC/MS) |
| 218 | 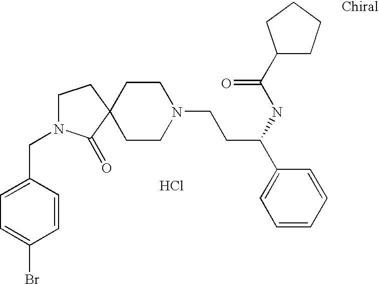 | (S)-cyclopentanecarboxylic acid {3-[2- (4-bromobenzyl)-1-oxo-2,8-diaza- spiro[4.5]dec-8-yl]-1-phenyl-propyl}- amide hydrochloride | 589.014 | 98+(LC/MS) |
| 219 | 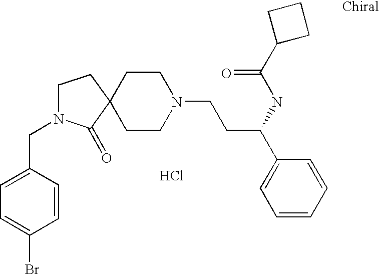 | (S)-cyclobutanecarboxylic acid {3-[2-(4- bromobenzyl)-1-oxo-2,8-diaza- spiro[4.5]dec-8-yl]-1-phenyl-propyl}- amide hydrochloride | 574.987 | 98+(LC/MS) |
| 220 | 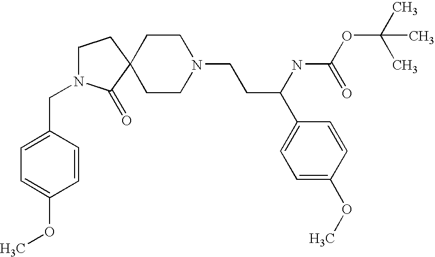 | [3-[2-(4-methoxybenzyl)-1-oxo-2,8- diaza-spiro[4.5]dec-8-yl]-1-(4- methoxyphenyl)-propyl]carbamic acid tert-butyl ester | 537.697 | 95% BY HPLC |
| 221 | 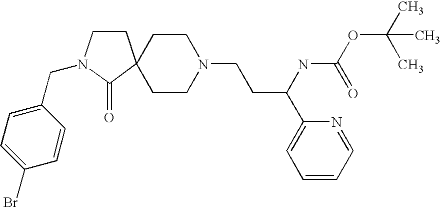 | {3-[2-(4-bromobenzyl)-1-oxo-2,8-diaza- spiro[4.5]dec-8-yl]-1-pyridin-2-yl- propyl}-carbamic acid tert-butyl ester | 557.529 | 99% BY HPLC |
| 222 | 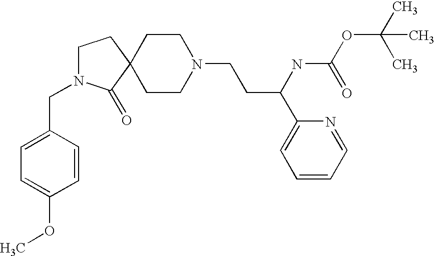 | {3-[2-(4-methoxybenzyl)-1-oxxo-2,8- diaza-spiro[4.5]dec-8-yl]-1-pyridin-2-yl- propyl}-carbamic acid tert-butyl ester | 508.659 | 99% BY HPLC |
| 223 | 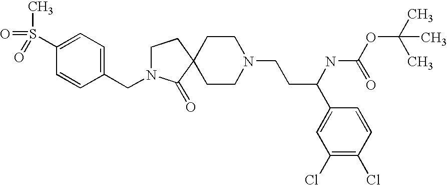 | {1-(3,4-dichlorophenyl)-3-[22-(4- methanesulfonylbenzyl)-1-oxo-2,8- diaza-spiro[44.5]dec-8-yl]-propyl}- carbamic acid tert-butyl ester | 624.626 | >95% (1H NMR) |
| 224 | 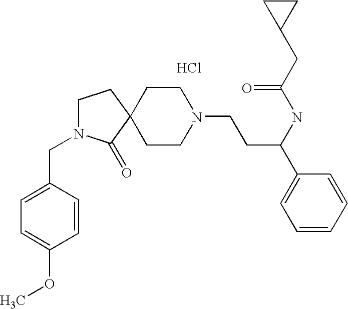 | 2-cyclopropyl-N-{3-[2-(4- methoxybenzyl)-1-oxxo-2,8-diaza- spiro[4.5]dec-8-yl]-1-phenyl-propyl}- acetamide hydrochloride | 526.117 | 98+(LC/MS) |
| 225 | 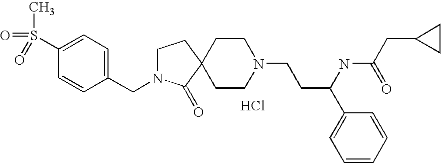 | 2-cyclopropyl-N-{3-[2-(4- methanesulfonylbenzyl)-1-oxo-2,8- diaza-spiro[4.5]dec-8-yl]-1-phenyl- propyl}-acetamide hydrochloride | 574.182 | 94.4% (LC/MS) |
| 226 | 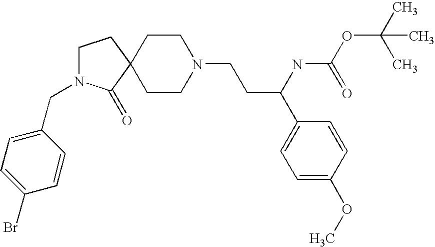 | [3-[2-(4-bromobenzyl)-1-oxo-2,8-diaza- spiro[4.5]dec-8-yl]-1-(4- methoxyphenyl)-propyl]-carbamic acid tert-butyl ester | 586.567 | 95% BY HPLC |
| 227 | 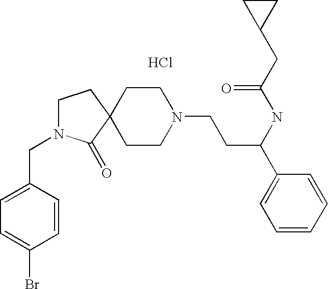 | N-{3-[2-(4-bromobenzyl)-1-oxo-2,8- diaza-spiro[4.5]dec-8-yl]-1-phenyl- propyl}-2-cyclopropyl-acetamide hydrochloride | 574.987 | 98+(LC/MS) |
| 228 | 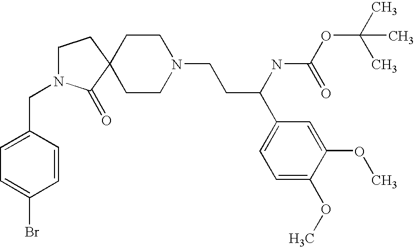 | [3-[2-(4-bromobenzyl)-1-oxo-2,8-diaza- spirro[4.5]dec-8-yl]-1-(3,4- dimethoxyphenyl)-propyl]-carbamic acid tert-butyl ester | 616.593 | 99% BY HPLC |
| 229 | 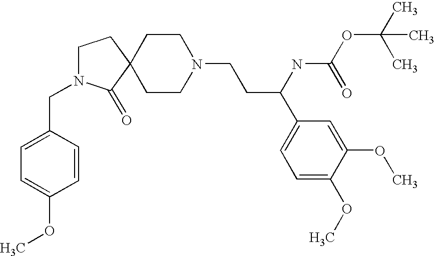 | {1-(3,4-dimethoxyphenyl)-3-[2-(4- methoxybenzyl)-1-oxo-2,8-diaza- spiro[4.5]dec-8-yl]-propyl}-carbamic acid tert-butyl ester | 567.722 | 97% BY HPLC |
| 230 | 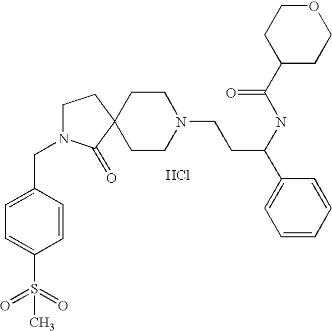 | Tetrahydro-pyran-4-carboxylic acid {3- [2-(4-methanesulfonylbenzyl)-1-oxo- diaza-spiro[4.5]dec-8-yl]-1-phenyl- propyl}-amide hydrochloride | 604.208 | >95% (HPLC) |
| 231 | 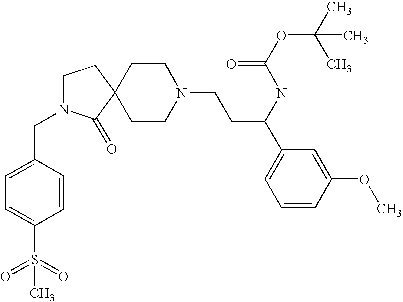 | [3-[2-(4-methanesulfonylbenzyl)-1-oxo- 2,8-diaza-spiro[4.5]dec-8-yl]-1-(3- methoxy-phenyl)-propyl]-carbamic acid tertbutyl ester | 585.762 | >99% (HPLC) |
| 232 | 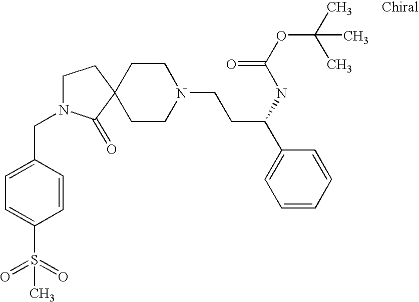 | (S)-{3-[2-(4-methanesulfonylbenzyl)-1- oxo-2,8-diaza-spiro[4.5]dec-8-yl]-1- phenyl-propyl}-carbamic acid tert-butyl ester | 555.736 | >99% (HPLC) |
| 233 | 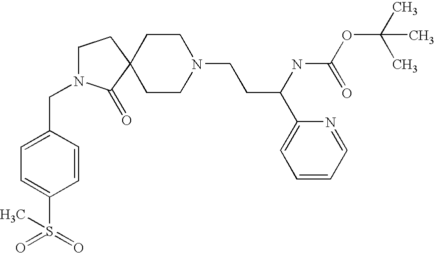 | {3-0[2-(4-methanesulfonylbenzyl)-1-oxo- 2,8-diaza-spiro[4.5]dec-8-yl]-1-pyridin- 2-yl-propyl}-carbamic acid tert-butyl ester | 556.724 | 95% BY HPLC |
| 234 | 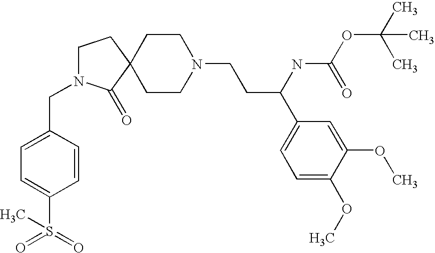 | {1-(3,4-dimethoxyphenyl)-3-[2-(4- meethanesulfonylbenzyl)-1-oxo-2,8- diaza-spiro[4.5]dec-8-yl]-propyl}- carbamic acid tert-butyl ester | 615.787 | 97% BY HPLC |
| 235 | 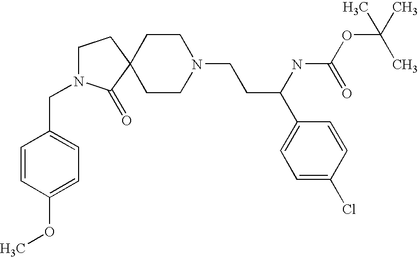 | {1-(4-chlorophenyl)-3-[2-(4- methoxybenzyl)-1-oxo-2,8-diaza- spiro[4.5]dec-8-yl]-propyl}-carbamic acid tert-butyl ester | 542.116 | >90% (HNMR) |
| 236 | 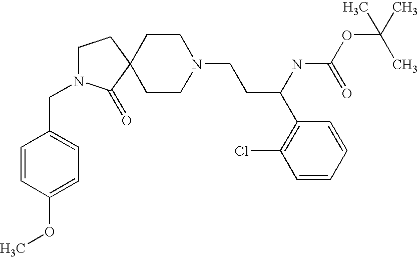 | {1-(2-chlorophenyl)-3-[2-(4- methoxybenzyl)-1-oxo-2,8-diaza- spiro[4.5]dec-8-yl]-propyl}-carbamic acid tert-butyl ester | 542.116 | >90% (HNMR) |
| 237 | 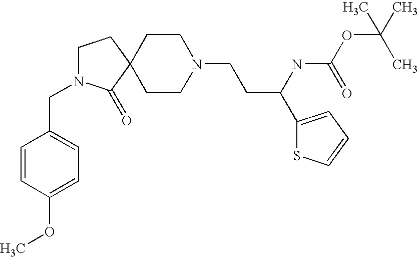 | {3-[2-(4-methoxybenzyl)-1-oxo-2,8- diaza-spiro[4.5]dec-8-yl]-1-thiophen-2- yl-propyl}-carbamic acid tert-butyl ester | 513.699 | >90% (HNMR) |
| 238 | 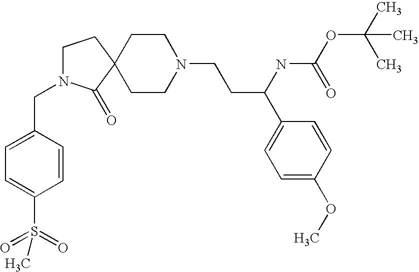 | [3-[2-(4-methanesulfonylbenzyl)-1-oxo- 2,8-diaza-spiro[4.5]dec-8-yl]-1-(4- methoxyphenyl)-propyl]-carbamic acid tert-butyl ester | 585.762 | 95% BY HPLC |
| 239 | 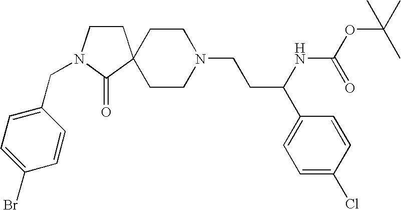 | [3-[2-(4-bromobenzyl)-1-oxo-2,8-diaza- spiro[4.5]dec-8-yl]-1-(4-chlorophenyl)- propyl]-carbamic acid tert-butyl ester | 590.986 | >90% (HNMR) |
| 240 | 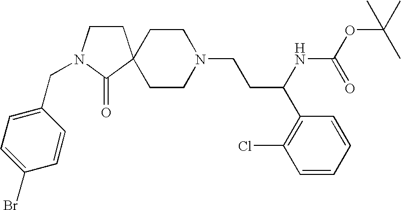 | [3-[2-(4-bromobenzyl)-1-oxo-2,8-diaza- spiro[4.5]dec-8-yl]-1-(2-chlorophenyl)- propyl]-carbamic acid tert-butyl ester | 590,986 | >90% (HNMR) |
| 241 | 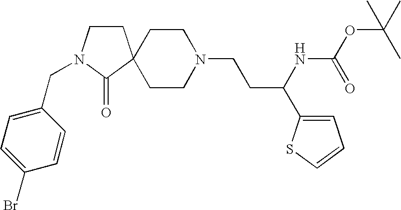 | {3-[2-(4-bromobenzyl)-1-oxo-2,8-diaza- spiro[4.5]dec-8-yl]-1-thiophen-2-yl- propyl}-carbamic acid tert-butyl ester | 562.569 | >90% (HNMR) |
| 242 | 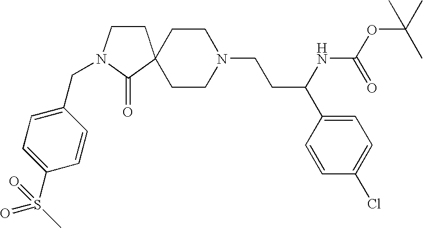 | {1-(4-chlorophenyl)-3-[2-(4- methanesulfonylbenzyl)-1-oxo-2,8- diaza-spiro[4.5]dec-8-yl]-propyl}- carbamic acid tert-butyl ester | 590.181 | >90% (HNMR) |
| 243 | 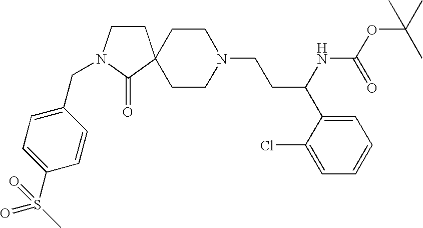 | {1-(2-chlorophenyl)-3-[2-(4- methanesulfonylbenzyl)-1-oxo-2,8- diaza-spiro[4.5]dec-8-yl]-propyl}- carbamic acid tert-butyl ester | 590.181 | >90% (HNMR) |
| 244 | 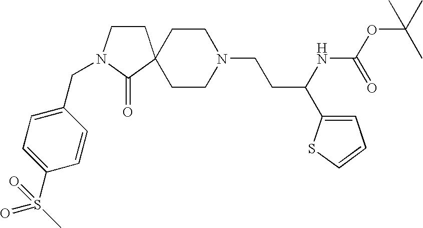 | {3-[2-(4-methanesulfonylbenzyl)-1-oxo- 2,8-diaza-spiro[4.5]dec-8-yl]-1- thiophen-2-yl-propyl}-carbamic acid tert-butyl ester | 561.764 | >90% (HNMR) |
| 245 | 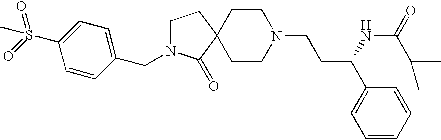 | (S)-N-{3-[2-(4-0methanesulfonylbenzyl)- 1-oxo-2,8-diaza-spiro[4.5]dec-8-yl]-1- phenyl-propyl}-isobutyramide | 525.71 | 98+(LC/MS) |
| 246 | 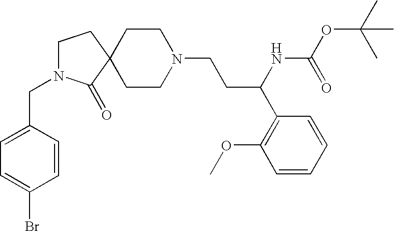 | [3-[2-(4-bromobenzyl)-1-oxo-2,8-diaza- spiro[4.5]dec-8-yl]-1-(2- methoxyphenyl)-propyl]-carbamic acid tert-butyl ester | 586.567 | 89% BY HPLC |
| 247 | 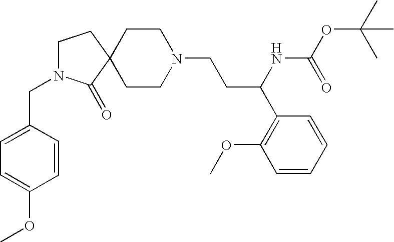 | [3-[2-(4-methoxy-benzyl)-1-oxo-2,8- diaza-spiro[4.5]dec-8-yl]-1-(2-methoxy- phenyl)-propyl]-carbamic acid tert-butyl ester | 537.697 | 86% BY HPLC |
| 248 | 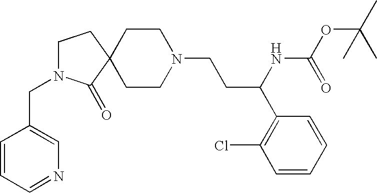 | [1-(2-chlorophenyl)-3-(1-oxo-2-pyridin- 3-ylmethyl-2,8-diaza-spiro[4.5]dec-8- yl)-propyl]-carbamic acid tert-butyl ester | 513.078 | >90% (HNMR) |
| 249 | 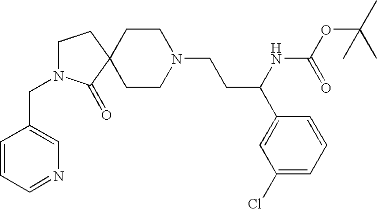 | [1-(3-chlorophenyl)-3-(1-oxo-2-pyridin- 3-ylmethyl-2,8-diaza-spiro[4.5]dec-8- yl)-propyl]-carbamic acid tert-butyl ester | 513.078 | >90% (HNMR) |
| 250 | 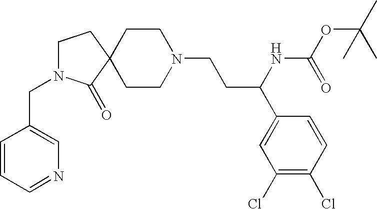 | [1-(3,4-dichlorophenyl)-3-(1-oxo-2- pyridin-3-ylmethyl-2,8-diaza- spiro[4.5]dec-8-yl)-preopyl]-carbamic acid tert-butyl ester | 547.523 | >90% (HNMR) |
| 251 | 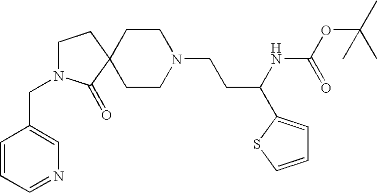 | [3-(1-oxo-2-pyridin-3-ylmethyl-2,8- diaza-spiro[4.5]dec-8-yl)-1-thiophen-2- yl-propyl]-carbamic acid tert-butyl ester | 484.661 | >90% (HNMR) |
| 252 | 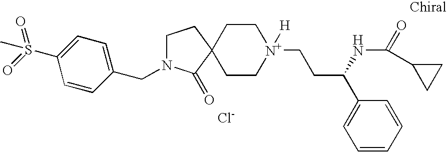 | (S)-8-[3-(cyclopropanecarbonyl-amino)- 3-phenyl-propyl]-2-(4- methanesulfonylbenzyl)-1-oxo-2,8- diaza-spiro[4.5]decane hydrochloride | 560.155 | 98+(LC/MS) |
| 253 | 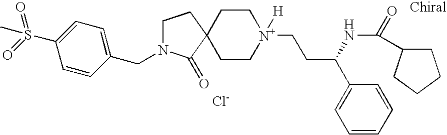 | (S)-8-[3-(cyclopentanecarbonyl-amino)- 3-phenyl-propyl]-2-(4- methanesulfonylbenzyl)-1-oxo-2,8- diaza-spiro[4.5]decane hydrochloride | 588.209 | 98+(LOC/MS) |
| 254 | 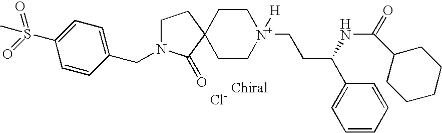 | (S)-8-[3-(cyclohexanecarbonyl-amino)- 3-phenyl-propyl]-2-(4- methanesulfonylbenzyl)-1-oxo-2,8- diaza-spiro[4.5]decane hydrochloride | 602.236 | 98+(LC/MS) |
| 255 | 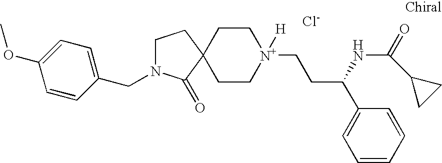 | (S)-8-[3-(cyclopropanecarbonyl-amino)- 3-phenyl-propyl]-2-(4-methoxybenzyl)- 1-oixo-2,8-diaza-spiro[4.5]decane hydrochloride | 512.09 | 98+(LC/MS) |
| 256 | 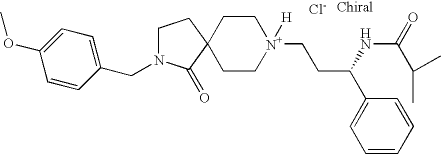 | (S)-8-3-isobutyrylamino-3-phenyl- propyl)-2-(4-methoxybenzyl)-1-oxo-2,8- diaza-spiro[4.5]decane hydrochloride | 514.016 | 98+(LC/MS) |
| 257 | 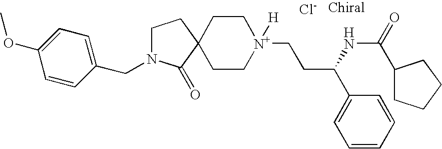 | (S)-8-[3-(cyclopentanecarbonyl-amino)- 3-phenyl-propyl]-2-(4-methoxybenzyl)- 1-oxo-2,8-diaza-spiro[4.5]decane hydrochloride | 540.144 | 98+(LC/MS) |
| 258 | 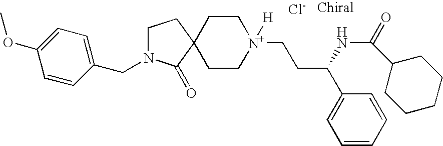 | (S)-8-[3-(cyclohexanecarbonyl-amino)- 3-phenyl-propyl]-2-(4-methoxybeenzyl)- 1-oxxo-2,8-diaza-spiro[4.5]decane hydrochloride | 554.171 | 98+(LC/MS) |
| 259 | 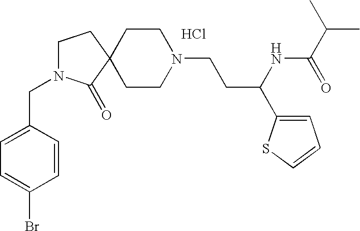 | N-{3-[2-(4-bromobenzyl)-1-oxo-2,8- diaza-spiro[4.5]dec-8-yl]-1-thiophen-2- yl-propyl}-isobutyramide hydrochloride | 569.005 | 98+(LC/MS) |
| 260 | 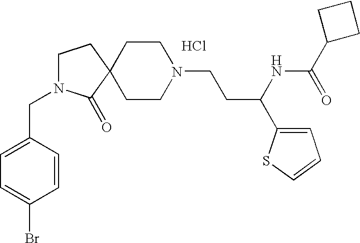 | Cyclobutanecarboxylic acid {3-[2-(4- bromobenzyl)-1-oxo-2,8-diaza- spiro[4.5]dec-8-yl]-1-thiophen-2-yl- propyl}-amide hydrochloride | 581.016 | 98+(LC/MS) |
| 261 | 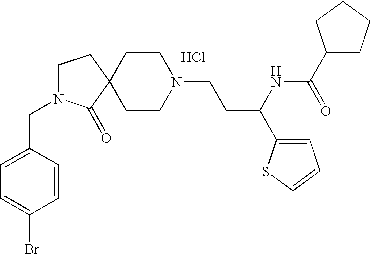 | Cyclopentanecarboxylic acid {3-[2-(4- bromobenzyl)-1-oxo-2,8-diaza- spiro[4.5]dec-8-yl]-1-thiophen-2-yl- propyl}-amide hydrochloride | 595.042 | 98+(LC/MS) |
| 262 | 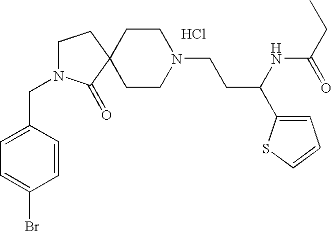 | N-{3-[2-(4-bromo-enzyl)-1-oxo-2,8- diaza-spiro[4.5]dec-8-yl]-1-thiophen-2- yl-propyl}-propionamide hydrochloride | 554.978 | 98+(LC/MS) |
| 263 | 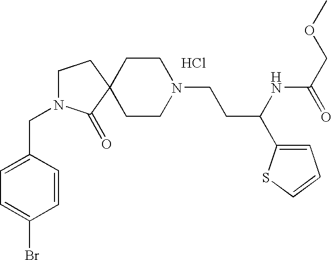 | N-{3-[2-(4-bromobenzyl)-1-oxo-2,8- diaza-sspiro[4.5]dec-8-yl]-1-thiophen-2- yl-propyl}-2-methoxy-acetamide hydrochloride | 570.977 | 98+(LC/MS) |
| 264 | 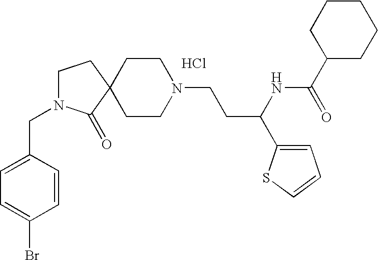 | Cyclohexanecarboxylic acid {3-[2-(4- bromobenzyl)-1-oxo-2,8-diaza- spiro[4.5]dec-8-yl]-1-thiophen-2-yl- propyl}-amide hydrochloride | 609.069 | 98+(LC/MS) |
| 265 | 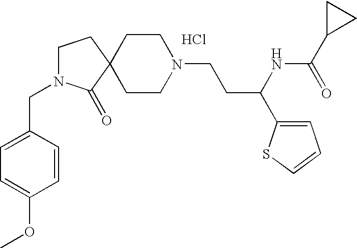 | Cyclopropanecarboxylic acid {3-[2-(4- methoxybenzyl)-1-oxo-2,8-diaza- spiro[4.5]dec-8-yl]-1-thiophen-2-yl- propyl}-amide hydrochloride | 518.118 | 98+(LC/MS) |
| 266 | 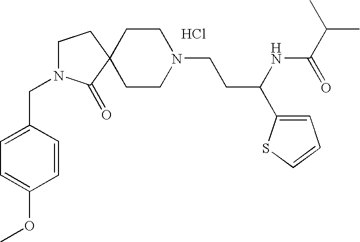 | N-{3-[2-(4-methoxybenzyl)-1-oxo-2,8- diaza-spiro[4.5]dec-8-yl]-1-thiophen-2- yl-propyl}-issobutyramide hydrochloride | 520.134 | 98+(LC/MS) |
| 267 | 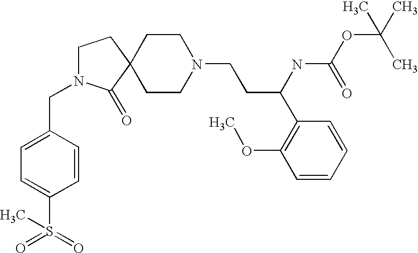 | [3-[2-(4-methasnesulfonylbenzyl)-1-oxo- 2,8-diaza-spiro[4.5]dec-8-yl]-1-(2- methoxyphenyl)-propyl]-carbamic acid tert-butyl ester | 585.762 | 89% BY HPLC |
| 268 | 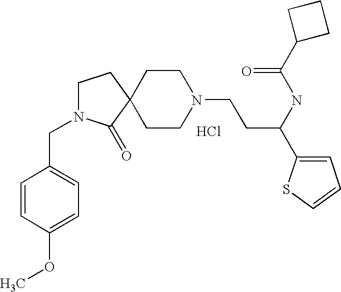 | Cyclobutanecarboxylic acid {3-0[2-(4- methoxybenzyl)-1-oxo-2,8-diaza- spiro[4.5]dec-8-yl]-1-thiophen-2-yl- propyl}-amide hydrochloride | 532.145 | 98+(LC/MS) |
| 269 | 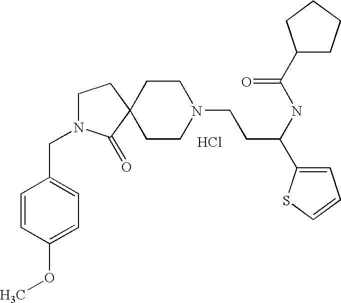 | Cyclopentanecarboxylic acid {3-[2-(4- methoxybenzyl)-1-oxo-2,8-diaza- spiro[4.5]dec-8-yl]-1-thiophen-2-yl- propyl}-amide hydrochloride | 546.172 | 98°(LC/MS) |
| 270 | 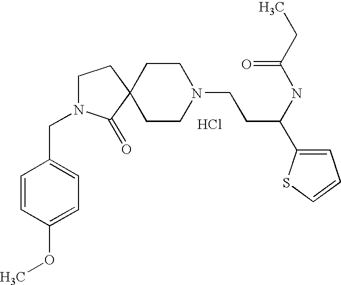 | N-{3-[2-(4-methoxybenzyl)-1-oxo-2,8- sdiaza-spiro[4.5]dec-8-yl]-1-thiophen-2- yl-propyl}-propionamide hydrochloride | 506.107 | 98+(LC/MS) |
| 271 | 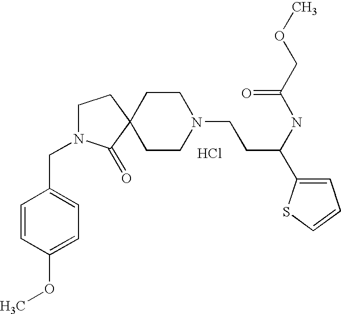 | 2-methoxy-N -{3-[2-(4-methoxybenzyl)- 1-oxo-2,8-diaza-spiro[4.5]dec-8-yl]-1- thiophen-2-yl-propyl}-acetamide hydrochloride | 522.106 | 98+(LC/MS) |
| 272 | 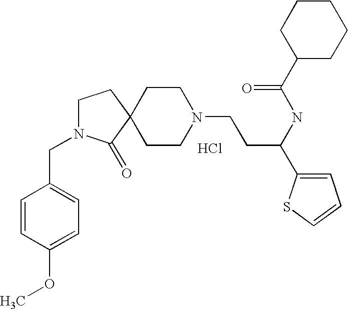 | Cyclohexanecarboxylicc acid {3-[2-(4- methoxybenzyl)-1-oxo-2,8-diaza- spiro[4.5]dec-8-yl]-1-thiophen-2-yl- propyl}-amide hydrochloride | 560.199 | 98+(LC/MS) |
| 273 | 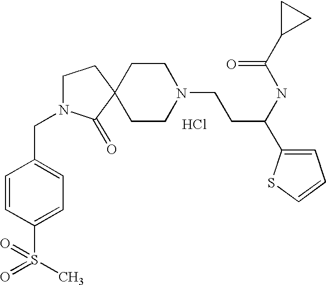 | Cyclopropane carboxylic acid {3-[2-(4- methanesulfonylbenzyl)-1-oxo-2,8- diaza-spiro[4.5]dec-8-yl]-1-thiophen-2- yl-propyl}-amide hydrochloride | 566.183 | 98+(LC/MS) |
| 274 | 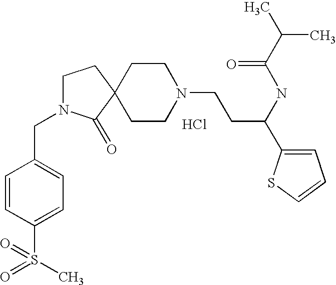 | N-{3-[2-(4-methanesulfonylbenzyl)-1- oxo-2,8-diaza-spiro[4.5]dec-8-yl]-1- thiophen-2-yl-propyl}-isobutyramide hydrochloride | 568.199 | 98+(LC/MS) |
| 275 | 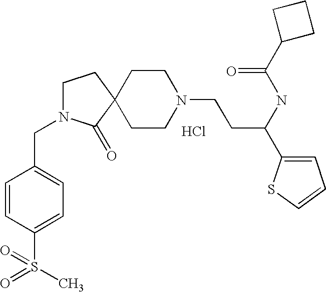 | Cyclobutanecarboxylic acid {3-[2-(4- methanesulfonylbenzyl)-1-oxo-2,8- diaza-spiro[4.5]dec-8-yl]-1-thiophen-2- yl-propyl}-amide hydrochloride | 580.21 | 95.2% (LC/MS) |
| 276 | 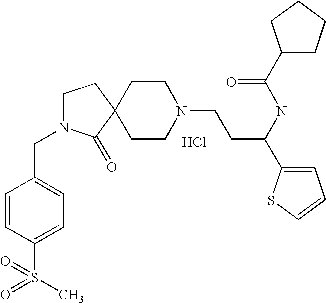 | Cyclopentanecarboxylic acid {3-[2-(4- methanesulfonylbenzyl)-1-oxxo-2,8- diaza-spiro[4.5]dec-8-yl]-1-thiophen-2- yl-propyl}-amide hydrochloride | 594.237 | 98+(LC/MS) |
| 277 | 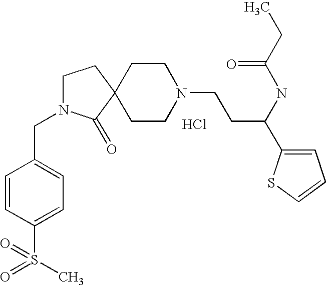 | N-{3-[2-(4-methanesulfonylbenzyl)-1- oxo-2,8-diaza-spiro[4.5υdec-8-yl]-1- thiophen-2-yl-propyl}-propionamide hydrochloride | 554.172 | 98+(LC/MS) |
| 278 | 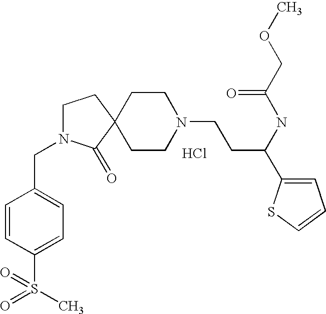 | N-{3-[2-(4-methanesulfonylbenzyl)-1- oxo-2,8-diaza-spiro[4.5]dec-8-yl]-1- thiophen-2-yl-propyl}-2-methoxy- acetamide hydrochloride | 570.171 | 98+(LC/MS) |
| 279 | 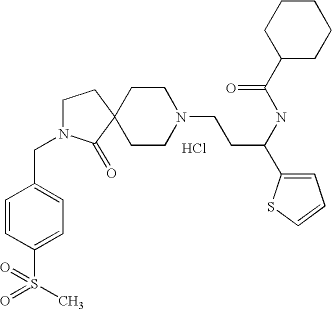 | Cyclohexanecarboxylic acid {3-[22-(4- methanesulfonylbenzyl)-1-oxo-2,8- diaza-spiro[4.5]dec-8-yl]-1-thiophen-2- yl-propyl}-amide hydrochloride | 608.624 | 98+(LC/MS) |
| 280 | 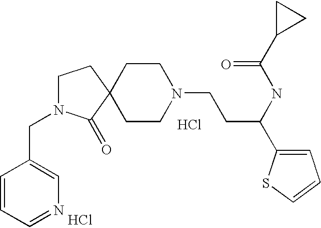 | Cyclohexanecarboxylic acid [3-(1-oxo- 2-pyridin-3-ylmethyl-2,8-diaza- spiro[4.5]dec-8-yl)-1-thiophen-2-yl- propyl]-amide dihydrochloride | 525.542 | 98+(LC/MS) |
| 281 | 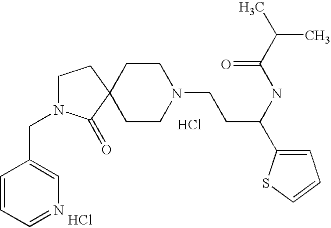 | N-[3-(1-oxxo-22-pyridin-3-ylmethyl-22,8- diaza-spiro[4.5]dec-8-yl)-1-thiophen-2- yl-propyl]-isobutyramide dihydrochloride | 527.557 | 98+(LC/MS) |
| 282 | 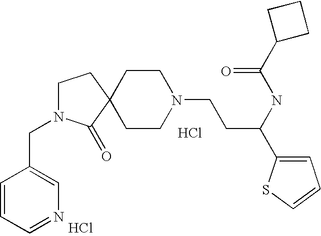 | Cyclobutanecarboxylic acid [3-(1-oxo- 2-pyridin-3-ylmethyl-2,8-diaza- spiro[4.5]dec-8-yl)-1-thiophen-2-yl- propyl]-amide dihydrochloride | 539.568 | 98+(LC/MS) |
| 283 | 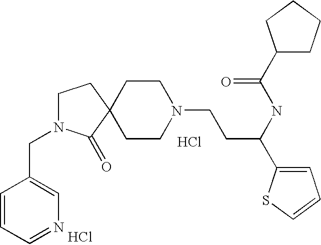 | Cyclopentanecarboxylic acid [3-(1-oxo- 2-pyridin-3-ylmethyl-2,8-diaza- spirro[4.5]dec-8-yl)-1-thiophen-2-yl- propyl]-amide dihydrochloride | 553.595 | 98+(LC/MS) |
| 284 | 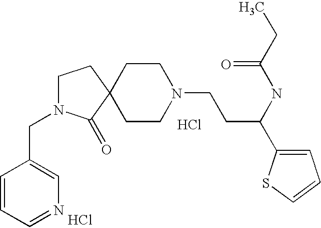 | N-[3-(1-oxo-2-pyridin-3-ylmethyl-2,8- diaza-spiro[4.5]dec-8-yl)-1-thiophen-2- yl-propyl]propionamide dihydrochloride | 513.531 | 80.8% (LC/MS) |
| 285 | 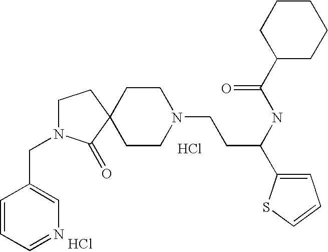 | Cyclohexanecarboxylic acid [3-(1-oxo- 2-pyridin-3-ylmethyl-2,8-diaza- spiro[4.5]dec-8-yl)-1-thiophen-2-yl- propyl]-amide dihydrochloride | 567.622 | 94.3% (LC/MS) |
| 286 | 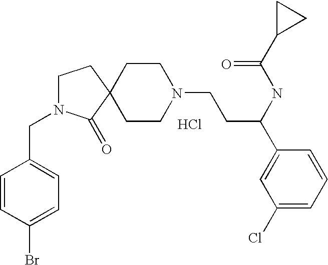 | Cyclopropanecarboxylic acid [3-[2-(4- bromobenzyl)-1-oxo-2,8-diaza- spiro[4.5]dec-8-yl]-1-(3-chlorophenyl)- propyl]-amide hydrochloride | 595.406 | 98+(LC/MS) |
| 287 | 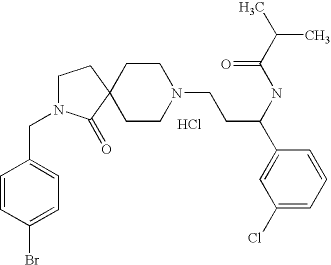 | N-[3-[2-(4-bromobenzyl)-1-oxo-2,8- diaza-spiro[4.5]dec-8-yl]-1-(3- chlorophenyl)-propyl]-isobutyramide hydrochloride | 597.421 | 98+(LC/MS) |
| 288 | 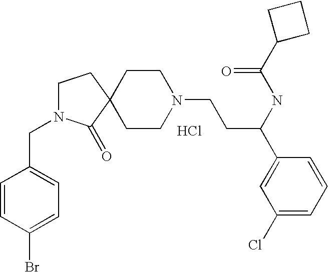 | Cyclobutanecarboxylic acid [3-[2-(4- bromobenzyl)-1-oxo-2,8-diaza- spiro[4.5]dec-8-yl]-1-(3-chlorophenyl)- propyl]-amide hydrochloride | 609.432 | 98+(LC/MS) |
| 289 | 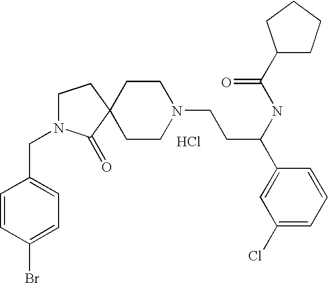 | Cyclopentanecarboxylic acid [3-[2-(4- bromobenzyl)-1-oxo-2,8-diaza- spiro[4.5]dec-8-yl]-1-(3-chlorophenyl)- propyl]-amide hydrochloride | 623.459 | 98+(LC/MS) |
| 290 | 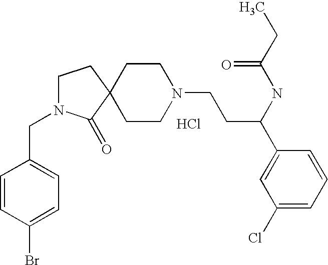 | N-[3-[2-(4-bromobenzyl)-1-oxo-2,8- diaza-spiro[4.5]dec-8-yl]-1-(3- chlorophenyl)-propyl]-propionamide hydrochloride | 583.395 | 98+(LC/MS) |
| 291 | 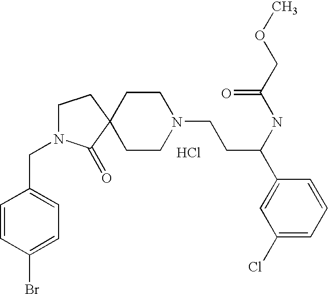 | N-[3-[2-(4-bromobenzyl)-1-oxo-2,8- diaza-spiro[4.5]dec-8-yl]-1-(3- chlorophenyl)-propyl]-2-methoxy- acetamide hydrochloride | 599.394 | 98+(LC/MS) |

Table 3a of compounds illustrates some additional compounds of the present invention that were synthesized using the procedure described in scheme 4.

TABLE 3a

| CPD # | MOL STRUCTURE | COMPOUND NAME | MOL WT | PURITY |
|---|---|---|---|---|
| 292 | 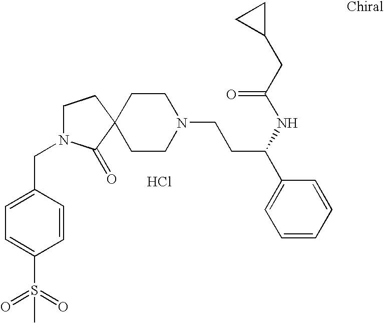 | 2-Cyclopropyl-N-{(S)-3-[2-(4- methanesulfonyl-benzyl)-1-oxo- 2,8-diaza-spiro[4.5]dec-8-yl]-1- phenyl-propyl}-acetamide hydrochloride | 574.182 | 99% (LC/MS) |
| 293 | 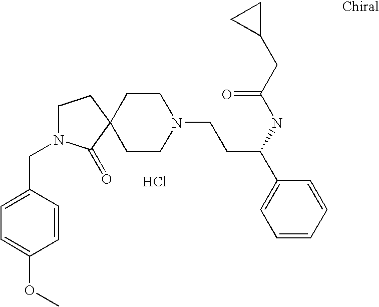 | 2-Cyclopropyl-N-{(S)-3-[2-(4- methoxybenzyl)-1-oxo-2,8-diaza- spiro[4.5]dec-8-yl]-1-phenyl- propyl}-acetamide hydrochloride | 526.117 | 99% (LC/MS) |
| 294 | 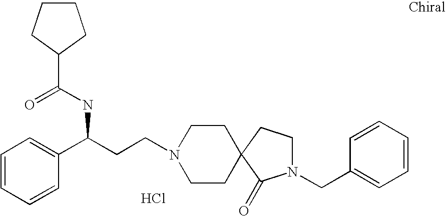 | Cyclopentanecarboxylic acid [(S)- 3-(2-benzy-1-oxo-2,8-diaza- spiro[4.5]dec-8-yl)-1-phenyl- propyl]-amide hydrochloride | 510.118 | 98+ (LC/MS) |
| 295 | 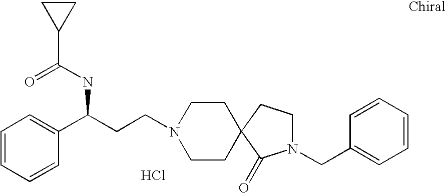 | Cyclopropanecarboxylic acid [(S)- 3-(2-benzyl-1-oxo-2,8-diaza- spiro[4.5]dec-8-yl)-1-phenyl- propyl]-amide hydrochloride | 482.064 | 91.6% (LC/MS) |
| 296 | 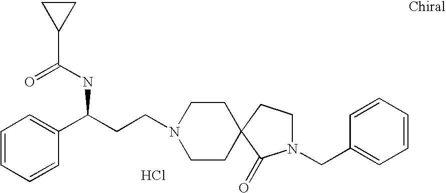 | Cyclohexancarboxylic acid [3-(2- (4-bromobenzyl)-1-oxo-2,8-diaza- spiro[4.5]dec-8-yl]-1-(3- chlorophenyl)-propyl]-amide hydroxhloride | 637.486 | 98% (LC/MS) |
| 297 | 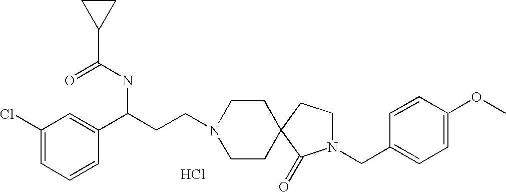 | Cyclopropanecarboxylic acid {1-(3- chloro-phenyl)-3-[2-(4-methoxy- benzyl)-1-oxo-2,8-diaza- spiro[4.5]dec-8-yl]-propyl}-amide hydrochloride | 546.535 | 98% (LC/MS) |
| 298 | 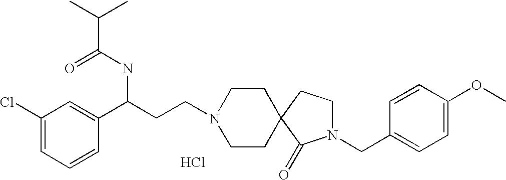 | N-{1-(3-Chloro-phenyl)-3-[2-(4- methoxy-benzyl)-1-oxo-2,8-diaza- spiro[4.5]dec-8-yl]-propyl}- isobutyramide hydrochloride | 548.551 | 98% (LC/MS) |
| 299 | 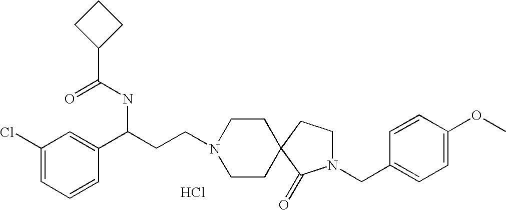 | Cyclobutanecarboxylic acid {1-(3- chlorophenyl)-3-[2-(4- methoxybenzyl)-1-oxo-2,8-diaza- spiro[4.5]dec-8-yl]propyl}-amide hydrochloride | 560.562 | 98% (LC/MS) |
| 300 | 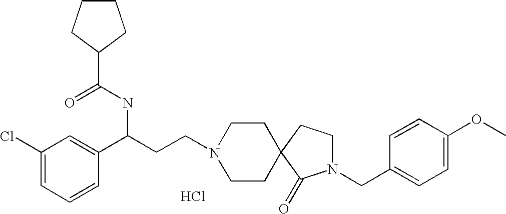 | Cyclopentanecarboxylic acid {1-(3- chlorophenyl)-3-[2-(4- methoxybenzyl)-1-oxo-2,87-diaza- spiro[4.5]dec-8-yl]-propyl}-amide hydrochloride | 574.589 | 98% (LC/MS) |
| 301 | 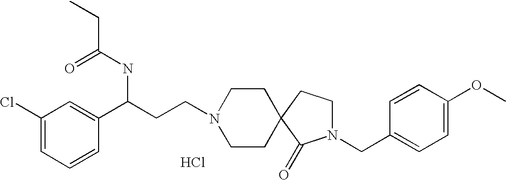 | N-{1-(3-Chlorophenyl)-3-[2-(4- methoxybenzyl)-1-oxo-2,8-diaza- spiro[4.5]dec-8-yl]-propyl}- propionamide hydropchloride | 543.524 | 95% (LC/MS) |
| 302 | 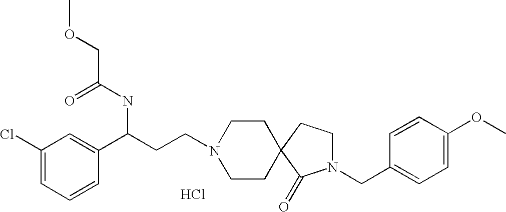 | N-{1-(3-Chlorophenyl)-3-[2-(4- methoxybenzyl)-1-oxo-2,8-diazaq- spiro[4.5]dec-8-yl]-propyl}-2- methoxy-acetamide hydrochloride | 550.523 | 98% (LC/MS) |
| 303 | 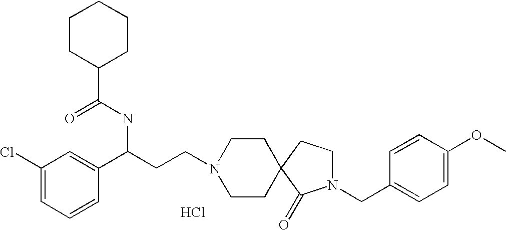 | Cyclohexanecarboxylic acid {1-(3- chlorophenyl)-3-[2-(4- methoxybenzyl)-1-oxo-2,8-diaza- spiro[4.5]dec-8-yl]propyl}-amide hydrochloride | 588.616 | 98% (LC/MS) |
| 304 | 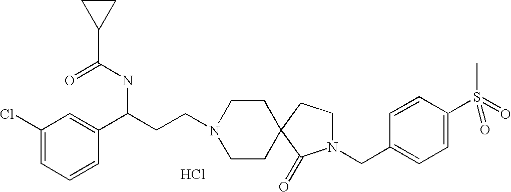 | Cyclopropylcarboxylic acid {1-(3- chlorophenyl)-3-[2-(4- methanesulfonyl-benzyl)-1-oxo- 2,8-diaza-spiro[4.5]dec-8-yl]- propyl}-amide hydrochloride | 594.6 | 98% (LC/MS) |
| 305 | 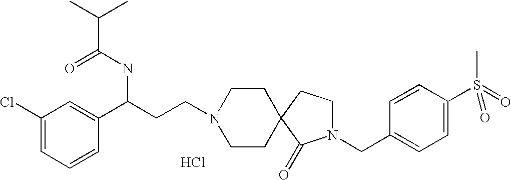 | N-{1-(3-Chlorophenyl)-3-[2-(4- methanesulfonylbenzyl)-1-oxo-2,8- diaza-spiro[4.5]dec-8-yl]-propyl}- isobutyramide hydrochloride | 596.616 | 98% (LC/MS) |
| 306 | 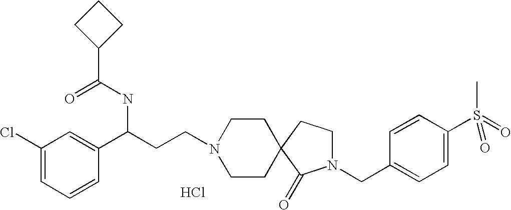 | Cyclobutanecarboxylic acid {1-(3- chlorophenyl)-3-[2-(4- methanesulfonylbenzyl)-1-oxo-2,8- diaza-spiro[4.5]dec-8-yl]-propyl}- amide hydrochloride | 608.627 | 98% (LC/MS) |
| 307 | 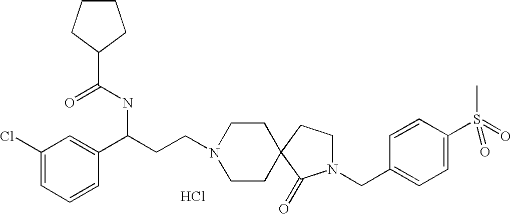 | Cyclopentanecarboxylic acid {1-(3- chlorophenyl)-3-[2-(4- methanesulfonylbenzyl)-1-oxo-2,8- diaza-spiro[4.5]dec-8-yl]propyl}- amide hydrochloride | 622.654 | 98% (LC/MS) |
| 308 | 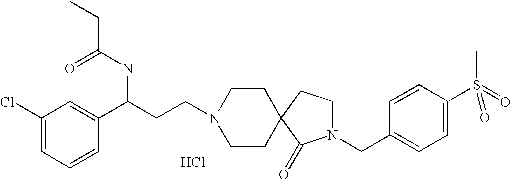 | N-{1-(3-Chlorophenyl)-3-[2-(4- methanesulfonylbenzyl)-1-oxo-2,8- diaza-spiro[4.5]dec-8-yl]-propyl}- propionamide hydrochloride | 582.589 | 98% (LC/MS) |
| 309 | 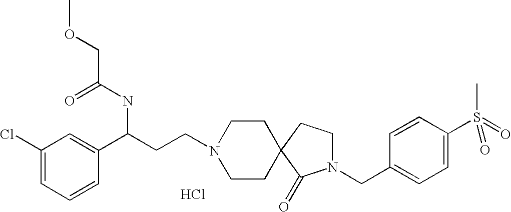 | N-{1-(3-Chlorophenyl)-3-[2-(4- methanesulfonylbenzyl)-1-oxo-2,8- diaza-spiro[4.5]dec-8-yl]-propyl}-2- methoxy-acetamide hydrochloride | 598.588 | 95% (LC/MS) |
| 310 | 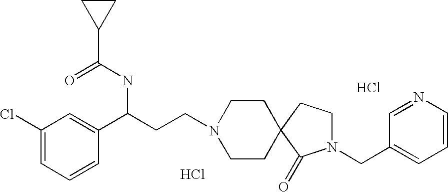 | Cyclopropanecarboxylic acid [1-(3- chlorophenyl)-3-(1-oxo-2-pyridin-3- ylmethyl-2,8-diaza-spiro[4.5]dec-8- yl)-propyl]-amide dihydrochloride | 553.958 | 93.2% (LC/MS) |
| 311 | 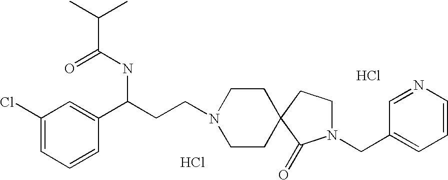 | N-[1-(3-Chlorophenyl)-3-(1-oxo-2- pyridin-3-ylmethyl-2,8-diaza- spiro[4.5]dec-8-yl)-propyl]- isobutyramide dihydrochloride | 555.974 | 94.1% (LC/MS) |
| 312 | 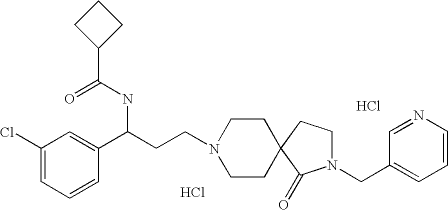 | Cyclobutanecarboxylic acid [1-(3- chlorophenyl)-3-(1-oxo-2-pyridin-3- ylmethyl-2,8-diaza-spiro[4.5]dec-8- yl)-propyl]-amide dihydrochloride | 567.985 | 98% (LC/MS) |
| 313 | 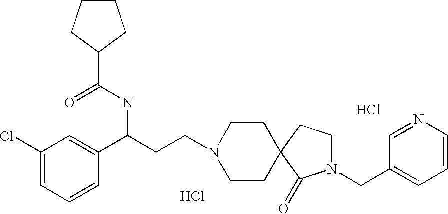 | Cyclopentanecarboxylic acid [1-(3- chloro-phenyl)-3-(1-oxo-2-pyridin- 3-ylmethyl-2,8-diaza-spiro[4.5]dec- 8-yl)-propyl]-amide dihydrochloride | 582.012 | 98% (LC/MS) |
| 314 | 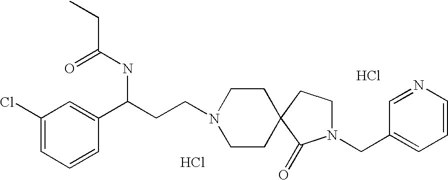 | N-[1-(3-Chlorophenyl)-3-(1-oxo-2- pyridin-3-ylmethyl-2,8-diaza- spiro[4.5]dec-8-yl)-propyl]- propionamide dihydrochloride | 541.948 | 93.8% (LC/MS) |
| 315 | 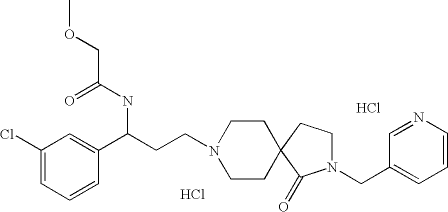 | N-[1-(3-Chlorophenyl)-3-(1-oxo-2- pyridin-3-ylmethyl-2,8-diaza- spiro[4.5]dec-8-yl)-propyl]-2- methoxy-acetamide dihydrochloride | 557.946 | 94.4% (LC/MS) |
| 316 | 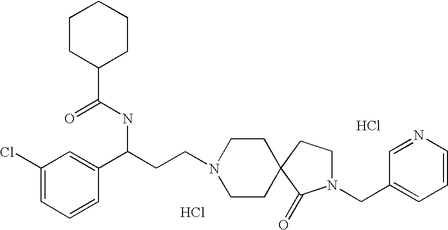 | Cyclohexanecarboxylic acid [1-(3- chlorophenyl)-3-(1-oxo-2-pyridin-3- ylmethyl-2,8-diaza-spiro[4.5]dec-8- yl)-propyl]-amide dihydrochloride | 596.039 | 94.7% (LC/MS) |
| 317 | 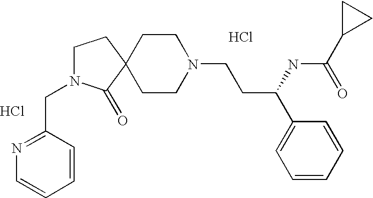 | Cyclopropanecarboxylic acid [(S)- 3-(1-oxo-2-pyridin-2-ylmethyl-2,8- diaza-spiro[4.5]dec-8-yl)-1-phenyl- propyl]-amide dihydrochloricde | 519.513 | 98% (LC/MS) |
| 318 | 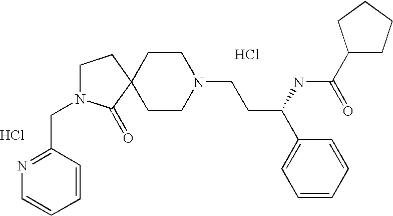 | Cyclopentanecarboxylic acid [(S)- 3-(1-oxo-2-pyridin-2-ylmethyl-2,8- diaza-spiro[45]dec-8-yl)-1-phenyl- propyl]-amide dihydrochloride | 547.567 | 98% (LC/MS) |
| 319 | 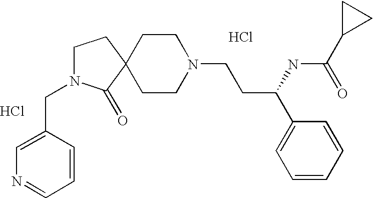 | Cyclopropanecarboxylic acid [(S)- 3-(1-oxo-2-pyridin-3-ylmethyl-2,8- diaza-spiro[4.5]dec-8-yl)-1-phenyl- propyl]-amide dihydrochloride | 519.513 | 98% (LC/MS) |
| 320 | 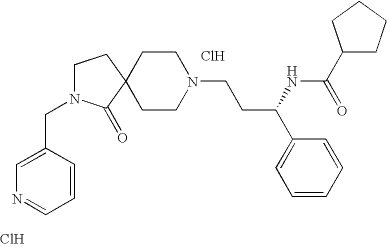 | Cyclopentanecarboxylic acid [(S)- 3-(1-oxo-2-pyridin-3-ylmethyl-2,8- diaza-spiro[4.5]dec-8-yl)-1-phenyl- propyl]-amide dihydrochloride | 547.567 | 98% (LC/MS) |
| 321 | 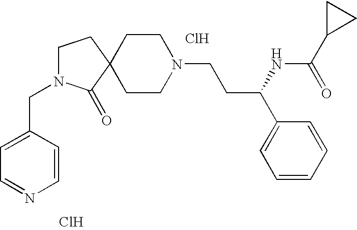 | Cyclopropanecarboxylic acid [(S)- 3-(1-oxo-2-pyridin-4-ylmethyl-2,8- diaza-spiro[4.5]dec-8-yl)-1-phenyl- propyl]-amide dihydrochloride | 519.513 | 98% (LC/MS) |
| 322 | 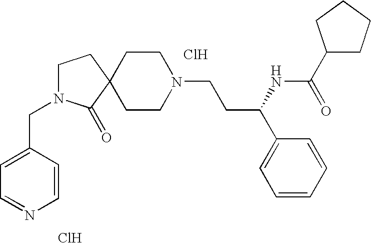 | Cyclopropanecarboxylic acid [(S)- 3-(1-oxo-2-pyridin-4-ylmethyl-2,8- diaza-spiro[4.5]dec-8-yl)-1-phenyl- propyl]-amide dihydrochloride | 547.567 | 98% (LC/MS) |
| 323 | 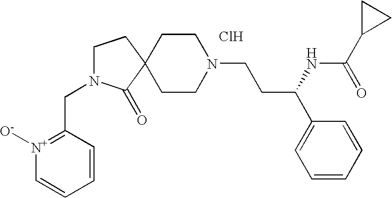 | Cyclopropanecarboxylic acid {(S)- 3-[1-oxo-2-(1-oxy-pyridin-2- ylmethyl)-2,8-diaza-spiro[4.5]dec- 8-yl]-1-phenyl-propyl}-amide hydrochloride | 499.052 | 98% (LC/MS) |
| 324 | 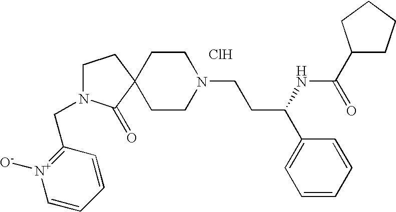 | Cyclopentanecarboxylic acid {(S)- 3-[1-oxo-2-(1-oxy-pyridin-2- ylmethyl)-2,8-diaza-spiro[4.5]dec- 8-yl]-1-phenyl-propyl}-amide hydrochloride | 527.105 | 98% (LC/MS) |
| 325 | 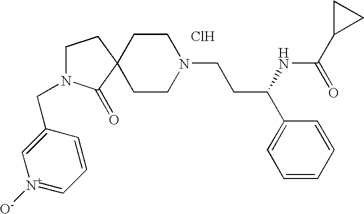 | Cyclopropanecarboxylic acid {(S)- 3-[1-oxo-2-(1-oxy-pyridin-3- ylmethyl)-2,8-diaza-spiro[4.5]dec- 8-yl]-1-phenyl-propyl}-amide hydrochloride | 499.052 | 98% (LC/MS) |
| 326 | 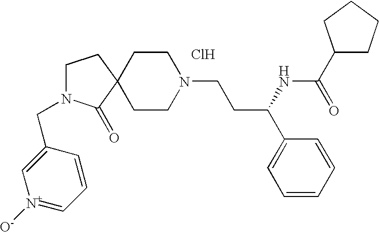 | Cyclopentanecarboxylic acid {(S)- 3-[1-oxo-2-(1-oxy-pyridin-3- ylmethyl)-2,8-diaza-spiro[4.5]dec- 8-yl]-1-phenyl-propyl}-amide hydrochloride | 527.105 | 98% (LC/MS) |
| 327 | 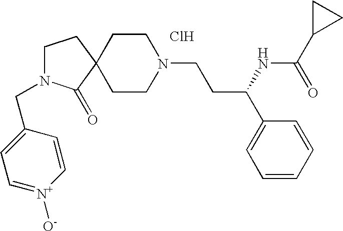 | Cyclopropanecarboxylic acid {(S)- 3-[1-oxo-2-(1-oxy-pyridin-4- ylmethyl)-2,8-diaza-spiro[4.5]dec- 8-yl]-1-phenyl-propyl}-amide hydrochloride | 499.052 | 98% (LC/MS) |
| 328 | 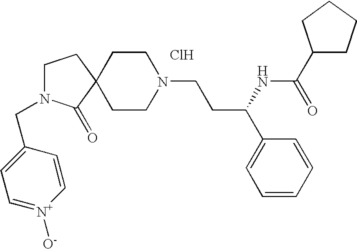 | Cyclopentanecarboxylic acid {(S)- 3-[1-oxo-2-(1-oxy-pyridin-4- ylmethyl)-2,8-diaza-spiro[4.5]dec- 8-yl]-1-phenyl-propyl}-amide hydrochloride | 527.105 | 98% (LC/MS) |
| 329 | 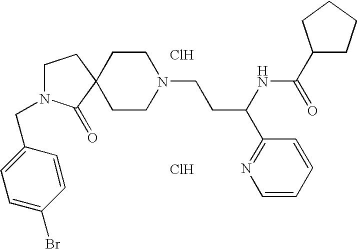 | Cyclopentanecarboxylic acid {3-[2- (4-bromo-benzyl)-1-oxo-2,8-diaza- spiro[4.5]dec-8-yl]-1-pyridin-2-yl- propyl}-amide dihydrochloride | 626.463 | 98% (LC/MS) |
| 330 | 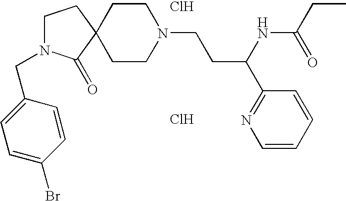 | N-{3-[2-(4-Bromo-benzyl)-1-oxo- 2,8-diaza-spiro[4.5]dec-8-yl]-1- pyridin-2-yl-propyl}-propionamide dihydrochloride | 586.398 | 98% (LC/MS) |
| 331 | 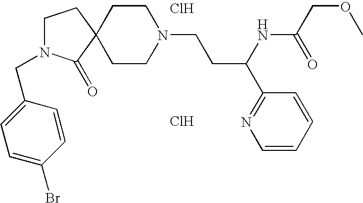 | N-{3-[2-(4-Bromo-benzyl)-1-oxo- 2,8-diaza-spiro[4.5]dec-8-yl]-1- pyridin-2-yl-propyl}-2-methoxy- acetamide dihydrochloride | 602.398 | 98% (LC/MS) |
| 332 | 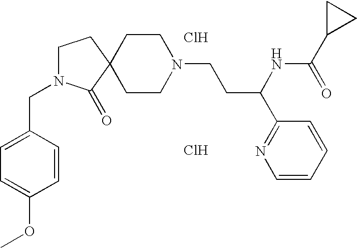 | Cyclopropanecarboxylic acid {3-[2- (4-methoxy-benzyl)-1-oxo-2,8- diaza-spiro[4.5]dec-8-yl]-1-pyridin- 2-yl-propyl}-amide dihydrochloride | 549.539 | 98% (LC/MS) |
| 333 | 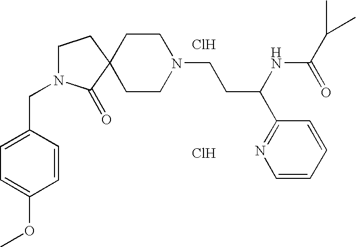 | N-{3-[2-(4-Methoxy-benzyl)-1-oxo- 2,8-diaza-spiro[4.5]dec-8-yl]-1- pyridin-2-yl-propyl}-isobutyramide dihydrochloride | 551.555 | 98% (LC/MS) |
| 334 | 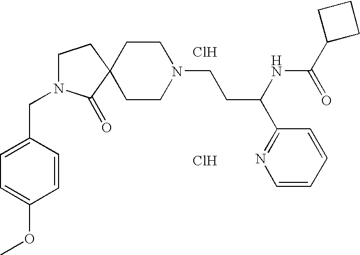 | Cyclobutanecarboxylic acid {3-[2- (4-methoxy-benzyl)-1-oxo-2,8- diaza-spiro[4.5]dec-8-yl]-1-pyridin- 2-yl-propyl}-amide dihydrochloride | 563.566 | 98% (LC/MS) |
| 335 | 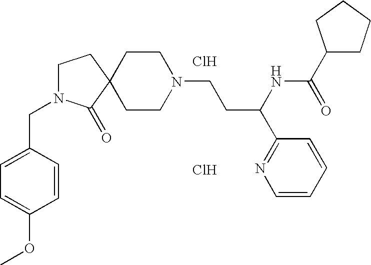 | Cyclopentanecarboxylic acid {3-[2- (4-methoxy-benzyl)-1-oxo-2,8- diaza-spiro[4.5]dec-8-yl]-1-pyridin- 2-yl-propyl}-amide dihydrochloride | 563.566 | 98% (LC/MS) |
| 336 | 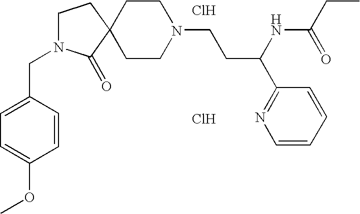 | N-{3-[2-(4-Methoxy-benzyl)-1-oxo- 2,8-diaza-spiro[4.5]dec-8-yl]-1- pyridin-2-yl-propyl}-propionamide dihydrochloride | 537.528 | 98% (LC/MS) |
| 337 | 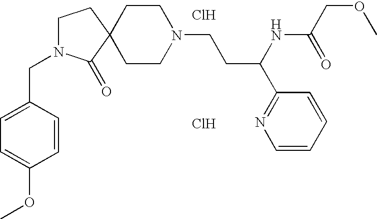 | 2-Methoxy-N-{3-[2-(4-methoxy- benzyl)-1-oxo-2,8-diaza- spiro[4.5]dec-8-yl]-1-pyridin-2-yl- propyl}-acetamide dihydrochloride | 553.527 | 98% (LC/MS) |
| 338 | 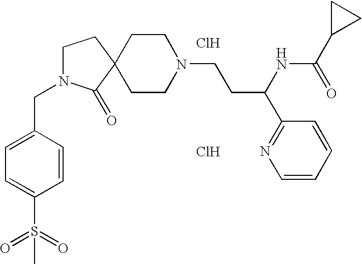 | Cyclopropanecarboxylic acid {3-[2- (4-methanesulfonyl-benzyl)-1-oxo- 2,8-diaza-spiro[4.5]dec-8-yl]-1- pyridin-2-yl-propyl}-amide dihydrochloride | 597.604 | 98% (LC/MS) |
| 339 | 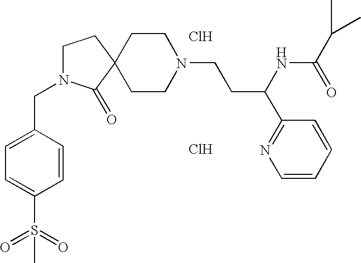 | N-{3-[2-(4-Methanesulfonyl- benzyl)-1-oxo-2,8-diaza- spiro[4.5]dec-8-yl]-1-pyridin-2-yl- propyl}-isobutylamide dihydrochloride | 599.62 | 98% (LC/MS) |
| 340 | 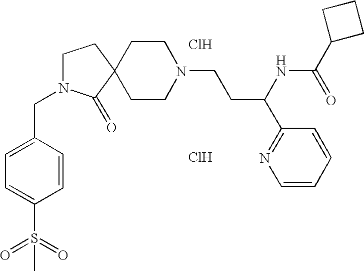 | Cyclobutanecarboxylic acid {3-[2- (4-methanesulfonyl-benzyl)-1-oxo- 2,8-diaza-spiro[4.5]dec-8-yl]-1- pyridin-2-yl-propyl}-amide dihydrochloride | 611.631 | 98% (LC/MS) |
| 341 | 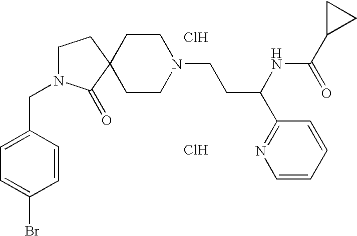 | Cyclopropanecarboxylic acid {3-[2- (4-bromo-benzyl)-1-oxo-2,8-diaza- spiro[4.5]dec-8-yl]-1-pyridin-2-yl- propyl}-amide dihydrochloride | 598.409 | 98% (LC/MS) |
| 342 | 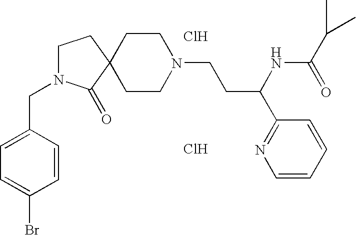 | N-{3-[2-(4-Bromo-benzyl)-1-oxo- 2,8-diaza-spiro[4.5]dec-8-yl]-1- pyridin-2-yl-propyl}-isobutyramide dihydrochloride | 600.425 | 98% (LC/MS) |
| 343 | 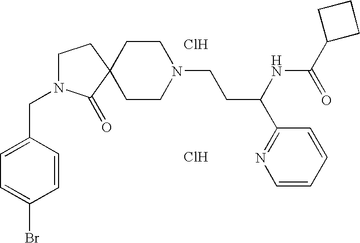 | Cyclobutanecarboxylic acid {3-[2- (4-bromo-benzyl)-1-oxo-2,8-diaza- spiro[4.5]dec-8-yl]-1-pyridin-02-yl- propyl}-amide dihydrochloride | 612.436 | 98% (LC/MS) |
| 344 | 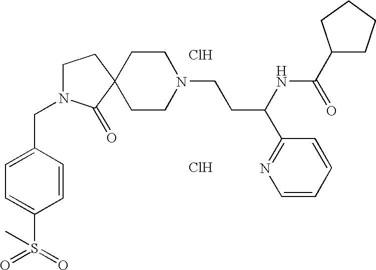 | Cyclopentanecarboxylic acid {3-[2- (4-methanesulfonyl-benzyl)-1-oxo- 2,8-diaza-spiro[4.5]dec-8-yl-]-1- pyridin-2-yl-propyl}-amide dihydrochloride | 625.658 | 98% (LC/MS) |
| 345 | 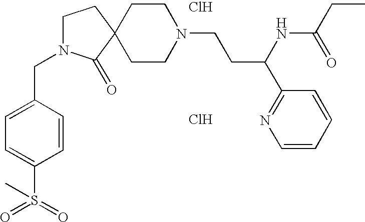 | N-{3-[2-(4-Methanesulfonyl- benzyl}-1-oxo-2,8-diaza- spiro[4.5]dec-8-yl]-1-pyridin-2-yl- propyl}-propionamide dihydrochloride | 585.593 | 95.2% (LC/MS) |
| 346 | 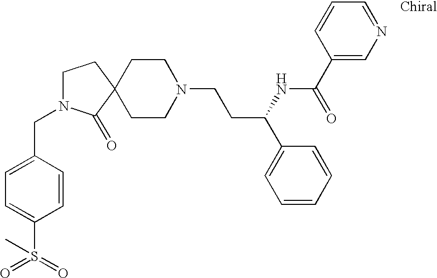 | N-{(S)-3-[2-(4-Methanesulfonyl- benzyl)-1-oxo-2,8-diaza- spiro[4.5]dec-8-yl]-1-phenyl- propyl}-nicotinamide | 560.715 | 91% (HPLC) |
| 347 | 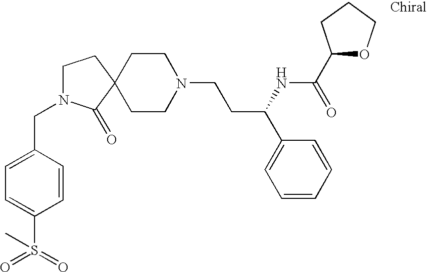 | (R)-Tetrahydro-furan-2-carboxylic acid {(S)-3-[2-(4-meethanesulfonyl- benzyl)-1-oxo-2,8-diaza- spiro[4.5]dec-8-yl]-1-phenyl- propyl}-amide | 553.72 | 99% (HPLC) |
| 348 | 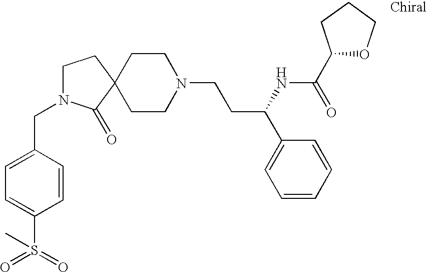 | (S)-Tetrahydro-furan-2-carboxylic acid {(S)-3-[2-(4-methanesulfonyl- benzyl)-1-oxo-2,8-diaza- spiro[4.5]dec-8-yl]-1-phenyl- propyl}-amide | 553.72 | 99% (HPLC) |
| 349 | 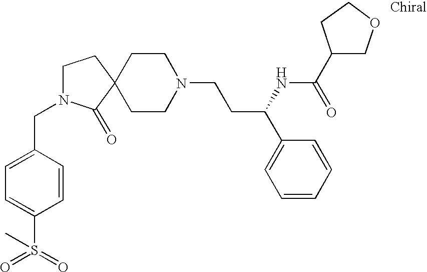 | Tetrahydro-furan-3-carboxylic acid {(S)-3-[2-(4-methansulfonyl- benzyl)-1-oxo-2,8-diaza- spiro[4.5]dec-8-yl]-1-phenyl- propyl}-amide | 553.72 | 99% (HPLC) |
| 350 | 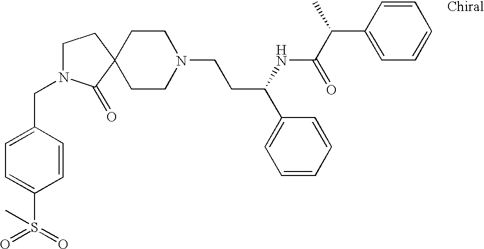 | (R)-N-{(S)-3-[2-(4-Methanesulfonyl- benzyl)-1-oxo-2,8-diaza- spiro[4.5]dec-8-yl]-1-phenyl- propyl}-2-phenyl-propionamide | 587.781 | 84% (HPLC) |
| 351 | 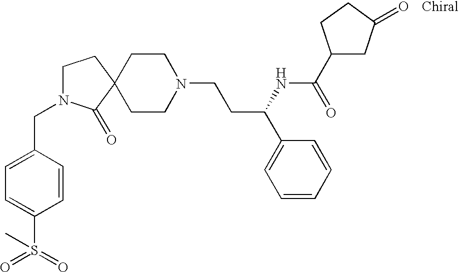 | 3-Oxo-cyclopentanecarboxylic acid {(S)-3-[2-(4-methanesulfonyl- benzyl)-1-oxo-2,8-diaza- spiro[4.5]dec-8-yl]-1-phenyl- propyl}-amide | 565.731 | 84% (HPLC) |
| 352 | 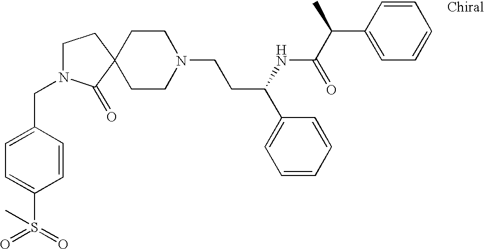 | (S)-N-{(S)-3-[2-(4-Methanesulfonyl- benzyl)-1-oxo-2,8-diaza- spiro[4.5]dec-8-yl]-1-phenyl- propyl}-2-phenyl-propionamide | 587.781 | 86% (HPLC) |
| 353 | 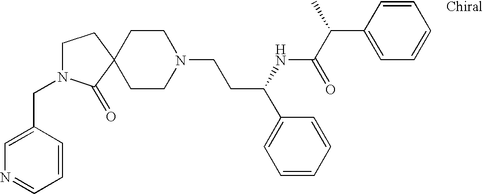 | (R)-N-[(S)-3-(1-Oxo-2-pyridin-3- ylmethyl-2,8-diaza-spiro[4.5]dec-8- yl)-1-phenyl-propyl]-2-phenyl- propionamide | 510.678 | 97% (HPLC) |
| 354 | 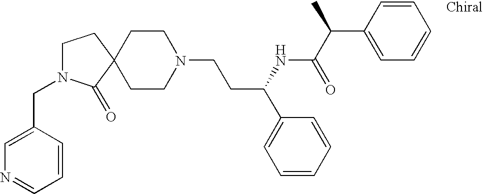 | (S)-N-[(S)-3-(1-Oxo-2-pyridin-3- ylmethyl-2,8-diaza-spiro[4.5]dec-8- yl)-1-phenyl-propyl]-2-phenyl- propionamide | 510.678 | 99% (HPLC) |
| 355 | 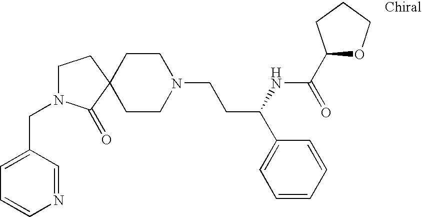 | (R)-Tetrahydro-furan-2-carboxylic acid [(S)-3-(1-oxo-2-pyridin-3- ylmethyl-2,8-diaza-spiro[4.5]dec-8- yl-1-phe3nyl-propyl]-amide | 476.617 | 97% (HPLC) |
| 356 | 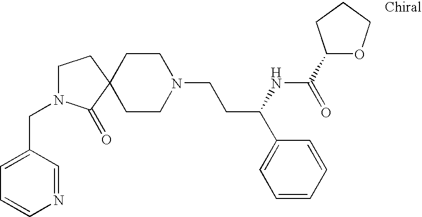 | (S)-Tetrahydro-furan-2-carboxylic acid [(S)-3-(1-oxo-22-pyridin-3- ylmethyl-2,8-diaza-spiro[4.5]dec-8- yl)-1-phenyl-propyl]-amide | 476.617 | 99% (HPLC) |
| 357 | 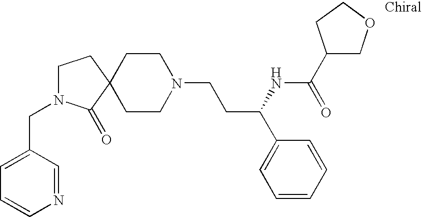 | Tetrahydro-furan-3-carboxylic acid [(S)-3-(1-oxo-2-pyridin-3-ylmethyl- 2,8-diaza-spiro[4.5]dec-8-yl)-1- phenyl-propyl]-amide | 476.617 | 98% (HPLC) |
| 358 | 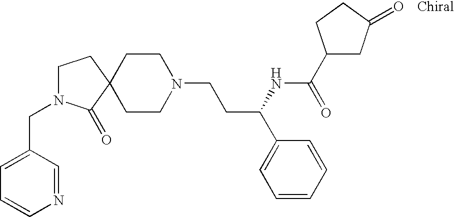 | 3-Oxo-cyclopentaecarboxylic acid [(S)-3-(1-oxo-2-pyridin-3-ylmethyl- 2,8-diaza-spiro[4.5]dec-8-yl)-1- phenyl-propyl]-amide | 488.628 | 96% (HPLC) |
| 359 | 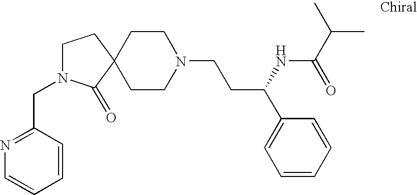 | N-[(S)-3-(2-Oxo-2-pyridin-2- ylmethyl-2,8-diaza-spiro[4.5]dec-8- yl)-1-phenyl-propyl]-isobutylamide | 448.607 | 97% (HPLC) |
| 360 | 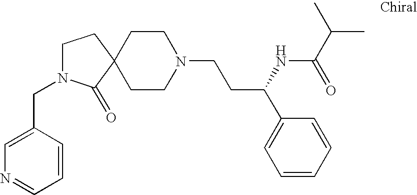 | N-[(S)-3-(1-Oxo-2-pyridin-3- ylmethyl-2,8-diaza-spiro[4.5]dec-8- yl)-1-phenyl-propyl]-isobutylamide | 448.607 | 99% (HPLC) |
| 361 | 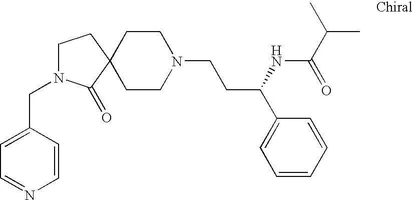 | N-[(S)-3-(1-Oxo-2-pyridin-4- ylmethyl-2,8-diaza-spiro[4.5]dec-8- yl)-1-phenyl-propyl]-isobutyramide | 448.607 | 98% (HPLC) |
| 362 | 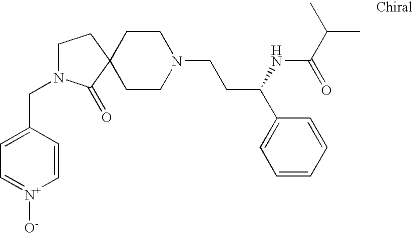 | N-{(S)-3-[1-Oxo-2-(1-oxy-pyridin-4- ylmethyl)-2,8-diaza-spiro[4.5]dec- 8-yl]-1-phenyl-propyl}- isobutyramide | 464.606 | 90% (HPLC) |
| 363 | 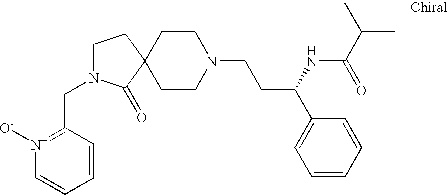 | N-{(S)-3-[1-Oxo-2-(1-oxy-pyridin-2- ylmethyl)-2,8-diaza-spiro[4.5]dec- 8-yl]-1-phenyl-propyl}- isobutyramide | 464.606 | 98% (HPLC) |
| 364 | 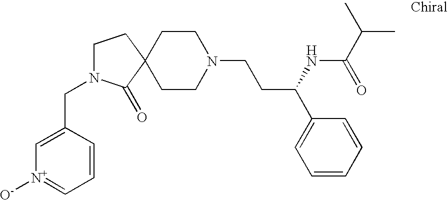 | N-{(S)-3-[1-Oxo-2-(1-oxy-pyridin-3- ylmethyl)-2,8-diaza-spiro[4.5]dec- 8-yl]-1-phenyl-propyl}- isobutyramide | 464.606 | 99% (HPLC) |
| 365 | 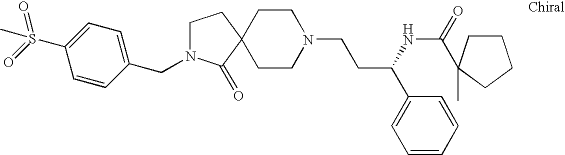 | 1-Methyl-cyclopentanecarboxylic acid {(S)-3-[2-(4-methanesulfonyl- benzyl)-1-oxo-2,8-diaza- spiro[4.5]dec-8-yl]-1-phenyl- propyl}-amide | 565.775 | 100% (LC/MS) |
| 366 | 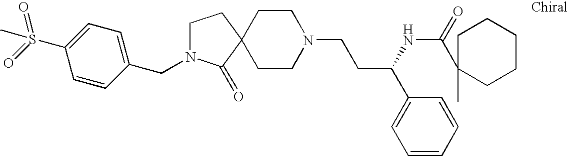 | 1-Methyl-cyclohexanecarboxylic acid{(S)-3-[2-(4-methanesulfonyl- benzyl)-1-oxo-2,8-diaza- spiro[4.5]dec-8-yl]-1-phenyl- propyl}-amide | 579.802 | >80% (LC/MS) |
| 367 | 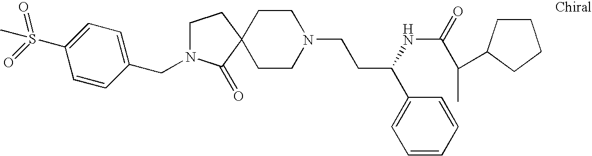 | 2-Cyclopentyl-N-{(S)-3-[2-(4- methanesulfonyl-benzyl)-1-oxo- 2,8-diaza-spiro[4.5]dec-8-yl]-1- phenyl-propyl}-propionamide | 579.802 | 100% (LC/MS) |
| 368 | 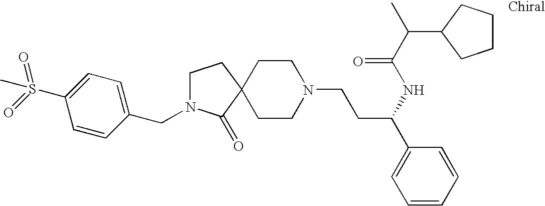 | 2-Cyclopentyl-N-{(S)-3-[2-(4- methanesulfonyl-benzyl)-1-oxo- 2,8-diaza-spiro[4.5]dec-8-yl]-1- phenyl-propyl}-propionamide | 579.802 | 100% (LC/MS) |
| 369 | 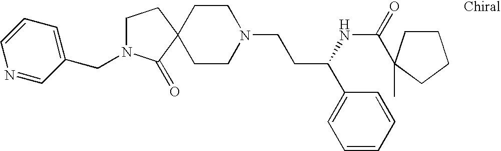 | 1-Methyl-cyclopentanecarboxylic acid [(S)-3-(1-oxo-2-pyridin-3- ylmethyl-2,8-diaza-spiro[4.5]dec-8- yl)-1-phenyl-propyl]-amide | 488.672 | 100% (LC/MS) |
| 370 | 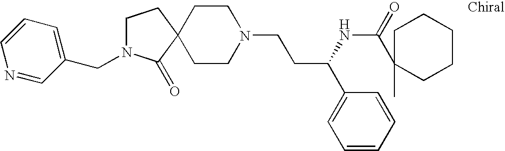 | 1-Methyl-cyclohexanecarboxylic acid[(S)-3-(1-oxo-2-pyridin-3- ylmethyl-2,8-diaza-spiro[4.5]dec-8- yl)-1-phenyl-propyl]-amide | 502.699 | 100% (LC/MS) |
| 371 | 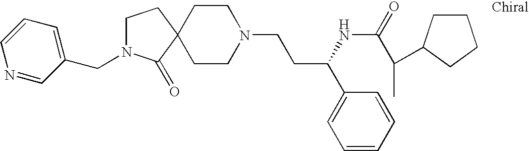 | 2-Cyclopentyl-N-[(S)-3-(1-oxo-2- pyridin-3-ylmethyl-2,8-diaza- spiro[4.5]dec-8-yl)-1-phenyl- propyl]-propionamide | 502.699 | 100% (LC/MS) |
| 372 | 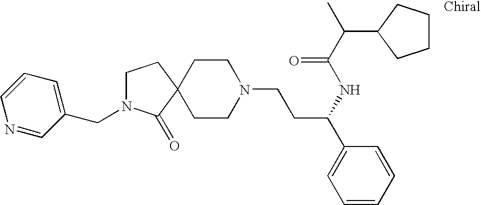 | 2-Cyclopentyl-N-[(S)-3-(1-oxo-2- pyridin-3-ylmethyl-2,8-diaza- spiro[4.5]dec-8-yl)-1-phenyl- propyl]-propionamide | 502.699 | 100% (LC/MS) |
| 373 | 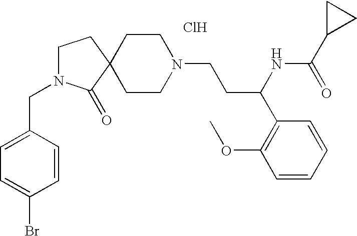 | Cyclopropanecarboxylic acid [3-[2- (4-bromo-benzyl)-1-oxo-2,8-diaza- spiro[4.5]dec-8-yl]-1-(2-methoxy- phenyl)-propyl]-amide hydrochloride | 590.986 | 98% (LC/MS) |
| 374 | 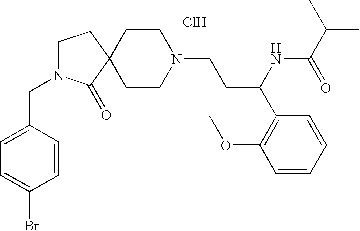 | N-[3-[2-(4-Bromo-benzyl)-1-oxo- 2,8-diaza-spiro[4.5]dec-8-yl]-1-(2- methoxy-phenyl)-propyl]- isobutyramide hydrochloride | 593.002 | 98% (LC/MS) |
| 375 | 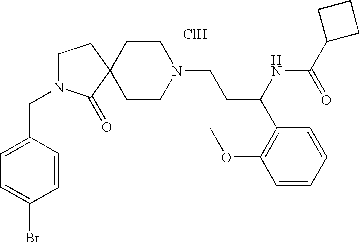 | Cyclobutanecarboxylic acid [3[2- (4-bromo-benzyl)-1-oxo-2,8-diaza- spiro[4.5]dec-8-yl]-1-(2-methoxy- phenyl)-propyl]-amide hydrochloride | 605.013 | 98% (LC/MS) |
| 376 | 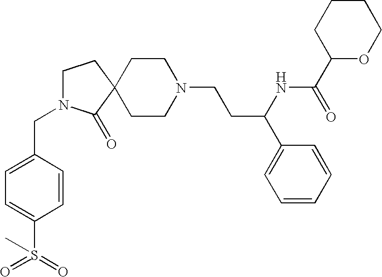 | Tetrahydro-pyran-2-carboxylic acid {(S)-3-[2-(4-methanesulfonyl- benzyl)-1-oxo-2,8-diaza- spiro[4.5]dec-8-yl]-1-phenyl- propyl}-amide | 567.747 | 99% (HPLC) |
| 377 | 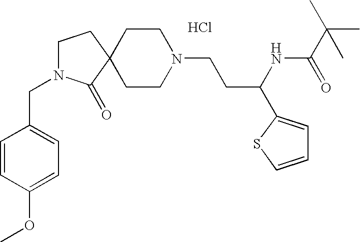 | N-{3-[2-(4-Methoxy-benzyl)-1-oxo- 2,8-diaza-spiro[4.5]dec-8-yl]-1- thiophen-2-yl-propyl}-2,2-dimethyl- propionamide hydrochloride | 534.161 | 98% (LC/MS) |
| 378 | 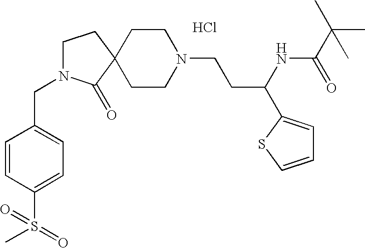 | N-{3-[2-(4-Methanesulfonyl- benzyl)-1-oxo-2,8-diaza- spiro[4.5]dec-8-yl]-1-thiophen-2-yl- propyl}-2,2-dimethyl-propionamide hydrochloride | 582.226 | 98% (LC/MS) |
| 379 | 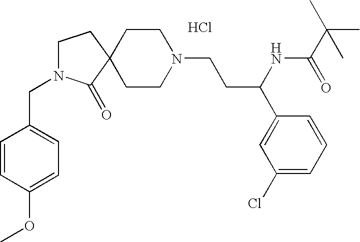 | N-{1-(3-Chloro-phenyl)-3-[2-(4- methoxy-benzyl)-1-oxo-2,8-diaza- spiro[4.5]dec-8-yl]-propyl}-2,2- dimethyl-propionamide hydrochloride | 562.578 | 93.9% (LC/MS) |
| 380 | 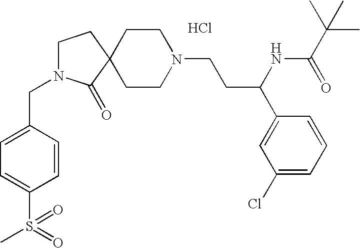 | N-{1-(3-Chloro-phenyl)-3-[2-(4- methanesulfonyl-benzyl)-1-oxo- 2,8-diaza-spiro[4.5]dec-8-yl]- propyl}-2,2-dimethyl-propionamide hydrochloride | 610.643 | 98% (LC/MS) |
| 381 | 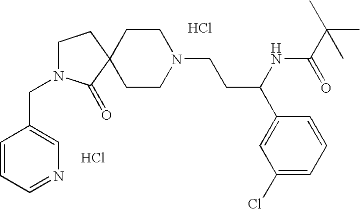 | N-[1-(3-Chloro-phenyl)-3-(1-oxo-2- pyridin-3-ylmethyl-2,8-diaza- spiro[4.5]dec-8-yl)-propyl]-2,2- dimethyl-propionamide dihydrochloride | 570.001 | 98% (LC/MS) |
| 382 | 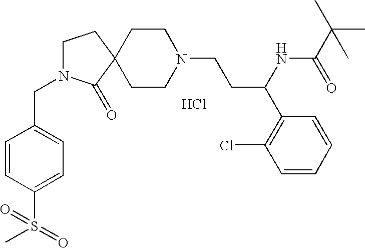 | N-{1-(2-Chloro-phenyl)-3-[2-(4- methanesulfonyl-benzyl)-1-oxo- 2,8-diaza-spiro[4.5]dec-8-yl]- propyl}-2,2-dimethyl-propionamide hydrochloride | 610.643 | 98% (LC/MS) |
| 383 | 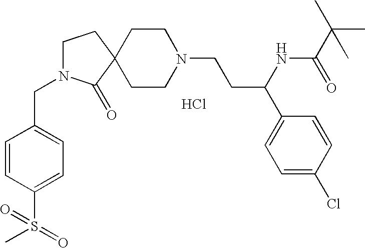 | N-{1-(4-Chloro-phenyl)-3-[2-(4- methanesulfonyl-benzyl)-1-oxo- 2,8-diaza-spiro[4.5]dec-8-yl]- propyl}-2,2-dimethyl-propionamide hydrochloride | 610.643 | 98% (LC/MS) |
| 384 | 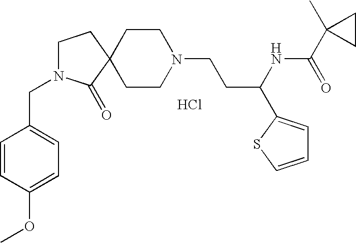 | `1-Methyl-cyclopropanecarboxylic acid {3-[2-(4-methoxy-benzyl)-1- oxo-2,8-diaza-spiro[4.5]dec-8-yl]-1- thiophen-2-yl-propyl}-amide hydrochloride | 532.145 | 98% (LC/MS) |
| 385 | 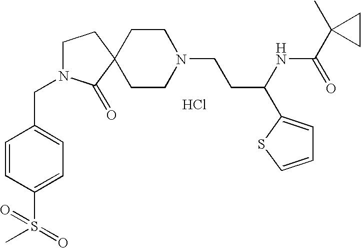 | 1-Methyl-cyclopropanecarboxylic acid {3-[2-(4-methanesulfonyl- benzyl)-1-oxo-2,8-diaza- spiro[4.5]dec-8-yl]-1-thiophen-2-yl- propyl}-amide hydrochloride | 580.21 | 98% (LC/MS) |
| 386 | 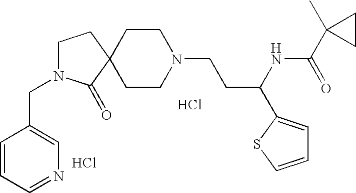 | 1-Methyl-cyclopropanecarboxylic acid [3-(1-oxo-2-pyridin-33-ylmethyl- 2,8-diaza-spiro[4.5]dec-8-yl)-1- thiophen-2-yl-propyl]-amide dihydrochloride | 539.568 | 98% (LC/MS) |
| 387 | 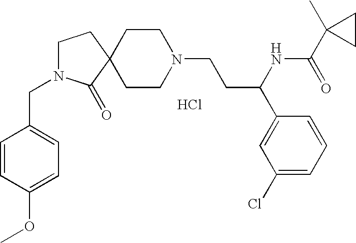 | 1-Methyl-cyclopropanecarboxylic acid {1-(3-chloro-phenyl)-3-[2-(4- methoxy-benzyl)-1-oxo-2,8-diaza- spiro[4.5]dec-8-yl]propyl}-amide hydrochloride | 560.562 | 92.4% (LC/MS) |
| 388 | 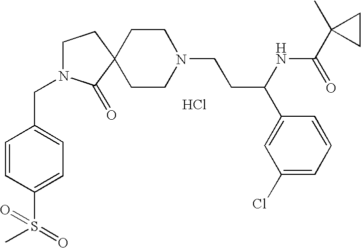 | 1-Methyl-cyclopropanecarboxylic acid {1-(3-chloro-phenyl)-3-[2-(4- methanesulfonyl-benzyl)-1-oxo- 2,8-diaza-spiro[4.5]dec-8-yl]- propyl}-amide hydrochloride | 608.627 | 98% (LC/MS) |
| 389 | 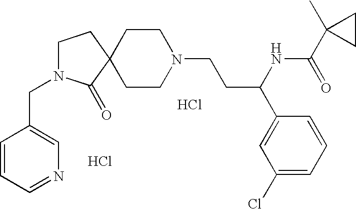 | 1-Methyl-cyclopropanecarboxylic acid [1-(3-chloro-phenyl)-3-(1-oxo- 2-pyridin-3-ylmethyl-2,8-diaza- spiro[4.5]dec-8-yl)-propyl]-amide dihydrochloride | 567.985 | 92.5% (LC/MS) |
| 390 | 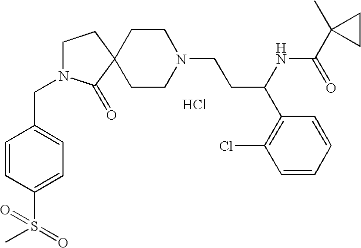 | 1-Methyl-cyclopropanecarboxylic acid {1-(2-chloro-phenyl)-3-[2-(4- methanesulfonyl-benzyl)-1-oxo- 2,8-diaza-spiro[4.5]dec-8-yl]- propyl}-amide hydrochloride | 608.627 | 98% (LC/MS) |
| 391 | 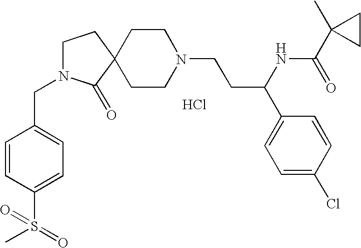 | 1-Methyl-cyclopropanecarboxylic acid {1-(4-chloro-phenyl)-3-[2-(4- methanesulfonyl-benzyl)-1-oxo- 2,8-diaza-spiro[4.5]dec-8-yl]- propyl}-amide hydrochloride | 608.627 | 98% (LC/MS) |
| 392 | 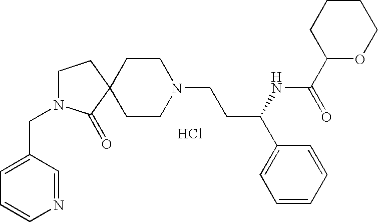 | Tetrahydro-pyran-2-carboxylic acid [(S)-3-(1-oxo-2-pyridin-3-ylmethyl- 2,8-diaza-spiro[4.5]dec-8-yl)-1- phenyl-propyl]-amide | 490.644 | 94% (HPLC) |
| 393 | 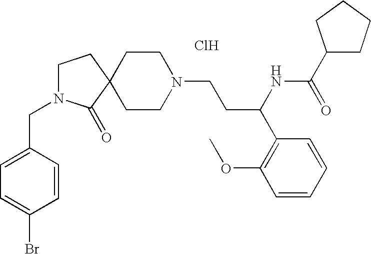 | Cyclopentaecarboxylic acid [3-[2- (4-bromo-benzyl)-1-oxo-2,8-diaza- spiro[4.5]dec-8-yl]-1-(2-methoxy- phenyl)-propyl]-amide hydrochloride | 619.04 | 98% (LC/MS) |
| 394 | 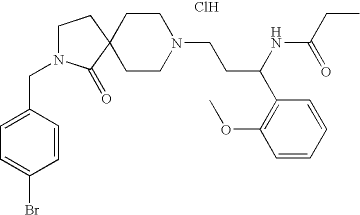 | N-[3-[2-(4-Bromo-benzyl)-1-oxo- 2,8-diaza-spiro[4.5]dec-8-yl]-1-(2- methoxy-phenyl)-propyl]- propionamide hydrochloride | 578.975 | 98% (LC/MS) |
| 395 | 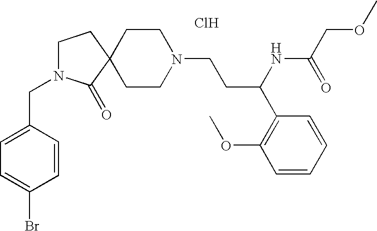 | N-[3-[2-(4-Bromo-benzyl)-1-oxo- 2,8-diaza-spiro[4.5]dec-8-yl]-1-(2- methoxy-phenyl)propyl]-2- methoxy-acetamide hydrochloride | 594.974 | 98% (LC/MS) |
| 396 | 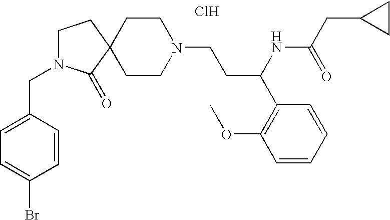 | N-[3-[2-(4-Bromo-benzyl)-1-oxo- 2,8-diaza-spiro[4.5]dec-8-yl]-1-(2- methoxy-phenyl)-propyl]-2- cyclopropyl-acetamide hydrochloride | 605.013 | 98% (LC/MS) |
| 397 | 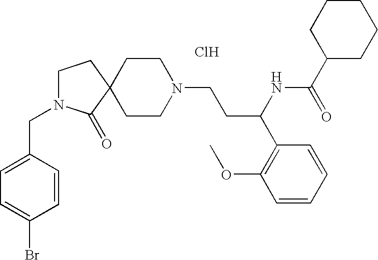 | Cyclohexanecarboxylic acid [3-[2- (4-bromo-benzyl)-1-oxo-2,8-diaza- spiro[4.5]dec-8-yl]-1-(2-methoxy- phenyl)-propyl]-amide hydrochloride | 633.067 | 98% (LC/MS) |
| 398 | 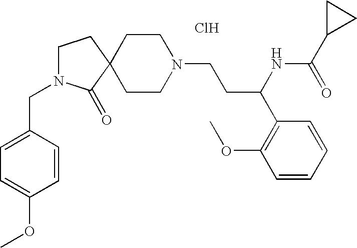 | Cyclopropanecarboxylic acid [3-[2- (4-methoxy-benzyl)-1-oxo-2,8 diaza-spiro[4.5]dec-8-yl]-1-(2- methoxy-phenyl)-propyl]-amide hydrochloride | 542.116 | 98% (LC/MS) |
| 399 | 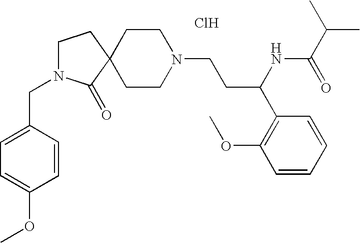 | N-[3-[2-(4-Methoxy-benzyl)-1-oxo- 2,8-diaza-spiro[4.5]dec-8-tl]-1-(2- methoxy-phenyl)-propyl]- issobutyramide hydrochloride | 544.132 | 98% (LC/MS) |
| 400 | 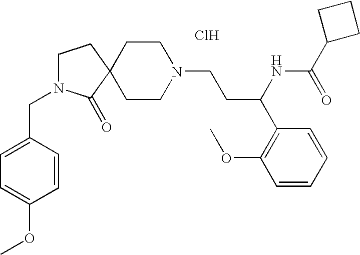 | Cyclobutanecarboxylic acid [3-[2- (4-methoxy-benzyl)-1-oxo-2,8- diaza-spiro[4.5]dec-8-yl]-1-(2- methoxy-phenyl)-propyl]-amide hydrochloride | 556.143 | 98% (LC/MS) |
| 401 | 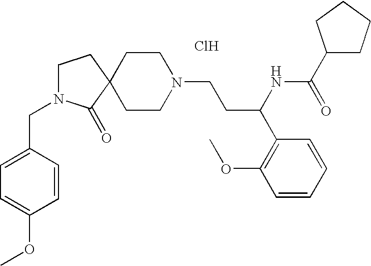 | Cyclopentanecarboxylic acid [3-[2- (4-methoxy-benzyl)-1-oxo-2,8- diaza-spiro[4.5]dec-8-yl]-1-(2- methoxy-phenyl)-propyl]-amide hydrochloride | 570.17 | 98% (LC/MS) |
| 402 | 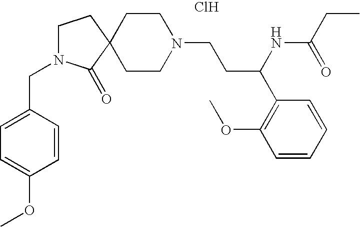 | N-[3-[2-(4-Methoxy-beenzyl)-1-oxo- 2,8-diaza-spiro[4.5]dec-8-yl]-1-(2- methoxy-phenyl)-propyl]- propionamide hydrochloride | 530.105 | 98% (LC/MS) |
| 403 | 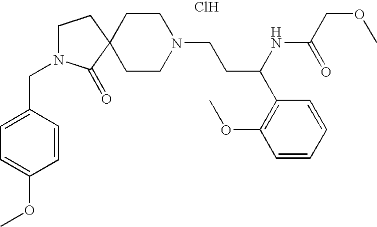 | 2-Methoxy-N-[3-[2-(4-methoxy- benzyl)-1-oxo-2,8-diaza- spiro[4.5]dec-8-yl]-1-(2-methoxy- phenyl)-propyl]-acetamide hydrochloride | 546.104 | 98% (LC/MS) |
| 404 | 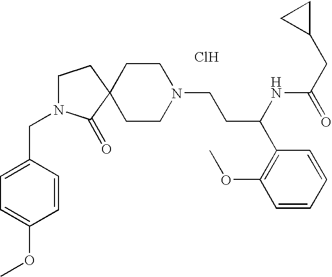 | 2-Cyclopropyl-N-[3[2-(4-methoxy- benzyl)-1-oxo-2,8-diaza- spiro[4.5]dec-8-yl]-1-(2-methoxy- phenyl)-propyl]-acetamide hydrochloride | 556.143 | 98% (LC/MS) |
| 405 | 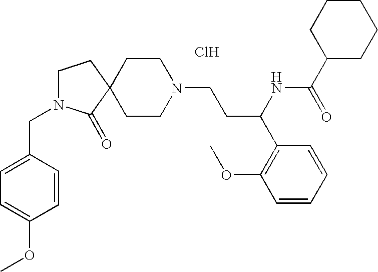 | Cyclohexanecarboxylic acid [3-[2- (4-methoxy-benzyl)-1-oxo-2,8- diaza-spiro[4.5]dec-8-yl]-1-(2- methoxy-phenyl)propyl]-amide hydrochloride | 584.196 | 98% (LC/MS) |
| 406 | 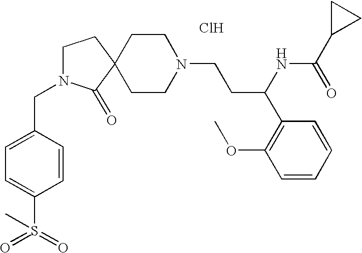 | Cyclopropanecarboxylic acid [3-[2- (4-methanesulfonyl-benzyl)-1-oxo- 2,8-diaza-spiro[4.5]dec-8-yl]-1-(2- methoxy-phenyl)-propyl]-amide hydrochloride | 590.181 | 98% (LC/MS) |
| 407 | 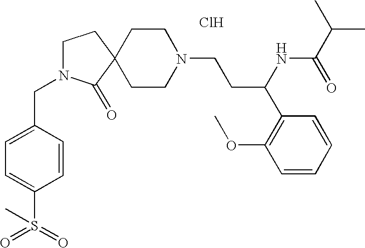 | N-[3-[2-(4-methanesulfonyl- benzyl)-1-oxo-2,8-diaza- spiro[4.5]dec-8-yl]-1-(2-methoxy- phenyl)-propyl]-isobnutyramide hydrochloride | 592.197 | 98% (LC/MS) |
| 408 | 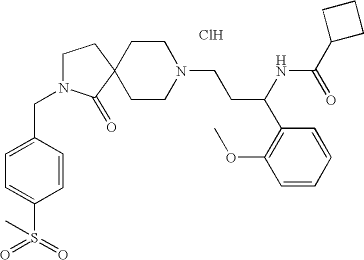 | Cyclobutanecarboxylic acid [3-[2- (4-methanesulfonyl-benzyl)-1-oxo- 2,8-diaza-spiro[4.5]dec-8-yl]-1-(2- meethoxy-phenyl)-propyl]-amide hydrochloride | 604.208 | 98% (LC/MS) |
| 409 | 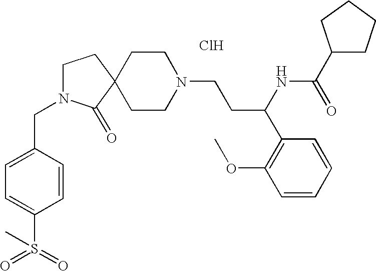 | Cyclopentanecarboxylic acid [3-[2- (4-methanesulfonyl-benzyl)-1-oxo- 2,8-diaza-spiro[4.5]dec-8-yl]-1-(2- methoxy-phenyl)-propyl]-amide hydrochloride | 618.235 | 98% (LC/MS) |
| 410 | 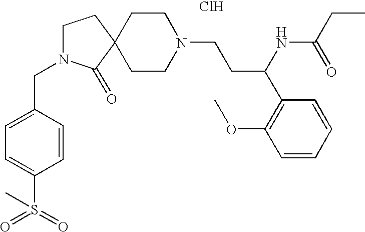 | N-[3-[2-(4-Methanesulfonyl- benzyl)-1-oxo-2,8-diaza- spiro[4.5]dec-8-yl]-1-(2-methoxy- phenyl)-propyl]-propionamide hydrochloride | 578.17 | 98% (LC/MS) |
| 411 | 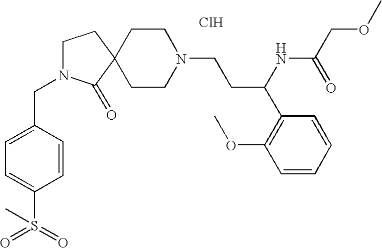 | N-[3-[2-(4-Methanesulfonyl- benzyl)-1-oxo-2,8-diaza- spiro[4.5]dec-8-yl]-1-(2-methoxy- phenyl)-propyl]-2-methoxy- acetamide hydrochloride | 594.169 | 98% (LC/MS) |
| 412 | 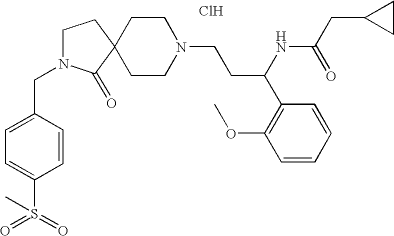 | 2-Cyclopropyl-N-[3-[2-(4- methanesulfonyl-benzyl)-1-oxo- 2,8-diaza-spiro[4.5]dec-8-yl]-1-(2- methoxy-phenyl)-propyl]-acetamide hydrochloride | 604.208 | 98% (LC/MS) |
| 413 | 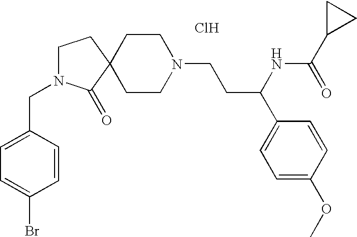 | Cyclopropanecarboxylic acid [3-[2- (4-bromo-benzyl)-1-oxo-2,8-diaza- spiro[4.5]dec-8-yl]-1-(4-methoxy- phenyl)-propyl]-amide hydrochloride | 590.986 | 98% (LC/MS) |
| 414 | 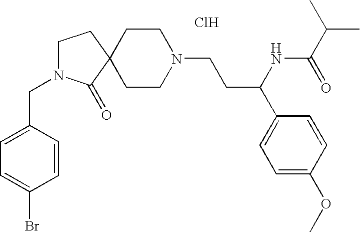 | N-[3-[2-(4-Bromo-benzyl)-1-oxo- 2,8-diaza-spiro[4.5]dec-8-yl]-1-(4- methoxy-phenyl)-propyl]- isobutyramide hydrochloride | 593.002 | 98% (LC/MS) |
| 415 | 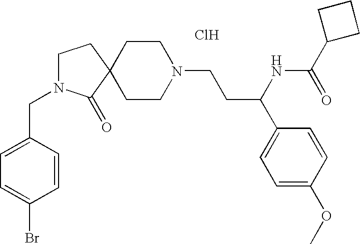 | Cyclobutanecarboxylic acid [3-[2- (4-bromo-benzyl)-1-oxo-2,8-diaza- spiro[4.5]dec-8-yl]-1-(4-methoxy- phenyl)-propyl]-amide hydrochloride | 605.013 | 98% (LC/MS) |
| 416 | 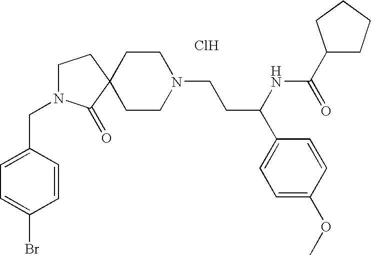 | Cyclopentanecarboxylic acid [3-[2- (4-bromo-benzyl)-1-oxo-2,8-diaza- spiro[4.5]dec-8-yl]-1-(4-methoxy- phenyl)-propyl]-amide hydrochloride | 619.04 | 98% (LC/MS) |
| 417 | 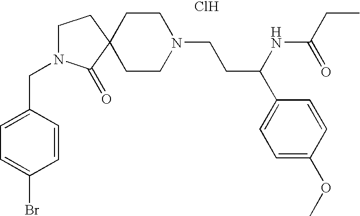 | N-[3-[2-(4-Bromo-benzyl)-1-oxo- 2,8-diaza-spiro[4.5]dec-8-yl]-1-(4- methoxy-phenyl)-propyl]- propionamide hydrochloride | 578.975 | 98% (LC/MS) |
| 418 | 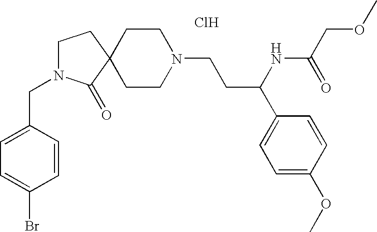 | N-[3-[2-(4-Bromo-benzyl)-1-oxo- 2,8-diaza-spiro[4.5]dec-8-yl]-1-(4- methoxy-phenyl)-propyl]-2- methoxy-acetamide hydrochloride | 594.974 | 98% (LC/MS) |
| 419 | 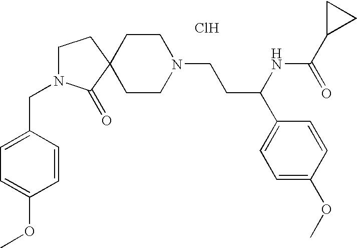 | Cyclopropanecarboxylic acid [3-[2- (4-methoxy-benzyl)-1-oxo-2,8- diaza-spiro[4.5]dec-8-yl]-1-(4- methoxy-phenyl)-propyl]-amide hydrochloride | 542.116 | 98% (LC/MS) |
| 420 | 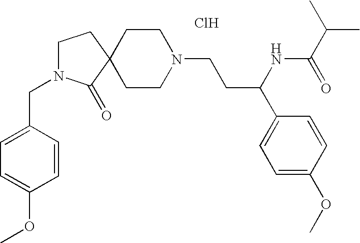 | N-[3-[2-(4-Methoxy-benzyl)-1-oxo- 2,8-diaza-spiro[4.5]dec-8-yl]-1-(4- methoxy-phenyl)-propyl]- isobutyramide hydrochloride | 544.132 | 98% (LC/MS) |
| 421 | 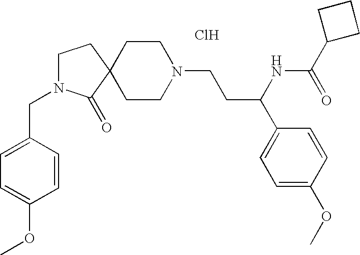 | Cyclobutanecarboxylic acid [3-[3- (4-methoxy-benzyl)-1-oxo-2,8- diaza-spiro[4.5]dec-8-yl]-1-(4- methoxy-phenyl)-propyl]-amide hydrochloride | 556.143 | 98% (LC/MS) |
| 422 | 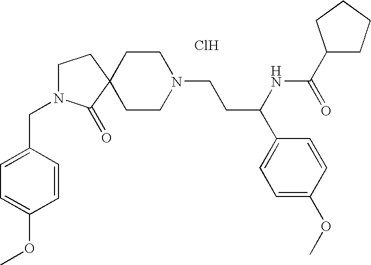 | Cyclopentanecarboxylic acid [3-[2- (4-methoxy-benzyl)-1-oxxo-2,8- diaza-spiro[4.5]dec-8-yl]-1-(4- methoxy-phenyl)-propyl]-amide hydrochloride | 570.17 | 98% (LC/MS) |
| 423 | 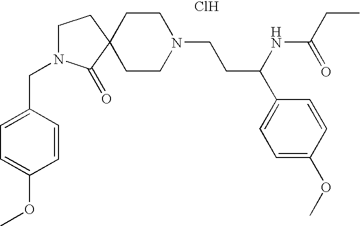 | N-[3-[2-(4-Methoxy-benzyl)-1-oxo- 2,8-diaza-spiro[4.5]dec-8-yl]-1-(4- methoxy-phenyl)-propyl]- propionamide hydrochloride | 530.105 | 98% (LC/MS) |
| 424 | 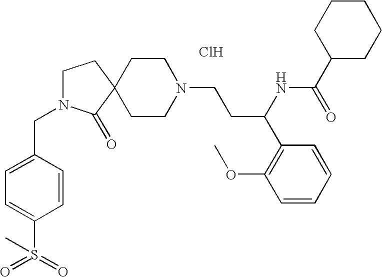 | Cyclohexanecarboxylic acid [3-[2- (4-methanesulfonyl-benzyl)-1-oxo- 2,8-diaza-spiro[4.5]dec-8-yl]-1-(2- methoxy-phenyl)-propyl]-amide hydrochloride | 632.261 | 98% (LC/MS) |
| 425 | 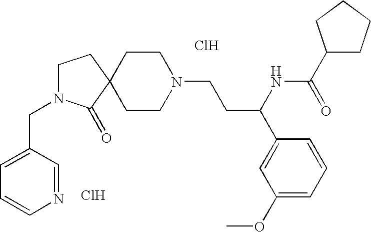 | Cyclopentanecarboxylic acid [1-(3- methoxy-phenyl)-3-(1-oxo-2- pyridin-3-ylmethyl-2,8-diaza- spiro[4.5]dec-8-yl)-propyl]-amide dihydrochloride | 577.593 | 98% (LC/MS) |
| 426 | 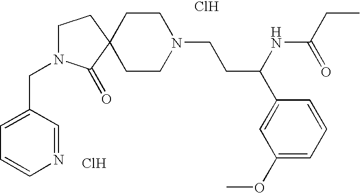 | N-[1-(3-Methoxy-phenyl)-3-(1-oxo- 2-pyridin-3-ylmethyl-2,8-diaza- spiro[4.5]dec-8-yl)-propyl]- propionamide dihydrochloride | 537.528 | 98% (LC/MS) |
| 427 | 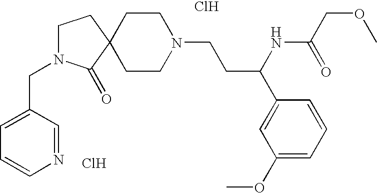 | 2-Methoxy-NM-[1-(3-methoxy- phenyl)-3-(1-oxo-2-pyridin-3- ylmeethyl-2,8-diaza-spiro[4.5]dec-8- yl)-propyl]-acetamide dihydrochloride | 553.527 | 98% (LC/MS) |
| 428 | 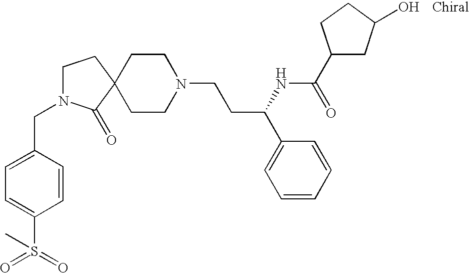 | 3-Hydroxy-cyclopentanecarboxylic acid {(S)-3-[2-(4-methanesulfonyl- benzyl)-1-oxo-2,8-diaza- spiro[4.5]dec-8-yl]-1-phenyl- propyl}-amide | 567.747 | 90% (HPLC) |
| 429 | 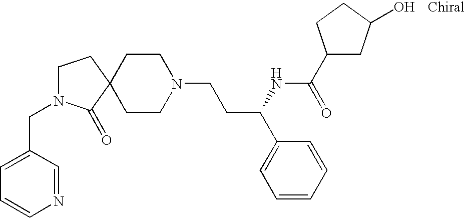 | 3-Hydroxy-cyclopentanecarboxylic acid [(S)-3-(1-oxo-2-pyridin-3- ylmethyl-2,8-diaza-spiro[4.5]dec-8- yl)-1-phenyl-propyl]-amide | 490.644 | 97% (HPLC) |
| 430 | 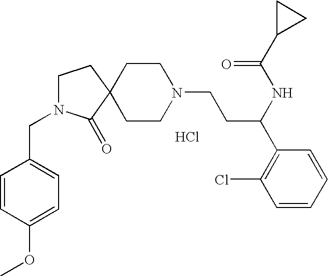 | Cyclopropanecarboxylic acid {1-(2- chloro-phenyl)-3-[2-(4-methoxy- benzyl)-1-oxo-2,8-diaza- spiro[4.5]dec-8-yl]-propyl}-amide hydrochloride | 546.535 | 98% (LC/MS) |
| 431 | 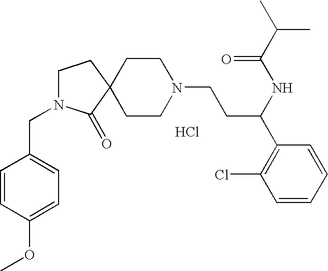 | N-{1-(2-Chloro-phenyl)-3-[2-(4- methoxy-benzyl)-1-oxo-2,8-diaza- spiro[4.5]dec-8-yl]-propyl}- isobutyramide hydrochloride | 548.551 | 98% (LC/MS) |
| 432 | 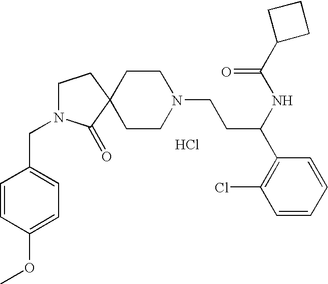 | Cyclobutanecarboxylic acid {1-(2- chloro-phenyl)-3-[2-(4-methoxy- benzyl)-1-oxo-2,8-diaza- spiro[4.5]dec-8-yl]propyl}-amide hydrochloride | 560.562 | 98% (LC/MS) |
| 433 | 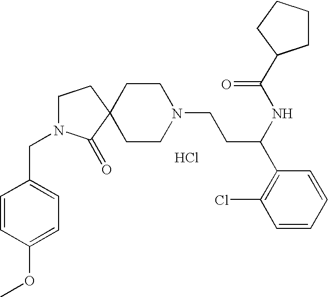 | Cyclopentanecarboxylic acid {1-(2- chloro-phenyl)-3-[2-(4-methoxy- benzyl)-1-oxo-2,8-diaza- spiro[4.5]dec-8-yl]propyl}-amide hydrochloride | 574.589 | 98% (LC/MS) |
| 434 | 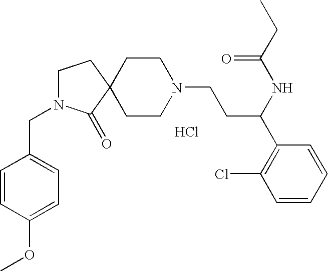 | N-{1-(2-Chloro-phenyl)-3-[2-(4- methoxy-benzyl)-1-oxo-2,8-diaza- spiro[4.5]dec-8-yl]propyl}- propionamide hydrochloride | 534.524 | 98% (LC/MS) |
| 435 | 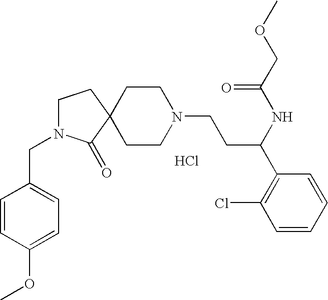 | N-{1-(2-Chloro-phenyl)-3-[2-(4- methoxy-benzyl)-1-oxo-2,8-diaza- spiro[4.5]dec-8-yl]propyl}-2- methoxy-acetamide hydrochloride | 550.523 | 98% (LC/MS) |
| 436 | 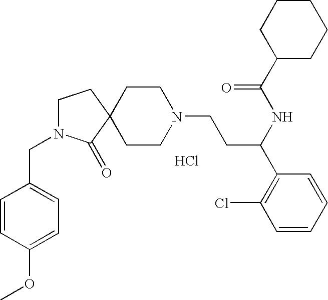 | Cyclohexanecarboxylic acid {1-(2- chloro-phenyl)-3-[2-(4-methoxy- benzyl)-1-oxo-2,8-diaza- spiro[4.5]dec-8-yl]-propyl}-amide hydrochloride | 588.616 | 98% (LC/MS) |
| 437 | 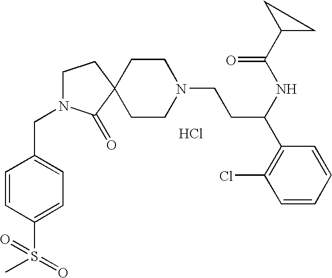 | Cyclopropanecarboxylic acid {1-(2- chloro-phenyl)-3-[2-(4- methanesulfonyl-benzyl)-1-oxo- 2,8-diaza-spiro[4.5]dec-8-yl]- propyl}-amide hydrochloride | 594.6 | 98% (LC/MS) |
| 438 | 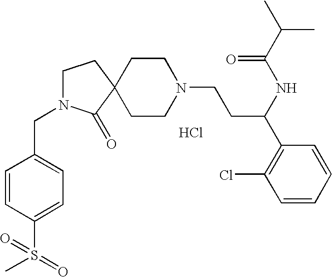 | N-{1-(2-Chloro-phenyl)-3-[2-(4- methanesulfonyl-benzyl)-1-oxo- 2,8-diaza-spiro[4.5]dec-8-yl]- propyl}-isobutyramide hydrochloride | 596.616 | 98% (LC/MS) |
| 439 | 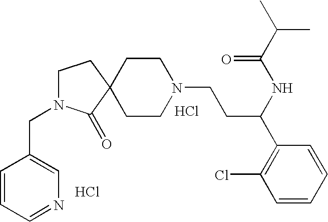 | Cyclobutanecarboxylic acid {1-(2- chloro-phenyl)-3-[2-(4- methanesulfonyl-benzyl)-1-oxo- 2,8-diaza-spiro[4.5]dec-8-yl]- propyl}-amide hydrochloride | 608.627 | 98% (LC/MS) |
| 440 | 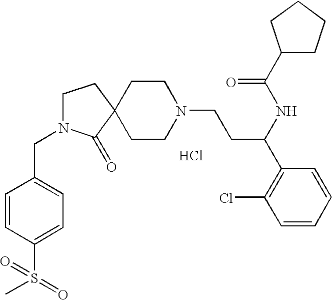 | Cyclopentanecarboxylic acid {1-(2- chloro-phenyl)-3-[2-(4- methanesulfonyl-benzyl)-1-oxo- 2,8-diaza-spiro[4.5]dec-8-yl]- propyl}-amide hydrochloride | 622.654 | 98% (LC/MS) |
| 441 | 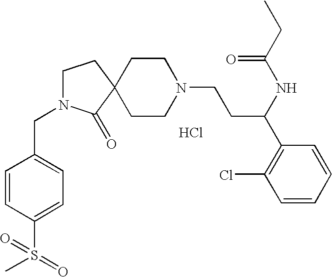 | N-{1-(2-Chloro-phenyl)-3-[2-(4- methanesulfonyl-benzyl)-1-oxo- 2,8-diaza-spiro[4.5]dec-8-yl]- propyl}-propionamide hydrochloride | 582.589 | 98% (LC/MS) |
| 442 | 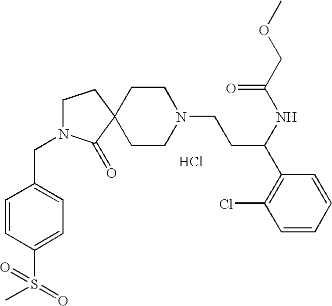 | N-{1-(2-Chloro-phenyl)-3-[2-(4- methanesulfonyl-benzyl)-1-oxo- 2,8-diaza-spiro[4.5]dec-8-yl]- propyl}-2-methoxy-acetamide hydrochloride | 598.588 | 98% (LC/MS) |
| 443 | 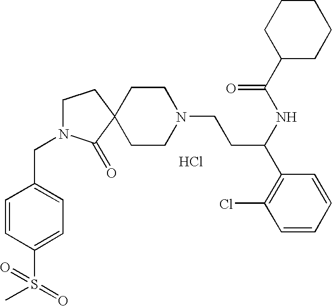 | Cyclohexanecarboxylic acid {1-(2- chloro-phenyl)-3-[2-(4- methanesulfonyl-benzyl)-1-oxo- 2,8-diaza-spiro[4.5]dec-8-yl]- propyl}-amide hydrochloride | 636.681 | 98% (LC/MS) |
| 444 | 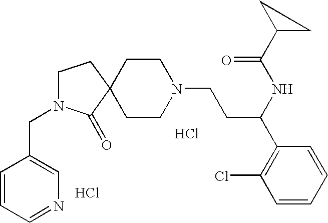 | Cyclopropanecarboxylic acid [1-(2- chloro-phenyl)-3-(1-oxo-2-pyridin- 3-ylmethyl-2,8-diaza-spiro[4.5]dec- 8-yl)-propyl]-amide hydrochloride | 553.958 | 98% (LC/MS) |
| 445 | 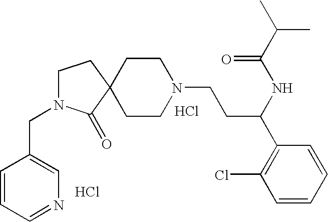 | N-[1-(2-Chloro-phenyl)-3-(1-oxo-2- pyridin-3-ylmethyl-2,8-diaza- spiro[4.5]dec-8-yl)-propyl]- isobutyramide dihydrochloride | 555.974 | 98% (LC/MS) |
| 446 | 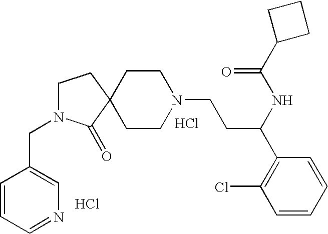 | Cyclobutanecarboxylic acid [1-(2- chloro-phenyl)-3-(1-oxo-2-pyridin- 3-ylmethyl-2,8-diaza-spiro[4.5]dec- 8-yl)-propyl]-amide dihydrochloride | 567.985 | 98% (LC/MS) |
| 447 | 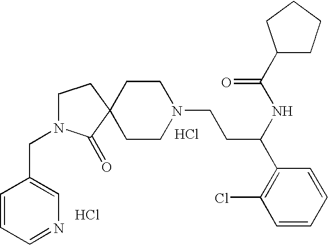 | Cyclopentanecarboxylic acid [1-(2- chloro-phenyl)-3-(1-oxo-2-pyridin- 3-ylmethyl-2,8-diaza-spiro[4.5]dec- 8-yl)-propyl]-amide dihydrochloride | 582.012 | 98% (LC/MS) |
| 448 | 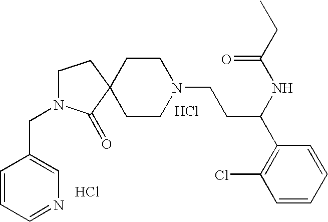 | N-[1-(2-Chloro-phenyl)-3-(1-oxo-2- pyridin-3-ylmethyl-2,8-diaza- spiro[4.5]dec-8-yl)-propyl]- propionamide dihydrochloride | 541.948 | 98% (LC/MS) |
| 449 | 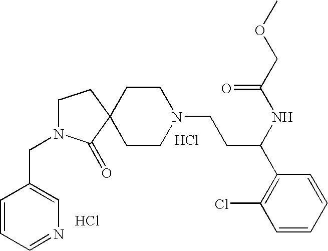 | N-[1-(2-Chloro-phenyl)-3-(1-oxo-2- pyridin-3-ylmethyl-2,8-diaza- spiro[4.5]dec-8-yl)-propyl]-2- methoxy-acetamide dihydrochloride | 557.946 | 93.6% (LC/MS) |
| 450 | 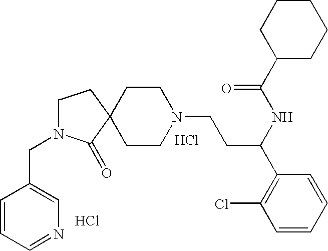 | Cyclohexanecarboxylic acid [1-(2- chloro-phenyl)-3-(1-oxo-2-pyridin- 3-ylmethyl-2,8-diaza-spiro[4.5]dec- 8-yl)-propyl]-amide dihydrochloride | 596.039 | 98+ (LC/MS) |
| 451 | 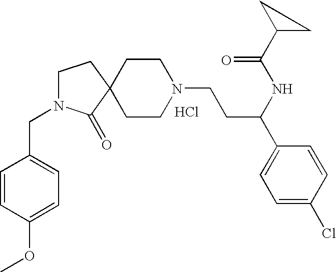 | Cyclopropanecarboxylic acid {1-(4- chloro-phenyl)-3-[2-(4-methoxy- benzyl)-1-oxo-2,8-diaza- spiro[4.5]dec-8-yl]-propyl}-amide hydrochloride | 546.535 | 98% (LC/MS) |
| 452 | 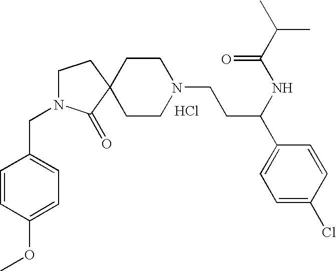 | N-{1-(4-Chloro-phenyl)-3-[2-(4- meethoxy-benzyl)-1-oxo-2,8-diaza- spiro[4.5]dec-8-yl]-propyl}- isobutyramide hydrochloride | 548.551 | 98% (LC/MS) |
| 453 | 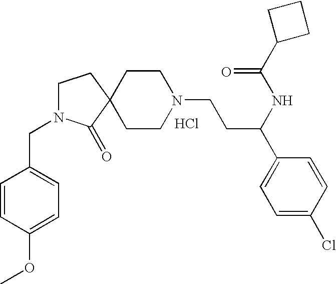 | Cyclobutanecarboxylic acid {1-(4- chloro-phenyl)-3-[2-(4-methoxy- benzyl)-1-oxo-2,8-diaza- spiro[4.5]dec-8-yl]-propyl}-amide hydrochloride | 560.562 | 98% (LC/MS) |
| 454 | 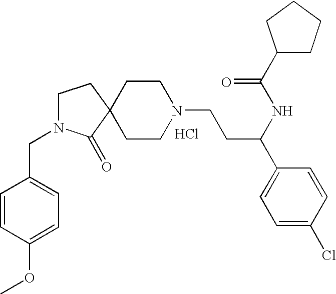 | Cyclopentanecarboxylic acid {1-(4- chloro-phenyl)-3-[2-(4-methoxy- benzyl)-1-oxo-2,8-diaza- spiro[4.5]dec-8-yl]-propyl}-amide hydrochloride | 574.589 | 98% (LC/MS) |
| 455 | 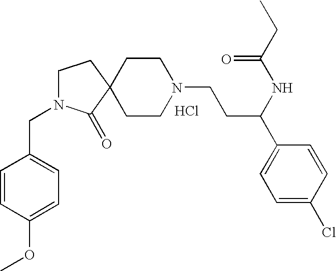 | N-{1-(4-Chloro-phenyl)-3-[2-(4- methoxy-benzyl)-1-oxo-2,8-diaza- spiro[4.5]dec-8-yl]propyl}- propionamide hydrochloride | 534.524 | 98% (LC/MS) |
| 456 | 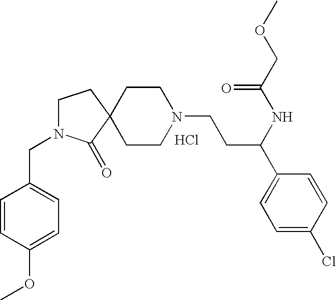 | N-{1-(4-Chloro-phenyl)-3-[2-(4- methoxy-benzyl)-1-oxo-2,8-diaza- spiro[4.5]dec-8-yl]-propyl}-2- methoxy-acetamide hydrochloride | 550.523 | 98% (LC/MS) |
| 457 | 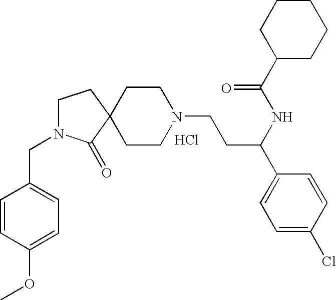 | Cyclohexanecarboxylic acid {1-(4- chloro-phenyl)-3-[2-(4-methoxy- benzyl)-1-oxo-2,8-diaza- spiro[4.5]dec-8-yl]-propyl}-amide hydrochloride | 588.616 | 98% (LC/MS) |
| 458 | 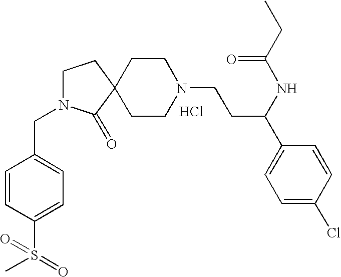 | N-{1-(4-Chloro-phenyl)-3-[2-(4- methanesulfonyl-benzyl)-1-oxo- 2,8-diaza-spiro[4.5]dec-8-yl]- propyl}-propionamide hydrochloride | 592.589 | 98% (LC/MS) |
| 459 | 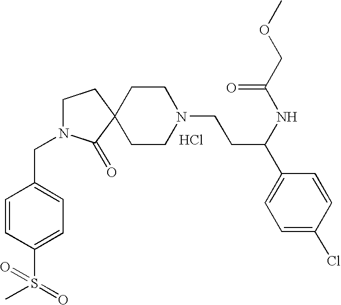 | N-{1-(4-Chloro-phenyl)-3-[2-(4- methanesulfonyl-benzyl)-1-oxo- 2,8-diaza-0spiro[4.5]dec-8-yl]- propyl}-2-methoxy-acetamide hydrochloride | 598.588 | 98% (LC/MS) |
| 460 | 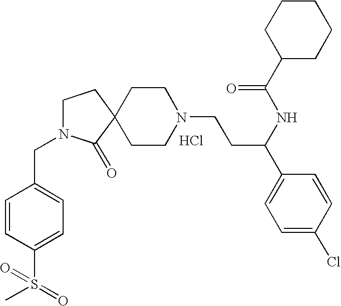 | Cyclohexanecarboxylic acid {1-(4- chloro-phenyl)-3-[2-(4- meethanesulfonyl-benzyl)-1-oxo- 2,8-diaza-spiro[4.5]dec-8-yl]- propyl}-amide hydrochloride | 636.681 | 98% (LC/MS) |
| 461 | 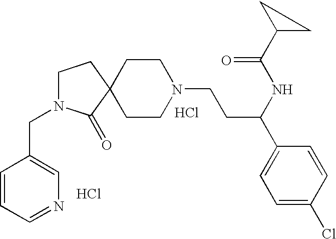 | Cyclopropanecarboxylic acid [1-(4- chloro-phenyl)-3-(1-oxo-2-pyridin- 3-ylmethyl-2,8-diaza-spiro[4.5]dec- 8-yl)-propyl]-amide dihydrochloride | 553.958 | 98% (LC/MS) |
| 462 | 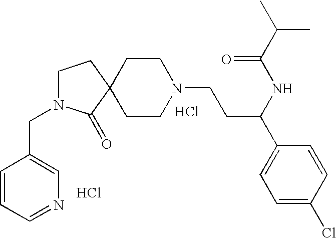 | N-[1-(4-Chloro-phenyl)-3-(1-oxo-2- pyridin-3-ylmethyl-2,8-diaza- spiro[4.5]dec-8-yl)-propyl- isobutyramide dihydrochloride | 555.974 | 98% (LC/MS) |
| 463 | 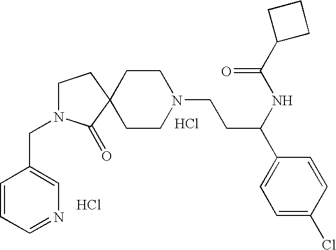 | Cyclobutanecarboxylic acid [1-(4- chloro-phenyl)-3-(1-oxo-2-pyridin- 3-ylmethyl-2,8-diaza-spiro[4.5]dec- 8-yl)-propyl]-amide dihydrochloride | 567.985 | 98% (LC/MS) |
| 464 | 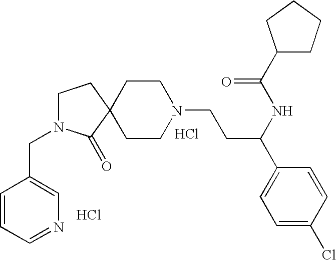 | Cyclopentanecarboxylic acid [1-(4- chloro-phenyl)-3-(1-oxo-2-pyridin- 3-ylmeethyl-2,8-diaza-spiro[4.5]dec- 8-yl)-propyl]-amide dihydrochloride | 582.012 | 95% (LC/MS) |
| 465 | 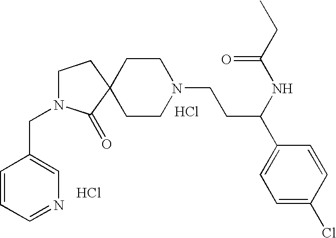 | N-[1-(4-Chloro-phenyl)-3-(1-oxo-2- pyridin-3-ylmethyl-2,8-diaza- spiro[4.5]dec-8-yl)-propyl]- propionamide dihydrochloride | 541.948 | 93.5% (LC/MS) |
| 466 | 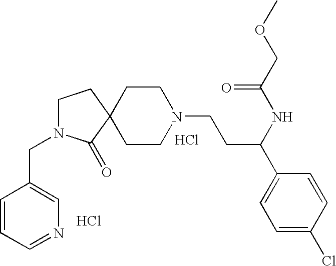 | N-[1-(4-Chloro-phenyl)-3-(1-oxo-2- pyridin-3-ylmethyl-2,8-diaza- spiro[4.5]dec-8-yl)-propyl]-2- methoxy-acetamide dihydrochloride | 557.946 | 98% (LC/MS) |
| 467 | 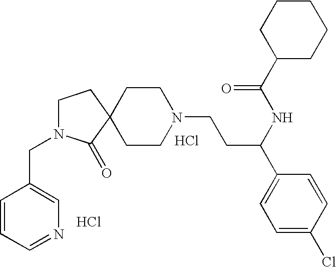 | Cyclohexanecarboxylic acid [1-(4- chloro-phenyl)-3-(1-oxo-2-pyridin- 3-ylmethyl-2,8-diaza-spiro[4.5]dec- 8-yl)-propyl]-amide dihydrochloride | 596.039 | 94.4% (LC/MS) |
| 468 | 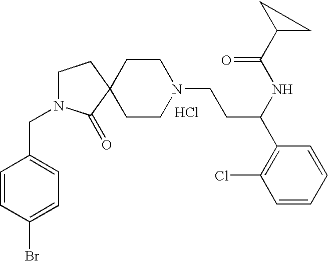 | Cyclopropanecarboxylic acid [3-[2- (4-bromo-benzyl)-1-oxo-2,8-diaza- spiro[4.5]dec-8-yl]-1-(2-chloro- phenyl)-propyl]-amide hydrochloride | 595.406 | 98% (LC/MS) |
| 469 | 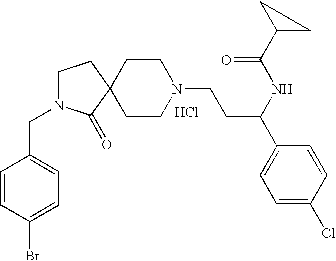 | Cyclopropanecarboxylic acid [3-[2- (4-bromo-benzyl)-1-oxo-2,8-diaza- spiro[4.5]dec-8-yl]-1-(4-chloro- phenyl)-propyl]-amide hydrochloride | 595.406 | 98% (LC/MS) |
| 470 | 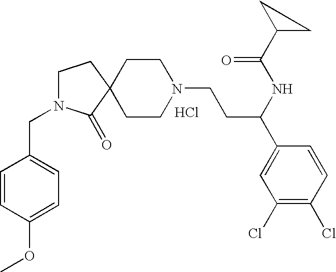 | Cyclopropanecarboxylic acid {1- (3,4-dichloro-phenyl)-3-[2-(4- methoxy-benzyl)-1-oxo-2,8-diaza- spiro[4.5]dec-8-yl]-propyl}-amide hydrochloride | 580.98 | 98% (LC/MS) |
| 471 | 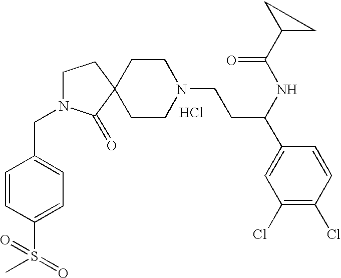 | Cyclopropanecarboxylic acid {1- (3,4-dichloro-phenyl)-3-[2-(4- methanesulfonyl-benzyl)-1-oxo- 2,8-diaza-spiro[4.5]dec-8-yl]- propyl}-amide hydrochloride | 629.045 | 98% (LC/MS) |
| 472 | 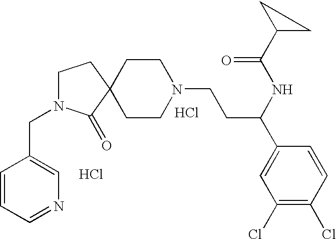 | Cyclopropanecarboxylic acid [1- (3,4-dichloro-phenyl)-3-(1-oxo-2- pyridin-3-ylmethyl-2,8-diaza- spiro[4.5]dec-8-yl)-propyl]-amide dihydrochloride | 588.404 | 98% (LC/MS) |
| 473 | 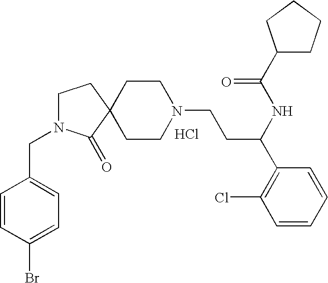 | Cyclopentanecarboxylic acid [3-[2- (4-bromo-benzyl)-1-oxo-2,8-diaza- spiro[4.5]dec-8-yl]-1-(2-chloro- phenyl)-propyl]-amide hydrochloride | 623.459 | 98% (LC/MS) |
| 474 | 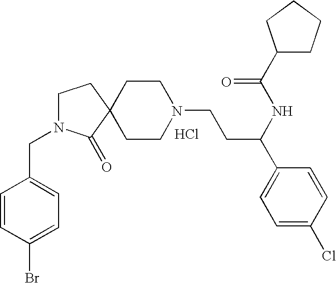 | Cyclopentanecarboxylic acid [3-[2- (4-bromo-benzyl)-1-oxo-2,8-diaza- spiro[4.5]dec-8-yl]-1-(4-chloro- phenyl)-propyl]-amide hydrochloride | 623.459 | 98% (LC/MS) |
| 475 | 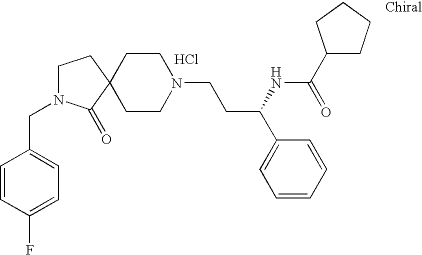 | Cyclopentanecarboxylic acid {(S)- 3-[2-(4-fluoro-benzyl)-1-oxo-2,8- diaza-spiro[4.5]dec-8-yl]-1-phenyl- propyl}-amide hydrochloride | 528.108 | 98% (LC/MS) |
| 476 | 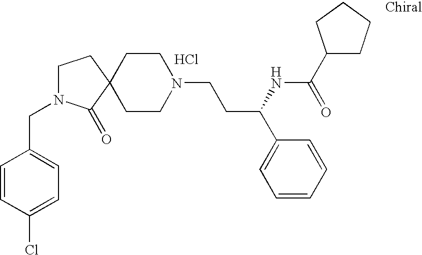 | Cyclopentanecarboxylic acid {(S)- 3[2-(4-chloro-benzyl)-1-oxo-2,8- diaza-spiro[4.5]dec-8-yl]-1-phenyl- propyl}-amide hydrochloride | 544.563 | 98% (LC/MS) |
| 477 | 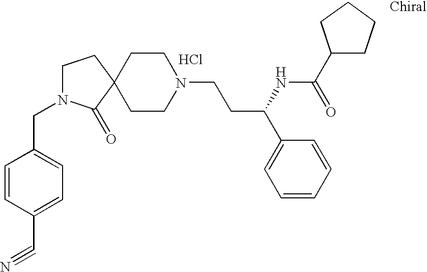 | Cyclopentanecarboxylic acid {(S)- 3-[2-(4-cyano-benzyl)-1-oxo-2,8- diaza-spiro[4.5]dec-8-yl]-1-phenyl- propyl}-amide hydrochloride | 535.128 | 98% (LC/MS) |
| 478 | 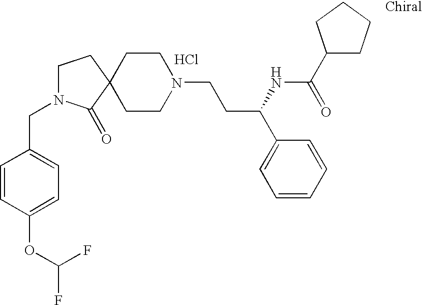 | Cyclopentanecarboxylic acid {(S)- 3-[2-(4-difluoromethoxy-benzyl)-1- oxo-2,8-diaza-spiro[4.5]dec-8-yl]-1- phenyl-propyl}-amide hydrochloride | 576.124 | 98% (LC/MS) |
| 479 | 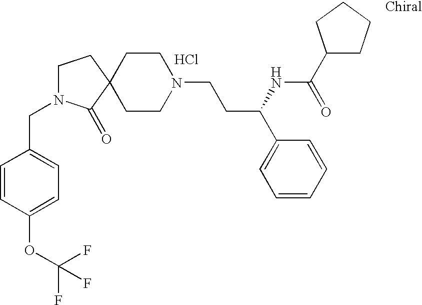 | Cyclopentanecarboxylic acid {(S)- 3-[1-oxo-2-(4-trifluoromethoxy- benzyl)-2,8-diaza-spiro[4.5]dec-8- yl]-1-phenyl-propyl}-amide hydrochloride | 594.114 | 98% (LC/MS) |
| 480 | 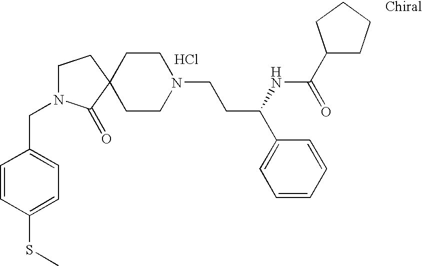 | Cyclopentanecarboxylic acid {(S)- 3-[2-(4-methylsulfanyl-benzyl)-1- oxo-2,8-diaza-spiro[4.5]dec-8-yl]-1- phenyl-propyl}-amide hydrochloride | 556.211 | 98% (LC/MS) |
| 481 | 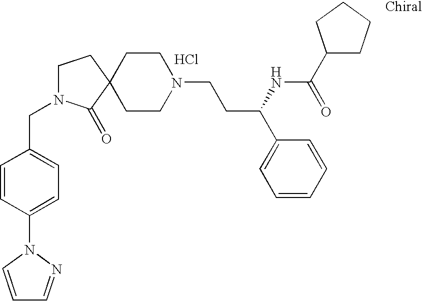 | Cyclopentanecarboxylic acid {(S)- 3-[1-oxo-2-(4-pyrazol-1-yl-benzyl)- 2,8-diaza-spiro[4.5]dec-8-yl]-1- phenyl-propyl}-amide hydrochloride | 576.181 | 98% (LC/MS) |
| 482 | 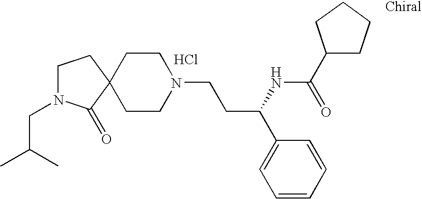 | Cyclopentanecarboxylic acid [(S)- 3-(2-isobutyl-1-oxo-2,8-diaza- spiro[4.5]dec-8-yl)-1-phenyl- propyl]-amide hydrochloride | 476.101 | 95% (LC/MS) |
| 483 | 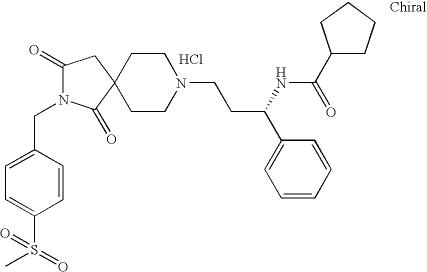 | Cyclopentanecarboxylic acid {(S)- 3-[2-(4-meethanesulfonyl-benzyl)-3- oxo-2,8-diaza-spiro[4.5]dec-8-yl]-1- phenyl-propyl}-amide hydrochloride | 588.209 | 98% (LC/MS) |
| 484 | 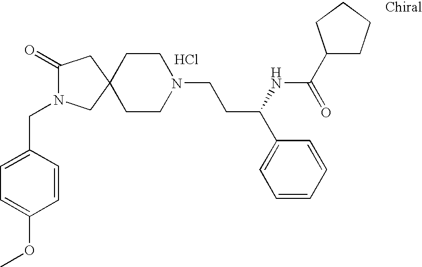 | Cyclopropanecarboxylic acid {(S)- 3-[2-(4-methoxy-benzyl)-3-oxo-2,8- diaza-spiro[4.5]dec-8-yl]-1-phenyl- propyl}-amide hydrochloride | 540.114 | 98% (LC/MS) |
| 485 | 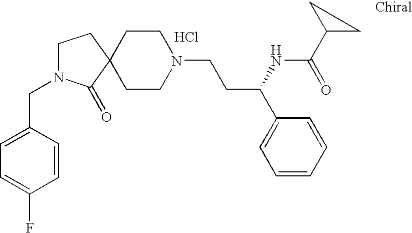 | Cyclopropanecarboxylic acid {(S)- 3-[2-(4-fluoro-benzyl)-1-oxo-2,8- diaza-spiro[4.5]dec-8-yl]-1-phenyl- propyl}-amide hydrochloride | 500.055 | 98% (LC/MS) |
| 486 | 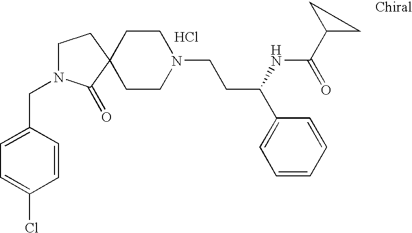 | Cyclopropanecarboxylic acid {(S)- 3-[2-(4-chloro-benzyl)-1-oxo-2,8- diaza-spiro[4.5]dec-8-yl]1-phenyl- propyl}-amide hydrochloride | 516.51 | 98% (LC/MS) |
| 487 | 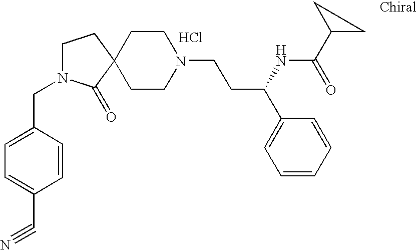 | Cyclopropanecarboxylic acid {(S)- 3-[2-(4-cyano-benzyl)-1-oxo-2,8- diaza-spiro[4.5]dec-8-yl]-1-phenyl- propyl}-amide hydrochloride | 507.074 | 98% (LC/MS) |
| 488 | 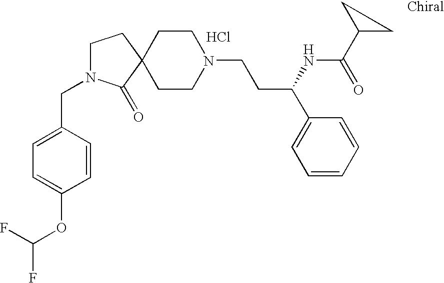 | Cyclopropanecarboxylic acid {(S)- 3-[2-(4-difluoromethoxy-benzyl)-1- oxo-2,8-diaza-spiro[4.5]dec-8-yl]-1- phenyl-propyl}-amide hydrochloride | 548.07 | 98% (LC/MS) |
| 489 | 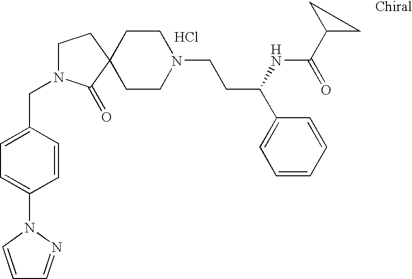 | Cyclopropanecarboxylic acid {(S)- 3-[1-oxo-2-(4-pyrazol-1-yl-benzyl)- 2,8-diaza-spiro[4.5]dec-8-yl]-1- phenyl-propyl}-amide hydrochloride | 548.127 | 98% (LC/MS) |
| 490 | 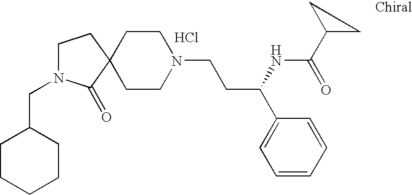 | Cyclopropanecarboxylic acid [(S)- 3-(2-cyclohexylmethyl-1-oxo-2,8- diaza-spiro[4.5]dec-8-yl)-1-phenyl- propyl]-amide hydrochloride | 488.112 | 99% (LC/MS) |
| 491 | 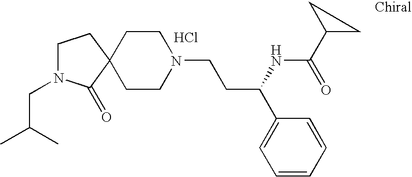 | Cyclopropanecarboxylic acid [(S)- 3-(2-isobutyl-1-oxo-2,8-diaza- spiro[4.5]dec-8-yl)-1-phenyl- propyl]-amide hydrochloride | 448.047 | 98% (LC/MS) |
| 492 | 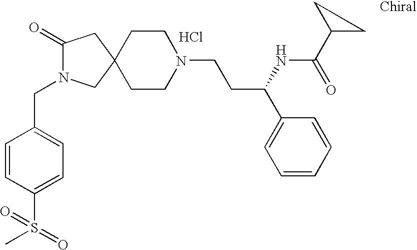 | Cyclopropanecarboxylic acid {(S)- 3-[2-(4-methanesulfonyl-benzyl)-3- oxo-2,8-diaza-spiro[4.5]dec-8-yl]-1- phenyl-propyl}-amide hydrochloride | 560.155 | 98% (LC/MS) |
| 493 | 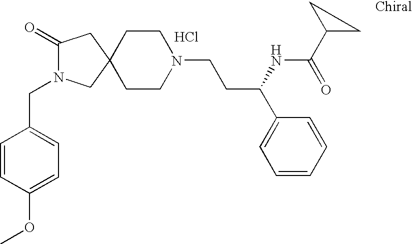 | Cyclopropanecarboxylic acid {(S)- 3-[2-(4-methoxy-benzyl)-3-oxo-2,8- diaza-spiro[4.5]dec-8-yl]-1-phenyl- propyl}-amide hydrochloride | 512.09 | 98% (LC/MS) |
| 494 | 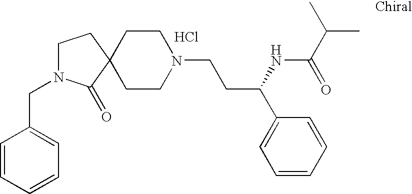 | N-[(S)-3-(2-Benzyl-1-oxo-2,8- diaza-spiro[4.5]dec-8-yl)-1-phenyl- propyl]-isobutyramide hydrochloride | 484.08 | 95% (LC/MS) |
| 495 | 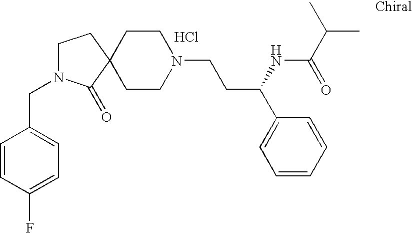 | N-{(S)-3-[2-(4-Fluoro-benzyl)-1- oxo-2,8-diaza-spirro[4.5]dec-8-yl]-1- phenyl-propyl}-isobutyramide hydrochloride | 502.07 | 90% (LC/MS) |
| 496 | 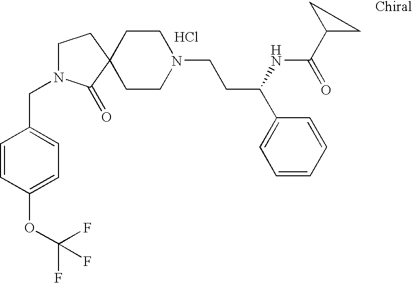 | Cyclopropanecarboxylic acid {(S)- 3-[1-oxxo-2-(4-trifluoromethoxy- benzyl)-2,8-diaza-spiro[4.5]dec-8- yl]-1-phenyl-propyl}-amide hydrochloride | 566.06 | 98% (LC/MS) |
| 497 | 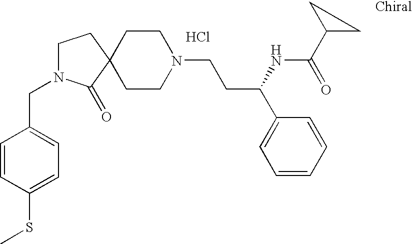 | Cyclopropanecarboxylic acid {(S)- 3-[2-(4-methylsulfanyl-benzyl)-1- oxo-2,8-diaza-spiro[4.5]dec-8-yl]-1- phenyl-propyl}-amide hydrochloride | 528.157 | 98% (LC/MS) |
| 498 | 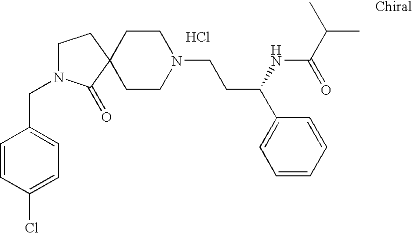 | N-{(S)-3-[2-(4-Chloro-benzyl)-1- oxo-2,8-diaza-spiro[4.5]dec-8-yl]-1- phenyl-propyl}-isobutyramide hydrochloride | 518.525 | 95% (LC/MS) |
| 499 | 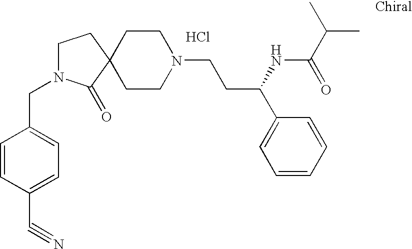 | N-{(S)-3-[2-(4-Cyano-benzyl)-1- oxo-2,8-diaza-spiro[4.5]dec-8-yl]-1- phenyl-propyl}-isobutyramide hydrochloride | 509.09 | 98% (LC/MS) |
| 500 | 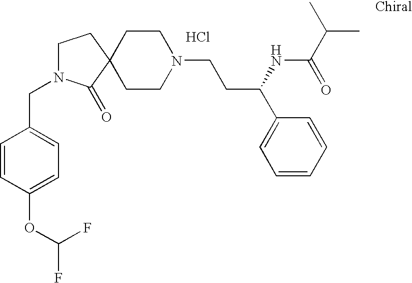 | N-{(S)(-3-[2-(4-Difluoromethoxy- benzyl)-1-oxo-2,8-diaza- spiro[4.5]dec-8-yl]-1-phenyl- propyl}-isobutyramide hydrochloride | 556.086 | 90% (LC/MS) |
| 501 | 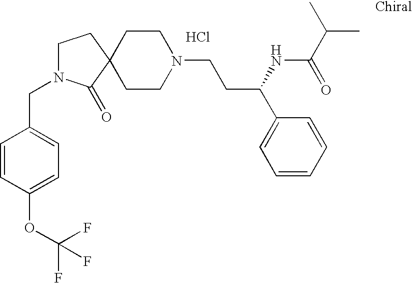 | N-{(S)-3-[1-Oxo-2-(4- trifluoromethoxy-benzyl)-2,8-diaza- spiro[4.5]dec-8-yl]-1-phenyl- propyl}-isobutyramide hydrochloride | 568.076 | 91.3% (LC/MS) |
| 502 | 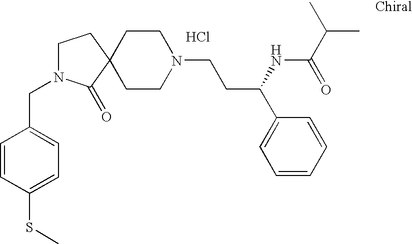 | N-{(S)-3-[2-(4-Methylsulfanyl- benzyl)-1-oxo-2,8-diaza- spiro[4.5]dec-8-yl]-1-phenyl- propyl}-isobutyramide hydrochloride | 530.173 | 90% (LC/MS) |
| 503 | 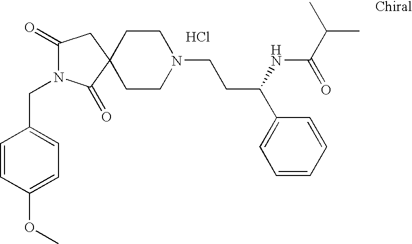 | N-{(S)-3-[2-(4-Methoxy-benzyl)-3- oxo-2,8-diaza-spiro[4.5]dec-8-yl]-1- phenyl-propyl}-isobutyramide hydrochloride | 514.106 | 95% (LC/MS) |
| 504 | 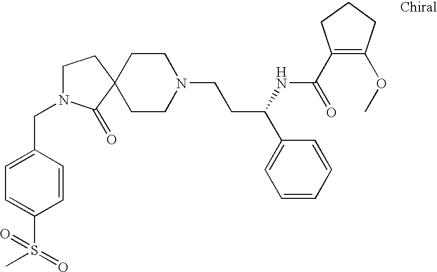 | 2-Methoxy-cyclopent-1- enecarboxylic acid {(S)-3-[2-(4- methanesulfonyl-benzyl)-1-oxo- 2,8-diaza-spiro[4.5]dec-8-yl]-1- phenyl-propyl}-amide | 579.758 | 87% (HPLC) |
| 505 | 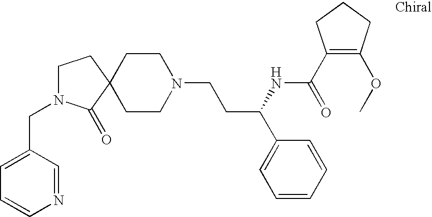 | 2-Methoxy-cyclopent-1- enecarboxylic acid [(S)-3-(1-oxo-2- pyridin-3-ylmethyl-2,8-diaza- spiro[4.5]dec-8-yl)-1-phenyl- propyl]-amide | 502.655 | 84% (HPLC) |
| 506 | 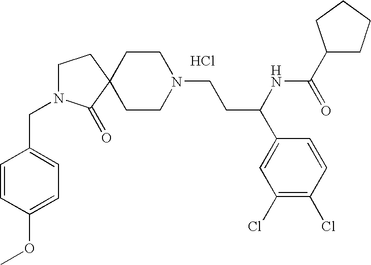 | Cyclopentanecarboxylic acid {1- (3,4-dichloro-phenyl)-3-[2-(4- methoxy-benzyl)-1-oxo-2,8-diaza- spiro[4.5]dec-8-yl]-propyl}-amide hydrochloride | 609.034 | 98% (LC/MS) |
| 507 | 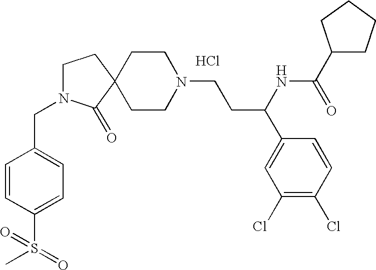 | Cyclopentanecarboxylic acid {1- (3,4-dichloro-phenyl)-3-(1-oxo-2- pyridin-3-ylmethyl-2,8-diaza- spiro[4.5]dec-8-yl)-propyl]-amide dihydrochloride | 616.457 | 98% (LC/MS) |
| 508 | 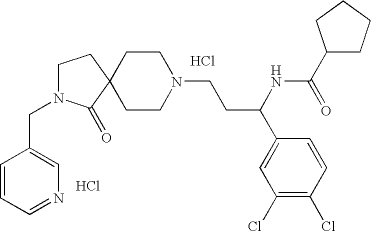 | Cyclopentanecarboxylic acid [1- (3,4-dichloro-phenyl)-3-(1-oxo-2- pyridin-3-ylmethyl-2,8-diaza- spiro[4.5]dec-8-yl)-propyl]-amide dihydrochloride | 616.457 | 98% (LC/MS) |
| 509 | 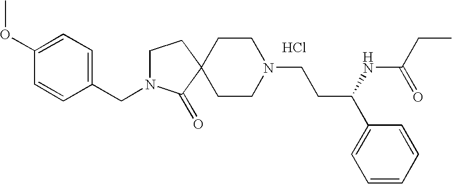 | N-{(S)-3-[2-(4-Methoxy-benzyl)-1- oxo-2,8-diaza-spiro[4.5]dec-8-yl]-1- phenyl-propyl}-propionamide hydrochloride | 500.09 | 100% (LC/MS) |
| 510 | 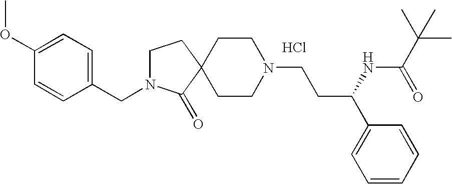 | N-{(S)-3-[2-(4-Methoxy-benzyl)-1- oxo-2,8-diaza-spiro[4.5]dec-8-yl]-1- phenyl-propyl}-2,2-dimethyl- propionamide hydrochloride | 528.14 | 100% (LC/MS) |
| 511 | 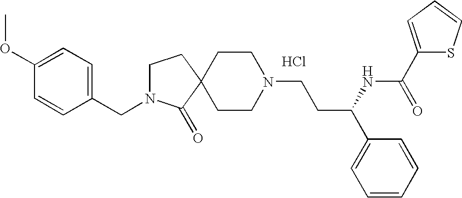 | Thiophene-2-cxarboxcylic acid {(S)-3- [2-(4-methoxy-benzyl)-1-oxo-2,8- diaza-spirto[4.5]dec-8-yl]-1-phenyl- propyl}-amide hydrochloride | 554.16 | 100% (LC/MS) |
| 512 | 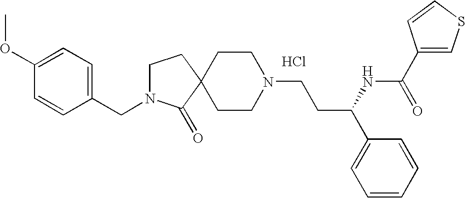 | Thiophene-3-carboxylic acid {(S)-3- [2-(4-methoxy-benzyl)-1-oxo-2,8- diaza-spiro[4.5]dec-8-yl]-1-phenyl- propyl}-amide hydrochloride | 554.16 | 100% (LC/MS) |
| 513 | 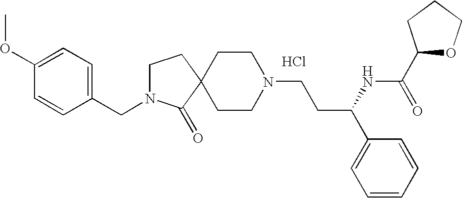 | (R)-Tetrahydro-furan-2-carboxylic acid {(S)-3-[2-(4-methoxy-benzyl)- 1-oxo-2,8-diaza-spiro[4.5]dec-8-yl]- 1-phenyl-propyl}-amide hydrochloride | 542.12 | 100% (LC/MS) |
| 514 | 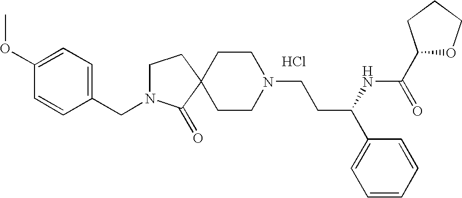 | (S)-Tetrahydro-furan-2-carboxylic acid {(S)-3-[2-(4-methoxy-benzyl)- 1-oxo-2,8-diaza-spirro[4.5]dec-8-yl]- 1-phenyl-propyl}-amide hydrochloride | 542.12 | 100% (LC/MS) |
| 515 | 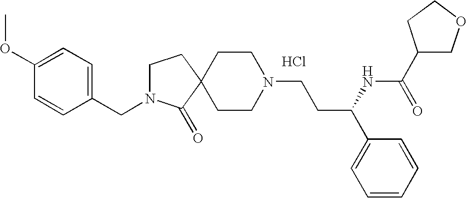 | Tetrahydro-furan-3-carboxylic acid {(S)-3-[2-(4-methoxy-benzyl)-1- oxo-2,8-diaza-spiro[4.5]dec-8-yl]-1- phenyl-propyl}-amide hydrochloride | 542.12 | 100% (LC/MS) |
| 516 | 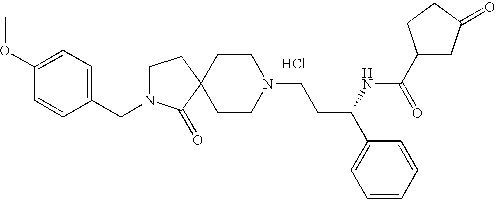 | 3-Oxo-cyclopentanecarboxylic acid {(S)-3-[2-(4-methoxy-benzyl)-1- oxo-2,8-diaza-spiro[4.5]dec-8-yl]-1- phenyl-propyl}-amide hydrochloride | 554.13 | 100% (LC/MS) |
| 517 | 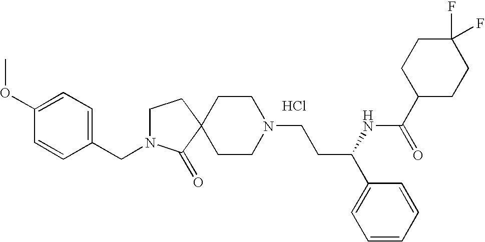 | 4,4-Difluoro-cyclohexanecarboxylic acid {(S)-3-[2-(4-methoxy-benzyl)- 1-oxo-2,8-diaza-spiro[4.5]dec-8-yl]- 1-phenyl-propyl}-amide hydrochloride | 590.16 | 100% (LC/MS) |
| 518 | 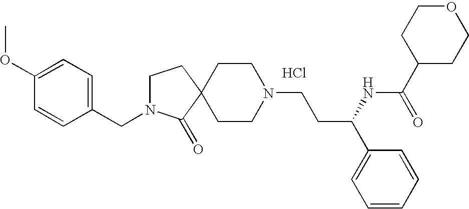 | Tetrahydro-pyran-4-carboxylic acid {(S)-3-[2-(4-methoxy-benzyl)-1- oxo-2,8-diaza-spiro[4.5]dec-8-yl]-1- phenyl-propyl}-amide hydrochloride | 557.14 | 100% (LC/MS) |
| 519 | 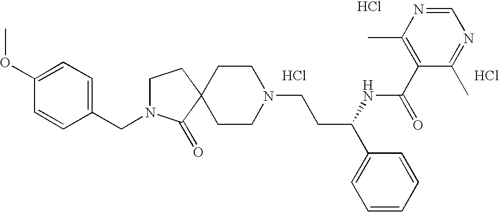 | 4,6-Dimethyl-pyrimidine-5- carboxylic acid {(S)-3-[2-(4- methoxy-benzyl)-1-oxo-2,8-diaza- spiro[4.5]dec-8-yl]-1-phenyl- propyl}-amide trihydrochloride | 651.08 | 100% (LC/MS) |
| 520 | 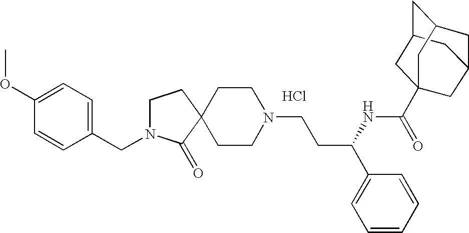 | Adamantane-1-carboxylic acid {(S)-3-[2-(4-methoxy-benzyl)-1- oxo-2,8-diaza-spiro[4.5]dec-8-yl]-1- phenyl-propyl}-amide hydrochloride | 606.25 | 90% (LC/MS) |
| 521 | 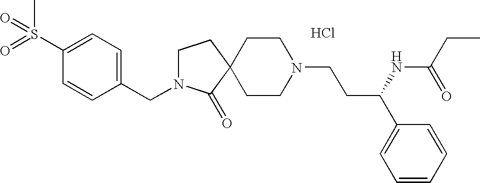 | N-{(S)-3-[2-(4-Methanesulfonyl- benzyl)-1-oxo-2,8-diaza- spiro[4.5]dec-8-yl]-1-phenyl- propyl}-propionamide hydrochloride | 548.15 | 96% (LC/MS) |
| 522 | 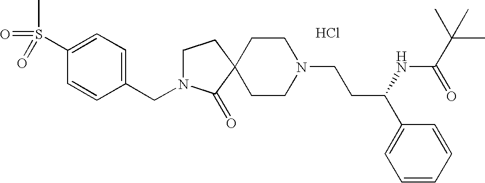 | N-{(S)-3-[2-(4-Methanesulfonyl- benzyl)-1-oxo-2,8-diaza- spiro[4.5]dec-8-yl]-1-phenyl- propyl}-2,2-dimethyl-propionamide hydrochloride | 576.2 | 100% (LC/MS) |
| 523 | 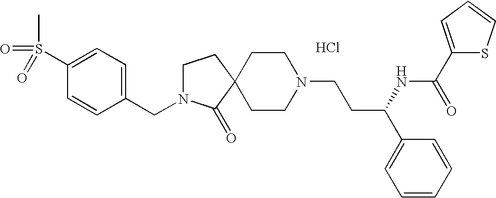 | Thiophene-2-carboxylic acid {(S)-3- [2-(4-methanesulfonyl-benzyl)-1- oxo-2,8-diaza-spiro[4.5]dec-8-yl]-1- phenyl-propyl}-amide hydrochloride | 602.22 | 100% (LC/MS) |
| 524 | 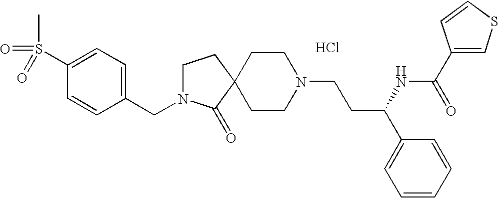 | Thiophene-3-carboxylic acid {(S)-3- [2-(4-methanesulfonyl-benzyl)-1- oxo-2,8-diaza-spiro[4.5]dec-8-yl]-1- phenyl-propyl}-amide hydrochloride | 602.22 | 100% (LC/MS) |
| 525 | 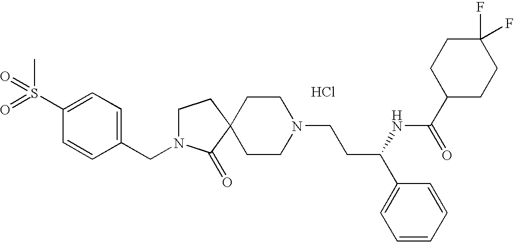 | 4,4-Difluoro-cyclohexanecarboxylic acid {(S)-3-[2-(4-methanesulfonyl- benzyl)-1-oxo-2,8-diaza- spiro[4.5]dec-8-yl]-1-phenyl- propyl}-amide hydrochloride | 638.22 | 100% (LC/MS) |
| 526 | 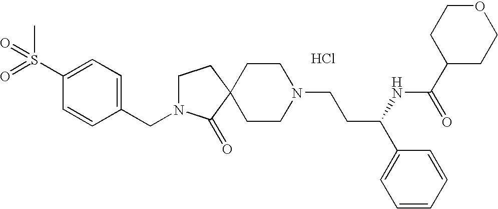 | Tetrahydro-pyran-4-carboxylic acid {(S)-3-[2-(4-methanesulfonyl- benzyl)-1-oxo-2,8-diaza- spiro[4.5]dec-8-yl]-1-phenyl- propyl}-amide hydrochloride | 603.82 | 100% (LC/MS) |
| 527 | 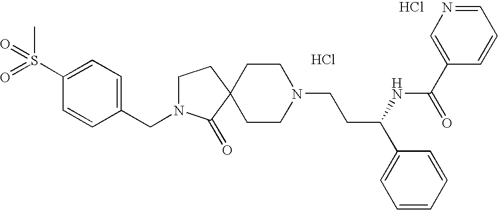 | N-{(S)-3-[2-(4-Methanesulfonyl- benzyl)-1-oxo-2,-diaza- spiro[4.5]dec-8-yl]-1-phenyl- propyl}-nicotinamide dihydrochloride | 633.64 | 100% (LC/MS) |
| 528 | 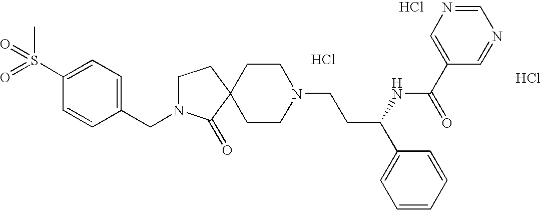 | Pyrimidine-5-carboxylic acid {(S)-3- [2-(4-methanesulfonyl-benzyl)-1- oxo-2,8-diaza-spiro[44.5]dec-8-yl]-1- phenyl-propyl}-amide trihydrochloride | 671.09 | 100% (LC/MS) |
| 529 | 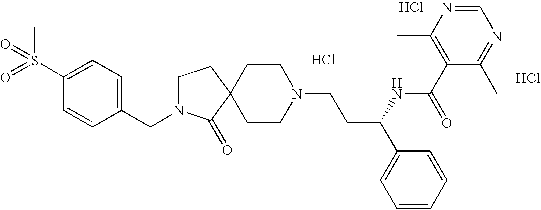 | 4,6-Dimethyl-pyrimidine-5- carboxylic acid {(S)-3-[2-(4- methanesulfonyl-beenzyl)-1-oxo- 2,8-diaza-spiro[4.5]dec-8-yl]-1- phenyl-propyl}-amide trihydrochloride | 699.14 | 90% (LC/MS) |
| 530 | 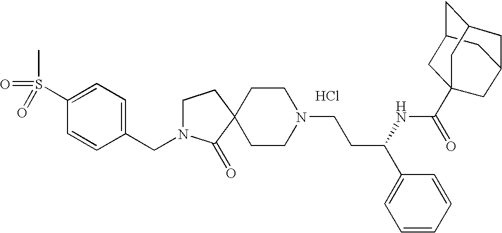 | Adamantene-1-carboxylic acid {(S)-3-[2-(4-methanesulfonyl- benzyl)-1-oxo-2,8-diaza- spiro[4.5]dec-8-yl]-1-phenyl- propyl}-amide hydrochloride | 654.32 | 100% (LC/MS) |
| 531 | 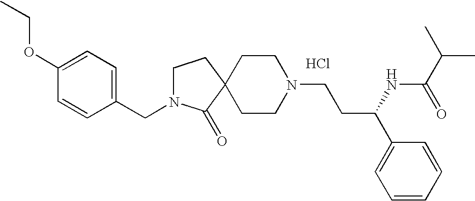 | N-{(S)-3-[2-(4-Ethoxy-benzyl)-1- oxo-2,8-diaza-spiro[4.5]dec-8-yl]-1- phenyl-propyl}-isobutyramide hydrochloride | 528.14 | 100% (LC/MS) |
| 532 | 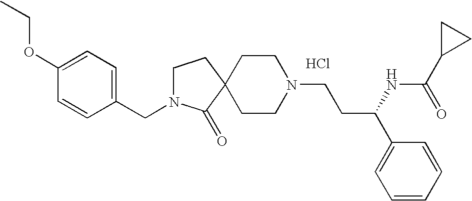 | Cyclopropanecarboxylic acid {(S)- 3-[2-(4-ethoxy-benzyl)-1-oxo-2,8- diaza-spiro[4.5]dec-8-yl]-1-phenyl- propyl}-amide hydrochloride | 526.12 | 100% (LC/MS) |
| 533 | 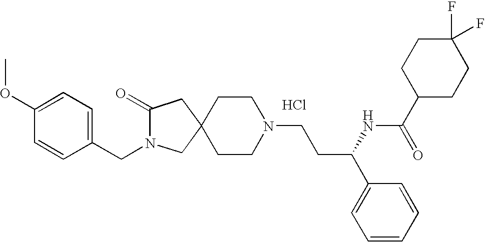 | 4,4-Difluoro-cyclohexanecarboxylic acod {(S)-3-[2-(4-methoxy-benzyl)- 3-oxo-2,8-diaza-spiro[4.5]dec-8-yl]- 1-phenyl-propyl}-amide hydrochloride | 590.16 | 100% (LC/MS) |
| 534 | 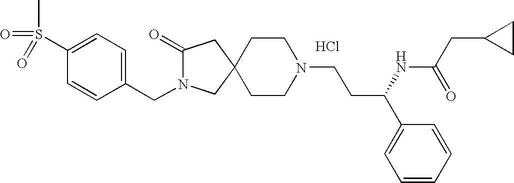 | 2-Cyclopropyl-N-{(S)-3-[2-(4- methanesulfonyl-bgenzyl)-3-oxo- 2,8-diaza-spiro[4.5]dec-8-yl]-1- phenyl-propyl}-acetamide hydrochloride | 574.19 | 100% (LC/MS) |
| 535 | 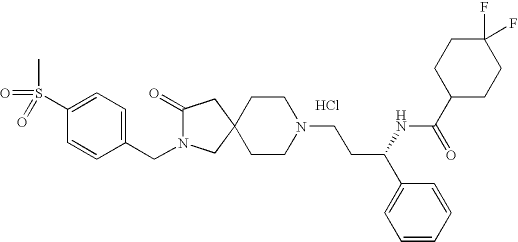 | 4,4-Difluoro-cyclohexanecarboxylic acid {(S)-3-[2-(4-methanesulfonyl- benzyl)-3-oxo-2,8-diaza- spiro[4.5]dec-8-yl]-1-phenyl- propyl}-amide hydrochloride | 638.22 | 100% (LC/MS) |

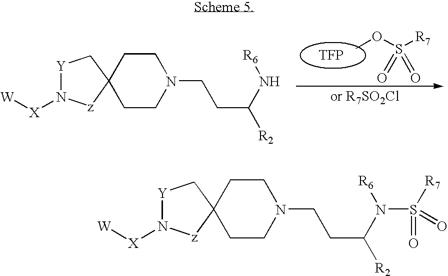

### EXAMPLE 9

### N-{3-[2-(4-Bromobenzyl)-1-oxo-2,8-diaza-spiro[4.5]dec-8-yl]-1-phenylpropyl}-benzenesulfonamide hydrochloride (Compound 536)

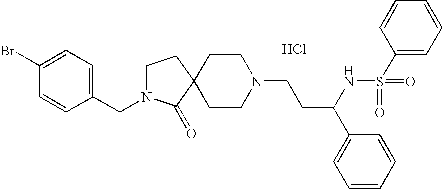 To 83 mg (100 μmol, loading of 1.2 mmol/g) of benzenesulfonate activated ester on polymeric 4-hydroxy-2,3,5,6-tetrafluorobenzamido (TFP) resin (see preparation in J. M. Salvino et al. J. Comb. Chem. 2000, 2, 691-697), preswollen with 0.5 mL of anhydrous DMF, was added 27.3 mg (60 μmol) of 8-(3-amino-3-phenylpropyl)-2-(4-bromobenzyl)-2,8-diaza-spiro[4.5]decan-1-one diluted in 1 mL of DMF. The reaction was agitated overnight at room temperature. The mixture was filtered and washed with DCM (2×2 mL) . The filtrates were collected and evaporated in vacuo. The crude was purified by semi-preparative HPLC (method A) yielding 10.6 mg (27.9%) of Compound 536 as a colorless solid.

¹H NMR (400 MHz, DMSO-d₆): δ[ppm] 9.64 (br s, 1H), 8.44 (d, 1H), 7.53 (m, 4H), 7.44 (t, 1H), 7.35 (m, 2H), 7.12 (m, 7H), 4.33 (m, 3H), 3.35 (m, 2H), 3.16 (t, 3H), 3.05 (m, 1H), 2.9 (m, 2H), 2.08 (m, 1H), 1.95 (m, 4H), 1.82 (m, 1H), 1.56 (br d, 2H).

LC/MS: m/z 598.1 (MH⁺).

Table 4 of compounds illustrates some of the compounds of the present invention that were synthesized using the procedure described in scheme 5.

TABLE 4

| CPD | | | | |
|---|---|---|---|---|
| # | MOL STRUCTURE | COMPOUND NAME | MOL WT | PURITY |
| 537 | 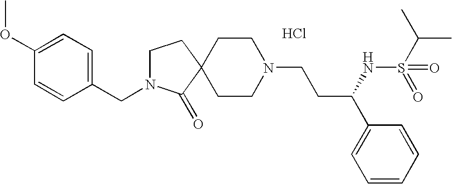 | Propane-2-sulfonic acid {(S)-3-[2-(4- methoy-benzyl)-1-oxo-2,8-diaza- spiro[4.5]dec-8-yl]-1-phenyl-propyl}- amide hydrochloride | 550.16 | 100% (LC/MS) |
| 538 | 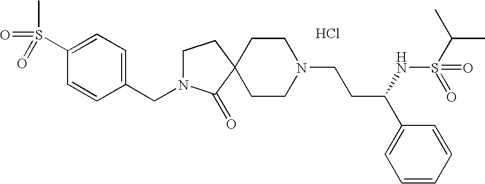 | Propane-2-sulfonic acid {(S)-3-[2-(4- methanesulfonyl-benzyl)-1-oxo-2,8- diaza-spiro[4.5]dec-8-yl]-1-phenyl- propyl}-amide hydrochloride | 598.23 | 90% (LC/MS) |

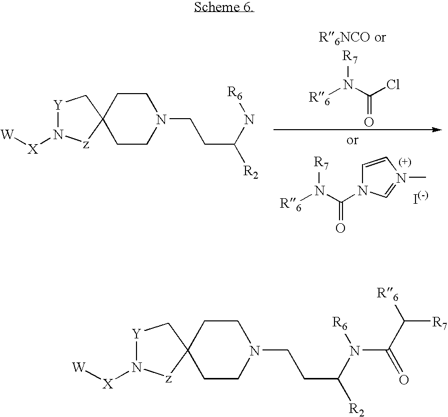

### EXAMPLE 10

### 1-{3-[2-(4-Bromobenzyl)-1-oxo-2,8-diaza-spiro[4.5]dec-8-yl]-1-phenylpropyl}-3-phenyl-urea hydrochloride (Compound 542)

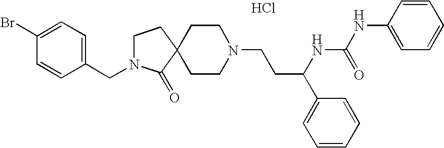 To 27.3 mg (60 μmol) of 8-(3-amino-3-phenylpropyl)-2-(4-bromobenzyl)-2,8-diaza-spiro[4.5]decan-1-one, diluted in 1 mL of anhydrous THF, was added 9.66 mg (80 μmol) of phenylisocyanate dissolved in 0.5 mL of anhydrous THF. The reaction mixture was agitated overnight at room temperature and evaporated in vacuo. The crude was purified by semi-preparative HPLC (method C) yielding 18.9 mg (51.5%) of Compound 542 as a pale yellow solid.

¹H NMR (400 MHz, DMSO-d₆): δ[ppm] 9.45 (br s, 1H), 8.64 (d, 1H), 7.51 (d, 2H), 7.37 (m, 6H), 7.28 (m, 1H), 7.21-7.13 (m, 4H), 6.93 (d, 1H), 6.87 (t×t, 1H), 4.8 (m, 1H), 4.33 (s, 2H), 3.48 (br t, 2H), 3.18-2.94 (m, 5H), 2.15 (m, 2H), 1.96 (m, 4H), 1.83 (m, 1H), 1.6 (br d, 2H).

Table 5 of compounds illustrates some of the compounds of the present invention that were synthesized using the procedure described in scheme 6.

TABLE 5

| CPD | | | | |
|---|---|---|---|---|
| # | MOL STRUCTURE | COMPOUND NAME | MOL WT | PURITY |
| 539 | 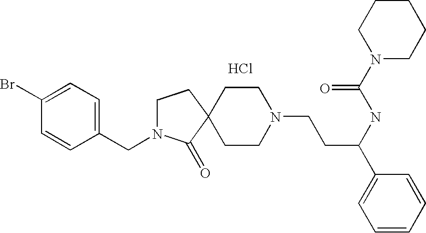 | Piperidine-1-carboxylic acid {3-[2-(4- bromobenzyl)-1-oxo-2,8-diaza- spiro[4.5]dec-8-yl]-1-phenyl-propyl}- amide hydrochloride | 604.03 | 93.5% (LC/MS) |
| 540 | 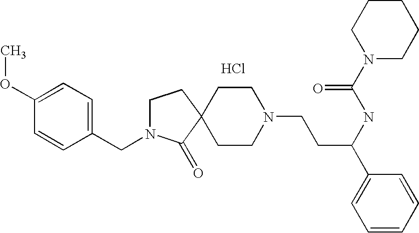 | Piperidine-1-carboxylic acid {3-[2-(4- methoxybenzyl)-1-oxo-2,8-diaza- spiro[4.5]dec-8-yl]-1-phenyl-propyl}- amide hydrochloride | 555.17 | 94.5% (LC/MS) |
| 541 | 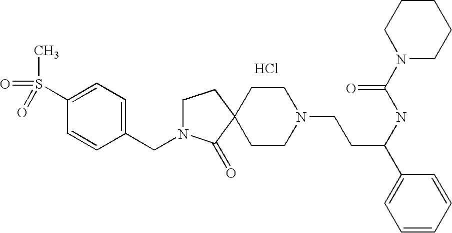 | Piperidine-1-carboxylic acid {3-[2-(4- methanesulfonylbenzyl)-1-oxo-2,8- diaza-spiro[4.5]dec-8-yl]-1-phenyl- propyl}-amide hydrochloride | 603.224 | 93.3% (LC/MS) |
| 542 | 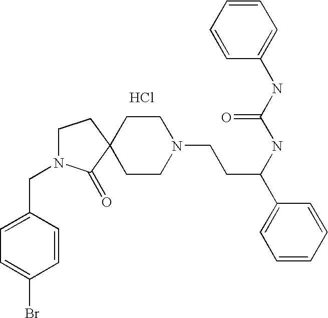 | 1-{3-[2-(4-bromophenyl)-1-oxo-2,8- diaza-spiro[4.5]dec-8-yl]-1-phenyl- propyl}-3-phenyl-urea hydrochloride | 612.008 | 98+(LC/MS) |
| 543 | 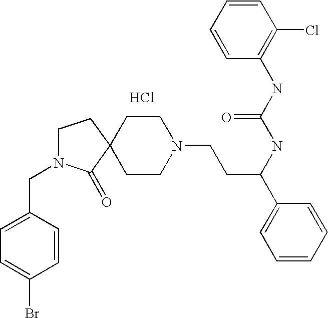 | 1-0{3-[2-(4-bromobhenzyl)-1-oxo-2,8- diaza-spiro[4.5]dec-8-yl]-1-phenyl- propyl}-3-(2-chloro-phenyl)-urea hydrochloride | 646.453 | 94.6% (LC/MS) |
| 544 | 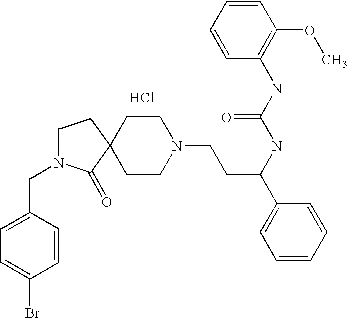 | 1-{3-[2-(4-bromobenzyl)-1-oxo-2,8- diaza-spiro[4.5]dec-8-yl]-1-phenyl- propyl}-3-(2-methoxy-phenyl)-urea hydrochloride | 642.034 | 98+(LC/MS) |
| 545 | 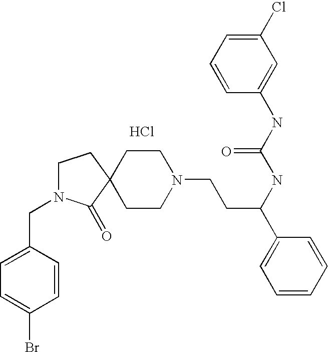 | 1-{3-[2-(4-bromobenzyl)-1-oxo-2,8- diaza-spiro[4.5]dec-8-yl]-1-phenyl- propyl}-3-(3-chloro-phenyl)-urea hydrochloride | 646.453 | 98+(LC/MS) |
| 546 | 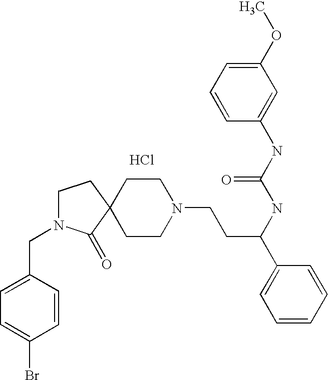 | 1-{3-[2-(4-bromobenzyl)-1-oxo-2,8- diaza-spiro[4.5]dec-8-yl]-1-phenyl- propyl}-3-(3-methoxy-phenyl)-urea hydrochloride | 642.034 | 98+(LC/MS) |
| 547 | 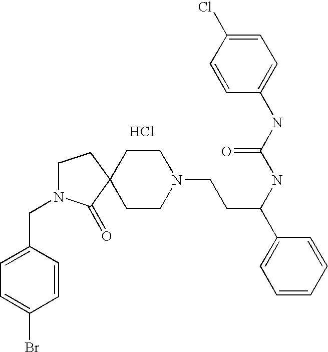 | 1-{3-([2-(4-bromobenzyl)-1-oxo-2,8- diaza-spiro[4.5]dec-8-yl]-1-phenyl- propyl}-3-(4-chloro-phenyl)-urea hydrochloride | 646.453 | 98+(LC/MS) |
| 548 | 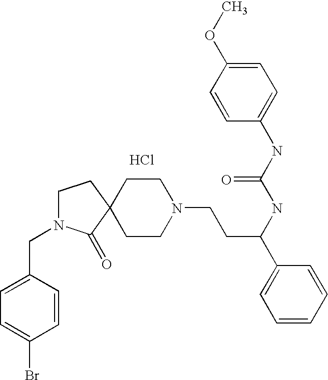 | 1-{3-[2-(4-bromobenzyl)-1-oxo-2,8- diaza-spiro[4.5]dec-8-yl]-1-phenyl- propyl}-3-(4-methoxy-phenyl)-urea hydrochloride | 642.034 | 98+(LC/MS) |
| 549 | 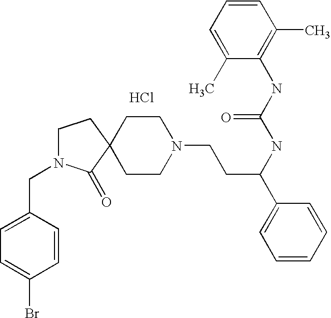 | 1-{3-[2-(4-bromobenzyl)-1-oxo-2,8- diaza-spiro[4.5]dec-8-yl]-1-phenyl- propyl}-3-(2,6-dimethyl-phenyl)-urea hydrochloride | 640.062 | 98+(LC/MS) |
| 550 | 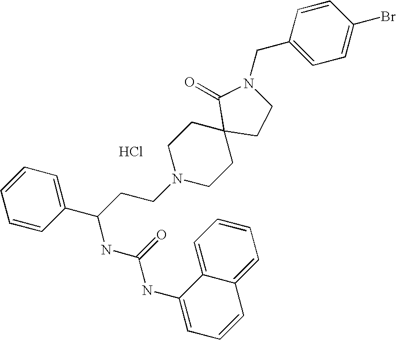 | 1-{3-[2-(4-bromobenzyl)-1-oxo-2,8- diaza-spiro[4.5]dec-8-yl]-1-phenyl- propyl}-3-naphtalen-1-yl-urea hydrochloride | 662.068 | 98+(LC/MS) |
| 551 | 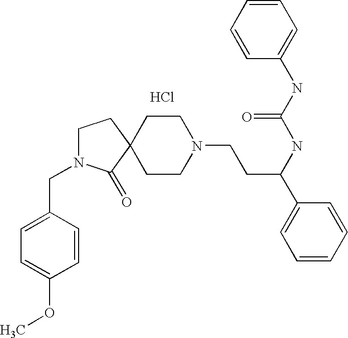 | 1-{3-[2-(4-methoxybenzyl)-1-oxo-2,8- diaza-spiro[4.5]dec-8-yl]-1-phenyl- propyl}-3-phenyl-urea hydrochloride | 563.138 | 98+(LC/MS) |
| 552 | 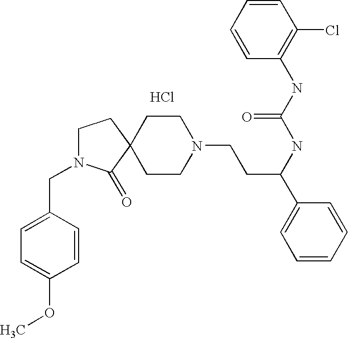 | 1-(2-chlorophenyl)-3-{3-[2-(4- methoxybenzyl)-1-oxo-2,8-diaza- spiro[4.5]dec-8-yl]-1-phenyl-propyl}- urea hydrochloride | 597.583 | 95+(LC/MS) |
| 553 | 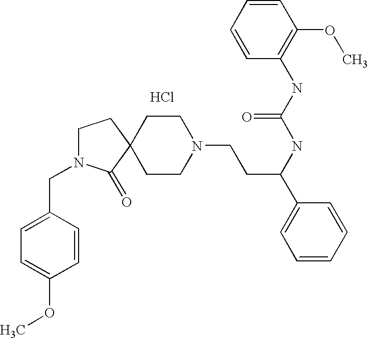 | 1-{3-[2-(4-methoxybenzyl)-1-oxo-2,8- diaza-spiro[4.5]dec-8-yl]-1-phenyl- propyl}-3-(2-methoxyphenyl)-urea hydrochloride | 593.164 | 98+(LC/MS) |
| 554 | 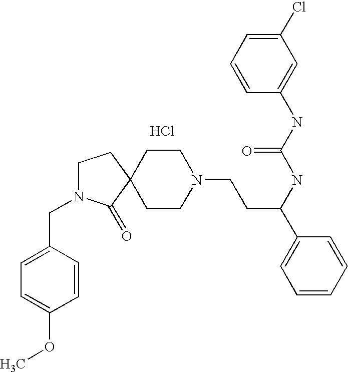 | 1-(3-chlorophenyl)-3-{3-[2-(4- methoxybenzyl)-1-oxo-2,8-diaza- spiro[4.5]dec-8-yl]-1-phenyl-propyl}- urea hydrochloride | 597.583 | 98+(LC/MS) |
| 555 | 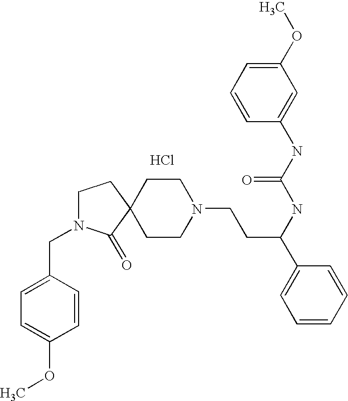 | 1-{3-[2-(4-methoxybenzyl)-1-oxo-2,8- diaza-spiro[4.5]dec-8-yl]-1-phenyl- propyl}-3-(3-methoxyphenyl)-urea hydrochloride | 593.164 | 98+(LC/MS) |
| 556 | 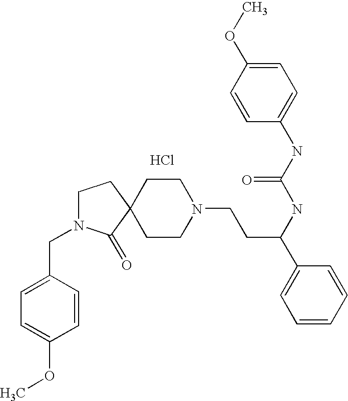 | -{3-[2-(4-methoxybenzyl)-1-oxo-2,8- diaza-spiro[4.5]dec-8-yl]-1-phenyl- propyl}-3-(4-methoxyphenyl)-urea hydrochloride | 593.164 | 98+(LC/MS) |
| 557 | 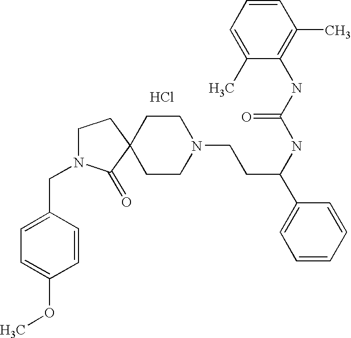 | 1-(2,6-dimethylphenyl)-33-{3-[2-(4- methoxybenzyl)-1-oxo-2,8-diaza- spiro[4.5]dec-8-yl]-1-phenyl-propyl}- urea hydrochloride | 591.192 | 98+(LC/MS) |
| 558 | 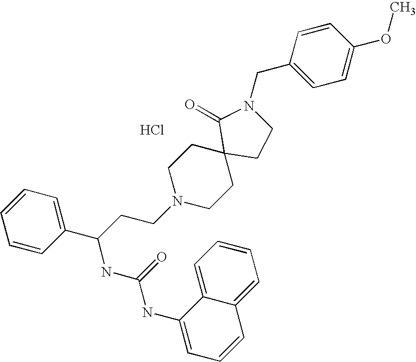 | 1-{3-[2-(4-methoxybenzyl)-1-oxo-2,8- diaza-spiro[4.5]dec-8-yl]-1-phenyl- propyl}-3-naphthalen-1-yl-urea hydrochloride | 613.198 | 98+(LC/MS) |
| 559 | 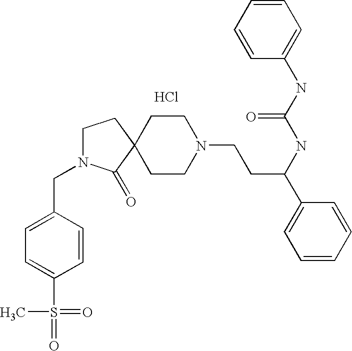 | 1-{3-[2-(4-methanesulfonylbenzyl)-1- oxo-2,8-diaza-spiro[4.5]dec-8-yl]-1- phenyl-propyl}-3-phenyl-urea hydrochloride | 611.203 | 98+(LC/MS) |
| 560 | 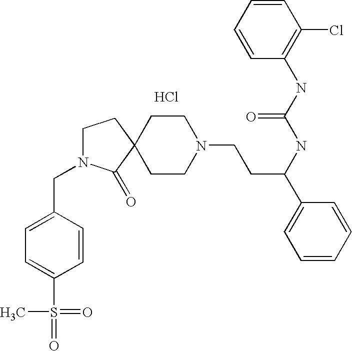 | 1-(2-chlorophenyl)-3-{3-[2-(4- methanesulfonylbenzyl)-1-oxo-2,8- diaza-spiro[4.5]dec-8-yl]-1-phenyl- propyl}-urea hydrochloride | 645.648 | 98+(LC/MS) |
| 561 | 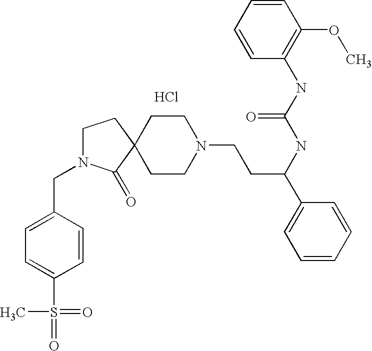 | 1-{3-[2-(4-methanesulfonylbenzyl)- oxo-2,8-diaza-spiro[4.5]dec-8-yl]-1- phenyl-propyl}-3-(2-methoxyphenyl)- urea hydrochloride | 641.229 | 98+(LC/MS) |
| 562 | 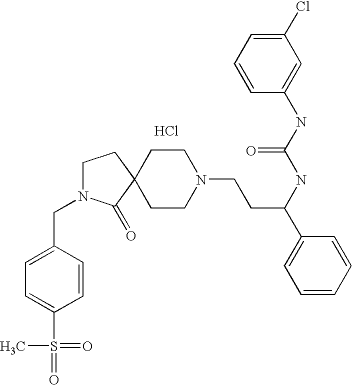 | 1-(3-chlorophenyl)-3-{3-[2-(4- methanesulfonylbernzyl)-1-oxo-2,8- diaza-spiro[4.5]dec-8-yl]-1-phenyl- propyl}-urea hydrochloride | 645.648 | 98+(LC/MS) |
| 563 | 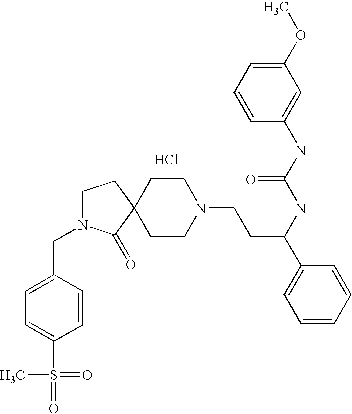 | 1-{3-[2-(4-methanesulfonylbeenzyl)-1- oxo-2,8-diaza-spiro[4.5]dec-8-yl]-1- phenyl-propyl}-3-(3-methoxyphenyl)- urea hydrochloride | 641.229 | 98+(LC/MS) |
| 564 | 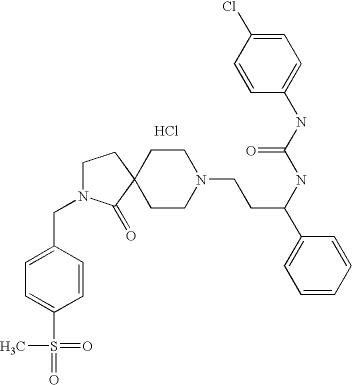 | 1-(4-chlorophenyl)-3-{3-[2-(4- methanesulfonylbenzyl)-1-oxo-2,8- diaza-spiro[4.5]dec-8-yl]-1-phenyl- propyl}-urea hydrochloride | 645.648 | 98+(LC/MS) |
| 565 | 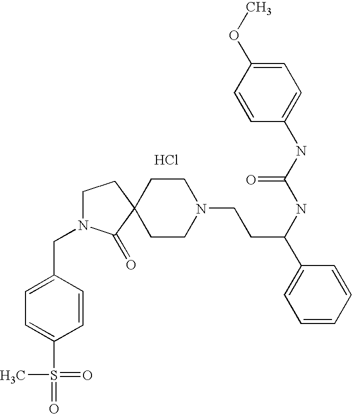 | 1-‡3-[22-(4-methanesulfonylbenzyl)-1- oxo-2,8-diaza-spiro[4.5]dec-8-yl]-1- phenyl-propyl}-3-(4-methoxyphenyl)- urea hydrochloride | 641.229 | 98+(LC/MS) |
| 566 | 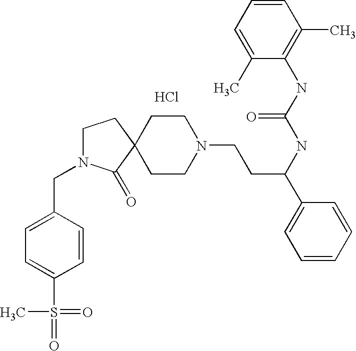 | 1-(2,6-dimethylphenyl)-3-{3-[2-(4- methanesulfonylbenzyl)-1-oxo-2,8- diaza-spiro[4.5]dec-8-yl]-1-phenyl- propyl}-urea hydrochloride | 639.527 | 98+(LC/MS) |
| 567 | 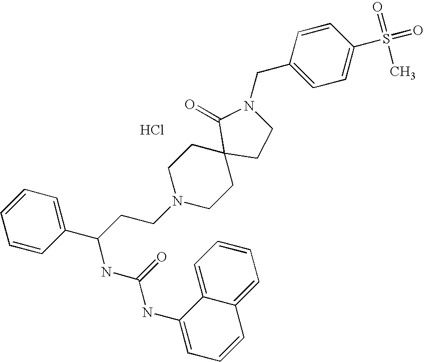 | 1-{3-[2-(4-methanesulfonylnemzyl)-1- oxo-2,8-diaza-spiro[4.5]dec-8-yl]-1- phenyl-propyl}-3-naphthalen-1-yl- urea hydrochloride | 661.263 | 98+(LC/MS) |

Table 5a of compounds illustrates some additional compounds of the present invention that were synthesized using the procedure described in scheme 6.

TABLE 5a

| CPD | | | | PUR- |
|---|---|---|---|---|
| # | MOL STRUCTURE | COMPOUND NAME | MOL WT | ITY |
| 568 | 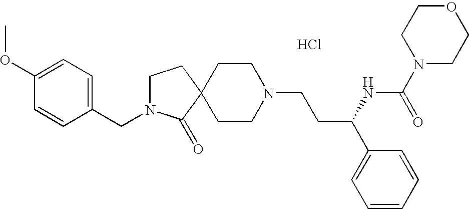 | Morpholine-4-carboxylic acid {(S)-3- [2-(4-methoxy-benzyl)-1-oxo-2,8- diaza-spiro[4.5]dec-8-yl]-1-phenyl- propyl}-amide hydrochloride | 557.14 | 100% (LC/ MS) |
| 569 | 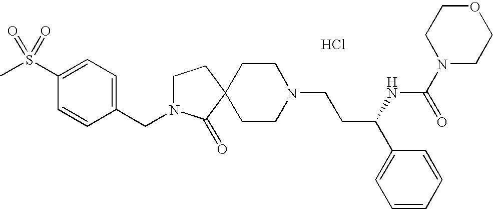 | Morpholine-4-carboxylic acid {(S)-3- [2-(4-methanesulfonyl-benzyl)-1-oxo- 2,8-diaza-spiro[4.5]dec-8-yl]-1- phenyl-propyl}-amide hydrochloride | 605.2 | 90% (LC/ MS) |
| 570 | 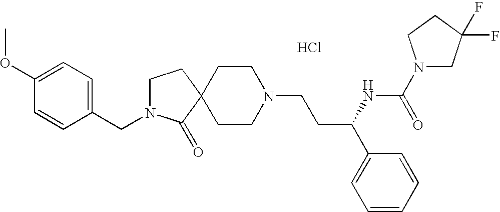 | 3,3-Difluoro-pyrolidine-1-carboxylicp acid {(S)-3-[2-(4-methoxy-benzyl)-1- oxo-2,8-diaza-spiro[4.5]dec-8-yl]-1- phenyl-propyl}-amide hydrochloiride | 577.12 | 100% (LC/ MS) |
| 571 | 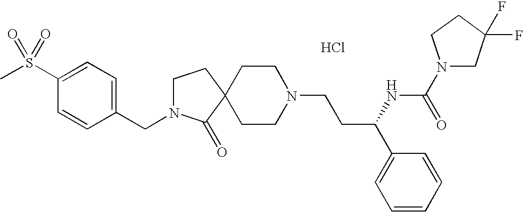 | 3,3-Difluoro-pyrrolidine-1-carboxylic acid {(S)-3-[2-(4-methanesulfonyl- benzyl)-1-oxo-2,8-diaza- spiro[4.5]dec-8-yl]-1-phenyl-propyl}- amide hydrochloride | 625.18 | 100% (LC/ MS) |

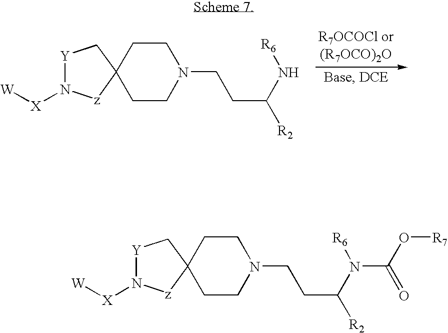

### EXAMPLE 11

### (3-[2-(4-Methanesulfonylbenzyl)-1-oxo-2,8-diaza-5 spiro[4.5]dec-8-yl]-1-phenylpropyl}-carbamic acid methyl ester hydrochloride (Compound 572)

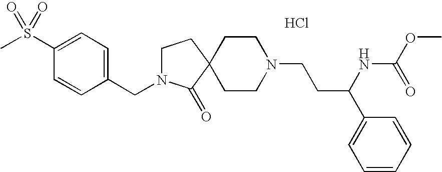 To a stirred solution of 8-(3-amino-3-phenylpropyl)-2-(4-methanesulfonylbenzyl)-2,8-diaza-spiro[4.5]decan-1-one (27.3 mg, 0.06 mmol) in DMF-DCE (1:1, 1.0 mL) was sequentially added triethylamine (0.01 mL, 0.072 mmol) and dimethyldicarbonate (0.07 mL, 0.066 mmol). The reaction mixture was stirred overnight at room temperature, concentrated and purified by semi-preparative HPLC (method D) affording Compound 572 (17.2 mg, 52%) as a white powder.

¹H NMR (400 MHz, DMSO-d₆): δ[ppm] 9.62 (br s, 1H), 7.88 (d, 2H), 7.44 (d, 2H), 7.33 (m, 4H), 7.25 (m, 1H), 4.59 (m, 1H), 4.47 (s, 2H), 3.50 (s, 3H), 3.42 (m, 2H), 3.22 (t, 2H), 3.19 (s, 3H), 3.09-2.91 (m, 4H), 2.12-1.95 (m, 6H), 1.86 (t, 1H), 20 1.64 (br d, 2H)

LC/MS: m/z 513.6 (MH⁺).

Table 6 of compounds illustrates some of the compounds of the present invention that were synthesized using the procedure described in scheme 7.

TABLE 6

| CPD | | | MOL | |
|---|---|---|---|---|
| # | MOL STRUCTURE | COMPOUND NAME | WT | PURITY |
| 572 | 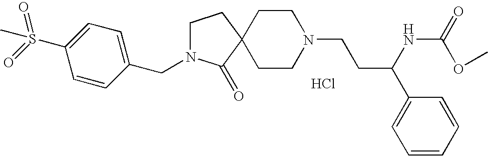 | {3-[2-(4-methanesulfonylbenzyl)-1- oxo-2,8-diaza-spiro[4.5]dec-8-yl]-1- phenyl-propyl}-carbamic methyl ester hydrochloride | 550.116 | 98+(LC/MS) |
| 573 | 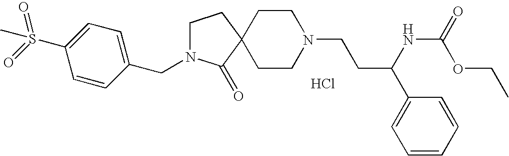 | {3-[2-(4-methanesulfonylbenzyl)-1- oxo-2,8-diaza-spiro[4.5]dec-8-yl]-1- phenyl-propyl}-carbamic acid ethyl ester hydrochloride | 564.143 | 98+(LC/MS) |

Table 6a of compounds illustrates some additional compounds of the present invention that were synthesized using the procedure described in scheme 7.

TABLE 6a

| CPD | | | | |
|---|---|---|---|---|
| # | MOL STRUCTURE | COMPOUND NAME | MOL WT | PURITY |
| 574 | 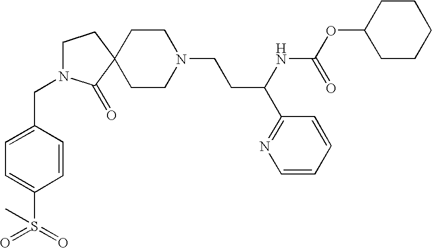 | {3-[2-(4-Methanesulfonyl-benzyl)-1- oxo-2,8-diaza-spiro[4.5]dec-8-yl]-1- pyridin-2-yl-propyl}-carbamic acid cyclohexyl ester | 582.77 | 83% (LC/MS) |
| 575 | 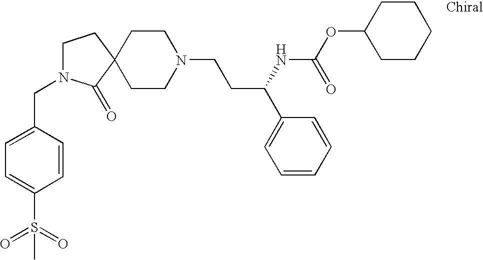 | {(S)-3-[2-(4-Methanesulfonyl- benzyl)-1-oxo-2,8-diaza- spiro[4.5]dec-8-yl]-1-phenyl- propyl}-carbamic acid cyclohexyl ester | 581.78 | 99% (LC/MS) |
| 576 | 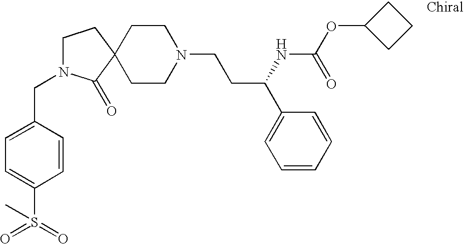 | {(S)-3-[2-(4-Methanesulfonyl- benzyl)-1-oxo-2,8-diaza- spiro[4.5]dec-8-yl]-1-phenyl- propyl}-carbamic acid cyclobutyl ester | 553.73 | 99% (LC/MS) |
| 577 | 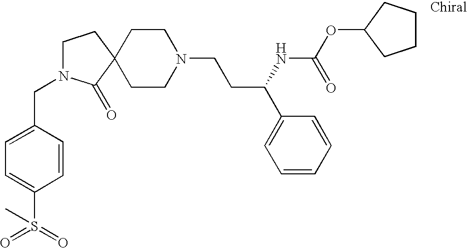 | {(S)-3-[2-(4-Methanesulfonyl- benzyl)-1-oxo-2,8-diaza- spiro[4.5]dec-8-yl]-1-phenyl- propyl}-carbamic acid cyclopentyl ester | 567.75 | 99% (LC/MS) |
| 578 | 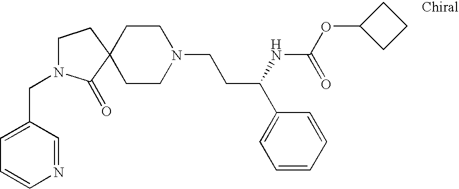 | [(S)-3-(1-Oxo-2-pyridin-3-ylmeethyl- 2,8-diaza-spiro[4.5]dec-8-yl)-1- phenyl-propyl]-carbamic acid cyclobutyl ester | 476.62 | 98% (LC/MS) |
| 579 | 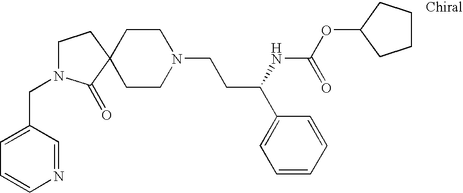 | [(S)-3-(1-Oxo-2-pyridin-3-ylmethyl- 2,8-diaza-spiro[4.5]dec-8-yl)-1- phenyl-propyl]-carbamic acid cyclopentyl ester | 490.65 | 99% (LC/MS) |
| 580 | 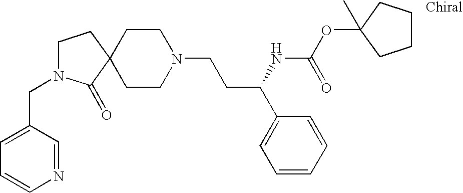 | [(S)-3-(1-Oxo-2-pyridin-3-ylmethyl- 2,8-diaza-spiro[4.5]dec-8-yl)-1- phenyl-propyl]-carbamic acid 1- methyl-cyclopentyl ester | 504.68 | 97% (LC/MS) |
| 581 | 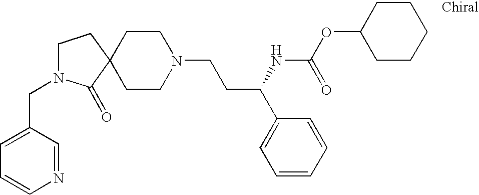 | [(S)-3-(1-Oxo-2-pyridin-3-ylmethyl- 2,8-diaza-spiro[4.5]dec-8-yl)-1- phenyl-propyl]-carbamic acid cyclohexyl ester | 504.68 | 100% (LC/MS) |
| 582 | 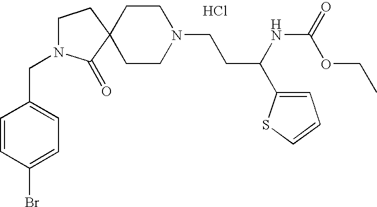 | {3-[2-(4-Bromo-benzyl)-1-oxo-2,8- diaza-spiro[4.5]dec-8-yl]-1- thiophen-2-yl-propyl}-carbamic acid ethyl ester hydrochloride | 570.98 | 98+(LC/MS) |
| 583 | 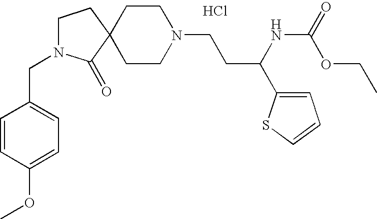 | {3-[2-(4-Methoxy-benzyl)-1-oxo- 2,8-diaza-spiro[4.5]dec-8-yl]-1- thiophen-2-yl-propyl}-carbamic acid ethyl ester hydrochloride | 522.11 | 98+(LC/MS) |
| 584 | 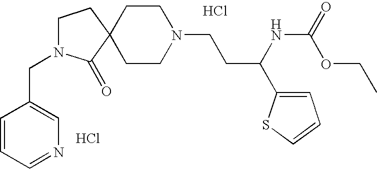 | [3-(1-Oxo-2-pyridin-3-ylmethyl-2,8- diaza-spiro[4.5]dec-8-yl)-1- thiophen-2-yl-propyl]-carbamic acid ethyl ester dihydrochloride | 529.53 | 98+(LC/MS) |
| 585 | 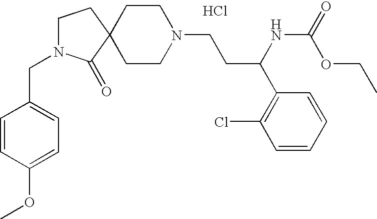 | {1-(2-Chloro-phenyl)-3-[2-(4- methoxy-benzyl)-1-oxo-2,8-diaza- spiro[4.5]dec-8-yl]propyl}- carbamic acid ethyl ester hydrochloride | 550.52 | 98+(LC/MS) |
| 586 | 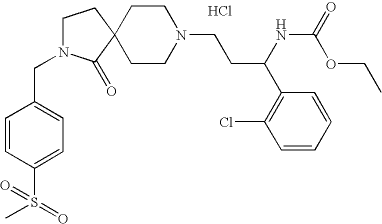 | {1-(2-Chloro-phenyl)-3-[2-(4- methanesulfonyl-benzyl)-1-oxo- 2,8-diaza-spiro[4.5]dec-8-yl]- propyl}-carbamic acid ethyl ester hydrochloride | 598.59 | 98+(LC/MS) |
| 587 | 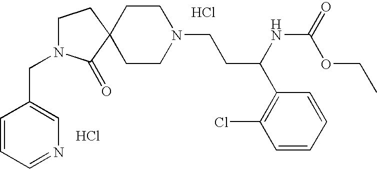 | [1-(2-Chloro-phenyl)-3-(1-oxo-2- pyridin-3-ylmethyl-2,8-diaza- spiro[4.5]dec-8-yl)-propyl]- carbamic acid ethyl ester dihydrochloride | 557.95 | 98+(LC/MS) |
| 588 | 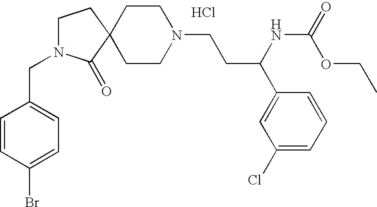 | [3-[2-(4-Bromo-benzyl)-1-oxo-2,8- diaza-spiro[4.5]dec-8-yl]-1-(2- chloro-phenyl)-propyl]-carbamic acid ethyl ester hydrochloride | 599.4 | 98+(LC/MS) |
| 589 | 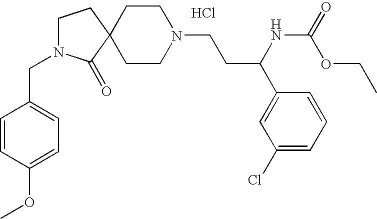 | {1-(3-Chloro-phenyl)-3-[2-(4- methoxy-benzyl)-1-oxo-2,8-diaza- spiro[4.5]dec-8-yl]propyl}- carbamic acid ethyl ester hydrochloride | 550.52 | 98+(LC/MS) |
| 590 | 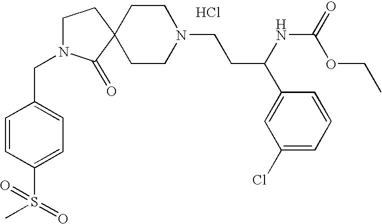 | {1-(3-Chloro-phenyl)-3-[2-(4- methanesulfonyl-benzyl)-1-oxo- 2,8-diaza-spiro[4.5]dec-8-yl]- propyl}-carbamic acid ethyl ester hydrochloride | 598.59 | 98+(LC/MS) |
| 591 | 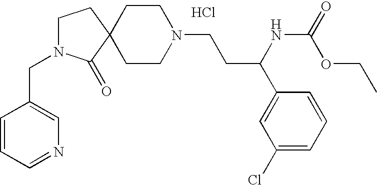 | [1-(3-Chloro-phenyl)-3-(1-oxo-2- pyridin-3-ylmethyl-2,8-diaza- spiro[4.5]dec-8-yl)-propyl]- carbamic acid ethyl ester dihydrochloride | 557.95 | 98+(LC/MS) |
| 592 | 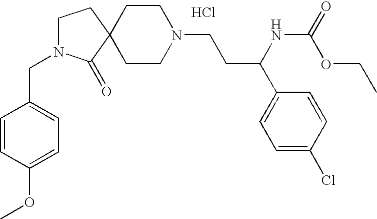 | {1-(4-Chloro-phenyl)-3-[2-(4- methoxy-benzyl)-1-oxo-2,8-diaza- spiro[4.5]dec-8-yl]-propyl}- varbamic acid ethyl ester hydrochloride | 550.52 | 98+(LC/MS) |
| 593 | 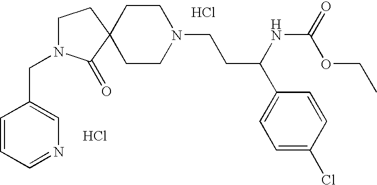 | [1-(4-Chloro-phenyl)-3-(1-oxo-2- pyridin-3-ylmethyl-2,8-diaza- spiro[4.5]dec-8-yl)-propyl]- carbamic acid ethyl ester dihydrochloride | 557.95 | 98+(LC/MS) |
| 594 | 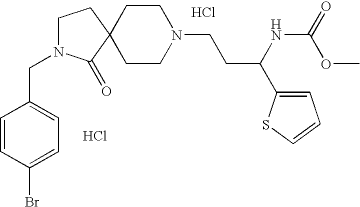 | {3-[2-(4-Bromo-benzyl)-1-oxo-2,8- diaza-spiro[4.5]dec-8-yl]-1- thiophen-2-yl-propyl}-carbamic acid methyl ester hydrochloride | 556.95 | 98+(LC/MS) |
| 595 | 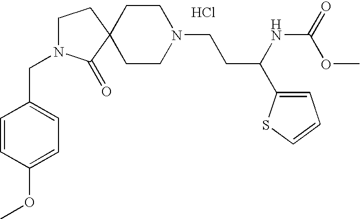 | {3-[2-(4-Methoxy-benzyl)-1-oxo- 2,8-diaza-spiro[4.5]dec-8-yl]-1- thiophen-2-yl-propyl}-carbamic acid methyl ester hydrochloride | 508.08 | 98+(LC/MS) |
| 596 | 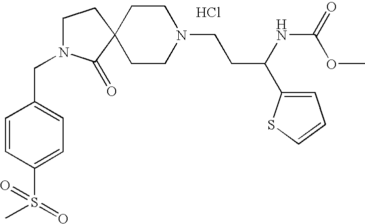 | {3-[2-(4-Methanesulfonyl=benzyl)-1- oxo-2,8-diaza-spiro[4.5]dec-8-yl]-1- thiophen-2-yl-propyl}-carbamic acid methyl ester hydrochloride | 556.14 | 98+(LC/MS) |
| 597 | 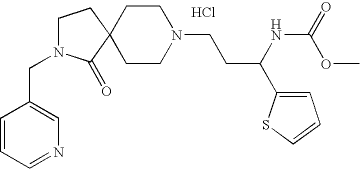 | [3-(1-Oxo-2-pyridin-3-ylmethyl-2,8- diaza-spiro[4.5]dec-8-yl)-1- thiophen-2-yl-propyl]-carbamic acid methyl ester dihydrochloride | 515.5 | 98+(LC/MS) |
| 598 | 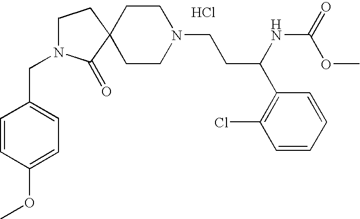 | {1-(2-Chloro-phenyl)-3-[2-(4- methoxy-benzyl)-1-oxo-2,8-diaza- spiro[4.5]dec-8-yl]-propyl}- carbamic acid methyl ester hydrochloride | 536.5 | 98+(LC/MS) |
| 599 | 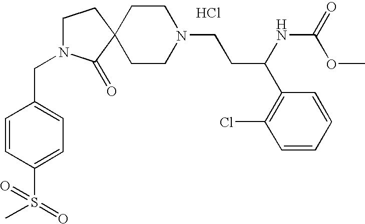 | {1-(2-Chloro-phenyl)-3-[2-(4- methanesulfonyl-benzyl)-1-oxo- 2,8-diaza-spiro[4.5]dec-8-yl]- propyl}-carbamic acid methyl ester hydrochloride | 554.56 | 98+(LC/MS) |
| 600 | 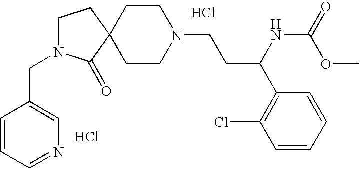 | [1-(2-Chloro-phenyl)-3-(1-oxo-2- pyridin-3-ylmethyl-2,8-diaza- spiro[4.5]dec-8-yl)-propyl]- carbamic acid methyl ester dihydrochloride | 543.92 | 98+(LC/MS) |
| 601 | 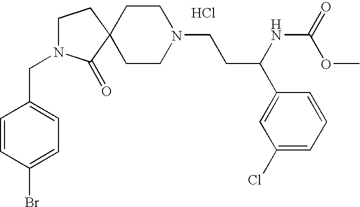 | [3-[2-(4-Bromo-benzyl)-1-oxo-2,8- diaza-spiro[4.5]dec-8-yl]-1-(3- chloro-phenyl)-propyl]-carbamic acid methyl ester hydrochloride | 585.37 | 98+(LC/MS) |
| 602 | 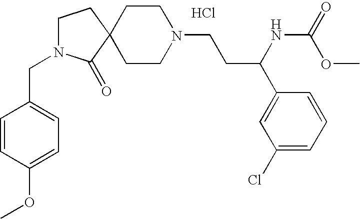 | {1-(3-Chloro-phenyl)-3-[2-(4- methoxy-benzyl)-1-oxo-2,8-diaza- spiro[4.5]dec-8-yl]-propyl}- carbamic acid methyl ester hydrochloride | 536.5 | 98+(LC/MS) |
| 603 | 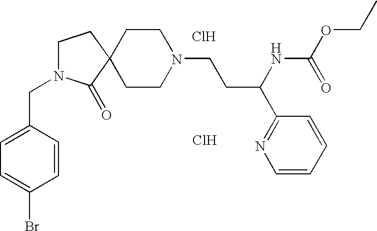 | {3-[2-(4-Bromo-benzyl)-1-oxo-2,8- diaza-spiro[4.5]dec-8-yl]-1-pyridin- 2-yl-propyl}-carbamic acid ethyl ester hydrochloride | 602.41 | 98% (LC/MS) |
| 604 | 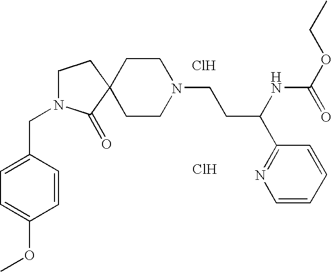 | {3-[2-(4-Methoxy-benzyl)-1-oxo- 2,8-diaza-spiro[4.5]dec-8-yl]-1- pyridin-2-yl-propyl}-carbamic acid ethyl ester dihydrochloride | 553.53 | 98% (LC/MS) |
| 605 | 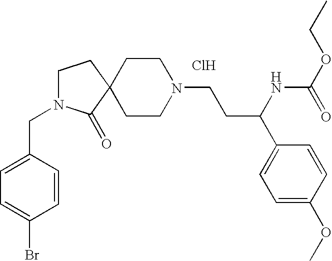 | [3-[2-(4-Bromo-benzyl)-1-oxo-2,8- diaza-spiro[4.5]dec-8-yl]-1-(4- methoxy-phenyl)-propyl]-carbamic acid ethyl ester hydrochloride | 594.99 | 98% (LC/MS) |
| 606 | 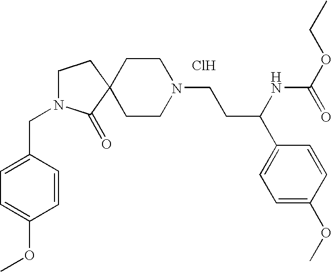 | [3-[2-(4-Methoxy-benzyl)-1-oxxo- 2,8-diaza-spiro[4.5]dec-8-yl]-1-(4- methoxy-phenyl)-propyl]-carbamic acid ethyl ester hydrochloride | 546.11 | 98% (LC/MS) |
| 607 | 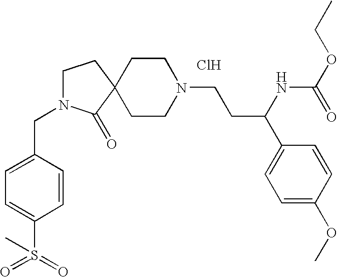 | [3-[2-(4-Methanesulfonyl-benzyl)-1- oxo-2,8-diaza-spiro[4.5]dec-8-yl]-1- (4-methoxy-phenyl)-propyl]- carbamic acid ethyl ester hydrochloride | 594.17 | 98% (LC/MS) |
| 608 | 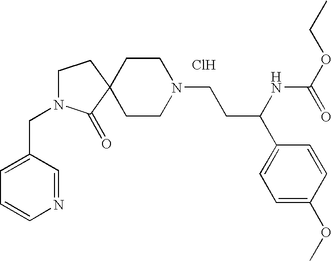 | [1-(4-Methoxy-phenyl)-3-(1-oxo-2- pyridin-3-ylmethyl-2,8-diaza- spiro[4.5]dec-8-yl)-propyl]- carbamic acid ethyl ester dihydrochloride | 553.53 | 98% (LC/MS) |
| 609 | 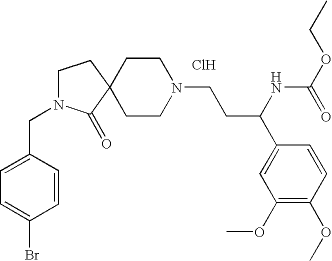 | [3-[2-(4-Bromo-benzyl)-1-oxo-2,8- diaza-spiro[4.5]dec-8-yl]-1-(3,4- dimethoxy-phenyl)-propyl]- carbamic acid ethyl ester hydrochloride | 625.01 | 98% (LC/MS) |
| 610 | 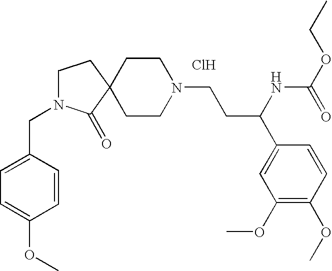 | {1-(3,4-Dimethoxy-phenyl)-3-[2-(4- methoxy-benzyl)-1-oxo-2,8-diaza- spiro[44.5]dec-8-yl]-propyl}- carbamic acid ethyl ester hydrochloride | 576.14 | 98% (LC/MS) |
| 611 | 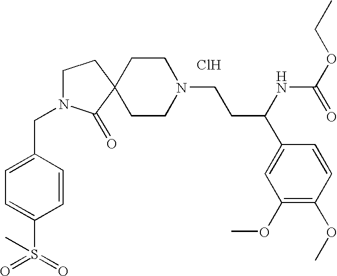 | {1-(3,4-Dimethoxy-phenyl)-3-[2-(4- methanesulfonyl-benzyl)-1-oxo- 2,8-diaza-spiro[4.5]dec-8-yl]- propyl}-carbamic acid ethyl ester hydrochloride | 624.2 | 98% (LC/MS) |
| 612 | 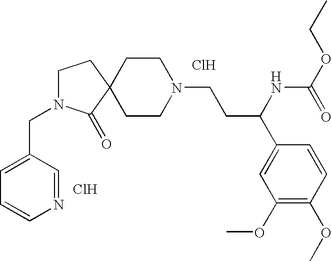 | [1-(3,4-Dimethoxy-phenyl)-3-(1- oxo-2-pyridin-3-ylmethyl-2,8-diaza- spiro[4.5]dec-8-yl)-propyl]- carbamic acid ethyl ester dihydrochloride | 583.56 | 98% (LC/MS) |
| 613 | 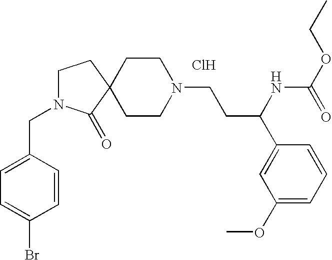 | [3-[2-(4-Bromo-benzyl)-1-oxo-2,8- diaza-spiro[4.5]dec-8-yl]-1-(3- methoxy-phenyl)-propyl]-carbamic acid ethyl ester hydrochloride | 594.99 | 98% (LC/MS) |
| 614 | 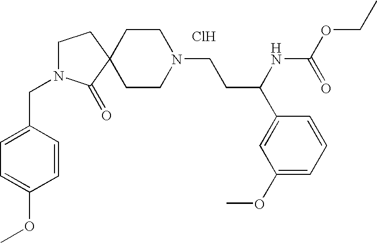 | [3-[2-(4-Methoxy-beenzyl)-1-oxo- 2,8-diaza-spiro[4.5]dec-8-yl]-1-(3- methoxy-phenyl)propyl]-carbamic acid ethyyl ester hydrochloride | 546.11 | 98% (LC/MS) |
| 615 | 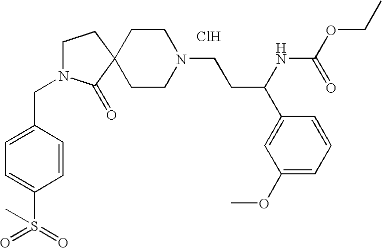 | [3-[2-(4-Methanesulfonyl-bhenzyl)-1- oxo-2,8-diaza-spiro[4.5]dec-8-yl]-1- (3-methoxy-phenyl)-propyl]- carbamic acid ethyl ester hydrochloride | 594.17 | 98% (LC/MS) |
| 616 | 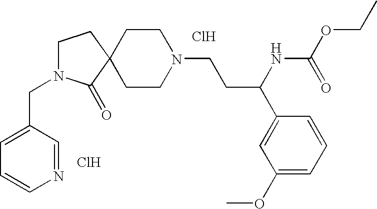 | [1-(3-Methoxy-phenyl)-3-(1-oxo-2- pyridin-3-ylmethyl-2,8-diaza- spiro[4.5]dec-8-yl)-propyl]- carbamic acid ethyl ester dihydrochloride | 553.53 | 98% (LC/MS) |
| 617 | 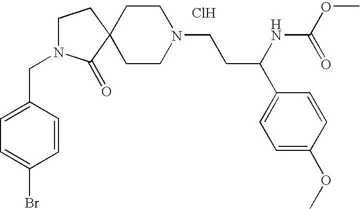 | [3-[2-(4-Bromo-benzyl)-1-oxo-2,8- diaza-spiro[4.5]dec-8-yl]-1-(4- methoxy-phenyl)-propyl]-carbamic acid methyl ester hydrochloride | 580.96 | 98% (LC/MS) |
| 618 | 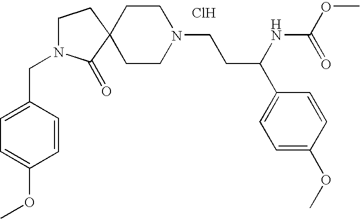 | [3-[2-(4-Methoxy-benzyl)-1-oxo- 2,8-diaza-spiro[4.5]dec-8-yl]-1-(4- methoxy-phenyl)-propyl]-carbamic acid methyl ester hydrochloride | 532.08 | 98% (LC/MS) |
| 619 | 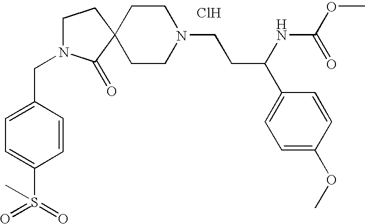 | [3-[2-(4-Methanesulfonyl-benzyl)-1- oxo-2,8-diaza-spiro[4.5]dec-8-yl]-1- (4-methoxy-phenyl)propyl]- carbamic acid methyl ester hydrochloride | 580.15 | 98% (LC/MS) |
| 620 | 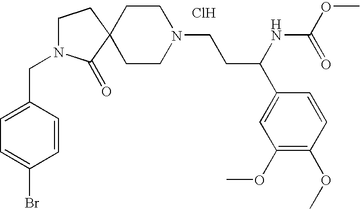 | [3-[2-(4-Bromo-benzyl)-1-oxo-2,8- diaza-spiro[4.5]dec-8-yl]-1-(3,4- dimeethoxy-phenyl)-propyl]- carbamic acid methyl ester hydrochloride | 610.99 | 98% (LC/MS) |
| 621 | 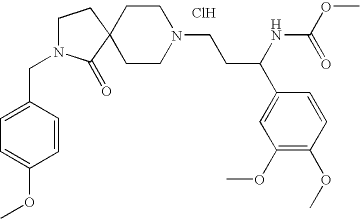 | {1-(3,4-Dimethoxy-phenyl)-3-[2-(4- meethoxy-benzyl)-1-oxo-2,8-diaza- spiro[4.5]dec-8-yl]-propyl}- carbamic acid meethyl ester hydrochloride | 562.11 | 98% (LC/MS) |
| 622 | 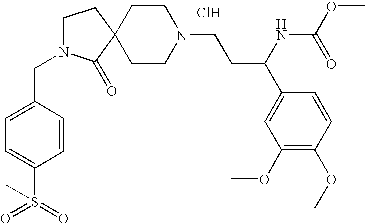 | {1-(3,4-Dimethoxy-phenyl)-3-[2-(4- methanesulfonyl-benzyl)-1-oxxo- 2,8-diaza-spiro[4.5]dec-8-yl]- propyl}-carbamic acid methyl ester hydrochloride | 610.17 | 98% (LC/MS) |
| 623 | 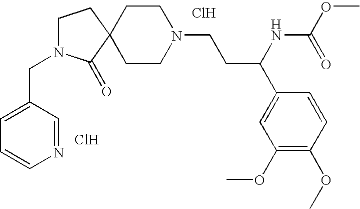 | [1-(3,4-Dimethoxy-phenyl)-3-(1- oxo-2-pyridin-3-ylmethyl-2,8-diaza- spiro[4.5]dec-8-yl)-propyl]- carbamic acid methyl ester dihydrochloride | 569.53 | 98% (LC/MS) |
| 624 | 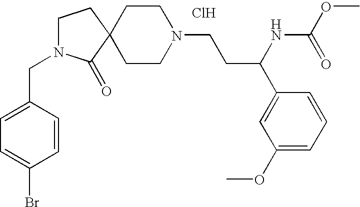 | [3-[2-(4-Bromo-benzyl)-1-oxxo-2,8- diaza-spiro[4.5]dec-8-yl]-1-(3- methoxy-phenyl)-propyl]-carbamic acid methyl ester hydrochloride | 580.96 | 98% (LC/MS) |
| 625 | 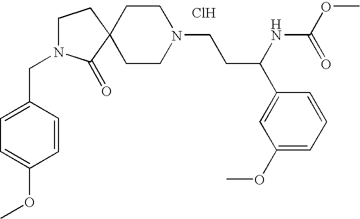 | [3-[2-(4-Methoxy-benzyl)-1-oxo- 2,8-diaza-spiro[4.5]dec-8-yl]-1-(3- methoxy-phenyl)-propyl]-carbamic acid methyl ester hydrochloride | 532.08 | 98% (LC/MS) |
| 626 | 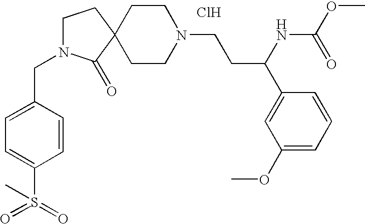 | [3-[2-(4-Methanesulfonyl-benzyl)-1- oxo-2,8-diaza-spiro[4.5]dec-8-yl]-1- (3-methoxy-phenyl)-propyl]- carbamic acid methyl ester hydrochloride | 580.15 | 98% (LC/MS) |
| 627 | 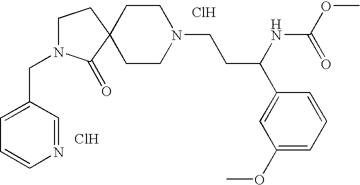 | [1-(3-Methoxy-phenyl)-3-(1-oxo-2- pyridin-3-ylmethyl-2,8-diaza- spiro[4.5]dec-8-yl)-propyl]- carbamic acid methyl ester dihydrochloride | 539.51 | 98% (LC/MS) |
| 628 | 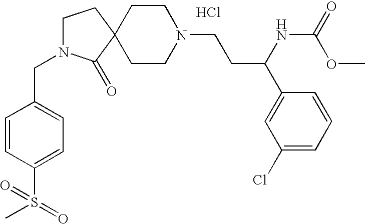 | {1-(3-Chloro-phenyl)-3-[2-(4- methanesulfonyl-benzyl)-1-oxo- 2,8-diaza-spiro[4.5]dec-8-yl]- propyl}-carbamic acid methyl ester hydrochloride | 584.56 | 98+(LC/MS) |
| 629 | 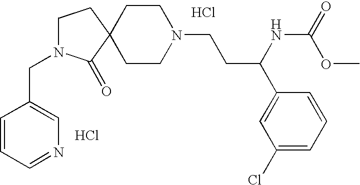 | [1-(3-Chloro-phenyl)-3-(1-oxo-2- pyridin-3-ylmethyl-2,8-diaza- spiro[4.5]dec-8-yl)-propyl]- carbamic acid methyl ester dihydrochloride | 543.92 | 98+(LC/MS) |
| 630 | 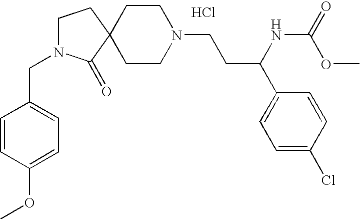 | {1-(4-Chloro-phenyl)-3-[2-(4- methoxy-benzyl)-1-oxo-2,8-diaza- spiro[4.5]dec-8-yl]propyl}- carbamic acid methyl ester hydrochloride | 536.5 | 98+(LC/MS) |
| 631 | 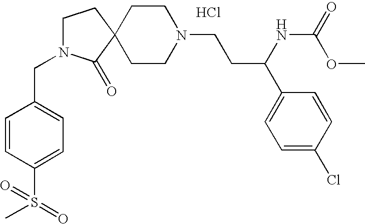 | {1-(4-Chloro-phenyl)-3-[2-(4- methanesulfonyl-benzyl)-1-oxo- 2,8-diaza-spiro[4.5]dec-8-yl]- propyl}-carbamic acid methyl ester hydrochloride | 584.56 | 98+(LC/MS) |
| 632 | 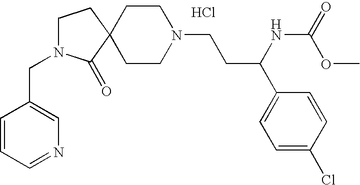 | [1-(4-Chloro-phenyl)-3-(1-oxo-2- pyridin-3-ylmethyl-2,8-diaza- spiro[4.5]dec-8-yl)-propyl]- carbamic acid methyl ester dihydrochloride | 543.92 | 98+(LC/MS) |
| 633 | 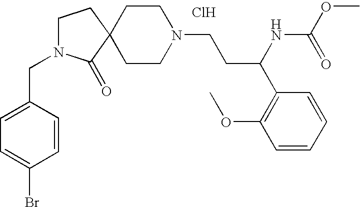 | [3-[2-(4-Bromo-benzyl)-1-oxo-2,8- diaza-spiro[4.5]dec-8-yl]-1-(2- methoxy-phenyl)-propyl]-carbamic acid methyl ester hydrochloride | 580.96 | 98+(LC/MS) |
| 634 | 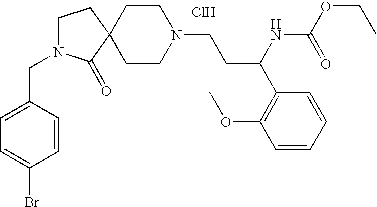 | [3-[2-(4-Bromo-benzyl)-1-oxo-2,8- diaza-spiro[4.5]dec-8-yl]-1-(2- methoxy-phenyl)-propyl]-carbamic acid ethyl ester hydrochloride | 594.99 | 98+(LC/MS) |
| 635 | 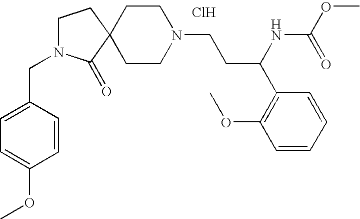 | [3-[2-(4-Methoxy-benzyl)-1-oxo- 2,8-diaza-spiro[4.5]dec-8-yl]-1-(2- methoxy-phenyl)-propyl]-carbamic acid methyl ester hydrochloride | 532.08 | 98+(LC/MS) |
| 636 | 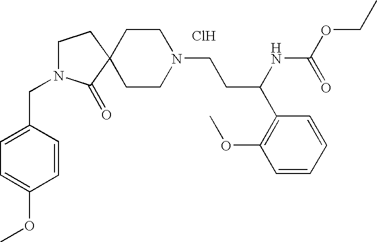 | [3-[2-(4-Methoxy-benzyl)-1-oxo- 2,8-diaza-spiro[4.5]dec-8-yl]-1-(2- methoxy-phenyl)-propyl]-carbamic acid ethyl ester hydrochloride | 546.11 | 98+(LC/MS) |
| 637 | 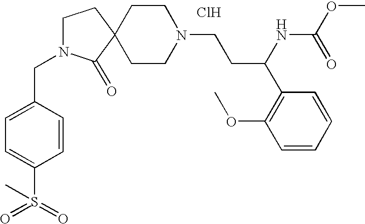 | [3-[2-(4-Methanesulfonyl-benzyl)-1- oxo-2,8-diaza-spiro[4.5]dec-8-yl]-1- (2-methoxy-phenyl)-propyl]- carbamic acid methyl ester hydrochloride | 580.15 | 98+(LC/MS) |
| 638 | 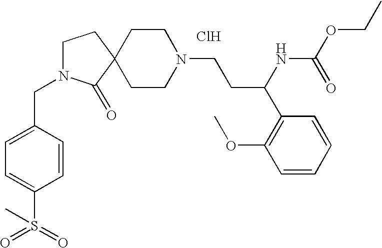 | [3-[2-(4-Methanesulfonyl-benzyl)-1- oxo-2,8-diaza-spiro[4.5]dec-8-yl]-1- (2-methoxy-phenyl)-propyl]- carbamic acid ethyl ester hydrochloride | 594.17 | 98+(LC/MS) |

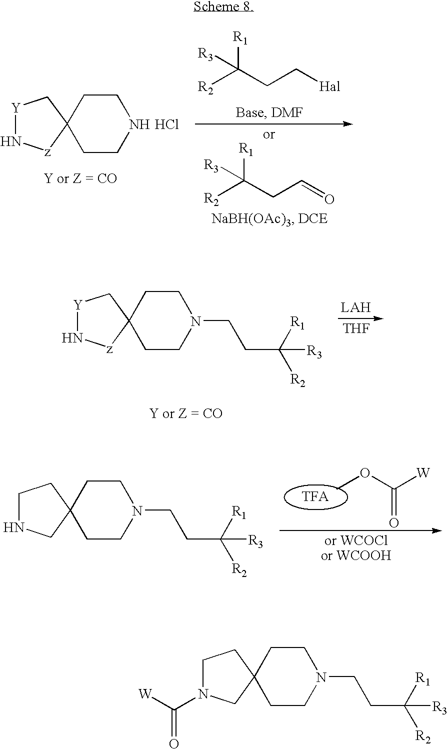

### Preparation 10

### 8-(3,3-Diphenylpropyl)-2,8-diaza-spiro[4.5]decan-1-one

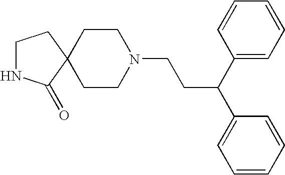 To a mixture of 1.49 g (7.8 mmol) of 2,8-diaza-spiro[4.5]decan-1-one hydrochloride, 2.39 g (8.6 mmol) of 3,3-diphenylpropyl bromide and 3.23 g (23.4 mmol) of potassium carbonate was added 40 mL of anhydrous DMF. The reaction mixture was stirred for 8 hours at 60° C. Then 10 mL of water was added and the solution was extracted with DCM (2×100 mL). The combined organic layers were dried over sodium sulfate, filtered and evaporated in vacuo. The yellow crude oil was purified by flash chromatography on silica gel (DCM/methanol 100:0 to 90:10) and 8-(3,3-diphenylpropyl)-2,8-diaza-spiro[4.5]decan-1-one was isolated as a pale yellow solid (1.22 g, 44.9%).

¹H NMR (400 MHz, DMSO-d₆): δ[ppm] 7.49 (br s, 1H) , 7.3-7.22 (m, 8H), 7.13 (m, 2H), 3.97 (t, 1H), 3.09 (t, 2H), 2.67 (m, H), 2.13 (m, 4H), 1.86 (m, 4H), 1.63 (t×d, 2H), 1.25 (br, 2H)

### Preparation 11

### 8-(3,3-Diphenylpropyl)-2,8-diaza-spiro[4.5]decane

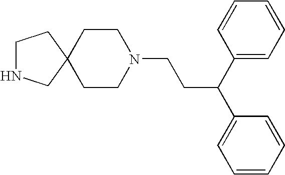 400 mg (1.14 mmol) of 8-(3,3-diphenylpropyl)-2,8-diaza-spiro[4.5]decan-1-one were dissolved in 15 mL of anhydrous THF followed by 3.44 mL of a 1M solution of LAH in THF. The reaction mixture was refluxed for 5 hours and cooled. 5 mL of water and 10 mL of aqueous solution of sodium hydroxide (1N) were successively added and the solution was stirred for an additional one hour before filtering on celite. The filtrate was extracted with DCM (2×10 mL). The organic layers were dried over sodium sulfate, filtered and evaporated in vacuo to yield 8-(3,3-diphenylpropyl)-2,8-diaza-spiro[4.5]decane as a yellow oil (365.1 mg, 95.7%).

¹H NMR (400 MHz, DMSO-d₆): δ[ppm] 7.29-7.22 (m, 8H), 7.12 (m, 2H), 3.95 (t, 1H), 3.34 (br s, 1H), 2.72 (t, 2H), 2.46 (s, 2H), 2.24-2.07 (m, 8H), 1.38 (m, 6H).

### EXAMPLE 12

### (4-Bromophenyl)-[8-(3,3-diphenylpropyl)-2,8-diaza-spiro[4.5]dec-2-yl]-methanone hydrochloride (Compound 639)

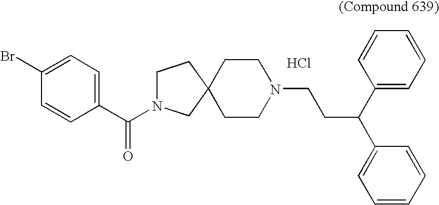 To 100 mg (100 μmol, loading of 1 mmol/g) of 4-bromophenylcarboxyl activated ester on polymeric 4-hydroxy-2,3,5,6-tetrafluorobenzamido (TFP) resin (see preparation in J. M. Salvino et al. J. Comb. Chem. 2000, 2, 691-697), preswollen with 0.5 mL of anhydrous DMF, was added 20 mg (60 μmol) of 8-(3,3-diphenylpropyl)-2,8-diaza-spiro[4.5]decane diluted in 1 mL of DMF. The reaction was agitated overnight at room temperature. The mixture was filtered and washed with DCM (2×2 mL). The filtrates were collected and evaporated in vacuo. The crude was purified by semi-preparative HPLC (method B) yielding Compound 639 as a colorless solid (9.8 mg, 29.5%).

¹H NMR (400 MHz, DMSO-d₆): δ[ppm] 9.9 (br s, 1H), 7.61 (d, 2H), 7.47 (d×d, 1H), 7.43 (d, 1H), 7.3 (m, 8H), 7.18 (t, 2H), 3.96 (t, 1H), 3.52-3.28 (m, 8H), 2.94 (m, 3H), 2.79 (m, 1H), 1.89-1.63 (m, 6H).

LC/MS: m/z 519.0 (MH⁺).

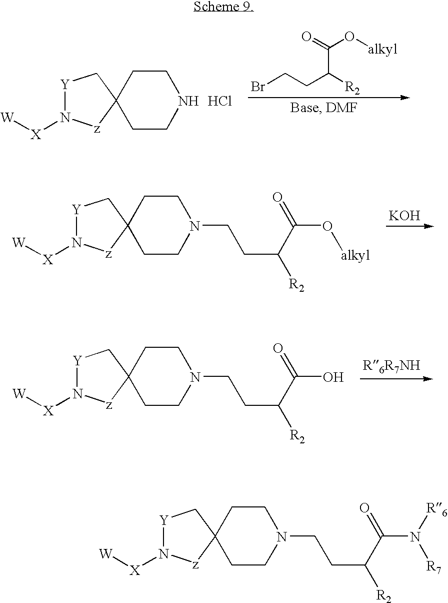

### EXAMPLE 13

### 4-[2-(4-Bromobenzyl)-1-oxo-2,8-diaza-spiro[4.5]dec-8-yl]-2-phenyl-butyric acid methyl ester (Compound 640)

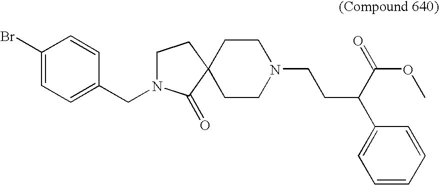

To a mixture of 500 mg (1.39 mmol) of 2-(4-bromobenzyl)-2,8-diaza-spiro[4.5]decan-1-one hydrochloride in 10 mL of anhydrous DMF was added 484 μL (2.78 mmol) of DIPEA followed by a solution of 4-bromo-2-phenyl-butyric acid methyl ester (357 mg, 1.39 mmol) in 4 mL of anhydrous DMF. Then 576 mg (4.17 mmol) of potassium carbonate were added and the reaction mixture was stirred overnight at 60° C. After cooling, the mixture was poured in water and extracted with ethyl acetate. The combined organic layers were washed with brine and dried over sodium sulfate. The crude was purified by flash chromatography on silica gel, eluting with ethyl acetate and DCM/methanol (9:1), and yielding 555 mg of Compound 640 as a colorless oil (80%).

¹H NMR (400 MHz, CD₂Cl₂): δ[ppm] 7.47 (d, 2H), 7.34-7.24 (m, 5H), 7.11 (d, 2H), 4.38 (s, 2H), 3.73 (m, 1H), 3.66 (s, 3H), 3.13 (t, 2H), 2.85 (m, 1H), 2.72 (m, 1H), 2.35-2.24 (m, 3H), 2.07-1.81 (m, 7H), 1.37 (br d, 2H).

### Preparation 12

### 4-[2-(4-Bromobenzyl)-1-oxo-2,8-diaza-spiro[4.5]dec-8-yl]-2-phenyl-butyric acid

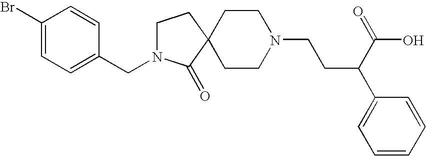 To a mixture of 521 mg (1.043 mmol) of 4-[2-(4-bromobenzyl)-1-oxo-2,8-diaza-spiro[4.5]dec-8-yl)-2-phenyl-butyric acid methyl ester in 10 mL of methanol was added 101 mg (1.56 mmol) of potassium hydroxide in 5 mL of water. The reaction mixture was refluxed for 5 hours, cooled to room temperature, diluted with water (10 mL) and treated with concentrated acetic acid. The mixture was stirred for 30 minutes and left at room temperature to allow crystallisation. 4-[2-(4-Bromobenzyl)-1-oxo-2,8-diaza-spiro[4.5]dec-8-yl]-2-phenyl-butyric acid was collected as a white solid (390 mg, 77%) by filtration.

¹H NMR (400 MHz, DMSO-d₆): δ[ppm] 7.51 (d, 2H), 7.32-7.2 (m, 5H), 7.12 (d, 2H), 4.32 (s, 2H), 3.58 (t, 1H), 3.43 (br s, 1H), 3.12 (t, 2H), 2.77 (m, 2H), 2.3-1.99 (m, 5H), 1.83 (t, 2H), 1.73 (m, 3H), 1.33 (d, 2H).

### EXAMPLE 14

### 4-[2- (4-Bromobenzyl)-1-oxo-2,8-diaza-spiro [4.5] dec-8-yl]-N-cyclohexyl-2-phenyl-butyramide hydrochloride (Compound 646)

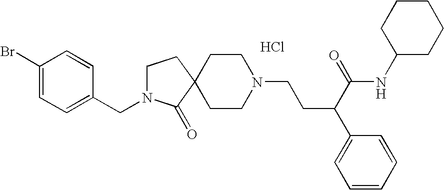 To a mixture of 24.2 mg (50 μmol) of 4-[2-(4-bromobenzyl)-1-oxo-2,8-diaza-spiro[4.5]dec-8-yl]-2-phenyl-butyric acid in 0.5 mL of DCE was added 6. 4 mg (65 vol) of cyclohexylamine in 0. 5 mL of DCE and 13. 4 mg (65 μmol) of DCC in 0.5 mL of DCE. The reaction mixture was stirred at room temperature for 16 hours and concentrated. The crude mixture was purified by semi-preparative HPLC (method E) yielding 11.9 g (39%) of Compound 646 as a pale yellowish solid.

¹H NMR (400 MHz, CD₃OD): δ[ppm] 7.49 (d, 2H) , 7.38-7.25 (m, 5H), 7.16 (d, 2H), 4.41 (d, 2H), 3.65-3.27 (m, 8H), 3.11-2.95 (m, 3H), 2.41 (m, 1H), 2.24-2.05 (m, 4H), 1.96-1.85 (m, 2H), 1.77-1.59 (m, 5H), 1.39-1.04 (m, 5H).

Table 7 of compounds illustrates some of the compounds of the present invention that were synthesized using the procedure described in scheme 9.

TABLE 7

| CPD | | | | |
|---|---|---|---|---|
| # | MOL STRUCTURE | COMPOUND NAME | MOL WT | PURITY |
| 640 | 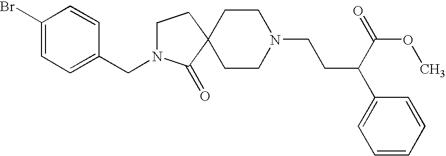 | 54-[2-(4-bromobenzyl)-1-oxo-2,8- diaza-spiro[4.5]dec-8-yl]-2-phenyl- butyric acid methyl ester | 499.446 | 100% (LC/MS) |
| 641 | 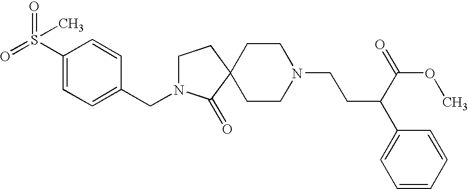 | 4-[2-(4-methanesulfonylbenzyl)-1- oxo-2,8-diaza-spiro[4.5]dec-8-yl]-1- phenyl-butyric acid methyl ester | 498.641 | 100% (LC/MS) |
| 642 | 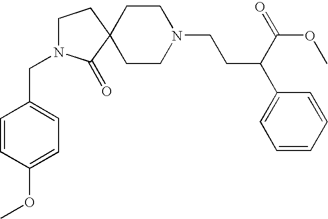 | 4-[2-(4-methoxybenzyl)-1-oxo-2,8- diaza-spiro[4.5]dec-8-yl]-2-phenyl- butyric acid methyl ester | 450.576 | >90% (HNMR) |
| 643 | 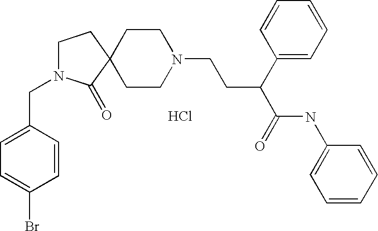 | 4-[2-(4-bromobenzyl)-1-oxo-2,8- diaza-spiro[4.5]dec-8-yl]-2,N- diphenyl-butyramide hydrochloride | 596.994 | 98+(LC/MS) |
| 644 | 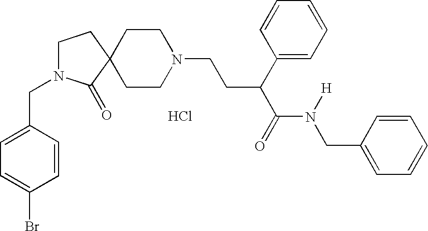 | N-benzyl-4-[2-(4-bromobenzyl)-1- oxo-2,8-diaza-spiro[4.5]dec-8-yl]-2- phenyl-butyramide hydrochloride | 611.02 | 98+(LC/MS) |
| 645 | 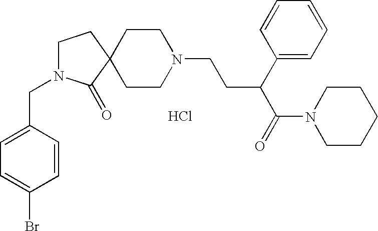 | 2-(4-bromobenzyl)-8-(4-oxo-3- phenyl-4-piperidin-1-yl-butyl)-2,8- diaza-spiro[4.5]decan-1-one hydrochloride | 589.104 | 98+(LC/MS) |
| 646 | 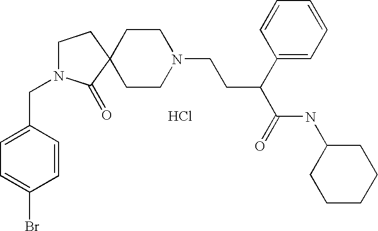 | 4-[2-(4-bromobenzyl)-1-oxo-2,8- diaza-spiro[4.5]dec-8-yl]-N- cyclohexyl-2-phenyl-butyramide hydrochloride | 603.041 | 98+(LC/MS) |
| 647 | 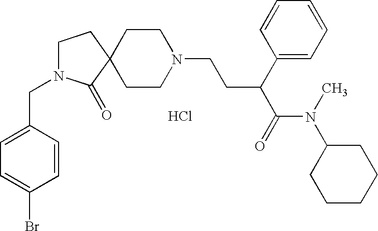 | 4-[2-(4-bromobenzyl)-1-oxo-2,8- diaza-spiro[4.5]dec-8-yl]-N- cyclohexyl-n-methyl-2-phenyl- butyramide hydrochloride | 617.068 | 98+(LC/MS) |
| 648 | 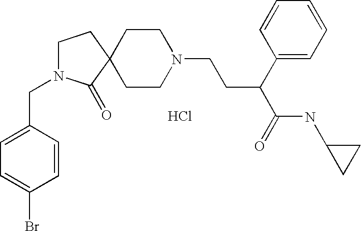 | 4-[2-(4-bromobenzyl)-1-oxo-2,8- diaza-spiro[4.5]dec-8-yl]-N- cyclopropyl-2-phenyl-butyramide hydrochloride | 560.961 | 98+(LC/MS) |
| 649 | 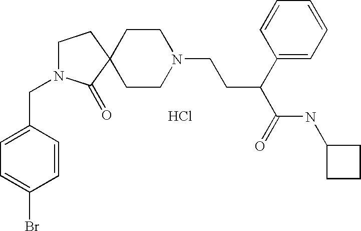 | 4-[2-(4-bromobenzyl)-1-oxo-2,8- diaza-spiro[4.5]dec-8-yl]-N- cyclobutyl-2-phenyl-butyramide hydrochloride | 574.987 | 98+(LC/MS) |
| 650 | 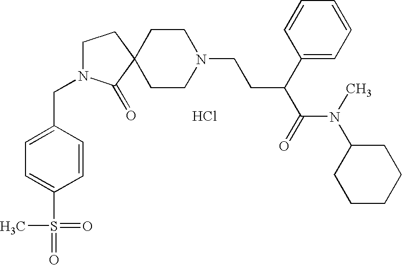 | N-cyclohexyl-4-[2-(4- methanesulfonylbenzyl)-1-oxo-2,8- diaza-spiro[4.5]dec-8-yl]-n-methyl- 2-phenyl-butyramide hydrochloride | 616.262 | 98+(LC/MS) |
| 651 | 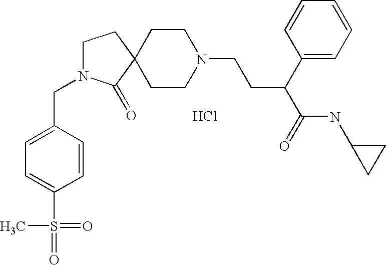 | N-cyclopropyl-4-[2-(4- methanesulfonylbnzyl)-1-oxo-2,8- diaza-spiro[4.5]dec-8-yl]-2-phenyl- butyramide hydrochloride | 560.155 | 98+(LC/MS) |
| 652 | 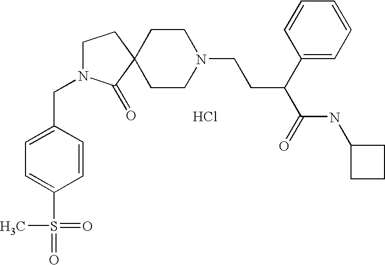 | N-cyclobutyl-44-[2-(4- methanesulfonylbenzyl)-1-oxo-2,8- diaza-spiro[4.5]dec-8-yl]-2-phenyl- butyramide hydrochloride | 574.182 | 98+(LC/MS) |
| 653 | 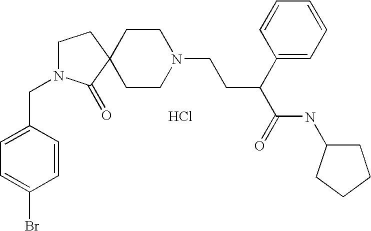 | 4-[2-(4-bromobenzyl)-1-oxo-2,8- diaza-spiro[4.5]dec-8-yl]-N- cyclopentyl-2-phenyl-butyramide hydrochloride | 589.014 | 98+(LC/MS) |
| 654 | 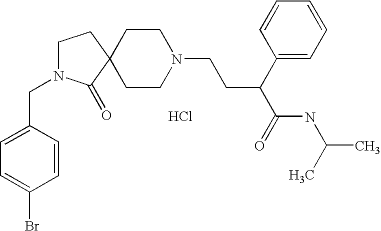 | 4-[2-(4-bromobenzyl)-1-oxo-2,8- diaza-spiro[4.5]dec-8-yl]-N- isopropyl-2-phenyl-butyramide hydrochloride | 562.976 | 98+(LC/MS) |
| 655 | 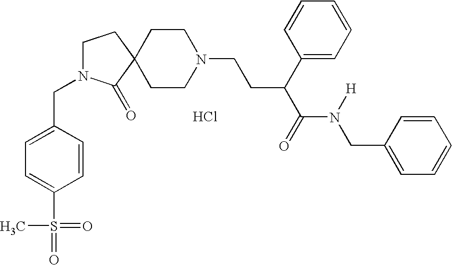 | N-benzyl-4-[2-(4- methanesulfonylbenzyl)-1-oxo-2,8- diaza-spiro[4.5]dec-8-yl]-2-phenyl- butyramide hydrochloride | 610.215 | 98+(LC/MS) |
| 656 | 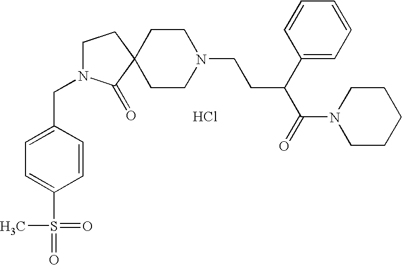 | 2-(4-methanesulfonylbenzyl)-8-(4- oxo-3-phenyl-4-piperidin-1-yl- butyl)-2,8-diaza-spiro[4.5]decan-1- one hydrochloride | 588.209 | 98+(LC/MS) |
| 657 | 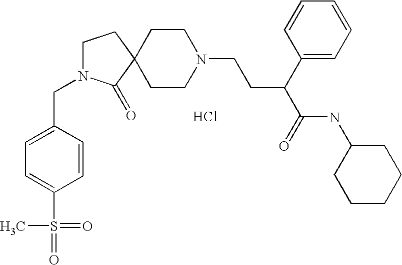 | N-cyclohexyl-4-[2-(4- methanesulfonylbenzyl)-1-oxo-2,8- diaza-spiro[4.5]dec-8-yl]-2-phenyl- butyramide hydrochloride | 602.236 | 98+(LC/MS) |
| 658 | 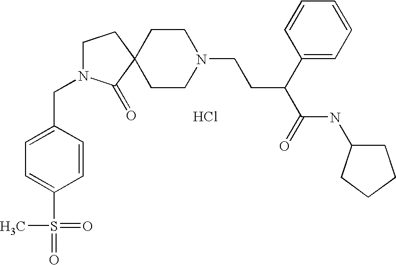 | N-cyclopentyl-4-[2-(4- methanesulfonylbenzyl)-1-oxo-2,8- diaza-spiro[4.5]dec-8-yl]-2-phenyl- butyramide hydrochloride | 588.209 | 98+(LC/MS) |
| 659 | 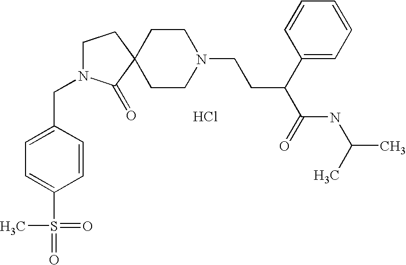 | N-isopropyl-4-[2-(4- methanesulfonylbenzyl)-1-oxo-2,8- diaza-spiro[4.5]dec-8-yl]-2-phenyl- butyramide hydrochloride | 562.171 | 98+(LC/MS) |
| 660 | 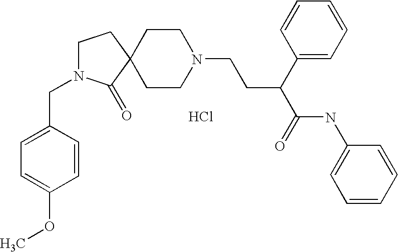 | 4-[2-(4-methoxybenzyl)-1-oxo-2,8- diaza-spiro[4.5]dec-8-yl]-2,N- diphenyl-butyramide hydrochloride | 548.123 | 90.6% (LC/MS) |
| 661 | 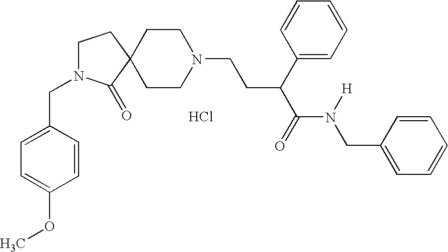 | N-benzyl-4-[2-(4-methoxybenzyl)- 1-oxo-2,8-diaza-spiro[4.5]dec-8-yl]- 2-phenyl-butyramide hydrochloride | 562.15 | 98+(LC/MS) |
| 662 | 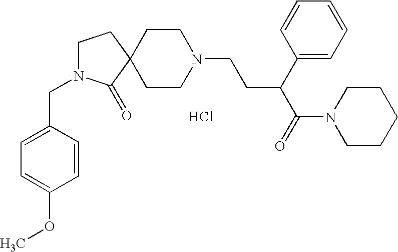 | 2-(4-methoxybenzyl)-8-(4-oxo-3- phenyl-4-piperidin-1-yl-butyl)-2,8- diaza-spiro[4.5]decan-1-one hydrochloride | 540.144 | 98+(LC/MS) |
| 663 | 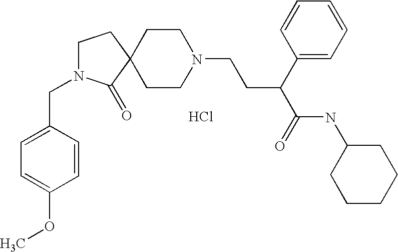 | N-cyclohexyl-4-[2-(4- methoxybenzyl)-1-oxo-2,8-diaza- spiro[4.5]dec-8-yl]-2-phenyl- butyramide hydrochloride | 554.171 | 98+(LC/MS) |
| 664 | 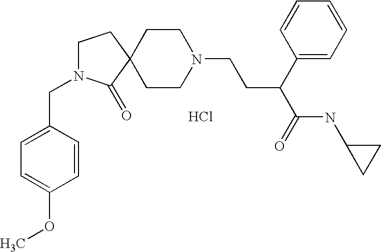 | N-cyclopropyl-4-[2-(4- methoxybenzyl)-1-oxo-2,8-diaza spiro[4.5]dec-8-yl]-2-phenyl- butyramide hydrochloride | 512.09 | 98+(LC/MS) |
| 665 | 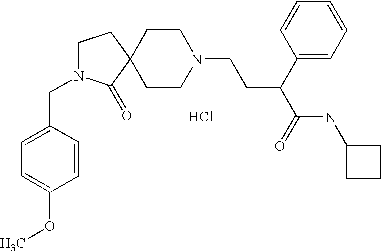 | N-cyclobutyl-4-[2-(4- methoxybenzyl)-1-oxo-2,8-diaza- spiro[4.5]dec-8-yl]-2-phenyl- butyramide hydrochloride | 526.117 | 98+(LC/MS) |
| 666 | 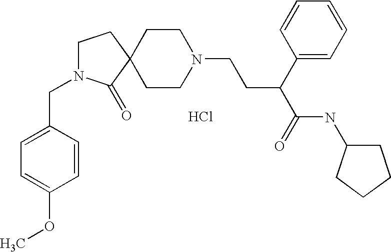 | N-cyclopentyl-4-[2-(4- methoxybenzyl)-1-oxo-2,8-diaza- spiro[4.5]dec-8-yl]-2-phenyl- butyramide hydrochloride | 540.144 | 98+(LC/MS) |
| 667 | 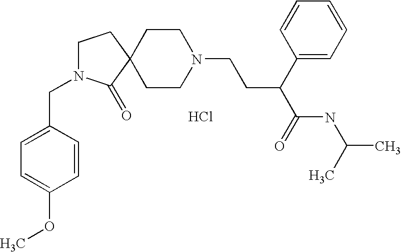 | N-isopropyl-4-[2-(4- methoxybenzyl)-1-oxo-2,8-diaza- spiro[4.5]dec-8-yl]-2-phenyl- butyramide hydrochloride | 514.106 | 98+(LC/MS) |

Table 8 of compounds illustrates some of the compounds of the present invention that can be synthesized using the procedure described in schemes 1-9.

TABLE 8

| CPD | | | |
|---|---|---|---|
| # | MOL STRUCTURE | COMPOUND NAME | MOL WT |
| 668 | 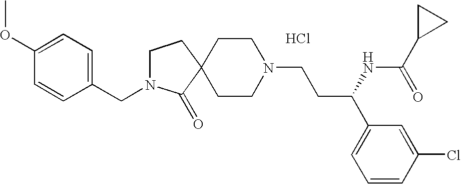 | Cyclopropanecarboxylic acid {(S)- 1-(3-chloro-phenyl)-3-[2-(4- methoxy-benzyl)-1-oxo-2,8-diaza- spiro[4.5]dec-8-ylp]propyl}-amide hydrochloride | 546.54 |
| 669 | 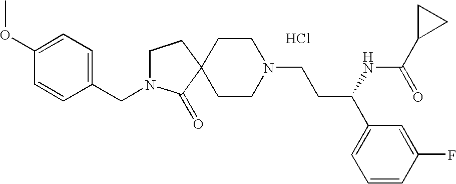 | Cyclopropanecarboxylic acid {(S)- 1-(3-fluoro-phenyl)-3-[2-(4- methoxy-benzyl)-1-oxo-2,8-diaza- spiro[4.5]dec-8-yl]-propyl}-amide hydrochloride | 530.09 |
| 670 | 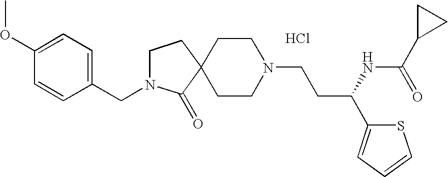 | Cyclopropanecarboxylic acid {(S)- 3-[2-(4-methoxy-benzyl)-1-oxo- 2,8-diaza-spiro[4.5]dec-8-yl]-1- thiophen-2-yl-propyl}-amide hydrochloride | 518.12 |
| 671 | 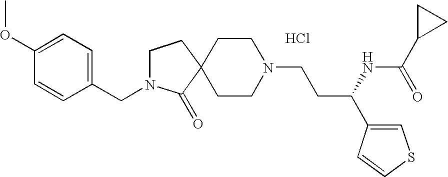 | Cyclopropanecarboxylic acid {(S)- 3-[2-(4-methoxy-benzyl)-1-oxo- 2,8-diaza-spiro[4.5]dec-8-yl]-1- thiophen-3-yl-propyl}-amide hydrochloride | 518.12 |
| 672 | 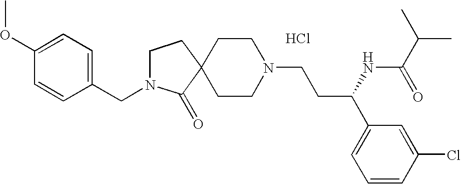 | N-{(S)-1-(3-Chloro-phenyl)-3-[2- (4-methoxy-benzyl)-1-oxo-2,8- diaza-spiro[4.5]dec-8-yl]prropyl}- isobutyramide hydrochloride | 548.56 |
| 673 | 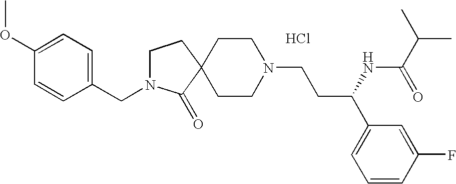 | N-{(S)-1-(3-Fluoro-phenyl)-3-[2- (4-methoxy-benzyl)-1-oxo-2,8- diaza-spiro[4.5]dec-8-yl]-propyl}- isobutyramide hydrochloride | 532.1 |
| 674 | 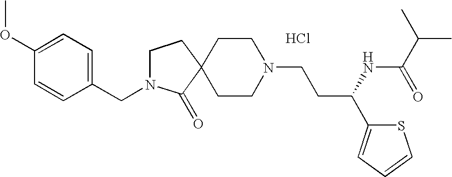 | N-{(S)-3-[2-(4-Methoxy-benzyl)-1- oxo-2,8-diaza-spiro[4.5]dec-8-yl]- 1-thiophen-2-yl-propyl}- isobutyramide hydrochloride | 520.14 |
| 675 | 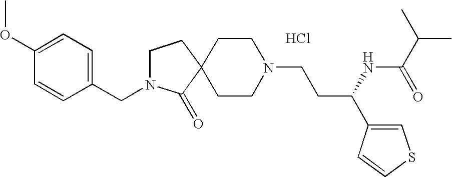 | N-{(S)-3-[2-(4-Methoxy-bnzyl)-1- oxo-2,8-diaza-spiro[4.5]dec-8-yl]- 1-thiophen-3-yl-propyl}- isobutyramide hydrochloride | 520.14 |
| 676 | 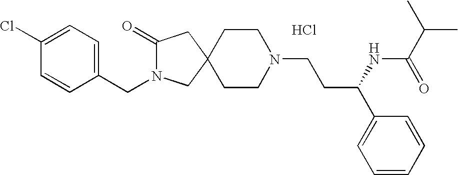 | N-{(S)-3-[2-(4-Chloro-benzyl)-3- oxo-2,8-diaza-spiro[4.5]dec-8-yl]- 1-phenyl-propyl}-isobutyramide hydrochloride | 518.53 |
| 677 | 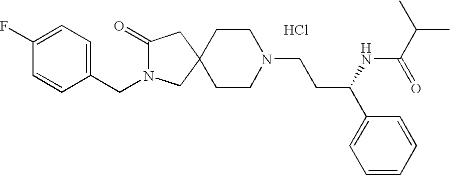 | N-{(S)(-3-[2-(4-Fluoro-benzyl)-3- oxo-2,8-diaza-spiro[44.5]dec-8-yl]- 1-phenyl-propyl}-isobutyramide hydrochloride | 502.08 |
| 678 | 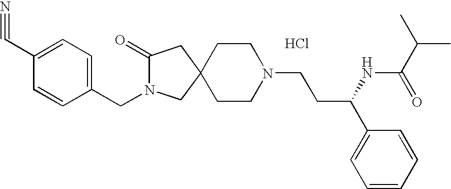 | N-{(S)-3-[2-(4-Cyano-benzyl)-3- oxo-2,8-diaza-spiro[4.5]dec-8-yl]- 1-phenyl-propyl}-isobutyramide hydrochloride | 509.1 |
| 679 | 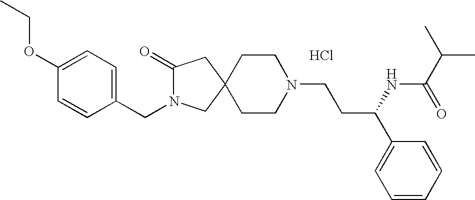 | N-{(S)-3-[2-(4-Ethoxy-benzyl)-3- oxo-2,8-diaza-spiro[4.5]dec-8-yl]- 1-phenyl-propyl}-isobutyramide hydrochloride | 528.1214 |
| 680 | 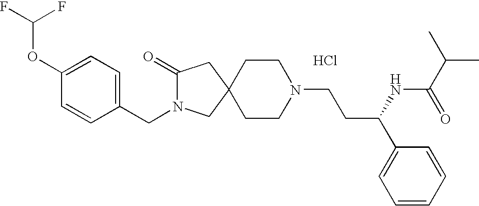 | N-{(S)-3-[2-(4-Difluoromethoxy- benzyl)-3-oxo-2,8-diaza- spiro[4.5]dec-8-yl]-1-phenyl- propyl}-isobutyramide hydrochloride | 550.09 |
| 681 | 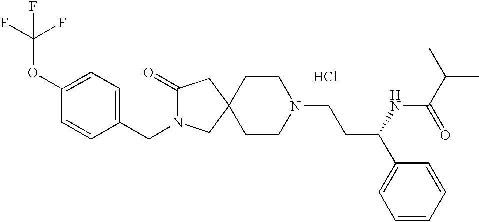 | N-{(S)-3-[3-Oco-2-(4- trifluoromethoxy-benzyl)-2,8- diaza-spiro[4.5]dec-8-yl]-1- phenyl-propyl}-isobutyramide hydrochloride | 568.08 |
| 682 | 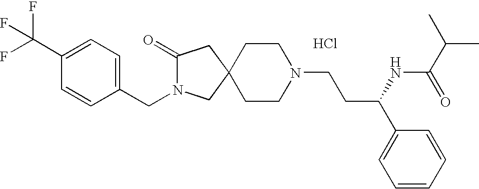 | N-{(S)-3-[3-Oxo-2-(4- trifluoromethyl-benzyl)-2,8-diaza- spiro[4.5]dec-8-yl]-1-phenyl- propyl}-isobutyramide hydrochloride | 552.08 |
| 683 | 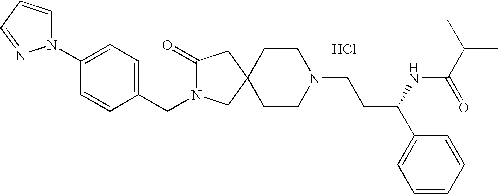 | N-{(S)-3-[3-Oxo-2-(4-pyrazol-1-yl- benzyl)-2,8-diaza-spiro[4.5]dec-8- yl]-1-phenyl-propyl}- issobutyramide hydrochloride | 550.15 |
| 684 | 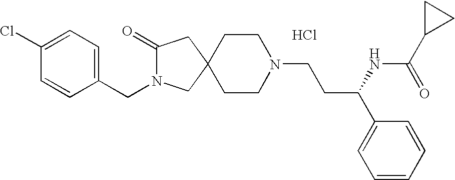 | Cyclopropanecarboxylic acid {(S)- 3-[2-(4-chloro-benzyl)-3-oxo-2,8- diaza-spiro[4.5]dec-8-yl]-1- phenyl-propyl}-amide hydrochloride | 516.52 |
| 685 | 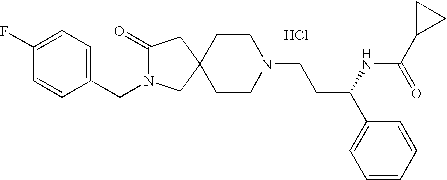 | Cyclopropanecarboxylic acid {(S)- 3-[2-(4-fluoro-benzyl)-2-oxo-2,8- dxiaza-spiro[4.5]dec-8-yl]-1- phenyl-propyl}-amide hydrochloride | 500.06 |
| 686 | 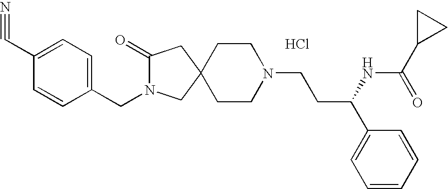 | Cyclopropanecarboxylic acid {(S)- 3-[2-(4-cyano-benzyl)-3-oxo-2,8- diaza-spiro[4.5]dec-8-yl]-1- phenyl-propyl}-amide hydrochloride | 507.08 |
| 687 | 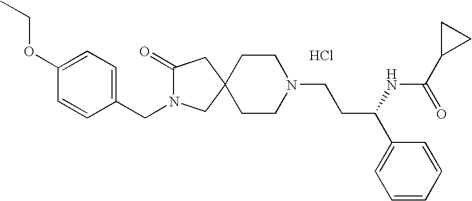 | Cyclopropanecarboxylic acid {(S)- 3-[2-(4-ethoxy-benzyl)-3-oxo-2,8- diaza-spiro[4.5]dec-8-yl]-1- phenyl-propyl}-amide hydrochloride | 526.12 |
| 688 | 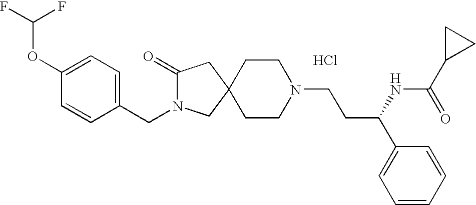 | Cyclopropanecarboxylic acid {(S)- 3-[2-(4-difluoromethoxy-benzyl)- 3-oxo-2,8-diaza-spiro[4.5]dec-8- yl]-1-phenyl-propyl}-amide dihydrochloride | 548.08 |
| 689 | 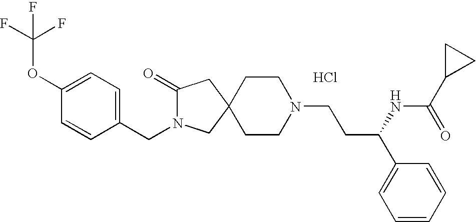 | Cyclopropanecarboxylic acid {(S)- 3-[2-(4-trifluoromethoxy-benzyl)- 3-oxo-2,8-diaza-spiro[4.5]dec-8- yl]-1-phenyl-propyl}-amide hydrochloride | 566.07 |
| 690 | 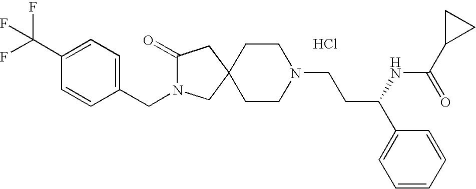 | Cyclopropanecarboxylic acid {(S)- 3-[2-(4-trifluoromethyl-benzyl)-3- oxo-2,8-diaza-spiro[4.5]dec-8-yl]- 1-phenyl-propyl}-amide hydrochloride | 550.07 |
| 691 | 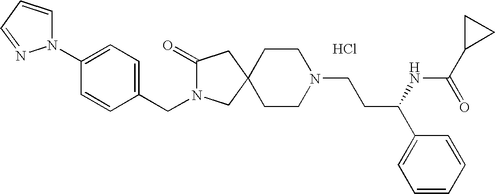 | Cyclopropanecarboxylic acid {(S)- 3-[3-oxo-2-(4-pyrazol-1-yl- benzyl)-2,8-diaza-spiro[4.5]dec-8- yl]-1-phenyl-propyl}-amide hydrochloride | 548.13 |
| 692 | 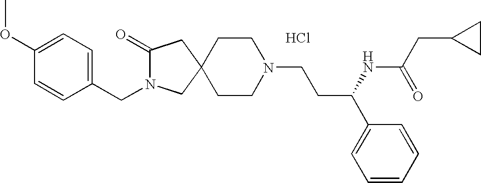 | 2-Cyclopropyl-N-{(S)-3-[2-(4- methoxy-benzyl)-3-oxo-2,8-diaza- spiro[4.5]dec-8-yl]-1-phenyl- propyl}-acetamide hydrochloride | 526.12 |
| 693 | 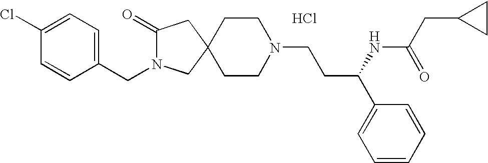 | N-{(S)-3-[2-(4-Chloro-benzyl)-3- oxo-2,8-diaza-spiro[4.5]dec-8-yl]- 1-phenyl-propyl}-2-cyclopropyl- acetamide hydrochloride | 530.54 |
| 694 | 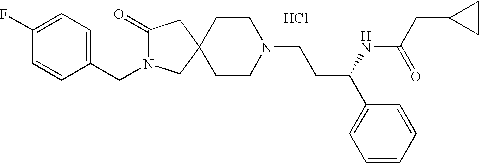 | N-{(S)-3-[2-(4-Fluoro-beenzyl)-3- oxo-2,8-diaza-spiro[44.5]dec-8-yl]- 1-phenyl-propyl}-2-cyclopropyl- acetamide hydrochloride | 514.09 |
| 695 | 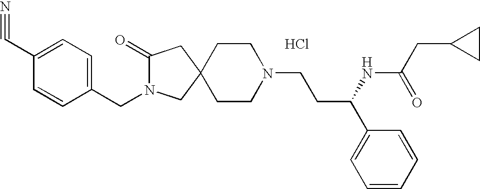 | N-{(S)-3-[2-(4-Cyano-benzyl)-3- oxo-2,8-diaza-spiro[4.5]dec-8-yl]- 1-phenyl-propyl}-2-cyclopropyl- acetamide hydrochloride | 521.11 |
| 696 | 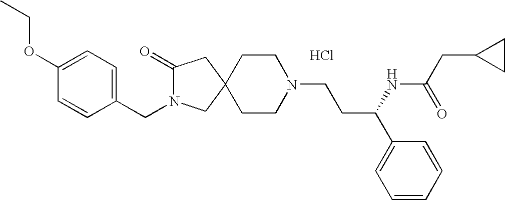 | N-{(S)-3-[2-(4-Ethoxy-benzyl)-3- oxo-2,8-diaza-spiro[4.5]dec-8-yl]- 1-phenyl-propyl}-2-cyclopropyl- acetamide hydrochloride | 540.15 |
| 697 | 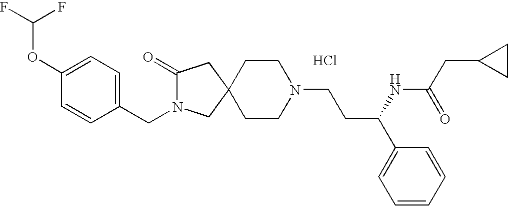 | N-{(S)-3-[2-(4-Difluoromethoxy- benzyl)-3-oxo-2,8-diaza- spiro[4.5]dec-8-yl]-1-phenyl- propyl}-2-cyclopropyl-acetamide hydrochloride | 562.1 |
| 698 | 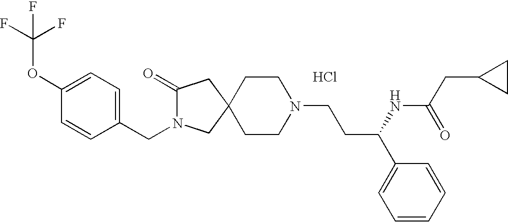 | N-{(S)-3-[2-(4-Trifluoromethoxy- benzyl)-3-oxo-2,8-diaza- spiro[4.5]dec-8-yl]-1-phenyl- propyl}-2-cyclopropyl-acetamide hydrochloride | 580.09 |
| 699 | 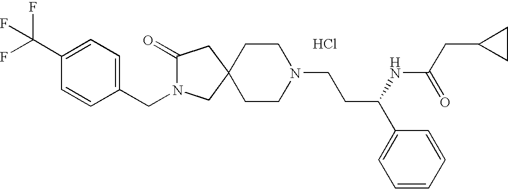 | N-{(S)-3-[2-(4-trifluoromethyl- benzyl)-3-oxo-2,8-diaza- spiro[4.5]dec-8-yl]-1-phenyl- propyl}-2-cyclopropyl-acetamide hydrochloride | 564.1 |
| 700 | 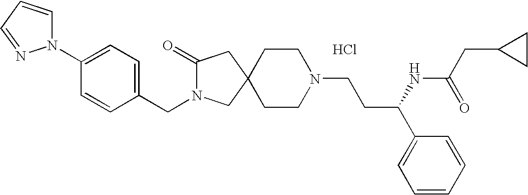 | 2-Cyclopropyl-N-{(S)-3-[3-oxo-2- (4-pyrazol-1-yl-benzyl)-2,8-diaza- spiro[4.5]dec-8-yl]-1-phenyl- propyl}-acetamide hydrochloride | 562.16 |
| 701 | 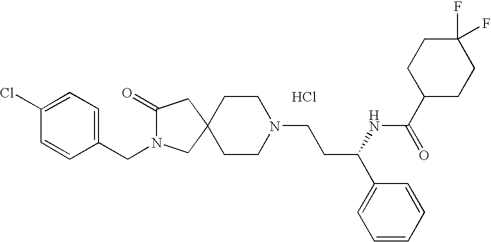 | 4,4-Difluoro- cyclohexanecarboxylic acid {(S)- 3-0[2-(4-chloro-benzyl)-3-oxo-2,8- diaza-spiro[4.5]dec-8-yl]-1- phenyl-propyl}-amide hydrochloride | 594.58 |
| 702 | 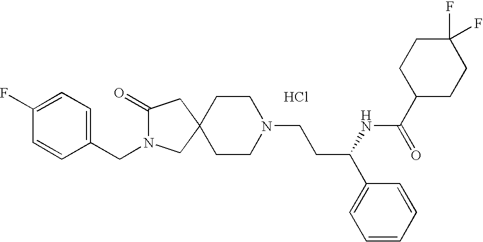 | 4,4-Difluoro- cyclohexanecarboxylic acid {(S)- 3-[2-(4-cyano-benzyl)-3-oxo-2,8- diaza-spiro[4.5]dec-8-yl]-1- phenyl-propyl}-amide hydrochloride | 578.12 |
| 703 | 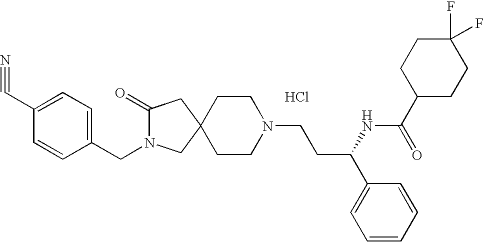 | 4,4-Difluoro- cyclohexanecarboxylic acid {(S)- 3-[2-(4-cyano-benzyl)-3-oxo-2,8- diaza-spiro[4.5]dec-8-yl]-1- phenyl-propyl}-amide hydrochloride | 585.14 |
| 704 | 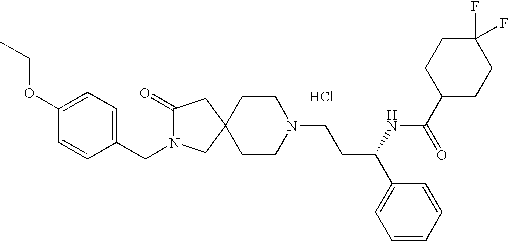 | 4,4-Difluoro- cyclohexanecarboxylic acid {(S)- 3-[2-(4-ethoxy-benzyl)-3-oxo-2,8- diaza-spiro[4.5]dec-8-yl]-1- phenyl-propyl}-amide hydrochloride | 604.19 |
| 705 | 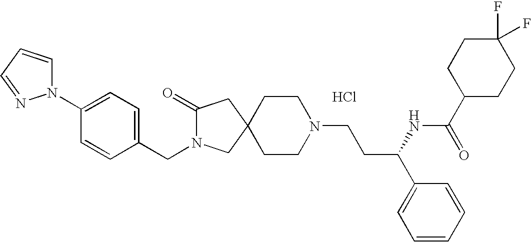 | 4,4-Difluoro- cyclohexanecarboxylic acid {(S)- 3-[3-oxo-2-(4-pyrazol-1-yl- benzyl)-2,8-diaza-spiro[4.5]dec-8- yl]-1-phenyl-propyl}-amide hydrochloride | 626.19 |
| 706 | 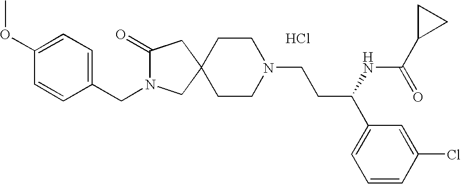 | Cyclopropanecarbocylic acid {(S)- 1-(3-chloro-phenyl)-3-[2-(4- methoxy-benzyl)-3-oxo-2,8-diaza- spiro[4.5]dec-8-yl]-propyl}-amide hydrochloride | 546.54 |
| 707 | 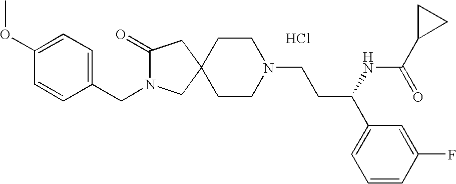 | Cyclopropanecarboxylic acid {(S)- 1-(3-fluoro-phenyl)-3-[2-(4- methoxy-benzyl)-3-oxo-2,8-diaza- spiro[4.5]dec-8-yl]-propyl}-amide hydrochloride | 530.09 |
| 708 | 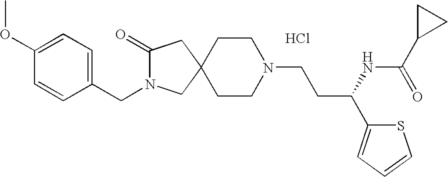 | Cyclopropanecarboxylic acid {(S)- 3-[2-(4-methoxy-benzyl)-3-oxo- 2,8-diaza-spiro[4.5]dec-8-yl]-1- thiophen-2-yl-propyl}-amide hydrochloride | 518.12 |
| 709 | 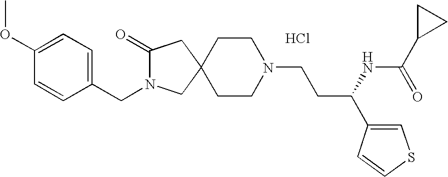 | Cyclopropanecarboxylic acid {(S)- 3-[2-(4-methoxy-benzyl)-3-oxo- 2,8-diaza-spiro[4.5]dec-8-yl]-1- thiophen-3-yl-propyl}-amide hydrochloride | 518.12 |
| 710 | 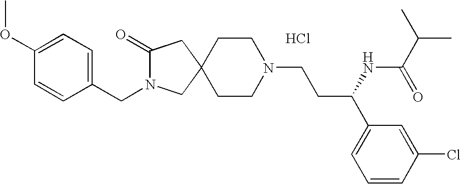 | N-{(S)-1-(3-Chloro-phenyl)-3-[2- (4-methoxy-beenzyl)-3-oxo-2,8- diaza-spiro[4.5]dec-8-yl]-propyl}- isobutyramide hydrochloride | 548.56 |
| 711 | 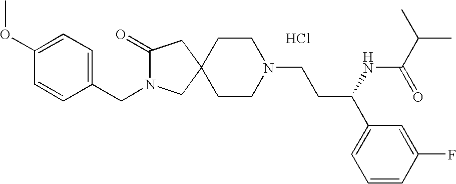 | N-{(S)-1-(3-Fluoro-phenyl)-3-[2- (4-methoxy-benzyl)-3-oxo-2,8- diaza-spiro[4.5]dec-8-yl]-propyl}- isobutyramide hydrochloride | 532.1 |
| 712 | 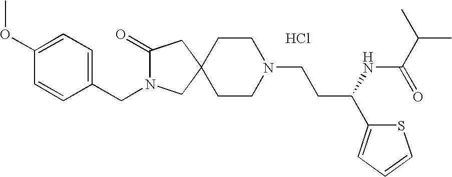 | N-{(S)-3-[2-(4-Methoxy-benzyl)-3- oxo-2,8-diaza-spiro[4.5]dec-8-yl]- 1-thiophen-2-yl-propyl}- isobutyramide hydrochloride | 520.14 |
| 713 | 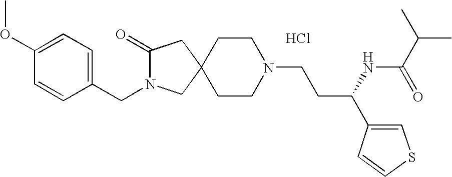 | N-{(S)-3-[2-(4-Methoxy-benzyl)-3- oxo-2,8-diaza-spiro[4.5]dec-8-yl]- 1-thiophen-3-yl-propyl}- isobutyramide hydrochloride | 520.14 |
| 714 | 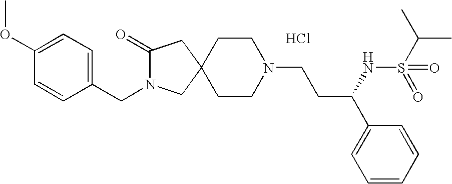 | Propane-2-sulfonic acid {(S)-3-[2- (4-methoxy-benzyl)-3-oxo-2,8- diaza-spiro[4.5]dec-8-yl]-1- phenyl-propyl}-amide hydrochloride | 550.16 |
| 715 | 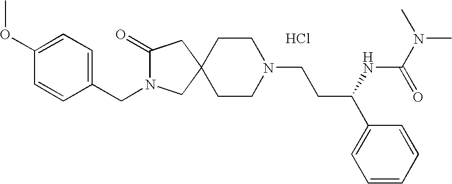 | 3-{(S)-3-[2-(4-Methoxy-benzyl)-3- oxo-2,8-diaza-spiro[4.5]dec-8-yl]- 1-phenyl-propyl}-1,1-dimethyl- urea hydrochloride | 515.1 |
| 716 | 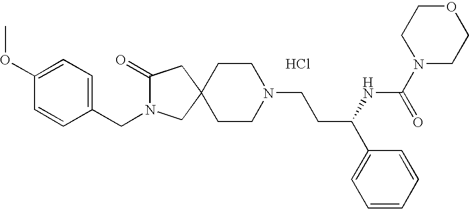 | Morpholine-4-carboxylic acid {(S)- 3-[2-(4-methoxy-benzyl)-3-oxo- 2,8-diaza-spiro[4.5]dec-8-yl]-1- phenyl-propyl}-amide hydrochloride | 557.14 |
| 717 | 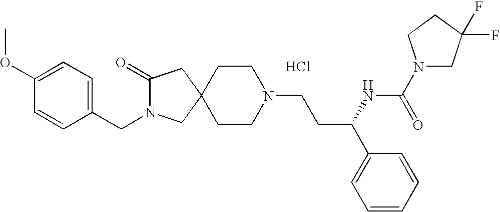 | 3,3-Difluoro-yrrolidine-1- carboxylic acid {(S)-3-[2-(4- methoxy-benzyl)-3-oxo-2,8-diaza- spiro[4.5]dec-8-yl]-1-phenyl- propyl}-amide hydrochloride | 577.12 |
| 718 | 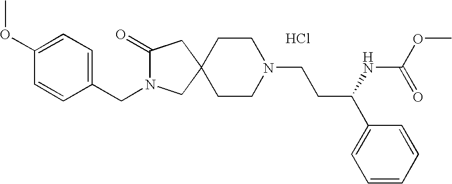 | {(S)-3-[2-(4-Methoxy-benzyl)-3- oxo-2,8-diaza-spiro[4.5]dec-8-yl]- 1-phenyl-propyl}-carbamic acid methyl ester hydrochloride | 502.06 |
| 719 | 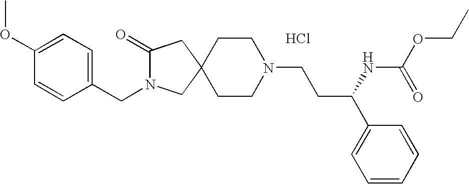 | {(S)-3-[2-(4-Methoxy-benzyl)-3- oxo-2,8-diaza-spiro[4.5]dec-8-yl]- 1-phenyl-propyl}-carbamic acid ethyl ester hydrochloride | 516.08 |

### EXAMPLE 15

The following assay methods are suitable for evaluating the compounds of the invention.

Chemokine Binding assay: Membranes (1 μg/well) from human embryonic kidney (HEK-293) cells expressing human CCR5 were incubated with 0.1 nM ¹²⁵I-labeled MIP-1α (Amersham) in the presence of varying concentrations of a test compound (10000-0.01 nM) in buffer (50 mM Hepes, pH 7.3/5 mM MgCl₂/1 mM CaCl₂/0.5% BSA) for 90 min at room temperature. Reaction mixtures (100 μL) were filtered through Multiscreen GFB filters (Millipore) and washed six times with cold wash buffer (50 mM Hepes, pH 7.3/0.5 M NaCl, 0.1% BSA). Bound ¹²⁵I-MIP-1α was quantitated by liquid scintillation counting. The nonspecific binding of ¹²⁵I-labeled MIP-1α to the membrane was determined based on the radioactivity from the wells added with 100 nM non-radiolabeled MIP-1α. IC₅₀ and K_{D} values were calculated by using GRAPHPAD PRISM software (Intuitive Software for Science, San Diego)

HIV-1 Replication in PBMC Cultures. Isolated PBMC were stimulated in vitro with 5 μg/ml phytohemagglutinin and 50 units/ml IL-2 for 3 days. The cells were resuspended at 4×10⁶/ml in complete medium (RPMI, 10% FBS/50 units/ml IL-2), seeded into 96-well plates (2×10⁵/well), incubated with inhibitor for 1 h at 37° C., and infected in triplicate with 25-100 tissue culture 50% infective dose (TCID₅₀) per well of the R5 HIV-1_{JR-FL} strain for 3-4 h. The cells were washed twice in PBS to remove residual virus and cultured in the presence of inhibitor for 4-6 days. HIV-1 replication was determined by the presence of viral RT activity in harvested supernatant fluid. The IC₅₀ values for the virus were determined by using GRAPHPAD PRISM software.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding U.S. Provisional Application Ser. No. 60/487,973, filed Jul. 18, 2003 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A compound according to formula (I): 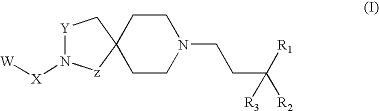
or a pharmaceutically acceptable salt, hydrate or solvate thereof,
wherein
Y, Z and X are each independently CH₂, C═O or CR₄R₅;
W is H, optionally substituted C-₁₋₁₀ alkyl (e.g. C₁₋₆ alkyl) optionally substituted C₂₋₁₀ alkenyl (e.g. C₂₋₆ alkenyl), optionally substituted C₂₋₁₀ alkynyl (e.g. C₂₋₆ alkynyl), optionally substituted C₆₋₁₂ aryl, optionally substituted 3 to 10 membered heterocycle, optionally substituted C₆₋₁₂ aralkyl or optionally substituted C₃₋₁₀ heteroaralkyl;
R₁ is H, OH, optionally substituted C₁₋₁₀ alkyl, optionally substituted C₂₋₁₀ alkenyl, optionally substituted C₂₋₁₀ alkynyl, optionally substituted C₆₋₁₂ aryl, NR₈R₉, optionally substituted O—C₁₋₆ alkyl, optionally substituted O—C₆₋₁₂ aryl, optionally substituted O—C₆₋₁₂ aralkyl, 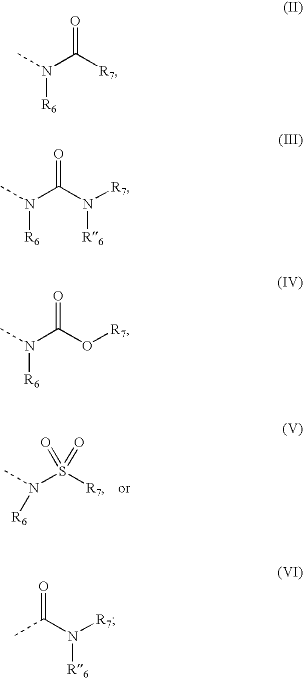
R₂ is optionally substituted C₁₋₁₀ alkyl, optionally substituted C₂₋₁₀ alkenyl, optionally substituted C₂₋₁₀ alkynyl, optionally substituted C₆₋₁₂ aryl or optionally substituted 3 to 10 membered heterocycle;
R₃ is H, optionally substituted C₁₋₁₀ alkyl, optionally substituted C₂₋₁₀ alkenyl, optionally substituted C₂₋₁₀ alkynyl, or optionally substituted C₆₋₁₂ aryl;
R₄ and R₅ are each independently H, optionally substituted C₁₋₁₀ alkyl, optionally substituted C₂₋₁₀ alkenyl, optionally substituted C₂₋₁₀ alkynyl, or optionally substituted C₆₋₁₂ aryl;
R₆ and R″₆ are each, independently, H, optionally substituted C₁₋₁₀ alkyl optionally substituted C₂₋₁₀ alkenyl, or optionally substituted C₂₋₁₀ alkynyl and R₇ is H, optionally substituted C₁₋₁₀ alkyl, optionally substituted C₂₋₁₀ alkenyl, optionally substituted C₂₋₁₀ alkynyl, optionally substituted C₆₋₁₂ aryl, optionally substituted 3 to 10 membered heterocycle, optionally substituted C₆₋₁₂ aralkyl or optionally substituted 3 to 10 membered heteroaralkyl, or R″₆ and R₇ can be taken together to form an optionally substituted 3 to 10 membered heterocycle; and
R₈ and R₉ are each independently H, optionally substituted C₁₋₁₀ alkyl, optionally substituted C₂₋₁₀ alkenyl, or optionally substituted C₂₋₁₀ alkynyl.

2. A compound according to claim 1, wherein said compound is of formula (Ia): 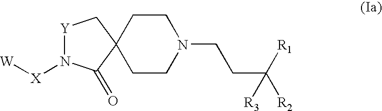
or a pharmaceutically acceptable salt, hydrate or solvate thereof.

3. A compound according to claim 1, wherein said compound is of formula (Ib): 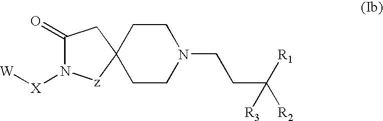
or a pharmaceutically acceptable salt, hydrate or solvate thereof.

4. A compound according to claim 1, wherein said compound is of formula (Ic): 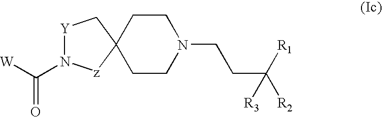
or a pharmaceutically acceptable salt, hydrate or solvate thereof.

5. A compound according to claim 1, wherein said compound is of formula (Id): 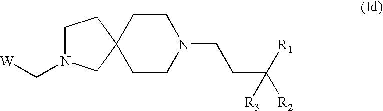
or a pharmaceutically acceptable salt, hydrate or solvate thereof.

6. A compound according to claim 1, wherein said compound is an (S)-enantiomer of formula (Ie): 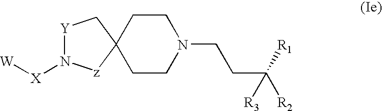
or a pharmaceutically acceptable salt, hydrate or solvate thereof.

7. A compound according to claim 1, wherein W is C₆₋₁₂ aryl or 3 to 10 membered heterocycle.

8. A compound according to claim 1, wherein W is C₆₋₁₂ aryl.

9. A compound according to claim 1, wherein W is 3 to 10 membered heterocycle.

10. A compound according to claim 1, wherein W is phenyl, phenyl substituted in the para (p) position, phenyl substituted with a halogen, phenyl substituted with Br, phenyl substituted with F, phenyl substituted with Cl, phenyl substituted with at least one halogen, phenyl substituted with a C₁₋₃ alkoxy, phenyl substituted with methoxy, phenyl substituted with SO₂C₁₋₃alkyl, phenyl substituted with methanesulfonyl, phenyl substituted with halogenated C₁₋₆ alkyl, phenyl substituted with CHF₂, phenyl substituted with halogenated C₁₋₆ alkoxy, phenyl substituted with OCF₃, or pyridine.

11. A compound according to claim 1, wherein R₁ is:

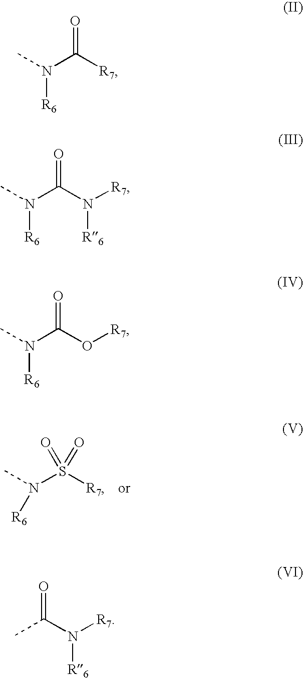

12. A compound according to claim 1, wherein R₁ is: 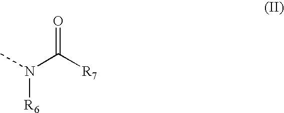
wherein R₇ is C₁₋₁₀ alkyl, C₆₋₁₂ aryl or 3 to 10 membered heterocycle.

13. A compound according to claim 1, wherein R₁ is: 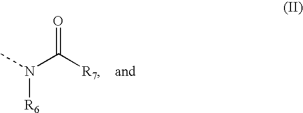
R₇ is methyl, ethyl, isopropyl, cyclopropyl, cyclobutyl, cyclopentyl, cyclohexyl, 4,4-difluorocyclohexyl, cycloheptyl, CH₂-cyclopropyl, CH₂-cyclobutyl, CH₂-cyclopentyl, or CH₂-cyclohexyl.

14. A compound according to claim 1, wherein R₁ is: 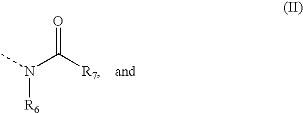
R₇ is phenyl, phenyl substituted with methyl, phenyl substituted with at least one methyl, phenyl substituted with a halogen, phenyl substituted with at least one halogen, phenyl substituted with Cl, phenyl substituted with Br, phenyl substituted with F, phenyl substituted with at least one Cl, or phenyl substituted with methoxy.

15. A compound according to claim 1, wherein R₁ is: 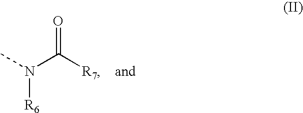
R₇ is benzyl, benzyl substituted with methyl, benzyl substituted with at least one methyl, benzyl substituted with a halogen, benzyl substituted with at least one halogen, benzyl substituted with Cl, benzyl substituted with Br, benzyl substituted with F, benzyl substituted with at least one Cl, benzyl substituted with methoxy, or pyridine.

16. A compound according to claim 1, wherein R₁ is: 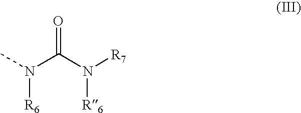
wherein R₆ and R ₆ are each independently H or C₁₋₄ alkyl, R₇ is C₆₋₁₂ aryl, or R ₆ and R₇ taken together form a 3 to 10 membered heterocycle.

17. A compound according to claim 1, wherein R₁ is: 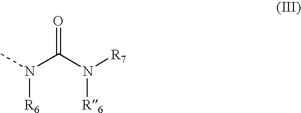
wherein
R₆ and R″₆ are each, independently, H or C₁₋₄ alkyl, and R₇ is phenyl, phenyl substituted with methyl, phenyl substituted with at least one methyl, phenyl substituted with a halogen, phenyl substituted with at least one halogen, phenyl substituted with Cl, phenyl substituted with Br, phenyl substituted with F, phenyl substituted with at least one Cl, phenyl substituted with methoxy, or naphthyl, or R″₆ and R₇ taken together form a piperidine.

18. A compound according to claim 1, wherein R₁ is: 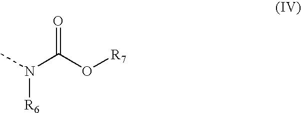
wherein R₆ is H or C₁₋₄ alkyl and R₇ is C₁₋₁₀ alkyl.

19. A compound according to claim 1, wherein R₁ is: 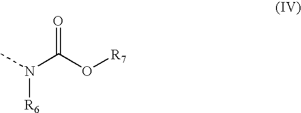
wherein R₆ is H or C₁₋₄ alkyl, and R₇ is methyl, ethyl, tert-butyl, cyclobutyl, cyclopentyl, or cyclohexyl.

20. A compound according to claim 1, wherein R₁ is: 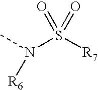
wherein R₆ is H or C₁₋₄ alkyl, and R₇ is optionally substituted C₁₋₁₀ alkyl, optionally substituted C₆₋₁₂ aryl or optionally substituted 3 to 10 membered heterocycle.

21. A compound according to claim 1, wherein R₁ is: 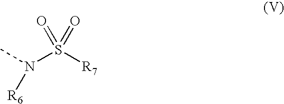
wherein R₆ is H or C₁₋₄ alkyl, and R₇ is optionally substituted phenyl or optionally substituted C₁₋₁₀ alkyl.

22. A compound according to claim 1, wherein R₁ is: 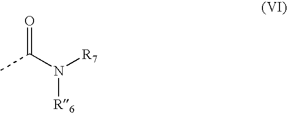
wherein R″₆ is H or C₁₋₄ alkyl, and R₇ is C₁₋₁₀ alkyl or C₆₋₁₂ aryl.

23. A compound according to claim 1, wherein R₁ is: 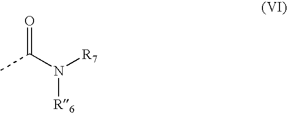
wherein R″₆ is H or C₁₋₄ alkyl, and R₇ is cyclohexyl or phenyl.

24. A compound according to claim 1, wherein R₂ is C₆₋₁₂ aryl or 3 to 10 membered heterocycle.

25. A compound according to claim 1, wherein R₂ is C₆₋₁₂ aryl.

26. A compound according to claim 1, wherein R₂ is phenyl, phenyl substituted with halogen, phenyl substituted with Cl, phenyl substituted with at least one halogen, phenyl substituted with methoxy, or phenyl substituted with at least one methoxy.

27. A compound according to claim 1, wherein R₂ is 3 to 10 membered heterocycle.

28. A compound according to claim 1, wherein R₂ is thienyl or pyridyl.

29. A compound according to claim 1, wherein R₃ is H or C₁₋₄ alkyl.

30. A compound according to claim 1, wherein R₃ is H or methyl.

31. A compound according to claim 1, wherein said compound is selected from:
2-(4-bromobenzyl)-8-(3-phenyl-propyl)-2,8-diaza-spiro[4.5]decan-1-one;
8-(3-phenylpropyl)-2-(4-trifluoromethyl-benzyl)-2,8-diaza-spiro[4.5]decan-1-one;
2-(4-chlorobenzyl)-8-(3-phenyl-propyl)-2,8-diaza-spiro[4.5]decan-1-one;
2-(4-fluorobenzyl)-8-(3-phenyl-propyl)-2,8-diaza-spiro[4.5)decan-1-one;
8-(3-phenyl-propyl)-2-(4-trifluoromethoxy-benzyl)-2,8-diaza-spiro[4.5]decan-1-one;
2-(4-methylbenzyl)-8-(3-phenyl-propyl)-2,8-diaza-spiro[4.5]decan-1-one;
4-[1-oxo-8-(3-phenyl-propyl)-2,8-diaza-spiro[4.5]dec-2-ylmethyl]-benzonitrile;
2-biphenyl-4-ylmethyl-8-(3-phenyl-propyl)-2,8-diaza-spiro[4.5]decan-1-one;
2-naphthalen-2-ylmethyl-8-(3-phenyl-propyl)-2,8-diaza-spiro[4.5]decan-1-one;
2-(4-bromobenzyl)-8-(3-phenyl-butyl)-2,8-diaza-spiro[4.5]decan-1-one;
2-(4-bromobenzyl)-8-(3,3-diphenyl-propyl)-2,8-diaza-spiro[4.5]decan-1-one;
8-(3,3-diphenyl-propyl)-2-(4-trifluoromethoxy-benzyl)-2,8-diaza-spiro[4.5]decan-1-one;
2-(4-bromobenzyl)-8-(3,3-diphenyl-propyl)-2,8-diaza-spiro[4.5]decan-3-one;
8-(3,3-diphenyl-propyl)-2-(3-phenyl-propyl)-2,8-diaza-spiro[4.5]decan-1-one;
8-(3,3-diphenyl-propyl)-2-pyridin-4-ylmethyl-2,8-diaza-spiro[4.5]decan-1-one;
8-(3,3-diphenyl-propyl)-2-(4-methoxy-benzyl)-2,8-diaza-spiro[4.5]decan-1-one;
8-(3,3-diphenyl-propyl)-2-(4-pyrazol-1-yl-benzyl)-2,8-diaza-spiro[4.5]decan-1-one;
2-benzothiazol-2-ylmethyl-8-(3,3-diphenyl-propyl)-2,8-diaza-spiro[4.5]decan-1-one;
8-(3,3-diphenyl-propyl)-2-(4-methanesulfonyl-benzyl)-2,8-diaza-spiro[4.5]decan-1-one;
8-(3,3-diphenyl-propyl)-2-(3-phenyl-allyl)-2,8-diaza-spiro[4.5]decan-1-one;
8-(3,3-diphenyl-propyl)-2-phenethyl-2,8-diaza-spiro[4.5]decan-1-one;
2-(4-benzyloxy-benzyl)-8-(3,3-diphenyl-propyl)-2,8-diaza-spiro[4.5]decan-1-one;
2-benzofuran-2-ylmethyl-8-(3,3-diphenyl-propyl)-2,8-diaza-spiro[4.5]decan-1-one;
8-(3,3-diphenyl-propyl)-2-(4-isopropyl-benzyl)-2,8-diaza-spiro[4.5]decan-1-one;
2-(5-chloro-benzo[b]thiophen-3-ylmethyl)-8-(3,3-diphenyl-propyl)-2,8-diaza-spiro[4.5]decan-1-one;
8-(3,3-diphenyl-propyl)-2-(4-nitro-benzyl)-2,8-diaza-spiro[4.5]decan-1-one;
2-(4-bromo-benzyl)-8-(3-pyridin-2-yl-propyl)-2,8-diaza-spiro[4.5]decan-1-one;
2-[1-(4-bromophenyl)-ethyl]-8-(3,3-diphenyl-propyl)-2,8-diaza-spiro[4.5]decan-1-one;
8-(3,3-diphenyl-propyl)-2-pyridin-3-ylmethyl-2,8-diaza-spiro[4.5]decan-1-one;
N-{4-[8-(3,3-diphenyl-propyl)-1-oxo-2,8-diaza-spiro[4.5]dec-2-ylmethyl]-phenyl}-acetamide;
8-(3,3-diphenyl-propyl)-2-(6-trifluoromethyl-pyridin-3-ylmethyl)-2,8-diaza-spiro[4.5]decan-1-one;
4-[8-(3,3-diphenyl-propyl)-1-oxo-2,8-diaza-spiro[4.5]dec-2-ylmethyl]-benzoic acid;
8-(3,3-diphenyl-propyl)-2-pyridin-2-ylmethyl-2,8-diaza-spiro[4.5]decan-1-one;
8-(3,3-diphenyl-propyl)-2-(4-trifluoromethylsulfanyl -benzyl)-2,8-diaza-spiro[4.5]decan-1-one;
8-(3,3-diphenyl-propyl)-2-(4-methyl-cyclohexylmethyl)-2,8-diaza-spiro[4.5]decan-1-one;
4-[8-(3,3-diphenyl-propyl)-1-oxo-2,8-diaza-spiro[4.5)dec-2-ylmethyl]-benzoic acid methyl ester;
8-(3,3-diphenyl-propyl)-2-(5-trifluoromethyl-furan-2-ylmethyl)-2,8-diaza-spiro[4.5]decan-1-one;
8-(3,3-diphenyl-propyl)-2-(4-iodo-benzyl)-2,8-diaza-spiro[4.5]decan-1-one;
2-(4-methanesulfonylbenzyl)-8-(3-phenyl-butyl)-2,8-diaza-spiro[4.5]decan-1-one;
2-(4-bromobenzyl)-8-[3-hydroxy-3-(2-methoxyphenyl)-3-phenyl-propyl]-2,8-diaza-spiro[4.5]decan-1-one;
2-(4-bromobenzyl)-8-[3-hydroxy-3-(3-methoxyphenyl)-3-phenyl-propyl]-2,8-diaza-spiro[4.5]decan-1-one;
2-(4-bromobenzyl)-8-(3-hydroxy-3-phenyl-3-thiophen-2-yl-propyl)-2,8-diaza-spiro[4.5]decan-1-one;
2-(4-bromobenzyl)-8-(3-hydroxy-3-phenyl-butyl)-2,8-diaza-spiro[4.5]decan-1-one;
2-(4-bromobenzyl)-8-[3-(2-methoxyphenyl)-3-phenyl-propyl]-2,8-diaza-spiro[4.5]decan-1-one;
2-(4-bromobenzyl)-8-[3-(3-chlorophenyl)-3-hydroxy-3-phenyl-propyl]-2,8-diaza-spiro[4.5]decan-1-one;
2-(4-bromobenzyl)-8-[3-(4-chlorophenyl)-3-hydroxy-3-phenyl-propyl]-2,8-diaza-spiro[4.5]decan-1-one;
2-(4-bromobenzyl)-8-[3-(3-chlorophenyl)-3-phenyl-propyl]-2,8-diaza-spiro[4.5]decan-1-one;
2-(4-bromobenzyl)-8-(3-phenyl-3-thiophen-2-yl-propyl)-2,8-diaza-spiro[4.5]decan-1-one;
2-(4-bromobenzyl)-8-[3-(4-chlorophenyl)-3-phenyl-propyl]-2,8-diaza-spiro[4.5)decan-1-one;
{3-[2-(4-bromobenzyl)-1-oxo-2,8-diaza-spiro[4.5]dec-8-yl]-1-phenyl-propyl}-carbamic acid tert-butyl ester;
N-{3-[2-(4-bromobenzyl)-1-oxo-2,8-diaza-spiro[4.5]dec-8-yl]-1-phenyl-propyl}-benzamide;
N-{3-[2-(4-bromobenzyl)-1-oxo-2,8-diaza-spiro[4.5]dec-8-yl)-1-phenyl-propyl}-2,6-dimethyl-benzamide;
cyclohexanecarboxylic acid {3-[2-(4-bromobenzyl)-1-oxo-2,8-diaza-spiro[4.5]dec-8-yl]-1-phenyl-propyl}-amide;
N-{3-[2-(4-bromobenzyl)-1-oxo-2,8-diaza-spiro[4.5]dec-8-yl)-1-phenyl-propyl]-2-phenyl-acetamide;
N-{3-[2-(4-bromobenzyl)-1-oxo-2,8-diaza-spiro[4.5]dec-8-yl]-1-phenyl-propyl}-2-(2,4,6-trimethyl-phenyl)-acetamide;
N-{3-[2-(4-bromobenzyl)-1-oxo-2,8-diaza-spiro[4.5]dec-8-yl]-1-phenyl-propyl}-3-phenyl-propionamide;
{3-[2-(4-bromobenzyl)-1-oxo-2,8-diaza-spiro[4.5]dec-8-yl]-1-phenyl-propyl}-methyl-carbamic acid tert-butyl ester;
N-{3-[2-(4-bromobenzyl)-1-oxo-2,8-diaza-spiro[4.5]dec-8-yl]-1-phenyl-propyl}-N-methyl-benzamide;
cyclohexanecarboxylic acid {3-[2-(4-bromo-benzyl)-1-oxo-2,8-diaza-spiro[4.5]dec-8-yl]-1-phenyl-propyl}-methyl-amide;
N-{3-[2-(4-bromobenzyl)-1-oxo-2,8-diaza-spiro[4.5]dec-8-yl]-1-phenyl-propyl)-N-methyl-2-phenyl-acetamide;
N-{3-[2-(4-bromo-benzyl)-1-oxo-2,8-diaza-spiro[4.5]dec-8-yl]-1-phenyl-propyl}-N-methyl-2-(2,4,6-trimethyl-phenyl)-acetamide;
N-{3-[2-(4-bromo-benzyl)-1-oxo-2,8-diaza-spiro[4.5]dec-8-yl]-1-phenyl-propyl}-N-methyl-2-(2,4,6-trimethyl-phenyl)-acetamide;
[3-(1-oxo-2-pyridin-3-ylmethyl-2,8-diaza-spiro[4.5]dec-8-yl)-1-phenyl-propyl]-carbamic acid tert-butyl ester;
{3-[2-(4-methanesulfonyl-benzyl)-1-oxo-2,8-diaza-spiro[4.5]dec-8-yl]-1-phenyl-propyl}-carbamic acid tert-butyl ester;
[3-[2-(4-bromobenzyl)-1-oxo-2,8-diaza-spiro[4.5]dec-8-yl]-1-(3-chloro-phenyl)-propyl]-carbamic acid tert-butyl ester;
N-{3-[2-(4-bromobenzyl)-1-oxo-2,8-diaza-spiro[4.5]dec-8-yl]-1-phenyl-propyl}-acetamide;
cyclopropanecarboxylic acid {3-[2-(4-bromo-benzyl)-1-oxo-2,8-diaza-spiro[4.5]dec-8-yl]-1-phenyl-propyl}-amide;
N-{3-[2-(4-bromo-benzyl)-1-oxo-2,8-diaza-spiro[4.5]dec-8-yl]-1-phenyl-propyl}-isobutyramide;
N-{3-[2-(4-bromobenzyl)-1-oxo-2,8-diaza-spiro[4.5]dec-8-yl]-1-phenyl-propyl}-3-methyl-butyramide;
N-{3-[2-(4-bromo-benzyl)-1-oxo-2,8-diaza-spiro[4.5]dec-8-yl]-1-phenyl-propyl}-2-chloro-benzamide;
N-{3-[2-(4-bromo-benzyl)-1-oxo-2,8-diaza-spiro[4.5]dec-8-yl]-1-phenyl-propyl}-2-methoxy-benzamide;
pyridine-2-carboxylic acid {3-[2-(4-bromobenzyl)-1-oxo-2,8-diaza-spiro[4.5]dec-8-yl]-1-phenyl-propyl}-amide;
N-{3-[2-(4-bromobenzyl)-1-oxo-2,8-diaza-spiro[4.5]dec-8-yl]-1-phenyl-propyl}-3-chloro-benzamide;
N-{3-[2-(4-bromo-benzyl)-1-oxo-2,8-diaza-spiro[4.5]dec-8-yl]-1-phenyl-propyl}-3-methoxy-benzamide;
N-{3-[2-(4-bromobenzyl)-1-oxo-2,8-diaza-spiro[4.5]dec-8-yl]-1-phenyl-propyl}-nicotinamide;
N-{3-[2-(4-bromobenzyl)-1-oxo-2,8-diaza-spiro[4.5]dec-8-yl]-1-phenyl-propyl}-4-chloro-benzamide;
N-{3-[2-(4-bromo-benzyl)-1-oxo-2,8-diaza-spiro[4.5]dec-8-yl]-1-phenyl-propyl}-4-methoxy-benzamide;
N-{3-[2-(4-bromobenzyl)-1-oxo-2,8-diaza-spiro[4.5]dec-8-yl]-1-phenyl-propyl}-isonicotinamide;
N-{3-[2-(4-bromobenzyl)-1-oxo-2,8-diaza-spiro[4.5]dec-8-yl)-1-phenyl-propyl}-3,4-dichloro-benzamide;
N-{3-[2-(4-bromo-benzyl)-1-oxo-2,8-diaza-spiro[4.5]dec-8-yl]-1-phenyl-propyl}-3,4-dimethoxy-benzamide;
N-{3-[2-(4-bromo-benzyl)-1-oxo-2,8-diaza-spiro[4.5]dec-8-yl]-1-phenyl-propyl}-2-(2-chloro-phenyl)-acetamide;
N-{3-[2-(4-bromobenzyl)-1-oxo-2,8-diaza-spiro[4.5]dec-8-yl]-1-phenyl-propyl}-2-(2-methoxy-phenyl)-acetamide;
N-{3-[2-(4-bromobenzyl)-1-oxo-2,8-diaza-spiro[4.5]dec-8-yl]-1-phenyl-propyl}-2-(3-chloro-phenyl)-acetamide;
N-{3-[2-(4-bromobenzyl)-1-oxo-2,8-diaza-spiro[4.5]dec-8-yl)-1-phenyl-propyl}-2-(3-methoxy-phenyl)-acetamide;
N-{3-[2-(4-bromo-benzyl)-1-oxo-2,8-diaza-spiro[4.5]dec-8-yl]-1-phenyl-propyl}-2-pyridin-3-yl-acetamide;
N-{3-[2-(4-bromobenzyl)-1-oxo-2,8-diaza-spiro[4.5]dec-8-yl]-1-phenyl-propyl}-2-(4-methoxy-phenyl)-acetamide;
N-{3-[2-(4-bromobenzyl)-1-oxo-2,8-diaza-spiro[4.5]dec-8-yl]-1-phenyl-propyl}-2-(3,4-dichloro-phenyl)-acetamide;
tetrahydro-pyran-4-carboxylic acid{3-[2-(4-bromobenzyl)-1-oxo-2,8-diaza-spiro[4.5]dec-8-yl]-1-phenyl-propyl}-amide;
cyclopentanecarboxylic acid {3-[2-(4-bromobenzyl)-1-oxo-2,8-diaza-spiro[4.5]dec-8-yl]-1-phenyl-propyl}-amide;
cyclobutanecarboxylic acid {3-[2-(4-bromobenzyl)-1-oxo-2,8-diaza-spiro[4.5]dec-8-yl]-1-phenyl-propyl}-amide;
cycloheptanecarboxylic acid {3-[2-(4-bromobenzyl)-1-oxo-2,8-diaza-spiro[4.5]dec-8-yl]-1-phenyl-propyl}-amide;
N-{3-[2-(4-bromo-benzyl)-1-oxo-2,8-diaza-spiro[4.5]dec-8-yl]-1-phenyl-propyl}-2-cyclohexyl-acetamide;
N-{3-[2-(4-methoxybenzyl)-1-oxo-2,8-diaza-spiro[4.5]dec-8-yl]-1-phenyl-propyl}-acetamide;
cyclopropanecarboxylic acid {3-[2-(4-methoxybenzyl)-1-oxo-2,8-diaza-spiro[4.5]dec-8-yl]-1-phenyl-propyl}-amide;
N-{3-[2-(4-methoxy-benzyl)-1-oxo-2,8-diaza-spiro[4.5]dec-8-yl]-1-phenyl-propyl}-isobutyramide;
N-{3-[2-(4-methoxybenzyl)-1-oxo-2,8-diaza-spiro[4.5]dec-8-yl]-1-phenyl-propyl}-3-methyl-butyramide;
2-chloro-N-{3-[2-(4-methoxybenzyl)-1-oxo-2,8-diaza-spiro[4.5]dec-8-yl]-1-phenyl-propyl}-benzamide;
2-methoxy-N-{3-[2-(4-methoxybenzyl)-1-oxo-2,8-diaza-spiro[4.5]dec-8-yl]-1-phenyl-propyl}-benzamide;
pyridine-2-carboxylic acid {3-[2-(4-methoxybenzyl)-1-oxo-2,8-diaza-spiro[4.5]dec-8-yl]-1-phenyl-propyl}-amide;
3-chloro-N-{3-[2-(4-methoxy-benzyl)-1-oxo-2,8-diaza-spiro[4.5]dec-8-yl]-1-phenyl-propyl}-benzamide;
3-methoxy-N-{3-[2-(4-methoxy-benzyl)-1-oxo-2,8-diaza-spiro[4.5]dec-8-yl]-1-phenyl-propyl}-benzamide;
N-{3-[2-(4-methoxybenzyl)-1-oxo-2,8-diaza-spiro[4.5)dec-8-yl]-1-phenyl-propyl}-nicotinamide;
4-chloro-N-{3-[2-(4-methoxybenzyl)-1-oxo-2,8-diaza-spiro[4.5]dec-8-yl]-1-phenyl-propyl}-benzamide;
4-methoxy-N-{3-[2-(4-methoxy-benzyl)-1-oxo-2,8-diaza-spiro[4.5]dec-8-yl]-1-phenyl-propyl}-benzamide;
N-{3-[2-(4-methoxybenzyl)-1-oxo-2,8-diaza-spiro[4.5]dec-8-yl]-1-phenyl-propyl}-isonicotinamide;
(R)-cyclohexanecarboxylic acid {3-[2-(4-bromobenzyl)-1-oxo-2,8-diaza-spiro [4.5]dec-8-yl]-l(R)-phenyl-propyl}-amide;
3,4-dichloro-N-{3-[2-(4-methoxybenzyl)-1-oxo-2,8-diaza-spiro[4.5]dec-8-yl]-1-phenyl-propyl}-benzamide;
3,4-dimethoxy-N-{3-[2-(4-methoxybenzyl)-1-oxo-2,8-diaza-spiro[4.5]dec-8-yl]-1-phenyl-propyl}-benzamide;
2-(2-chloro-phenyl)-N-{3-[2-(4-methoxybenzyl)-1-oxo-2,8-diaza-spiro[4.5)dec-8-yl]-1-phenyl-propyl}-acetamide;
N-{3-[2-(4-methoxybenzyl)-1-oxo-2,8-diaza-spiro[4.5]dec-8-yl]-1-phenyl-propyl}-2-(2-methox-phenyl)-acetamide;
2-(3-chlorophenyl)-N-13-[2-(4-methoxybenzyl)-1-oxo-2,8-diaza-spiro[4.5]dec-8-yl]-1-phenyl-propyl}-acetamide;
N-13-[2-(4-methoxybenzyl)-1-oxo-2,8-diaza-spiro[4.5]dec-8-yl]-1-phenyl-propyl}-2-(3-methoxyphenyl)-acetamide;
N-(3-[2-(4-methoxybenzyl)-1-oxo-2,8-diaza-spiro[4.5]dec-8-yl]-1-phenyl-propyl}-2-pyridin-3-yl-acetamide;
N-{3-[2-(4-methoxybenzyl)-1-oxo-2,8-diaza-spiro[4.5]dec-8-yl]-1-phenyl-propyl}-2-(4-methoxyphenyl)-acetamide;
2-(3,4-dichlorophenyl)-N-{3-[2-(4-methoxybenzyl)-1-oxo-2,8-diaza-spiro[4.5]dec-8-yl]-1-phenyl-propyl}-acetamide;
tetrahydro-pyran-4-carboxylic acid{3-[2-(4-methoxybenzyl)-1-oxo-2,8-diaza-spiro[4.5]dec-8-yl]-1-phenyl-propyl}-amide;
cyclopentanecarboxylic acid {3-[2-(4-methoxybenzyl)-1-oxo-2,8-diaza-spiro[4.5]dec-8-yl]-1-phenyl-propyl}-amide;
cyclobutanecarboxylic acid {3-[2-(4-methoxybenzyl)-1-oxo-2,8-diaza-spiro[4.5]dec-8-yl]-1-phenyl-propyl}-amide;
cycloheptanecarboxylic acid {3-[2-(4-methoxybenzyl)-1-oxo-2,8-diaza-spiro[4.5]dec-8-yl]-1-phenyl-propyl}-amide;
2-cyclohexyl-N-{3-[2-(4-methoxybenzyl)-1-oxo-2,8-diaza-spiro[4.5]dec-8-yl]-1-phenyl-propyl}-acetamide;
(S)-cyclohexanecarboxylic acid {3-[2-(4-bromobenzyl)-1-oxo-2,8-diaza-spiro[4.5]dec-8-yl]-1-phenyl-propyl}-amide;
N-{3-[2-(4-bromobenzyl)-1-oxo-2,8-diaza-spiro[4.5]dec-8-yl]-1-phenyl-propyl}-2-cyclopentyl-acetamide;
furan-2-carboxylic acid {3-[2-(4-bromobenzyl)-1-oxo-2,8-diaza-spiro[4.5]dec-8-yl]-1-phenyl-propyl}-amide;
N-{3-[2-(4-bromobenzyl)-1-oxo-2,8-diaza-spiro[4.5]dec-8-yl]-1-phenyl-propyl}-2-ethyl-butyramide;
thiophene-2-carboxylic acid {3-[2-(4-brom-benzyl)-1-oxo-2,8-diaza-spiro[4.5]dec-8-yl]-1-phenyl-propyl}-amide;
N-{3-[2-(4-bromobenzyl)-1-oxo-2,8-diaza-spiro[4.5]dec-8-yl]-1-phenyl-propyl}-2-(3,4-dimethoxyphenyl)-acetamide;
2-cyclopentyl-N-{3-[2-(4-methoxybenzyl)-1-oxo-2,8-diaza-spiro[4.5]dec-8-yl]-1-phenyl-propyl}-acetamide;
furan-2-carboxylic acid {3-[2-(4-methox-benzyl)-1-oxo-2,8-diaza-spiro[4.5]dec-8-yl]-1-phenyl-propyl}-amide;
2-ethyl-N-{3-[2-(4-methoxybenzyl)-1-oxo-2,8-diaza-spiro[4.5]dec-8-yl]-1-phenyl-propyl}-butyramide;
thiophene-2-carboxylic acid {3-[2-(4-methoxybenzyl)-1-oxo-2,8-diaza-spiro[4.5]dec-8-yl]-1-phenyl-propyl}-amide;
2-(3,4-dimethoxy-phenyl)-N-{3-[2-(4-methoxybenzyl)-1-oxo-2,8-diaza-spiro[4.5]dec-8-yl)-1-phenyl-propyl}-acetamide;
cyclohexanecarboxylic acid {3-[2-(4-methoxybenzyl)-1-oxo-2,8-diaza-spiro[4.5]dec-8-yl]-1-phenyl-propyl}-amide;
N-{3-[2-(4-methoxybenzyl)-1-oxo-2,8-diaza-spiro[4.5]dec-8-yl]-1-phenyl-propyl}-benzamide;
N-{3-[2-(4-methoxybenzyl)-1-oxo-2,8-diaza-spiro[4.5]dec-8-yl]-1-phenyl-propyl}-2-phenyl-acetamide;
N-{3-[2-(4-methanesulfonylbenzyl)-1-oxo-2,8-diaza-spiro[4.5]dec-8-yl]-1-phenyl-propyl)-acetamide;
cyclopropanecarboxylic acid {3-[2-(4-methanesulfonylbenzyl)-1-oxo-2,8-diaza-spiro[4.5]dec-8-yl]-1-phenyl-propyl}amide;
N-{3-[2-(4-methanesulfonyl-benzyl)-1-oxo-2,8-diaza-spiro[4.5]dec-8-yl]-1-phenyl-propyl)-isobutyramide;
N-{3-[2-(4-methanesulfonylbenzyl)-1-oxo-2,18-diaza-spiro[4.5]dec-8-yl]-1-phenyl-propyl}-3-methyl-butyramide;
2-chloro-N-{3-[2-(4-methanesulfonylbenzyl)-1-oxo-2,8-diaza-spiro[4.5]dec-8-yl]-1-phenyl-propyl}-benzamide;
N-{3-[2-(4-methanesulfonylbenzyl)-1-oxo-2,8-diaza-spiro[4.5]dec-8-yl]-1-phenyl-propyl)-2-methoxy-benzamide;
pyridine-2-carboxylic acid {3-[2-(4-methanesulfonylbenzyl)-1-oxo-2,8-diaza-spiro[4.5]dec-8-yl]-1-phenyl-propyl}-amide;
3-chloro-N-{3-[2-(4-methanesulfonylbenzyl)-1-oxo-2,8-diaza-spiro[4.5]dec-8-yl]-1-phenyl-propyl}-benzamide;
N-{3-[2-(4-methanesulfonylbenzyl)-1-oxo-2,8-diaza-spiro[4.5]dec-8-yl]-1-phenyl-propyl}-3-methoxy-benzamide;
N-{3-[2-(4-methanesulfonylbenzyl)-1-oxo-2,8-diaza-spiro[4.5]dec-8-yl]-1-phenyl-propyl)-nicotinamide;
4-chloro-N-{3-[2-(4-methanesulfonylbenzyl)-1-oxo-2,8-diaza-spiro[4.5]dec-8-yl]-1-phenyl-propyl}-benzamide;
N-{3-[2-(4-methanesulfonylbenzyl)-1-oxo-2,8-diaza-spiro[4.5]dec-8-yl]-1-phenyl-propyl}-4-methoxy-benzamide;
N-{3-[2-(4-methanesulfonylbenzyl)-1-oxo-2,8-diaza-spiro[4.5]dec-8-yl]-1-phenyl-propyl}-isonicotinamide;
3,4-dichloro-N-{3-[2-(4-methanesulfonylbenzyl)-1-oxo-2,8-diaza-spiro[4.5]dec-8-yl]-1-phenyl-propyl}-benzamide;
N-{3-[2-(4-methanesulfonylbenzyl)-1-oxo-2,8-diaza-spiro[4.5]dec-8-yl]-1-phenyl-propyl}-3,4-dimethoxy-benzamide;
2-(2-chlorophenyl)-N-{3-[2-(4-methanesulfonylbenzyl)-1-oxo-2,8-diaza-spiro[4.5]dec-8-yl]-1-phenyl-propyl}-acetamide;
N-{3-[2-(4-methanesulfonylbenzyl)-1-oxo-2,8-diaza-spiro[4.5]dec-8-yl]-1-phenyl-propyl}-2-(2-methoxyphenyl)-acetamide;
2-(3-chlorophenyl)-N-{3-[2-(4-methanesulfonylbenzyl)-1-oxo-2,8-diaza-spiro[4.5]dec-8-yl]-1-phenyl-propyl}-acetamide;
N-{3-[2-(4-methanesulfonylbenzyl)-1-oxo-2,8-diaza-spiro[4.5]dec-8-yl]-1-phenyl-propyl}-2-(3-methoxyphenyl)-acetamide;
N-{3-[2-(4-methanesulfonylbenzyl)-1-oxo-2,8-diaza-spiro[4.5]dec-8-yl]-1-phenyl-propyl}-benzamide;
(S)-cyclohexanecarboxylic acid (3-(2-benzyl-1-oxo-2,8-diaza-spiro[4.5]dec-8-yl)-1-phenyl-propyl]-amide;
N-{3-[2-(4-methanesulfonylbenzyl)-1-oxo-2,8-diaza-spiro[4.5]dec-8-yl]-1-phenyl-propyl}-2-(4-methoxyphenyl)-acetamide;
N-{3-[2-(4-methanesulfonylbenzyl)-1-oxo-2,8-diaza-spiro[4.5]dec-8-yl]-1-phenyl-propyl}-2-phenyl-acetamide;
2-(3,4-dichlorophenyl)-N-{3-[2-(4-methanesulfonylbenzyl)-1-oxo-2,8-diaza-spiro[4.5]dec-8-yl]-1-phenyl-propyl}-acetamide;
cyclopentanecarboxylic acid {3-(2-(4-methanesulfonylbenzyl)-1-oxo-2,8-diaza-spiro[4.5]dec-8-yl]-1-phenyl-propyl}-amide;
cyclobutanecarboxylic acid {3-[2-(4-methanesulfonylbenzyl)-1-oxo-2,8-diaza-spiro[4.5]dec-8-yl]-1-phenyl-propyl}-amide;
cycloheptanecarboxylic acid {3-[2-(4-methanesulfonylbenzyl)-1-oxo-2,8-diaza-spiro[4.5]dec-8-yl]-1-phenyl-propyl}-amide;
2-cyclohexyl-N-{3-[2-(4-methanesulfonylbenzyl)-1-oxo-2,8-diaza-spiro[4.5]dec-8-yl]-1-phenyl-propyl}-acetamide;
2-cyclopentyl-N-{3-[2-(4-methanesulfonylbenzyl)-1-oxo-2,8-diaza-spiro[4.5]dec-8-yl]-1-phenyl-propyl}-acetamide;
furan-2-carboxylic acid {3-[2-(4-methanesulfonylbenzyl)-1-oxo-2,8-diaza-spiro[4.5]dec-8-yl]-1-phenyl-propyl}-amide;
2-ethyl-N-{3-[2-(4-methanesulfonylbenzyl)-1-oxo-2,8-diaza-spiro[4.5]dec-8-yl]-1-phenyl-propyl}-butyramide;
thiophene-2-carboxylic acid {3-[2-(4-methanesulfonylbenzyl)-1-oxo-2,8-diaza-spiro[4.5]dec-8-yl]-1-phenyl-propyl}-amide;
2-(3,4-dimethoxyphenyl)-N-{3-[2-(4-methanesulfonyl-benzyl)-1-oxo-2,8-diaza-spiro[4.5]dec-8-yl]-1-phenyl-propyl}-acetamide;
cyclohexanecarboxylic acid {3-[2-(4-methanesulfonylbenzyl)-1-oxo-2,8-diaza-spiro[4.5]dec-8-yl]-1-phenyl-propyl)-amide;
4-methyl-cyclohexanecarboxylic acid {3-[2-(4-methanesulfonylbenzyl)-1-oxo-2,8-diaza-spiro[4.5]dec-8-yl]-1-phenyl-propyl}-amide;
2-methoxy-N-[3-(1-oxo-2-pyridin-3-ylmethyl-2,8-diaza-spiro[4.5]dec-8-yl)-1-phenyl-propyl]-benzamide;
3-chloro-N-[3-(1-oxo-2-pyridin-3-ylmethyl-2,8-diaza-spiro[4.5]dec-8-yl)-1-phenyl-propyl]-benzamide;
4-chloro-N-[3-(1-oxo-2-pyridin-3-ylmethyl-2,8-diaza-spiro[4.5]dec-8-yl)-1-phenyl-propyl]-benzamide;
4-methoxy-N-[3-(1-oxo-2-pyridin-3-ylmethyl-2,8-diaza-spiro[4.5]dec-8-yl)-1-phenyl-propyl]-benzamide;
cyclohexanecarboxylic acid [3-[2-(4-bromobenzyl)-1-oxo-2,8-diaza-spiro[4.5]dec-8-yl]-1-(3-chloro-phenyl)-propyl]-amide;
N-[3-[2-(4-bromobenzyl)-1-oxo-2,8-diaza-spiro[4.5]dec-8-yl]-1-(3-chlorophenyl)-propyl]-benzamide;
N-[3-[2-(4-bromobenzyl)-1-oxo-2,8-diaza-spiro[4.5]dec-8-yl]-1-(3-chlorophenyl)-propyl]-2-phenyl-acetamide;
{1-(3-chlorophenyl)-3-[2-(4-methoxybenzyl)-1-oxo-2,8-diaza-spiro[4.5]dec-8-yl]-propyl}-carbamic acid tert-butyl ester;
1-(3,4-dichlorophenyl)-3-[2-(4-methoxybenzyl)-1-oxo-2,8-diaza-spiro[4.5]dec-8-yl]-propyl}-carbamic acid tert-butyl ester;
N-[3-(1-oxo-2-pyridin-3-ylmethyl-2,8-diaza-spiro[4.5]dec-8-yl)-1-phenyl-propyl]-acetamide;
cyclopropanecarboxylic acid [3-(1-oxo-2-pyridin-3-ylmethyl-2,8-diaza-spiro[4.5]dec-8-yl)-1-phenyl-propyl]-amide;
N-[3-(1-oxo-2-pyridin-3-ylmethyl-2,8-diaza-spiro[4.5]dec-8-yl)-1-phenyl-propyl]-isobutyramide;
3-methyl-N-[3-(1-oxo-2-pyridin-3-ylmethyl-2,8-diaza-spiro[4.5]dec-8-yl)-1-phenyl-propyl]-butyramide;
2-chloro-N-[3-(1-oxo-2-pyridin-3-ylmethyl-2,8-diaza-spiro[4.5]dec-8-yl)-1-phenyl-propyl]-benzamide;
pyridine-2-carboxylic acid [3-(1-oxo-2-pyridin-3-ylmethyl-2,8-diaza-spiro[4.5]dec-8-yl)-1-phenyl-propyl]-amide;
3-methoxy-N-[3-(1-oxo-2-pyridin-3-ylmethyl-2,8-diaza-spiro[4.5]dec-8-yl)-1-phenyl-propyl]-benzamide;
N-[3-(1-oxo-2-pyridin-3-ylmethyl-2,8-diaza-spiro[4.5]dec-8-yl)-1-phenyl-propyl]-nicotinamide;
N-[3-(1-oxo-2-pyridin-3-ylmethyl-2,8-diaza-spiro[4.5]dec-8-yl)-1-phenyl-propyl]-isonicotinamide;
3,4-dichloro-N-[3-(1-oxo-2-pyridin-3-ylmethyl-2,8-diaza-spiro[4.5]dec-8-yl)-1-phenyl-propyl]-benzamide;
3,4-dimethoxy-N-[3-(1-oxo-2-pyridin-3-ylmethyl-2,8-diaza-spiro[4.5]dec-8-yl)-1-phenyl-propyl]-benzamide;
2-(2-chlorophenyl)-N-[3-(1-oxo-2-pyridin-3-ylmethyl-2,8-diaza-spiro[4.5]dec-8-yl)-1-phenyl-propyl]-acetamide;
2-(2-methoxyphenyl)-N-[3-(1-oxo-2-pyridin-3-ylmethyl-2,8-diaza-spiro[4.5]dec-8-yl)-1-phenyl-propyl]-acetamide;
N-[3-(1-oxo-2-pyridin-3-ylmethyl-2,8-diaza-spiro[4.5]dec-8-yl)-1-phenyl-propyl]-benzamide;
2-(3-chloro-phenyl)-N-[3-(1-oxo-2-pyridin-3-ylmethyl-2,8-diaza-spiro[4.5]dec-8-yl)-1-phenyl-propyl]-acetamide;
2-(3-methoxyphenyl)-N-[3-(1-oxo-2-pyridin-3-ylmethyl-2,8-diaza-spiro[4.5]dec-8-yl)-1-phenyl-propyl]-acetamide;
2-(4-methoxyphenyl)-N-[3-(1-oxo-2-pyridin-3-ylmethyl-2,8-diaza-spiro[4.5]dec-8-yl)-1-phenyl-propyl]-acetamide;
N-[3-(1-oxo-2-pyridin-3-ylmethyl-2,8-diaza-spiro[4.5]dec-8-yl)-1-phenyl-propyl]-2-phenyl-acetamide;
2-(3,4-dichloro-phenyl)-N-[3-(1-oxo-2-pyridin-3-ylmethyl-2,8-diaza-spiro[4.5]dec-8-yl)-1-phenyl-propyl]-acetamide;
cyclopentanecarboxylic acid [3-(1-oxo-2-pyridin-3-ylmethyl-2,8-diaza-spiro[4.5]dec-8-yl)-1-phenyl-propyl]-amide;
(1-(3-chlorophenyl)-3-[2-(4-methanesulfonylbenzyl)-1-oxo-2,8-diaza-spiro[4.5]dec-8-yl]-propyl}-carbamic acid tert-butyl ester;
cyclobutanecarboxylic acid [3-(1-oxo-2-pyridin-3-ylmethyl-2,8-diaza-spiro[4.5]dec-8-yl)-1-phenyl-propyl]-amide;
cycloheptanecarboxylic acid [3-(1-oxo-2-pyridin-3-ylmethyl-2,8-diaza-spiro[4.5]dec-8-yl)-1-phenyl-propyl]-amide;
2-cyclohexyl-N-[3-(1-oxo-2-pyridin-3-ylmethyl-2,8-diaza-spiro[4.5]dec-8-yl)-1-phenyl-propyl]-acetamide;
2-cyclopentyl-N-[3-(1-oxo-2-pyridin-3-ylmethyl-2,8-diaza-spiro[4.5]dec-8-yl)-1-phenyl-propyl]-acetamide;
furan-2-carboxylic acid [3-(1-oxo-2-pyridin-3-ylmethyl-2,8-diaza-spiro[4.5]dec-8-yl)-1-phenyl-propyl]-amide;
2-ethyl-N-[3-(1-oxo-2-pyridin-3-ylmethyl-2,8-diaza-spiro[4.5]dec-8-yl)-1-phenyl-propyl]-butyramide;
thiophene-2-carboxylic acid [3-(1-oxo-2-pyridin-3-ylmethyl-2,8-diaza-spiro[4.5]dec-8-yl)-1-phenyl-propyl]-amide;
2-(3,4-dimethoxyphenyl)-N-[3-(1-oxo-2-pyridin-3-ylmethyl-2,8-diaza-spiro[4.5]dec-8-yl)-1-phenyl-propyl]-acetamide;
cyclohexanecarboxylic acid [3-(1-oxo-2-pyridin-3-ylmethyl-2,8-diaza-spiro[4.5]dec-8-yl)-1-phenyl-propyl]-amide;
4-methyl-cyclohexanecarboxylic acid [3-(1-oxo-2-pyridin-3-ylmethyl-2,8-diaza-spiro[4.5]dec-8-yl)-1-phenyl-propyl]-amide;
[3-[2-(4-bromobenzyl)-1-oxo-2,8-diaza-spiro[4.5]dec-8-yl]-1-(3-methoxyphenyl)-propyl]-carbamic acid tert-butyl ester;
[3-[2-(4-methoxybenzyl)-1-oxo-2,8-diaza-spiro[4.5]dec-8-yl]-1-(3-methoxyphenyl)-propyl]-carbamic acid tert-butyl ester;
(S)-N-{3-[2-(4-bromobenzyl)-1-oxo-2,8-diaza-spiro[4.5]dec-8-yl]-1-phenyl-propyl}-acetamide;
(S)-cyclopropanecarboxylic acid {3-[2-(4-bromobenzyl)-1-oxo-2,8-diaza-spiro[4.5]dec-8-yl]-1-phenyl-propyl}-amide;
(S)-N-{3-[2-(4-bromobenzyl)-1-oxo-2,8-diaza-spiro[4.5]dec-8-yl]-1-phenyl-propyl}-isobutyramide;
(S)-N-{3-[2-(4-bromobenzyl)-1-oxo-2,8-diaza-spiro[4.5]dec-8-yl]-1-phenyl-propyl}-isobutyramide;
(S)-N-{3-[2-(4-bromobenzyl)-1-oxo-2,8-diaza-spiro[4.5]dec-8-yl]-1-phenyl-propyl}-3-methyl-butyramide;
(S)-cyclopentanecarboxylic acid {3-[2-(4-bromobenzyl)-1-oxo-2,8-diaza-spiro[4.5]dec-8-yl]-1-phenyl-propyl}-amide;
(S)-cyclobutanecarboxylic acid {3-[2-(4-bromobenzyl)-1-oxo-2,8-diaza-spiro[4.5]dec-8-yl]-1-phenyl-propyl}-amide;
[3-[2-(4-methoxybenzyl)-1-oxo-2,8-diaza-spiro[4.5]dec-8-yl]-1-(4-methoxyphenyl)-propyl]-carbamic acid tert-butyl ester;
(3-[2-(4-bromobenzyl)-1-oxo-2,8-diaza-spiro[4.5]dec-8-yl]-1-pyridin-2-yl-propyl}-carbamic acid tert-butyl ester;
{3-[2-(4-methoxybenzyl)-1-oxo-2,8-diaza-spiro[4.5]dec-8-yl]-1-pyridin-2-yl-propyl}-carbamic acid tert-butyl ester;
(1-(3,4-dichlorophenyl)-3-[2-(4-methanesulfonylbenzyl)-1-oxo-2,8-diaza-spiro[4.5]dec-8-yl]-propyl}-carbamic acid tert-butyl ester;
2-cyclopropyl-N-{3-[2-(4-methoxybenzyl)-1-oxo-2,8-diaza-spiro[4.5]dec-8-yl]-1-phenyl-propyl}-acetamide;
2-cyclopropyl-N-{3-[2-(4-methanesulfonylbenzyl)-1-oxo-2,8-diaza-spiro[4.5]dec-8-yl]-1-phenyl-propyl}-acetamide;
[3-[2-(4-bromobenzyl)-1-oxo-2,8-diaza-spiro[4.5]dec-8-yl]-1-(4-methoxyphenyl)-propyl]-carbamic acid tert-butyl ester;
N-{3-[2-(4-bromobenzyl)-1-oxo-2,8-diaza-spiro[4.5]dec-8-yl]-1-phenyl-propyl}-2-cyclopropyl-acetamide;
[3-[2-(4-bromobenzyl)-1-oxo-2,8-diaza-spiro[4.5]dec-8-yl]-1-(3,4-dimethoxyphenyl)-propyl]-carbamic acid tert-butyl ester;
1-(3,4-dimethoxyphenyl)-3-[2-(4-methoxybenzyl)-1-oxo-2,8-diaza-spiro[4.5]dec-8-yl]-propyl}-carbamic acid tert-butyl ester;
tetrahydro-pyran-4-carboxylic acid {3-[2-(4-methanesulfonylbenzyl)-1-oxo-2,8-diaza-spiro[4.5]dec-8-yl]-1-phenyl-propyl}-amide;
[3-[2-(4-methanesulfonylbenzyl)-1-oxo-2,8-diaza-spiro[4.5]dec-8-yl]-1-(3-methoxy-phenyl)-propyl]-carbamic acid tertbutyl ester;
(S)-{3-[2-(4-methanesulfonylbenzyl)-1-oxo-2,8-diaza-spiro[4.5]dec-8-yl]-1-phenyl-propyl}-carbamic acid tert-butyl ester;
{3-[2-(4-methanesulfonylbenzyl)-1-oxo-2,8-diaza-spiro[4.5]dec-8-yl]-1-pyridin-2-yl-propyl}-carbamic acid tert-butyl ester;
{1-(3,4-dimethoxyphenyl)-3-[2-(4-methanesulfonylbenzyl)-1-oxo-2,8-diaza-spiro[4.5]dec-8-yl]-propyl}-carbamic acid tert-butyl ester;
{1-(4-chlorophenyl)-3-[2-(4-methoxybenzyl)-1-oxo-2,8-diaza-spiro[4.5]dec-8-yl]-propyl}-carbamic acid tert-butyl ester;
{1-(2-chlorophenyl)-3-[2-(4-methoxybenzyl)-1-oxo-2,8-diaza-spiro[4.5]dec-8-yl]-propyl}-carbamic acid tert-butyl ester;
(3-[2-(4-methoxybenzyl)-1-oxo-2,8-diaza-spiro[4.5]dec-8-yl]-1-thiophen-2-yl-propyl}-carbamic acid tert-butyl ester;
[3-[2-(4-methanesulfonylbenzyl)-1-oxo-2,8-diaza-spiro[4.5)dec-8-yl]-1-(4-methoxyphenyl)-propyl]-carbamic acid tert-butyl ester;
[3-[2-(4-bromobenzyl)-1-oxo-2,8-diaza-spiro[4.5]dec-8-yl)-1-(4-chlorophenyl)-propyl]-carbamic acid tert-butyl ester;
[3-[2-(4-bromobenzyl)-1-oxo-2,8-diaza-spiro[4.5]dec-8-yl]-1-(2-chlorophenyl)-propyl]-carbamic acid tert-butyl ester;
{3-[2-(4-bromobenzyl)-1-oxo-2,8-diaza-spiro[4.5]dec-8-yl]-1-thiophen-2-yl-propyl}-carbamic acid tert-butyl ester;
{1-(4-chlorophenyl)-3-[2-(4-methanesulfonylbenzyl)-1-oxo-2,8-diaza-spiro[4.5)dec-8-yl]-propyl}-carbamic acid tert-butyl ester;
{1-(2-chlorophenyl)-3-[2-(4-methanesulfonylbenzyl)-1-oxo-2,8-diaza-spiro[4.5]dec-8-yl]-propyl}-carbamic acid tert-butyl ester;
(3-[2-(4-methanesulfonylbenzyl)-1-oxo-2,8-diaza-spiro[4.5]dec-8-yl]-1-thiophen-2-yl-propyl}-carbamic acid tert-butyl ester;
(S)-N-{3-[2-(4-methanesulfonylbenzyl)-1-oxo-2,8-diaza-spiro[4.5]dec-8-yl]-1-phenyl-propyl}-isobutyramide;
[3-[2-(4-bromobenzyl)-1-oxo-2,8-diaza-spiro[4.5]dec-8-yl]-1-(2-methoxyphenyl)-propyl]-carbamic acid tert-butyl ester;
[3-[2-(4-methoxy-benzyl)-1-oxo-2,8-diaza-spiro[4.5]dec-8-yl]-1-(2-methoxy-phenyl)-propyl]-carbamic acid tert-butyl ester;
[1-(2-chlorophenyl)-3-(1-oxo-2-pyridin-3-ylmethyl-2,8-diaza-spiro[4.5]dec-8-yl)-propyl]-carbamic acid tert-butyl ester;
[1-(3-chlorophenyl)-3-(1-oxo-2-pyridin-3-ylmethyl-2,8-diaza-spiro[4.5]dec-8-yl)-propyl]-carbamic acid tert-butyl ester;
[1-(3,4-dichlorophenyl)-3-(1-oxo-2-pyridin-3-ylmethyl-2,8-diaza-spiro[4.5]dec-8-yl)-propyl]-carbamic acid tert-butyl ester;
[3-(1-oxo-2-pyridin-3-ylmethyl-2,8-diaza-spiro[4.5]dec-8-yl)-1-thiophen-2-yl-propyl]-carbamic acid tert-butyl ester;
(S)-8-[3-(cyclopropanecarbonyl-amino)-3-phenyl-propyl]-2-(4-methanesulfonylbenzyl)-1-oxo-2,8-diaza-spiro[4.5]decane;
(S)-8-[3-(cyclopentanecarbonyl-amino)-3-phenyl-propyl]-2-(4-methanesulfonylbenzyl)-1-oxo-2,8-diaza-spiro[4.5]decane;
(S)-8-[3-(cyclohexanecarbonyl-amino)-3-phenyl-propyl]-2-(4-methanesulfonylbenzyl)-1-oxo-2,8-diaza-spiro[4.5]decane;
(S)-8-[3-(cyclopropanecarbonyl-amino)-3-phenyl-propyl]-2-(4-methoxybenzyl)-1-oxo-2,8-diaza-spiro[4.5]decane;
(S)-8-(3-isobutyrylamino-3-phenyl-propyl)-2-(4-methoxybenzyl)-1-oxo-2,8-diaza-spiro[4.5]decane;
(S)-8-[3-(cyclopentanecarbonyl-amino)-3-phenyl-propyl]-2-(4-methoxybenzyl)-1-oxo-2,8-diaza-spiro[4.5]decane;
(S)-8-[3-(cyclohexanecarbonyl-amino)-3-phenyl-propyl]-2-(4-methoxybenzyl)-1-oxo-2,8-diaza-spiro[4.5]decane;
N-{3-[2-(4-bromobenzyl)-1-oxo-2,8-diaza-spiro[4.5]dec-8-yl]-1-thiophen-2-yl-propyl}-isobutyramide;
cyclobutanecarboxylic acid {3-[2-(4-bromobenzyl)-1-oxo-2,8-diaza-spiro[4.5]dec-8-yl]-1-thiophen-2-yl-propyl}-amide;
cyclopentanecarboxylic acid {3-[2-(4-bromobenzyl)-1-oxo-2,8-diaza-spiro[4.5]dec-8-yl]-1-thiophen-2-yl-propyl}-amide;
N-{3-[2-(4-bromo-enzyl)-1-oxo-2,8-diaza-spiro[4.5]dec-8-yl]-1-thiophen-2-yl-propyl}-propionamide;
N-{3-[2-(4-bromobenzyl)-1-oxo-2,8-diaza-spiro[4.5]dec-8-yl]-1-thiophen-2-yl-propyl}-2-methoxy-acetamide;
cyclohexanecarboxylic acid {3-[2-(4-bromobenzyl)-1-oxo-2,8-diaza-spiro[4.5]dec-8-yl]-1-thiophen-2-yl-propyl}-amide;
cyclopropanecarboxylic acid {3-[2-(4-methoxybenzyl)-1-oxo-2,8-diaza-spiro[4.5]dec-8-yl]-1-thiophen-2-yl-propyl}-amide;
N-{3-[2-(4-methoxybenzyl)-1-oxo-2,8-diaza-spiro[4.5]dec-8-yl]-1-thiophen-2-yl-propyl}-isobutyramide;
[3-[2-(4-methanesulfonylbenzyl)-1-oxo-2,8-diaza-spiro[4.5]dec-8-yl]-1-(2-methoxyphenyl)-propyl]-carbamic acid tert-butyl ester;
cyclobutanecarboxylic acid {3-[2-(4-methoxybenzyl)-1-oxo-2,8-diaza-spiro[4.5]dec-8-yl]-1-thiophen-2-yl-propyl}-amide;
cyclopentanecarboxylic acid {3-[2-(4-methoxybenzyl)-1-oxo-2,8-diaza-spiro[4.5)dec-8-yl]-1-thiophen-2-yl-propyl}-amide;
N-{3-[2-(4-methoxybenzyl)-1-oxo-2,8-diaza-spiro[4.5]dec-8-yl]-1-thiophen-2-yl-propyl}-propionamide;
2-methoxy-N-{3-[2-(4-methoxybenzyl)-1-oxo-2,8-diaza-spiro[4.5]dec-8-yl]-1-thiophen-2-yl-propyl}-acetamide;
cyclohexanecarboxylic acid {3-[2-(4-methoxybenzyl)-1-oxo-2,8-diaza-spiro[4.5]dec-8-yl]-1-thiophen-2-yl-propyl}-amide;
cyclopropane carboxylic acid{3-[2-(4-methanesulfonylbenzyl)-1-oxo-2,8-diaza-spiro[4.5]dec-8-yl)-1-thiophen-2-yl-propyl}-amide;
N-{3-[2-(4-methanesulfonylbenzyl)-1-oxo-2,8-diaza-spiro[4.5]dec-8-yl]-1-thiophen-2-yl-propyl}-isobutyramide;
cyclobutanecarboxylic acid {3-[2-(4-methanesulfonylbenzyl)-1-oxo-2,8-diaza-spiro[4.5]dec-8-yl]-1-thiophen-2-yl-propyl}-amide;
cyclopentanecarboxylic acid {3-[2-(4-methanesulfonylbenzyl)-1-oxo-2,8-diaza-spiro[4.5]dec-8-yl1-1-thiophen-2-yl-propyl}-amide;
N-{3-[2-(4-methanesulfonylbenzyl)-1-oxo-2,8-diaza-spiro[4.5]dec-8-yl]-1-thiophen-2-yl-propyl}-propionamide;
N-{3-[2-(4-methanesulfonylbenzyl)-1-oxo-2,8-diaza-spiro[4.5)dec-8-yl]-1-thiophen-2-yl-propyl}-2-methoxy-acetamide;
cyclohexanecarboxylic acid {3-[2-(4-methanesulfonylbenzyl)-1-oxo-2,8-diaza-spiro[4.5]dec-8-yl]-1-thiophen-2-yl-propyl}-amide;
cyclohexanecarboxylic acid [3-(1-oxo-2-pyridin-3-ylmethyl-2,8-diaza-spiro[4.5]dec-8-yl)-1-thiophen-2-yl-propyl]-amide;
N-[3-(1-oxo-2-pyridin-3-ylmethyl-2,8-diaza-spiro[4.5]dec-8-yl)-1-thiophen-2-yl-propyl]-isobutyramide;
cyclobutanecarboxylic acid [3-(1-oxo-2-pyridin-3-ylmethyl-2,8-diaza-spiro[4.5]dec-8-yl)-1-thiophen-2-yl-propyl]-amide;
cyclopentanecarboxylic acid [3-(1-oxo-2-pyridin-3-ylmethyl-2,8-diaza-spiro[4.5]dec-8-yl)-1-thiophen-2-yl-propyl]-amide;
N-[3-(1-oxo-2-pyridin-3-ylmethyl-2,8-diaza-spiro[4.5]dec-8-yl)-1-thiophen-2-yl-propyl]-propionamide;
cyclohexanecarboxylic acid [3-(1-oxo-2-pyridin-3-ylmethyl-2,8-diaza-spiro[4.5]dec-8-yl)-1-thiophen-2-yl-propyl]-amide;
cyclopropanecarboxylic acid [3-[2-(4-bromobenzyl)-1-oxo-2,8-diaza-spiro[4.5]dec-8-yl]-1-(3-chlorophenyl)-propyl]-amide;
N-[3-[2-(4-bromobenzyl)-1-oxo-2,8-diaza-spiro[4.5]dec-8-yl]-1-(3-chlorophenyl)-propyl]-isobutyramide;
cyclobutanecarboxylic acid [3-[2-(4-bromobenzyl)-1-oxo-2,8-diaza-spiro[4.5]dec-8-yl]-1-(3-chlorophenyl)-propyl]-amide;
cyclopentanecarboxylic acid [3-[2-(4-bromobenzyl)-1-oxo-2,8-diaza-spiro[4.5]dec-8-yl]-1-(3-chlorophenyl)-propyl]-amide;
N-[3-[2-(4-bromobenzyl)-1-oxo-2,8-diaza-spiro[4.5]dec-8-yl]-1-(3-chlorophenyl)-propyl]-propionamide;
N-[3-[2-(4-bromobenzyl)-1-oxo-2,8-diaza-spiro[4.5]dec-8-yl]-1-(3-chlorophenyl)-propyl]-2-methoxy-acetamide;
piperidine-1-carboxylic acid {3-[2-(4-bromobenzyl)-1-oxo-2,8-diaza-spiro[4.5]dec-8-yl]-1-phenyl-propyl}-amide;
piperidine-1-carboxylic acid {3-[2-(4-methoxybenzyl)-1-oxo-2,8-diaza-spiro[4.5]dec-8-yl]-1-phenyl-propyl}-amide;
piperidine-1-carboxylic acid {3-[2-(4-methanesulfonylbenzyl)-1-oxo-2,8-diaza-spiro[4.5]dec-8-yl]-1-phenyl-propyl}amide;
1-{3-[2-(4-bromobenzyl)-1-oxo-2,8-diaza-spiro[4.5]dec-8-yl]-1-phenyl-propyl}-3-phenyl-urea;
1-{3-[2-(4-bromobenzyl)-1-oxo-2,8-diaza-spiro[4.5]dec-8-yl]-1-phenyl-propyl}-3-(2-chloro-phenyl)-urea;
1-{3-[2-(4-bromobenzyl)-1-oxo-2,8-diaza-spiro[4.5]dec-8-yl]-1-phenyl-propyl}-3-(2-methoxy-phenyl)-urea;
1-{3-[2-(4-bromobenzyl)-1-oxo-2,8-diaza-spiro[4.5]dec-8-yl]-1-phenyl-propyl}-3-(3-chloro-phenyl)-urea;
1-{3-[2-(4-bromobenzyl)-1-oxo-2,8-diaza-spiro[4.5]dec-8-yl]-1-phenyl-propyl}-3-(3-methoxy-phenyl)-urea;
1-{3-[2-(4-bromobenzyl)-1-oxo-2,8-diaza-spiro[4.5]dec-8-yl]-1-phenyl-propyl}-3-(4-chloro-phenyl)-urea;
1-{3-[2-(4-bromobenzyl)-1-oxo-2,8-diaza-spiro[4.5]dec-8-yl]-1-phenyl-propyl}-3-(4-methoxy-phenyl)-urea;
1-{3-[2-(4-bromobenzyl)-1-oxo-2,8-diaza-spiro[4.5]dec-8-yl]-1-phenyl-propyl}-3-(2,6-dimethyl-phenyl)-urea;
1-{3-[2-(4-bromobenzyl)-1-oxo-2,8-diaza-spiro[4.5]dec-8-yl]-1-phenyl-propyl}-3-naphthalen-1-yl-urea;
1-{3-[2-(4-methoxybenzyl)-1-oxo-2,8-diaza-spiro[4.5]dec-8-yl]-1-phenyl-propyl}-3-phenyl-urea;
1-(2-chlorophenyl)-3-{3-[2-(4-methoxybenzyl)-1-oxo-2,8-diaza-spiro[4.5]dec-8-yl]-1-phenyl-propyl}-urea;
1-{3-[2-(4-methoxybenzyl)-1-oxo-2,8-diaza-spiro[4.5]dec-8-yl]-1-phenyl-propyl}-3-(2-methoxyphenyl)-urea;
1-(3-chlorophenyl)-3-{3-[2-(4-methoxybenzyl)-1-oxo-2,8-diaza-spiro[4.5]dec-8-yl]-1-phenyl-propyl}-urea;
1-{3-[2-(4-methoxybenzyl)-1-oxo-2,8-diaza-spiro[4.5]dec-8-yl]-1-phenyl-propyl}-3-(3-methoxyphenyl)-urea;
{3-[2-(4-methoxybenzyl)-1-oxo-2,8-diaza-spiro[4.5]dec-8-yl]-1-phenyl-propyl}-3-(4-methoxyphenyl)-urea;
1-(2,6-dimethylphenyl)-3-{3-[2-(4-methoxybenzyl)-1-oxo-2,8-diaza-spiro[4.5]dec-8-yl]-1-phenyl-propyl}-urea;
1-{3-[2-(4-methoxybenzyl)-1-oxo-2,8-diaza-spiro[4.5]dec-8-yl]-1-phenyl-propyl}-3-naphthalen-1-yl-urea;
1-{3-[2-(4-methanesulfonylbenzyl)-1-oxo-2,8-diaza-spiro[4.5]dec-8-yl]-1-phenyl-propyl}-3-phenyl-urea;
1-(2-chlorophenyl)-3-{3-[2-(4-methanesulfonylbenzyl)-1-oxo-2,8-diaza-spiro[4.5]dec-8-yl]-1-phenyl-propyl}-urea;
1-{3-[2-(4-methanesulfonylbenzyl)--oxo-2,8-diaza-spiro[4.5]dec-8-yl]-1-phenyl-propyl}-3-(2-methoxyphenyl)-urea;
1-(3-chlorophenyl)-3-{3-[2-(4-methanesulfonylbenzyl)-1-oxo-2,8-diaza-spiro[4.5]dec-8-yl]-1-phenyl-propyl}-urea;
1-{3-[2-(4-methanesulfonylbenzyl)-1-oxo-2,8-diaza-spiro[4.5]dec-8-yl]-1-phenyl-propyl}-3-(3-methoxyphenyl)-urea;
1-(4-chlorophenyl)-3-{3-[2-(4-methanesulfonylbenzyl)-1-oxo-2,8-diaza-spiro[4.5]dec-8-yl]-1-phenyl-propyl}-urea;
1-{3-[2-(4-methanesulfonylbenzyl)-1-oxo-2,8-diaza-spiro[4.5)dec-8-yl]-1-phenyl-propyl}-3-(4-methoxyphenyl)-urea;
1-(2,6-dimethylphenyl)-3-{3-[2-(4-methanesulfonylbenzyl)-1-oxo-2,8-diaza-spiro[4.5]dec-8-yl]-1-phenyl-propyl}-urea;
1-{3-[2-(4-methanesulfonylbenzyl)-1-oxo-2,8-diaza-spiro[4.5]dec-8-yl]-1-phenyl-propyl}-3-naphthalen-1-yl-urea;
{3-[2-(4-methanesulfonylbenzyl)-1-oxo-2,8-diaza-spiro[4.5]dec-8-yl]-1-phenyl-propyl}-carbamic acid methyl ester;
{3-[2-(4-methanesulfonylbenzyl)-1-oxo-2,8-diaza-spiro[4.5]dec-8-yl]-1-phenyl-propyl}-carbamic acid ethyl ester;
4-[2-(4-bromobenzyl)-1-oxo-2,8-diaza-spiro[4.5)dec-8-yl]-2-phenyl-butyric acid methyl ester;
4-[2-(4-methanesulfonylbenzyl)-1-oxo-2,8-diaza-spiro[4.5]dec-8-yl]-2-phenyl-butyric acid methyl ester;
4-[2-(4-methoxybenzyl)-1-oxo-2,8-diaza-spiro[4.5]dec-8-yl]-2-phenyl-butyric acid methyl ester;
4-[2-(4-bromobenzyl)-1-oxo-2,8-diaza-spiro[4.5]dec-8-yl]-2,N-diphenyl-butyramide;
N-benzyl-4-[2-(4-bromobenzyl)-1-oxo-2,8-diaza-spiro[4.5]dec-8-yl]-2-phenyl-butyramide;
2-(4-bromobenzyl)-8-(4-oxo-3-phenyl-4-piperidin-1-yl-butyl)-2,8-diaza-spiro[4.5]decan-1-one;
4-[2-(4-bromobenzyl)-1-oxo-2,8-diaza-spiro[4.5]dec-8-yl)-N-cyclohexyl-2-phenyl-butyramide;
4-[2-(4-bromobenzyl)-1-oxo-2,8-diaza-spiro[4.5]dec-8-yl]-N-cyclohexyl-N-methyl-2-phenyl-butyramide;
4-[2-(4-bromobenzyl)-1-ox6-2,8-diaza-spiro[4.5]dec-8-yl]-N-cyclopropyl-2-phenyl-butyramide;
4-[2-(4-bromobenzyl)-1-oxo-2,8-diaza-spiro[4.5]dec-8-yl]-N-cyclobutyl-2-phenyl-butyramide;
N-cyclohexyl-4-[2-(4-methanesulfonylbenzyl)-1-oxo-2,8-diaza-spiro[4.5]dec-8-yl]-N-methyl-2-phenyl-butyramide;
N-cyclopropyl-4-[2-(4-methanesulfonylbenzyl)-1-oxo-2,8-diaza-spiro[4.5]dec-8-yl]-2-phenyl-butyramide;
N-cyclobutyl-4-[2-(4-methanesulfonylbenzyl)-1-oxo-2,8-diaza-spiro[4.5]dec-8-yl]-2-phenyl-butyramide;
4-[2-(4-bromobenzyl)-1-oxo-2,8-diaza-spiro[4.5]dec-8-yl]-N-cyclopentyl-2-phenyl-butyramide;
4-[2-(4-bromobenzyl)-1-oxo-2,8-diaza-spiro[4.5]dec-8-yl]-N-isopropyl-2-phenyl-butyramide;
N-benzyl-4-[2-(4-methanesulfonylbenzyl)-1-oxo-2,8-diaza-spiro[4.5]dec-8-yl]-2-phenyl-butyramide;
2-(4-methanesulfonylbenzyl)-8-(4-oxo-3-phenyl-4-piperidin-1-yl-butyl)-2,8-diaza-spiro[4.5]decan-1-one;
N-cyclohexyl-4-[2-(4-methanesulfonylbenzyl)-1-oxo-2,8-diaza-spiro[4.5]dec-8-yl]-2-phenyl-butyramide;
N-cyclopentyl-4-[2-(4-methanesulfonylbenzyl)-1-oxo-2,8-diaza-spiro[4.5]dec-8-yl]-2-phenyl-butyramide;
N-isopropyl-4-[2-(4-methanesulfonylbenzyl)-1-oxo-2,8-diaza-spiro[4.5]dec-8-yl]-2-phenyl-butyramide;
4-[2-(4-methoxybenzyl)-1-oxo-2,8-diaza-spiro[4.5]dec-8-yl]-2,N-diphenyl-butyramide;
N-benzyl-4-[2-(4-methoxybenzyl)-1-oxo-2,8-diaza-spiro[4.5]dec-8-yl]-2-phenyl-butyramide;
2-(4-methoxybenzyl)-8-(4-oxo-3-phenyl-4-piperidin-1-yl-butyl)-2,8-diaza-spiro[4.5]decan-1-one;
N-cyclohexyl-4-[2-(4-methoxybenzyl)-1-oxo-2,8-diaza-spiro[4.5]dec-8-yl]-2-phenyl-butyramide;
N-cyclopropyl-4-[2-(4-methoxybenzyl)-1-oxo-2,8-diaza-spiro[4.5]dec-8-yl]-2-phenyl-butyramide;
N-cyclobutyl-4-[2-(4-methoxybenzyl)-1-oxo-2,8-diaza-spiro[4.5]dec-8-yl]-2-phenyl-butyramide;
N-cyclopentyl-4-[2-(4-methoxybenzyl)-1-oxo-2,8-diaza-spiro[4.5]dec-8-yl]-2-phenyl-butyramide;
N-isopropyl-4-[2-(4-methoxybenzyl)-1-oxo-2,8-diaza-spiro[4.5]dec-8-yl]-2-phenyl-butyramide;
and pharmaceutically acceptable salts, hydrates and solvates thereof.

32. A compound according to claim 1, wherein said compound is selected from:
Cyclopropanecarboxylic acid {(S)-1-(3-chloro-phenyl)-3-[2-(4-methoxy-benzyl)-1-oxo-2,8-diaza-spiro[4.5]dec-8-yl]-propyl}-amide;
Cyclopropanecarboxylic acid {(S)-1-(3-fluoro-phenyl)-3-[2-(4-methoxy-benzyl)-1-oxo-2,8-diaza-spiro[4.5]dec-8-yl]-propyl}-amide;
Cyclopropanecarboxylic acid {(S)-3-[2-(4-methoxy-benzyl)-1-oxo-2,8-diaza-spiro[4.5]dec-8-yl]-1-thiophen-2-yl-propyl}-amide;
Cyclopropanecarboxylic acid {(S)-3-[2-(4-methoxy-benzyl)-1-oxo-2,8-diaza-spiro[4.5]dec-8-yl]-1-thiophen-3-yl-propyl}-amide;
N-{(S)-1-(3-Chloro-phenyl)-3-[2-(4-methoxy-benzyl)-1-oxo-2,8-diaza-spiro[4.5]dec-8-yl]-propyl}-isobutyramide;
N-{(S)-1-(3-Fluoro-phenyl)-3-[2-(4-methoxy-benzyl)-1-oxo-2,8-diaza-spiro[4.5]dec-8-yl]-propyl}-isobutyramide;
N-{(S)-3-[2-(4-Methoxy-benzyl)-1-oxo-2,8-diaza-spiro[4.5]dec-8-yl]-1-thiophen-2-yl-propyl}-isobutyramide;
N-{(S)-3-[2-(4-Methoxy-benzyl)-1-oxo-2,8-diaza-spiro[4.5]dec-8-yl]-1-thiophen-3-yl-propyl}-isobutyramide;
N-{(S)-3-[2-(4-Chloro-benzyl)-3-oxo-2,8-diaza-spiro[4.5]dec-8-yl]-1-phenyl-propyl}-isobutyramide;
N-{(S)-3-[2-(4-Fluoro-benzyl)-3-oxo-2,8-diaza-spiro[4.5]dec-8-yl]-1-phenyl-propyl}-isobutyramide;
N-{(S)-3-[2-(4-Cyano-benzyl)-3-oxo-2,8-diaza-spiro[4.5]dec-8-yl]-1-phenyl-propyl}-isobutyramide;
N-{(S)-3-[2-(4-Ethoxy-benzyl)-3-oxo-2,8-diaza-spiro[4.5]dec-8-yl]-1-phenyl-propyl}-isobutyramide;
N-{(S)-3-[2-(4-Difluoromethoxy-benzyl)-3-oxo-2,8-diaza-spiro[4.5]dec-8-yl]-1-phenyl-propyl}-isobutyramide;
N-{(S)-3-[3-Oxo-2-(4-trifluoromethoxy-benzyl)-2,8-diaza-spiro[4.5]dec-8-yl]-1-phenyl-propyl}-isobutyramide;
N-{(S)-3-[3-Oxo-2-(4-trifluoromethyl-benzyl)-2,8-diaza-spiro[4.5]dec-8-yl]-1-phenyl-propyl}-isobutyramide;
N-{(S)-3-[3-Oxo-2-(4-pyrazol-1-yl-benzyl)-2,8-diaza-spiro[4.5]dec-8-yl]-1-phenyl-propyl}-isobutyramide;
Cyclopropanecarboxylic acid {(S)-3-[2-(4-chloro-benzyl)-3-oxo-2,8-diaza-spiro[4.5]dec-8-yl]-1-phenyl-propyl}-amide;
Cyclopropanecarboxylic acid {(S)-3-[2-(4-fluoro-benzyl)-3-oxo-2,8-diaza-spiro[4.5]dec-8-yl]-1-phenyl-propyl}-amide;
Cyclopropanecarboxylic acid {(S)-3-[2-(4-cyano-benzyl)-3-oxo-2,8-diaza-spiro[4.5]dec-8-yl]-1-phenyl-propyl}-amide;
Cyclopropanecarboxylic acid {(S)-3-[2-(4-ethoxy-benzyl)-3-oxo-2,8-diaza-spiro[4.5]dec-8-yl]-1-phenyl-propyl}-amide;
Cyclopropanecarboxylic acid {(S)-3-[2-(4-difluoromethoxy-benzyl)-3-oxo-2,8-diaza-spiro[4.5]dec-8-yl]-1-phenyl-propyl}-amide;
Cyclopropanecarboxylic acid {(S)-3-[2-(4-trifluoromethoxy-benzyl)-3-oxo-2,8-diaza-spiro[4.5]dec-8-yl]-1-phenyl-propyl}-amide;
Cyclopropanecarboxylic acid {(S)-3-[2-(4-trifluoromethyl-benzyl)-3-oxo-2,8-diaza-spiro[4.5]dec-8-yl]-1-phenyl-propyl}-amide;
Cyclopropanecarboxylic acid {(S)-3-[3-oxo-2-(4-pyrazol-1-yl-benzyl)-2,8-diaza-spiro[4.5]dec-8-yl]-1-phenyl-propyl}-amide;
2-Cyclopropyl-N-{(S)-3-[2-(4-methoxy-benzyl)-3-oxo-2,8-diaza-spiro[4.5]dec-8-yl]-1-phenyl-propyl}-acetamide;
N-{(S)-3-[2-(4-Chloro-benzyl)-3-oxo-2,8-diaza-spiro[4.5]dec-8-yl]-1-phenyl-propyl}-2-cyclopropyl-acetamide;
N-{(S)-3-[2-(4-Fluoro-benzyl)-3-oxo-2,8-diaza-spiro[4.5]dec-8-yl]-1-phenyl-propyl}-2-cyclopropyl-acetamide;
N-{(S)-3-[2-(4-Cyano-benzyl)-3-oxo-2,8-diaza-spiro[4.5]dec-8-yl]-1-phenyl-propyl}-2-cyclopropyl-acetamide;
N-{(S)-3-[2-(4-Ethoxy-benzyl)-3-oxo-2,8-diaza-spiro[4.5]dec-8-yl]-1-phenyl-propyl}-2-cyclopropyl-acetamide;
N-{(S)-3-[2-(4-Difluoromethoxy-benzyl)-3-oxo-2,8-diaza-spiro[4.5]dec-8-yl]-1-phenyl-propyl}-2-cyclopropyl-acetamide;
N-{(S)-3-[2-(4-Trifluoromethoxy-benzyl)-3-oxo-2,8-diaza-spiro[4.5]dec-8-yl]-1-phenyl-propyl}-2-cyclopropyl-acetamide;
N-{(S)-3-[2-(4-Trifluoromethyl-benzyl)-3-oxo-2,8-diaza-spiro[4.5]dec-8-yl]-1-phenyl-propyl}-2-cyclopropyl-acetamide;
2-Cyclopropyl-N-{(S)-3-[3-oxo-2-(4-pyrazol-1-yl-benzyl)-2,8-diaza-spiro[4.5]dec-8-yl]-1-phenyl-propyl}-acetamide;
4,4-Difluoro-cyclohexanecarboxylic acid {(S)-3-[2-(4-chloro-benzyl)-3-oxo-2,8-diaza-spiro[4.5]dec-8-yl]-1-phenyl-propyl}-amide;
4,4-Difluoro-cyclohexanecarboxylic acid {(S)-3-[2- (4-fluoro-benzyl)-3-oxo-2,8-diaza-spiro[4.5]dec-8-yl]-1-phenyl-propyl}-amide;
4,4-Difluoro-cyclohexanecarboxylic acid {(S)-3-[2-(4-cyano-benzyl)-3-oxo-2,8-diaza-spiro[4.5]dec-8-yl]-1-phenyl-propyl}-amide;
4,4-Difluoro-cyclohexanecarboxylic acid {(S)-3-[2-(4-ethoxy-benzyl)-3-oxo-2,8-diaza-spiro[4.5]dec-8-yl]-1-phenyl-propyl}-amide;
4,4-Difluoro-cyclohexanecarboxylic acid {(S)-3-[3-oxo-2-(4-pyrazol-1-yl-benzyl)-2,8-diaza-spiro[4.5]dec-8-yl]-1-phenyl-propyl}-amide;
Cyclopropanecarboxylic acid {(S)-1-(3-chloro-phenyl)-3-[2-(4-methoxy-benzyl)-3-oxo-2,8-diaza-spiro[4.5]dec-8-yl]-propyl}-amide;
Cyclopropanecarboxylic acid {(S)-1-(3-fluoro-phenyl)-3-[2-(4-methoxy-benzyl)-3-oxo-2,8-diaza-spiro[4.5]dec-8-yl]-propyl}-amide;
Cyclopropanecarboxylic acid {(S)-3-[2-(4-methoxy-benzyl)-3-oxo-2,8-diaza-spiro[4.5]dec-8-yl]-1-thiophen-2-yl-propyl}-amide;
Cyclopropanecarboxylic acid {(S)-3-[2-(4-methoxy-benzyl)-3-oxo-2,8-diaza-spiro[4.5]dec-8-yl]-1-thiophen-3-yl-propyl}-amide;
N-{(S)-1-(3-Chloro-phenyl)-3-[2-(4-methoxy-benzyl)-3-oxo-2,8-diaza-spiro[4.5]dec-8-yl]-propyl}-isobutyramide;
N-{(S)-1-(3-Fluoro-phenyl)-3-[2-(4-methoxy-benzyl)-3-oxo-2,8-diaza-spiro[4.5]dec-8-yl]-propyl}-isobutyramide;
N-{(S)-3-[2-(4-Methoxy-benzyl)-3-oxo-2,8-diaza-spiro[4.5]dec-8-yl]-1-thiophen-2-yl-propyl}-isobutyramide;
N-{(S)-3-[2-(4-Methoxy-benzyl)-3-oxo-2,8-diaza-spiro[4.5]dec-8-yl]-1-thiophen-3-yl-propyl}-isobutyramide;
Propane-2-sulfonic acid {(S)-3-[2-(4-methoxy-benzyl)-3-oxo-2,8-diaza-spiro[4.5]dec-8-yl]-1-phenyl-propyl}-amide;
3-{(S)-3-[2-(4-Methoxy-benzyl)-3-oxo-2,8-diaza-spiro[4.5]dec-8-yl]-1-phenyl-propyl}-1,1-dimethyl-urea;
Morpholine-4-carboxylic acid {(S)-3-[2-(4-methoxy-benzyl)-3-oxo-2,8-diaza-spiro[4.5]dec-8-yl]-1-phenyl-propyl}-amide;
3,3-Difluoro-pyrrolidine-1-carboxylic acid {(S)-3[2-(4-methoxy-benzyl)-3-oxo-2,8-diaza-spiro[4.5]dec-8-yl]-1-phenyl-propyl}-amide;
(S)-3-[2-(4-Methoxy-benzyl)-3-oxo-2,8-diaza-spiro[4.5]dec-8-yl]-1-phenyl-propyl}-carbamic acid methyl ester;
{(S)-3-[2-(4-Methoxy-benzyl)-3-oxo-2,8-diaza-spiro[4.5]dec-8-yl]-1-phenyl-propyl}-carbamic acid ethyl ester;
and pharmaceutically acceptable salts, hydrates or solvates thereof.

33. A compound according to claim 1, wherein:
Y, Z and X are each, independently, CH₂, C═O or CR₄R₅;
W is H, C₁₋₆ alkyl, C₆₋₁₂ aryl, 3 to 10 membered heterocycle, C₆₋₁₂ aralkyl or C₃₁₀ heteroaralkyl;
R₁ is H, C₁₋₆ alkyl, C₆₋₁₂ aryl, OH, NR₈R₉, O—C₁₋₆ alkyl, O—C₆₋₁₂ aryl, O—C₆₋₁₂ aralkyl, 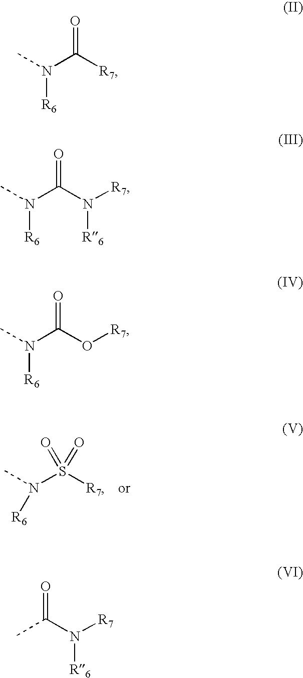
R₂ is C₁₋₁₀ alkyl, C₆₋₁₂ aryl or 3 to 10 membered heterocycle;
R₃ is H, C₁₋₆ alkyl or C₆₋₁₂ aryl;
R₄ and R₅ are each, independently, H, C₁₋₆ alkyl or C₆₋₁₂ aryl;
R₆ is H or C₁₋₄ alkyl;
R₇ is H, C₁₋₁₀ alkyl, C₆₋₁₂ aryl, 3 to 10 membered heterocycle,
C₆₋₁₂ aralkyl or 3 to 10 membered heteroaralkyl; or
R″₆ and R₇ can also be taken together to form a 3 to 10 membered heterocycle; and
R₈ and R₉ are each, independently, H or C₁₋₆ alkyl.

34. A method of modulating chemokine receptor activity in a subject comprising administering to the subject an effective amount of a compound of according to claim 1.

35. A method of preventing or treating at least one disease associated with the modulation of chemokine receptor activity in a subject in need of such treatment comprising administering to the subject a therapeutically effective amount of a compound of according to claim 1.

36. A method of preventing or treating at least one inflammatory disease, immunoregulatory disease, organ transplantation reaction, or infectious disease in a subject in need of such treatment comprising administering to the subject a therapeutically effective amount of a compound of according to claim 1.

37. A method of preventing or treating an HIV infection in a subject in need of such treatment comprising administering to the subject a therapeutically effective amount of a compound of according to claim 1.

38. A method of preventing or treating at least one disease associated with the modulation of CCR5 chemokine receptor activity in a subject in need of such treatment comprising administering to the subject a therapeutically effective amount of a compound of according to claim 1.

39. A method of blocking cellular entry of HIV in a subject in need thereof comprising administering to the subject an effective amount of a compound according to claim 1 to block HIV from cellular entry in said subject.

40. A method of delaying the onset of AIDS or treating AIDS in a subject in need of such treatment comprising administering to the subject a therapeutically effective amount of a compound according to claim 1.

41. A method of preventing or treating of a disease associated with the modulation of chemokine receptor activity in a subject in need of such treatment comprising administering to the subject a therapeutically effective amount of a combination of a compound according to claim 1 and at least one further therapeutic agent.

42. A method of preventing or treating of a disease associated with the modulation of CCR5 chemokine receptor activity in a subject in need of such treatment comprising administering to the subject a therapeutically effective amount of a combination of a compound according to claim 1 and at least one further therapeutic agent.

43. A method of blocking cellular entry of HIV in a subject or for the prevention or treatment of HIV infections in a subject in need of such treatment comprising administering to the subject a therapeutically effective amount of a combination of a compound according to claim 1 and at least one further therapeutic agent.

44. A method for delaying the onset of AIDS or treating AIDS in a subject in need of such treatment comprising administering to the subject a therapeutically effective amount of a combination of a compound according to claim 1 and at least one further therapeutic agent.

45. A method according to claim 34, wherein said compound is administered in an amount of 0.1-750 mg/kg of body weight per day.

46. A method according to claim 35, wherein said compound is administered in an amount of 0.1-750 mg/kg of body weight per day.

47. A method according to claim 36, wherein said compound is administered in an amount of 0.1-750 mg/kg of body weight per day.

48. A method according to claim 37, wherein said compound is administered in an amount of 0.1-750 mg/kg of body weight per day.

49. A method according to claim 38, wherein said compound is administered in an amount of 0.1-750 mg/kg of body weight per day.

50. A method according to claim 39, wherein said compound is administered in an amount of 0.1-750 mg/kg of body weight per day.

51. A method according to claim 40, wherein said compound is administered in an amount of 0.1-750 mg/kg of body weight per day.

52. A method according to claim 41, wherein said compound is administered in an amount of 0.1-750 mg/kg of body weight per day.

53. A method according to claim 42, wherein said compound is administered in an amount of 0.1-750 mg/kg of body weight per day.

54. A method according to claim 43, wherein said compound is administered in an amount of 0.1-750 mg/kg of body weight per day.

55. A method according to claim 44, wherein said compound is administered in an amount of 0.1-750 mg/kg of body weight per day.

56. A method according to claim 34, wherein said subject is a human.

57. A method according to claim 35, wherein said subject is a human.

58. A method according to claim 36, wherein said subject is a human.

59. A method according to claim 37, wherein said subject is a human.

60. A method according to claim 38, wherein said subject is a human.

61. A method according to claim 39, wherein said subject is a human.

62. A method according to claim 40, wherein said subject is a human.

63. A method according to claim 41, wherein said subject is a human.

64. A method according to claim 42, wherein said subject is a human.

65. A method according to claim 43, wherein said subject is a human.

66. A method according to claim 44, wherein said subject is a human.

67. A pharmaceutical composition comprising a compound according to claim 1 and a pharmaceutically acceptable carrier.

68. A composition according to claim 67, further comprising at least one further therapeutic agent.

69. A pharmaceutical combination comprising a compound according to claim 1 and at least one further therapeutic agent.

70. A combination according to claim 69, wherein said at least one further therapeutic agent is an antiviral agent.

71. A combination according to claim 69, wherein said at least one further therapeutic agent is selected from nucleoside and nucleotide analog reverse transcriptase inhibitors, non-nucleoside reverse transcriptase inhibitors, protease inhibitors, attachment and fusion inhibitors, integrase inhibitors and maturation inhibitors.

72. A combination according to claim 70, wherein said antiviral agent is selected from 3TC (lamivudine, Epivir®), AZT (zidovudine, Retrovir®), Emtricitabine (Coviracil®, formerly FTC), d4T (2′,3′-dideoxy-2′,3′-didehydro-thymidine, stavudine and Zerit®), tenofovir (Viread®), 2′,3′-dideoxyinosine (ddl, didanosine, Videx®), 2′,3′-dideoxycytidine (ddC, zalcitabine, Hivid®), Combivir® (AZT/3TC or zidovudine/lamivudine combination), Trivizir® (AZT/3TC/abacavir or zidovudine/lamivudine/abacavir combination), abacavir (1592U89, Ziagen®), SPD-754, ACH-126,443 (Beta-L-Fd4C), Alovudine (MIV-310), DAPD (amdoxovir), Racivir, 9-[(2-hydroxymethyl)-1,3-dioxolan-4-yllguanine and 2-amino-9-[(2-hydroxymethyl)-1,3-dioxolan-4-yl]adenine.

73. A combination according to claim 70, wherein said antiviral agent is selected from Nevirapine (Viramune®, NVP, BI-RG-587), delavirdine (Rescriptor®, DLV), efavirenz (DMP 266, Sustiva®), (+)-Calanolide A, Capravirine (AG1549, formerly S-1153), DPC083, MIV-150, TMC120, TMC125 or BHAP (delavirdine), calanolides or L-697,661 (2-Pyridinone 3benzoxazolMeNH derivative).

74. A combination according to claim 70, wherein said antiviral agent is selected from nelfinavir (Viracept®, NFV), amprenavir (141W94, Agenerase®), indinavir (MK-639, IDV, Crixivan®), saquinavir (Invirase®, Fortovase®, SQV), ritonavir (Norvir®, RTV), lopinavir (ABT-378, Kaletra®), Atazanavir (BMS232632), mozenavir (DMβ-450), fosamprenavir (GW433908), R0033-4649, Tipranavir (PNU-140690), TMC114 and VX-385.

75. A combination according to claim 70, wherein said antiviral agent is selected from T-20 (enfuvirtide, Fuzeon®), T-1249, Schering C (SCH-C), Schering D (SCH-D), FP21399, PRO-140, PRO 542, PRO 452, TNX-355, GW873140 (AK602), TAK-220, UK-427,857 or soluble CD4, CD4 fragments, CD4-hybrid molecules, BMS-806, BMS-488043, AMD3100, AMD070 and KRH-2731.

76. A combination according to claim 70, wherein said antiviral agent is selected from S-1360, L-870,810, L-870,812 and C-2507.

77. A combination according to claim 70, wherein said antiviral agent is PA-457.

78. A combination according to claim 70, wherein said antiviral agent is azodicarbonamide (ADA).

79. A combination according to claim 70, wherein said antiviral agent is HGTV43.

80. A combination according to claim 70, wherein said antiviral agent is selected from interleukin-2 (IL-2, Aldesleukin, Proleukin), granulocyte macrophage colony stimulating factor (GM-CSF), erythropoietin, Multikine, Ampligen, thymomodulin, thymopentin, foscarnet, HE2000, Reticulose, Murabutide, Resveratrol, HRG214, HIV-1 Immunogen (Remune) and EP HIV-1090.

81. A combination according to claim 70, wherein said antiviral agent is selected from 2′,3′-dideoxyadenosine, 3′-deoxythymidine, 2′,3′-dideoxy-2′,3′-didehydrocytidine,ribavirin, acyclovir, ganciclovir, alpha-beta-and gamma-interferon, probenecid, TIBO drugs, HEPT, and TSAO derivatives.
